(19) United States
(12) Patent Application Publication
(10) Pub. No.: US 2025/0367324 A1
(43) Pub. Date: Dec. 4, 2025

(54) ENGINEERED ENVELOPED NANOPARTICLES (ENPS) AS A DELIVERY SYSTEM FOR NUCLEIC ACID-BASED CARGOES

(71) Applicants: California Institute of Technology, Pasadena, CA (US); The J. David Gladstone Institutes, a Testamentary Trust Established under the Will of, San Francisco, CA (US)

(72) Inventors: Magnus A.G. Hoffmann, Pasadena, CA (US); Jost Vielmetter, Pasadena, CA (US); Michael A. Anaya, Pasadena, CA (US); Kai G. Zinn, Pasadena, CA (US); Tracie Luu, San Francisco, CA (US)

(21) Appl. No.: 19/223,492

(22) Filed: May 30, 2025

Related U.S. Application Data
(60) Provisional application No. 63/654,753, filed on May 31, 2024.

(51) Int. Cl. A61K 48/00; C12N 15/88

(52) U.S. Cl. CPC A61K 48/0058; A61K 48/0041; C12N 15/88

(57) ABSTRACT

Disclosed herein include methods, compositions, and kits suitable for use in e.g., nucleic acid delivery. Provided are compositions (e.g., nucleic acid compositions) comprising polynucleotide(s) encoding a fusion protein, a dimerization fusion protein, a soluble RBP, an adapter fusion protein, and/or a cell fusion protein. The compositions also comprise one or more polynucleotides comprising one or more RNA cargo molecules. The fusion protein, dimerization fusion protein, and/or adapter fusion protein can comprise an endosomal sorting complex required for transport (ESCRT)-recruiting domain (ERD). Recruitment of one or more ESCRT proteins results in secretion of enveloped nanoparticles (ENPs) from a cell in which at least one of a fusion protein, a dimerization fusion protein, and/or an adapter fusion protein are expressed. The ENPs can comprise the one or more RNA cargo molecules. There are also provided populations of ENPs in some embodiments.

### RELATED APPLICATIONS

The present application claims priority to U.S. Provisional Application No. 63/654,753, filed May 31, 2024. The content of this related application is incorporated herein by reference in its entirety for all purposes.

### STATEMENT REGARDING FEDERALLY SPONSORED R&D

This invention was made with government support under Grant No. OD033362 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ-365873-US_SequenceListing, created May 28, 2025, which is 258 kilobytes in size. The information in the electronic format of the Sequence Listing is incorporated herein by reference in its entirety.

### BACKGROUND

### Field

The present disclosure relates generally to the field of nucleic acid delivery.

### Description of the Related Art

Nanoparticle-based drug delivery systems have two main functions: i) protect small molecule-, protein-, and nucleic acid-based therapeutics from degradation and clearance inside the body to enhance their bioavailability; ii) target the delivery of therapeutic payloads to disease-specific tissues to increase their local therapeutic concentration and minimize adverse effects in healthy tissues. Cell-derived extracellular vesicles (EVs) such as exosomes are being evaluated as next-generation drug delivery platform due to their intrinsic capabilities for packaging biomolecular cargoes and tissue-homing. However, large-scale production of EVs remains a challenge. Cells typically secrete EVs at low rates and chemical and physical methods to increase EV production are toxic to cells, which limits the feasibility of continuous EV production in bioreactors. Moreover, engineering EVs to display specific surface markers and/or encapsulate therapeutic cargoes is challenging as EV formation is complex and requires incorporation of numerous proteins on the surface and interior of EVs.

A new technology that promotes efficient production of self-assembling enveloped nanoparticles (ENPs) has recently been described (See, US Patent Application Publication US20220402977, the content of which is hereby incorporated by reference in its entirety). ENP assembly is induced by inserting an ESCRT-recruiting domain (ERD) into the cytoplasmic tail of a cell surface protein (CSP) that recruits cellular proteins from the Endosomal Sorting Complex Required for Transport (ESCRT) pathway to induce ENP budding and release. The original ERD sequence was derived from the ESCRT- and ALIX-binding region (EABR) of the human CEP55 protein. Cryo-electron tomograms showed that ERD ENPs are surrounded by a lipid bilayer, 40-60 nm in diameter, and densely coated with the engineered CSP-ERD fusion protein. The ERD technology improves ENP production by 10- to 100-fold compared to existing nanoparticle platforms such as lentiviral Gag-based virus-like particles, and ENP budding is not toxic to cells, enabling continuous ENP production in large-scale bioreactors. There is a need for nanoparticle-based (e.g., ENP) delivery of nucleic acid cargoes, e.g., RNA cargoes.

### SUMMARY

Disclosed herein include compositions. In some embodiments, the composition comprises: a nucleic acid composition comprising a polynucleotide encoding a fusion protein and one or more polynucleotides comprising one or more cargo RNA molecules each comprising a packing signal, wherein the fusion protein comprises a cell-surface protein (CSP), an RNA-binding protein (RBP), and an endosomal sorting complex required for transport (ESCRT)-recruiting domain (ERD), wherein the RBP is capable of binding the packing signal, and wherein a plurality of fusion proteins are capable of self-assembling into an enveloped nanoparticle (ENP) secreted from a cell in which the plurality of fusion proteins are expressed, thereby generating a population of ENPs comprising the fusion protein and the one or more cargo RNA molecules, optionally the nucleic acid composition further comprises a polynucleotide encoding a soluble RBP capable of binding the packing signal.

In some embodiments, the fusion protein is capable of being presented on the surface of the cell in which the fusion protein is expressed. In some embodiments, the self-assembly of an ENP does not require an exogenous nucleic acid other than the nucleic acid composition. In some embodiments, the cell is: a cell of a subject; an in vivo cell, an ex vivo cell, or an in situ cell; and/or an adherent cell or a suspension cell. In some embodiments, upon secretion from a cell of a subject, the ENPs are capable of distributing within one or more tissues of the subject. In some embodiments, the one or more tissues comprise adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue, fat tissue, or any combination thereof. In some embodiments, the one or more tissues comprise diseased tissues, e.g., cancerous or infected tissues.

Disclosed herein include compositions. In some embodiments, the composition comprises: a population of enveloped nanoparticles (ENPs), wherein each of the ENPs comprises: (i) a plurality of fusion proteins each comprising a cell-surface protein (CSP), an RNA-binding protein (RBP), and an endosomal sorting complex required for transport (ESCRT)-recruiting domain (ERD); and (ii) one or more cargo RNA molecules each comprising a packing signal. In some embodiments, the population of ENPs is derived from expression of a nucleic acid composition of the disclosure. In some embodiments, the ENPs comprise a lipid bilayer, e.g., a lipid bilayer derived from the cell from which the ENP was secreted.

In some embodiments, the packing signal and the RBP are derived from a viral, archaeal, bacterial, or mammalian packing signal and RBP, or variants thereof. In some embodiments: the packing signal comprises a Ku binding hairpin and the RBP and/or the soluble RBP is Ku; the packing signal comprises a telomerase Sm7 binding motif and the RBP and/or the soluble RBP is Sm7; the packing signal comprises an MS2 phage operator stem-loop and the RBP and/or the soluble RBP is MS2 Coat Protein (MCP); the packing signal comprises a PP7 phage operator stem-loop and the RBP and/or the soluble RBP is PP7 Coat Protein (PCP); the packing signal comprises an SfMu phage Com stem-loop and the RBP and/or the soluble RBP is Com RNA binding protein; the packing signal comprises a PUF binding site (PBS) and the RBP and/or the soluble RBP is Pumilio/fem-3 mRNA binding factor (PUF); the packing signal comprises Psi and the RBP and/or the soluble RBP is gag, optionally derived from MMLV, HIV, SIV, FIV, HTLV, or Foamy viruses; the packing signal comprises regulatory RNA CsrB and the RBP and/or the soluble RBP is CsrA of E. coli; the packing signal comprises PS9 and the RBP and/or the soluble RBP is N protein of a coronavirus, optionally SARS-COV-2; and/or the packing signal comprises Box C/D binding motif and the RBP and/or the soluble RBP is ribosomal protein L7Ae of archae.

In some embodiments the packing signal comprises regulatory RNA CsrB and the RBP and/or the soluble RBP is CsrA of E. coli. In some embodiments, the packing signal comprises a nucleotide sequence having at least 65%, 70%, 75%, 80%, 85%, 90%, 95%, 98%, or 99% sequence identity to the nucleotide sequence of SEQ ID NO: 134 and the RBP and/or the soluble RBP comprises an amino acid sequence having at least 65%, 70%, 75%, 80%, 85%, 90%, 95%, 98%, or 99% sequence identity to the amino acid sequence of SEQ ID NO: 128. In some embodiments, the packing signal comprises an MS2 phage operator stem-loop and the RNA binding protein is MS2 Coat Protein (MCP). In some embodiments, the packing signal comprises a nucleotide sequence having at least 65%, 70%, 75%, 80%, 85%, 90%, 95%, 98%, or 99% sequence identity to the nucleotide sequence of SEQ ID NO: 152 and the RBP and/or the soluble RBP comprises an amino acid sequence having at least 65%, 70%, 75%, 80%, 85%, 90%, 95%, 98%, or 99% sequence identity to the amino acid sequence of SEQ ID NO: 148. In some embodiments, the packing signal comprises PS9 and the RBP and/or the soluble RBP is N protein of SARS-COV-2. In some embodiments, the packing signal comprises a nucleotide sequence having at least 65%, 70%, 75%, 80%, 85%, 90%, 95%, 98%, or 99% sequence identity to the nucleotide sequence of SEQ ID NO: 140 and the RBP and/or the soluble RBP comprises an amino acid sequence having at least 65%, 70%, 75%, 80%, 85%, 90%, 95%, 98%, or 99% sequence identity to the amino acid sequence of SEQ ID NO: 136. In some embodiments, the packing signal comprises Box C/D binding motif and the RBP and/or the soluble RBP is ribosomal protein L7Ae of archae. In some embodiments, the packing signal comprises a nucleotide sequence having at least 65%, 70%, 75%, 80%, 85%, 90%, 95%, 98%, or 99% sequence identity to the nucleotide sequence of SEQ ID NO: 146 and the RBP and/or the soluble RBP comprises an amino acid sequence having at least 65%, 70%, 75%, 80%, 85%, 90%, 95%, 98%, or 99% sequence identity to the amino acid sequence of SEQ ID NO: 142. In some embodiments, the packing signal comprises regulatory RNA CsrB and the RBP and/or the soluble RBP is CsrA of E. coli. In some embodiments, the packing signal comprises the nucleotide sequence of SEQ ID NO: 134 and the RBP and/or the soluble RBP comprises the amino acid sequence of SEQ ID NO: 128. In some embodiments, the packing signal comprises an MS2 phage operator stem-loop and the RNA binding protein is MS2 Coat Protein (MCP). In some embodiments, the packing signal comprises the nucleotide sequence of SEQ ID NO: 152 and the RBP and/or the soluble RBP comprises the amino acid sequence of SEQ ID NO: 148. In some embodiments, the packing signal comprises PS9 and the RBP and/or the soluble RBP is N protein of SARS-COV-2. In some embodiments, the packing signal comprises the nucleotide sequence of SEQ ID NO: 140 and the RBP and/or the soluble RBP comprises the amino acid sequence of SEQ ID NO: 136. In some embodiments, the packing signal comprises Box C/D binding motif and the RBP and/or the soluble RBP is ribosomal protein L7Ae of archaea. In some embodiments, the packing signal comprises the nucleotide sequence of SEQ ID NO: 146 and the RBP and/or the soluble RBP comprises the amino acid sequence of SEQ ID NO: 142. In some embodiments, the packing signal is situated at the 5′ end or the 3′ end of at least one of the one or more RNA cargo molecules. In some embodiments, the at least one of the one or more RNA cargo molecules comprise an mRNA, and the packing signal is situated within the 5′ or 3′ UTR of the mRNA.

In some embodiments, the ERD is capable of recruiting one or more ESCRT proteins to the cytoplasmic tail of the fusion protein. In some embodiments, the recruitment of ESCRT proteins via the ERD is capable of inducing the self-assembly and budding of ENPs. In some embodiments, the ERD is located at the C-terminus of the fusion protein, the N-terminus of the fusion protein, or between the N-terminus and the C-terminus of the fusion protein. In some embodiments, the ERD is capable of interacting with ESCRT proteins TSG101, NEDD4, and/or ALIX. In some embodiments, the ERD comprises or is derived from: a human protein; a nonhuman protein, optionally a nonmammalian protein, further optionally a chicken protein, a mouse protein, a lizard protein, a reptile protein, a hamster protein, or a goldfish protein; the ESCRT and ALIX binding region (EABR) of the human CEP55 protein, optionally residues 170-213; Syntenin-1, rat Galectin-3 (rGalectin-3), Hrs, and/or CD2AP; a viral protein, optionally a fragment of a viral protein, further optionally a retroviral protein, herpes simplex viral protein, vaccinia viral protein, hepadnaviral protein, togaviral protein, flaviviral protein, arenaviral protein, coronaviral protein, orthomyxoviral protein, paramyxoviral protein, bunyaviral protein, bornaviral protein, rhabdoviral protein or filoviral protein, optionally a Gag protein, further optionally derived from EIAV, HTLV-1, MLV, or MPMV, optionally EIAV p9 and/or HIV-1 p6; and/or an Ebola protein, optionally EBOV VP40. In some embodiments, the ERD comprises one or more TSG101-binding motifs, one or more ALIX-binding motifs, one or more Nedd4-recruiting motifs, or any combination thereof. In some embodiments, any two of the one or more TSG101-binding motifs, the one or more ALIX-binding motifs, or the one or more Nedd4-recruiting motifs are the same or different.

In some embodiments, the ERD comprises an amino acid sequence having at least 65%, 70%, 75%, 80%, 85%, 90%, 95%, 98%, or 99% sequence identity to the amino acid sequence of SEQ ID NO: 2. In some embodiments, the ERD comprises the amino acid sequence of SEQ ID NO: 2. In some embodiments, the ERD comprises or is derived from a non-human galectin protein, e.g., a rat galectin protein. In some embodiments, the ERD comprises an amino acid sequence having at least 65%, 70%, 75%, 80%, 85%, 90%, 95%, 98%, or 99% sequence identity to an amino acid sequence selected from the group consisting of SEQ ID NOs: 3-39.

In some embodiments, the fusion protein comprises an endocytosis-preventing motif (EPM) capable of preventing endocytosis of the fusion protein. In some embodiments, the EPM: tethers the fusion protein to the cytoskeleton, thereby preventing localization to coated pits and endocytosis; enhances ENP assembly, ENP production, and/or ENP secretion; and/or prevents endocytosis of the fusion protein, thereby extending the time the fusion protein remains at the plasma membrane to interact with ESCRT proteins. In some embodiments, the EPM: increases the abundance and/or density of fusion proteins on and/or in the ENP by at least about 2-fold as compared to an ENP comprising a fusion protein that does not comprise the EPM; and/or increases the number of ENPs secreted by a cell by at least about 2-fold as compared to a cell expressing a fusion protein that does not comprise the EPM. In some embodiments, the EPM comprises or is derived from a portion of murine low-affinity gamma Fc region receptor II isoform FcRII-B1. In some embodiments, the EPM comprises all or a portion of the cytoplasmic tail of FcRII-B1. In some embodiments, the EPM comprises an amino acid sequence having at least 65%, 70%, 75%, 80%, 85%, 90%, 95%, 98%, or 99% sequence identity to the amino acid sequence of SEQ ID NO: 1. In some embodiments, the EPM comprises the amino acid sequence of SEQ ID NO: 1. In some embodiments, the fusion protein does not comprise an endocytosis-preventing motif (EPM).

In some embodiments, the fusion protein comprises, from N-terminus to C-terminus: the CSP, a first optional linker, the RBP, a second optional linker, and the ERD. In some embodiments, the fusion protein comprises, from N-terminus to C-terminus: the CSP, the EPM, the first optional linker, the RBP, the second optional linker, and the ERD. In some embodiments, the first and/or second linker: is a flexible linker, a rigid linker, or a hybrid linker; is hydrophilic or hydrophobic; is between 1 and 250 amino acids; comprises one or more flexible amino acid residues, e.g., about 1 to about 250 flexible amino acid residues. In some embodiments, the flexible amino acid residues comprise glycine, serine, or a combination thereof; and/or comprises 3 repeating amino acid subunits or more.

In some embodiments, the fusion protein comprises: an amino acid sequence having at least 65%, 70%, 75%, 80%, 85%, 90%, 95%, 98%, or 99% sequence identity to any one of the amino acid sequences selected from the group consisting of SEQ ID NOs: 129, 137, 143, and 149; or an amino acid sequence selected from the group consisting of SEQ ID NOs: 129, 137, 143, and 149. In some embodiments: (i) the fusion protein comprises the amino acid sequence of SEQ ID NO: 129, the packing signal comprises the sequence of SEQ ID NO: 134 and the soluble RBP comprises the sequence of SEQ ID NO: 128; (ii) the fusion protein comprises the amino acid sequence of SEQ ID NO: 149, the packing signal comprises the sequence of SEQ ID NO: 152, and the soluble RBP comprises the sequence of SEQ ID NO: 148; (iii) the fusion protein comprises the amino acid sequence of SEQ ID NO: 137, the packing signal comprises the sequence of SEQ ID NO: 140, and the soluble RBP comprises the sequence of SEQ ID NO: 136; and/or (iv) the fusion protein comprises the amino acid sequence of SEQ ID NO: 143, the packing signal comprises the sequence of SEQ ID NO: 146, and the soluble RBP comprises the sequence of SEQ ID NO: 142.

Disclosed herein include compositions. In some embodiments, the composition comprises: a nucleic acid composition comprising: (i) a first polynucleotide encoding a dimerization fusion protein, wherein the dimerization fusion protein comprises a cell surface protein (CSP) and a heterologous cytoplasmic tail, optionally the dimerization fusion protein further comprises an RNA-binding protein (RBP) and/or an endosomal sorting complex required for transport (ESCRT)-recruiting domain (ERD); (ii) a second polynucleotide encoding an adapter fusion protein comprising an adapter domain capable of binding the heterologous cytoplasmic tail to form a heterodimer, an optional RBP, and an optional endosomal sorting complex required for transport (ESCRT)-recruiting domain (ERD); and (iii) one or more third polynucleotides comprising one or more cargo RNA molecules each comprising a packing signal, wherein the RBP of (i) and (ii) are each capable of binding the packing signal, wherein binding of the adapter domain to the heterologous cytoplasmic tail is capable of recruiting one or more ESCRT proteins to the heterodimer, thereby inducing a plurality of dimerization fusion proteins to self-assemble into an enveloped nanoparticle (ENP) secreted from a cell in which the dimerization fusion protein and adapter fusion protein are expressed, thereby generating a population of ENPs comprising the dimerization fusion protein and the one or more cargo RNA molecules, optionally the nucleic acid composition further comprises a fourth polynucleotide encoding a soluble RBP capable of binding the packing signal.

In some embodiments, the CSP is a targeting protein capable of targeting the ENP to a target cell. In some embodiments, the nucleic acid composition further comprises a fifth polynucleotide encoding a cell fusion protein. In some embodiments, the cell fusion protein is capable of inducing the fusion of a lipid envelope of the ENP and a lipid bilayer of the target cell.

In some embodiments, the dimerization fusion protein, the cell fusion protein, or both, are capable of being presented on the surface of a cell in which the dimerization fusion protein and/or the cell fusion protein are expressed. In some embodiments, the self-assembly of an ENP does not require an exogenous nucleic acid other than the nucleic acid composition. In some embodiments, the cell is: a cell of a subject; an in vivo cell, an ex vivo cell, or an in situ cell; and/or an adherent cell or a suspension cell. In some embodiments, upon secretion from a cell of a subject, the ENPs are capable of distributing within one or more tissues of the subject. In some embodiments, the one or more tissues comprise adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue, fat tissue, or any combination thereof. In some embodiments, the one or more tissues comprise diseased tissues, e.g., cancerous or infected tissues.

Disclosed herein include compositions. In some embodiments, the composition comprises: a population of enveloped nanoparticles (ENPs), wherein each of the ENPs comprises: (i) a plurality of dimerization fusion proteins each comprising a heterologous cytoplasmic tail and a CSP, optionally the CSP is a targeting protein capable of targeting the ENPs to a target cell; (ii) one or more cargo RNA molecules each comprising a packing signal; and optionally (iii) a plurality of cell fusion proteins. In some embodiments, the ENPs are derived from expression of the nucleic acid composition of the disclosure. In some embodiments, the ENPs comprise a lipid bilayer, e.g., a lipid bilayer derived from the cell from which the ENP was secreted.

In some embodiments, the packing signal and the RBP are derived from a viral, archaeal, bacterial, or mammalian packing signal and RBP, or variants thereof. In some embodiments: the packing signal comprises a Ku binding hairpin and the RBP and/or the soluble RBP is Ku; the packing signal comprises a telomerase Sm7 binding motif and the RBP and/or the soluble RBP is Sm7; the packing signal comprises an MS2 phage operator stem-loop and the RBP and/or the soluble RBP is MS2 Coat Protein (MCP); the packing signal comprises a PP7 phage operator stem-loop and the RBP and/or the soluble RBP is PP7 Coat Protein (PCP); the packing signal comprises an SfMu phage Com stem-loop and the RBP and/or the soluble RBP is Com RNA binding protein; the packing signal comprises a PUF binding site (PBS) and the RBP and/or the soluble RBP is Pumilio/fem-3 mRNA binding factor (PUF); the packing signal comprises Psi and the RBP and/or the soluble RBP is gag, optionally derived from MMLV, HIV, SIV, FIV, HTLV, or Foamy viruses; the packing signal comprises regulatory RNA CsrB and the RBP and/or the soluble RBP is CsrA of E. coli; the packing signal comprises PS9 and the RBP and/or the soluble RBP is N protein of a coronavirus, optionally SARS-COV-2; and/or the packing signal comprises Box C/D binding motif and the RBP and/or the soluble RBP is ribosomal protein L7Ae of archaea.

In some embodiments the packing signal comprises regulatory RNA CsrB and the RBP and/or the soluble RBP is CsrA of E. coli. In some embodiments, the packing signal comprises a nucleotide sequence having at least 65%, 70%, 75%, 80%, 85%, 90%, 95%, 98%, or 99% sequence identity to the nucleotide sequence of SEQ ID NO: 134 and the RBP and/or the soluble RBP comprises an amino acid sequence having at least 65%, 70%, 75%, 80%, 85%, 90%, 95%, 98%, or 99% sequence identity to the amino acid sequence of SEQ ID NO: 128. In some embodiments, the packing signal comprises an MS2 phage operator stem-loop and the RBP is MS2 Coat Protein (MCP). In some embodiments, the packing signal comprises a nucleotide sequence having at least 65%, 70%, 75%, 80%, 85%, 90%, 95%, 98%, or 99% sequence identity to the nucleotide sequence of SEQ ID NO: 152 and the RBP and/or the soluble RBP comprises an amino acid sequence having at least 65%, 70%, 75%, 80%, 85%, 90%, 95%, 98%, or 99% sequence identity to the amino acid sequence of SEQ ID NO: 148. In some embodiments, the packing signal comprises PS9 and the RBP and/or the soluble RBP is N protein of SARS-COV-2. In some embodiments, the packing signal comprises a nucleotide sequence having at least 65%, 70%, 75%, 80%, 85%, 90%, 95%, 98%, or 99% sequence identity to the nucleotide sequence of SEQ ID NO: 140 and the RBP and/or the soluble RBP comprises an amino acid sequence having at least 65%, 70%, 75%, 80%, 85%, 90%, 95%, 98%, or 99% sequence identity to the amino acid sequence of SEQ ID NO: 136. In some embodiments, the packing signal comprises Box C/D binding motif and the RBP and/or the soluble RBP is ribosomal protein L7Ae of archaea. In some embodiments, the packing signal comprises a nucleotide sequence having at least 65%, 70%, 75%, 80%, 85%, 90%, 95%, 98%, or 99% sequence identity to the nucleotide sequence of SEQ ID NO: 146 and the RBP and/or the soluble RBP comprises an amino acid sequence having at least 65%, 70%, 75%, 80%, 85%, 90%, 95%, 98%, or 99% sequence identity to the amino acid sequence of SEQ ID NO: 142. In some embodiments the packing signal comprises regulatory RNA CsrB and the RBP and/or the soluble RBP is CsrA of E. coli. In some embodiments, the packing signal comprises the nucleotide sequence of SEQ ID NO: 134 and the RBP and/or the soluble RBP comprises the amino acid sequence of SEQ ID NO: 128. In some embodiments, the packing signal comprises an MS2 phage operator stem-loop and the RNA binding protein is MS2 Coat Protein (MCP). In some embodiments, the packing signal comprises the nucleotide sequence of SEQ ID NO: 152 and the RBP and/or the soluble RBP comprises the amino acid sequence of SEQ ID NO: 148. In some embodiments, the packing signal comprises PS9 and the RBP and/or the soluble RBP is N protein of SARS-COV-2. In some embodiments, the packing signal comprises the nucleotide sequence of SEQ ID NO: 140 and the RBP and/or the soluble RBP comprises the amino acid sequence of SEQ ID NO: 136. In some embodiments, the packing signal comprises Box C/D binding motif and the RBP and/or the soluble RBP is ribosomal protein L7Ae of archaea. In some embodiments, the packing signal comprises the nucleotide sequence of SEQ ID NO: 146 and the RBP and/or the soluble RBP comprises the amino acid sequence of SEQ ID NO: 142. In some embodiments, the packing signal is situated at the 5′ end or the 3′ end of at least one of the one or more RNA cargo molecules. In some embodiments, the at least one of the one or more RNA cargo molecules comprise an mRNA, and the packing signal is situated within the 5′ or 3′ UTR of the mRNA.

In some embodiments, the ERD is capable of recruiting one or more ESCRT proteins to the cytoplasmic tail of the dimerization fusion protein. In some embodiments, the recruitment of ESCRT proteins via the ERD is capable of inducing the self-assembly and budding of ENPs. In some embodiments, the ERD is located at the C-terminus of the adapter fusion protein and/or the heterologous fusion protein, the N-terminus of the adapter fusion protein and/or the heterologous fusion protein, or between the C-terminus and the N-terminus of the adapter fusion protein and/or the heterologous fusion protein. In some embodiments, the ERD is capable of interacting with the ESCRT proteins TSG101, NEDD4, and/or ALIX. In some embodiments, the ERD comprises or is derived from: a human protein; a nonhuman protein, optionally a nonmammalian protein, further optionally a chicken protein, a mouse protein, a lizard protein, a reptile protein, a hamster protein, or a goldfish protein; the ESCRT and ALIX binding region (EABR) of the human CEP55 protein, optionally residues 170-213; Syntenin-1, rat Galectin-3 (rGalectin-3), Hrs, and/or CD2AP; a viral protein, optionally a fragment of a viral protein, further optionally a retroviral protein, herpes simplex viral protein, vaccinia viral protein, hepadnaviral protein, togaviral protein, flaviviral protein, arenaviral protein, coronaviral protein, orthomyxoviral protein, paramyxoviral protein, bunyaviral protein, bornaviral protein, rhabdoviral protein or filoviral protein, optionally a Gag protein, further optionally derived from EIAV, HTLV-1, MLV, or MPMV, optionally EIAV p9 and/or HIV-1 p6; and/or an Ebola protein, optionally EBOV VP40. In some embodiments, the ERD comprises one or more TSG101-binding motifs, one or more ALIX-binding motifs, one or more Nedd4-recruiting motifs, or any combination thereof. In some embodiments, any two of the one or more TSG101-binding motifs, the one or more ALIX-binding motifs, or the one or more Nedd4-recruiting motifs are the same or different.

In some embodiments, the ERD comprises an amino acid sequence having at least 65%, 70%, 75%, 80%, 85%, 90%, 95%, 98%, or 99% sequence identity to the amino acid sequence of SEQ ID NO: 2. In some embodiments, the ERD comprises the amino acid sequence of SEQ ID NO: 2. In some embodiments, the ERD comprises or is derived from a non-human galectin protein, e.g., a rat galectin protein. In some embodiments, the ERD comprises an amino acid sequence having at least 65%, 70%, 75%, 80%, 85%, 90%, 95%, 98%, or 99% sequence identity to an amino acid sequence selected from the group consisting of SEQ ID NOs: 3-39.

In some embodiments, the heterologous cytoplasmic tail and/or adapter domain is derived from a mammalian, reptilian, avian, amphibian, or fish protein. In some embodiments, the heterologous cytoplasmic tail and/or adapter domain comprises or is derived from at least a portion of LAT, PAG, LCK, FYN, LAX, CD2, CD3, CD4, CD5, CD7, CD8a, PD1, SRC, or LYN. In some embodiments, the dimerization fusion protein and/or the adapter fusion protein further comprise an endogenous cytoplasmic tail, e.g., N-terminal to the heterologous cytoplasmic tail.

In some embodiments, the heterologous cytoplasmic tail and/or the adapter domain are each selected from the group comprising DHD9 heterodimer a, DHD13_XAAA heterodimer a, DHD13_XAXA heterodimer a, DHD13_XAAX heterodimer a, DHD13_2: 341 heterodimer a, DHD13_AAAA heterodimer a, DHD13_BAAA heterodimer a, DHD13_4: 123 heterodimer a, DHD13_1: 234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34_XAAXA heterodimer a, DHD34_XAXXA heterodimer a, DHD34 XAAAA heterodimer a, DHD36 heterodimer a, DHD37_ABXB heterodimer a, DHD37 BBBB heterodimer a, DHD37 XBXB heterodimer a, DHD37_AXXB heterodimer a, DHD37 3:124 heterodimer a, DHD37_1: 234 heterodimer a, DHD37_AXBB heterodimer a, DHD37 XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94 3:214 heterodimer a, DHD94_2: 143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102 1:243 heterodimer a, DHD 103 heterodimer a, DHD103_1: 423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD 144 heterodimer a, DHD9 heterodimer b, DHD13_XAAA heterodimer b, DHD13_XAXA heterodimer b, DHD13_XAAX heterodimer b, DHD13_2: 341 heterodimer b, DHD13_AAAA heterodimer b, DHD13_BAAA heterodimer b, DHD13 4:123 heterodimer b, DHD13_1: 234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34_XAAXA heterodimer b, DHD34_XAXXA heterodimer b, DHD34_XAAAA heterodimer b, DHD36 heterodimer b, DHD37_ABXB heterodimer b, DHD37 BBBB heterodimer b, DHD37 XBXB heterodimer b, DHD37_AXXB heterodimer b, DHD37_3: 124 heterodimer b, DHD37_1: 234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3: 214 heterodimer b, DHD94_2: 143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1: 243 heterodimer b, DHD103 heterodimer b, DHD103_1: 423 heterodimer b, DHD104 heterodimer b, DHD105 heterodimer b, DHD 106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD 128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD 150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD 17 heterodimer b, DHD131 heterodimer b, DHD 132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD 142 heterodimer b, DHD143 heterodimer b, DHD144 heterodimer b, portions thereof, derivatives thereof, or any combination thereof.

In some embodiments, the heterologous cytoplasmic tail and/or the adapter domain comprises or is derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACHI, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB 1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, the heterologous cytoplasmic tail comprises or is derived from ACIDpl or BASEp1. In some embodiments, the heterologous cytoplasmic tail comprises or is derived from N5 or N6. In some embodiments, the heterologous cytoplasmic tail comprises the sequence of any one of SEQ ID NOs: 116-117 and 121-122. In some embodiments, the adapter domain comprises or is derived from ACIDpl or BASEp1. In some embodiments, the heterologous cytoplasmic tail comprises or is derived from N5 or N6. In some embodiments, the adapter domain comprises the sequence of any one of SEQ ID NOs: 116-117 and 121-122.

In some embodiments, the heterologous cytoplasmic tail comprises or is derived from a cytoplasmic tail (CT) of CD4. In some embodiments, the adapter domain comprises or is derived from Lck tyrosine kinase. In some embodiments, the CD4 CT comprises the sequence of SEQ ID NO: 95. In some embodiments, the adapter domain comprises the sequence of SEQ ID NO: 43. In some embodiments, the adapter fusion protein comprises, from N-terminus to C-terminus: the adapter domain, a first optional linker, the RBP, a second optional linker, and the ERD. In some embodiments, the first and/or second linker: is a flexible linker, a rigid linker, or a hybrid linker; is hydrophilic or hydrophobic; is between 1 and 250 amino acids; comprises one or more flexible amino acid residues, e.g., about 1 to about 250 flexible amino acid residues, further optionally the flexible amino acid residues comprise glycine, serine, or a combination thereof; and/or comprises 3 repeating amino acid subunits or more. In some embodiments, the adapter fusion protein comprises an amino acid sequence having at least 65%, 70%, 75%, 80%, 85%, 90%, 95%, 98%, or 99% sequence identity to any one of the amino acid sequences selected from the group consisting of SEQ ID NOs: 157, 159, 162, and 164.

In some embodiments, the heterologous cytoplasmic tail is derived from or comprises a cytoplasmic tail of a cell surface protein. In some embodiments, the adapter domain is (1) capable of binding the heterologous cytoplasmic tail derived from or comprising the cytoplasmic tail of said cell surface protein and (2) capable of targeting the adapter fusion protein to the plasma membrane. In some embodiments, the adapter domain comprises or is derived from Lck tyrosine kinase. In some embodiments, the Lck tyrosine kinase comprises a myristolylation motif. In some embodiments, myristoylation drives membrane anchoring of the adapter fusion protein to the plasma membrane. In some embodiments, the cell surface protein is or is derived from a human protein, a non-human mammalian protein, an avian protein, a reptile protein, a fish protein, an amphibian protein, a viral protein, or a bacterial protein and/or the adapter domain is or is derived from a human protein, a non-human mammalian protein, an avian protein, a reptile protein, a fish protein, an amphibian protein, a viral protein, or a bacterial protein.

In some embodiments, the dimerization fusion protein comprises an endocytosis-preventing motif (EPM) capable of preventing endocytosis of the dimerization fusion protein. In some embodiments, the EPM: tethers the dimerization fusion protein to the cytoskeleton, thereby preventing localization to coated pits and endocytosis; enhances ENP assembly, ENP production, and/or ENP secretion; and/or prevents endocytosis of the dimerization fusion protein, and/or the adapter fusion protein, thereby extending the time the dimerization fusion protein, and/or the adapter fusion protein remains at the plasma membrane to interact with ESCRT proteins. In some embodiments, the EPM: increases the abundance and/or density of dimerization fusion proteins, adapter fusion proteins, one or more RNA cargo molecules, and/or cell surface proteins on and/or in the ENP by at least about 2-fold as compared to an ENP comprising a dimerization fusion protein that does not comprise the EPM; and/or increases the number of ENPs secreted by a cell by at least about 2-fold as compared to a cell expressing a dimerization fusion protein that does not comprise the EPM. In some embodiments, the EPM comprises or is derived from a portion of murine low-affinity gamma Fc region receptor II isoform FcRII-B1. In some embodiments, the EPM comprises all or a portion of the cytoplasmic tail of FcRII-B1. In some embodiments, the EPM comprises an amino acid sequence having at least 65%, 70%, 75%, 80%, 85%, 90%, 95%, 98%, or 99% sequence identity to the amino acid sequence of SEQ ID NO: 1. In some embodiments, the EPM comprises the amino acid sequence of SEQ ID NO: 1. In some embodiments, the dimerization fusion protein does not comprise an endocytosis-preventing motif (EPM).

In some embodiments, the dimerization fusion protein comprises, from N-terminus to C-terminus: the CSP, the heterologous cytoplasmic tail, a first flexible linker, and the EPM. In some embodiments, the dimerization fusion protein comprises, from N-terminus to C-terminus: the CSP, the heterologous cytoplasmic tail, a first flexible linker, the EPM, a second flexible linker, and the RBP. In some embodiments, the first and/or second linker: is a flexible linker, a rigid linker, or a hybrid linker; is hydrophilic or hydrophobic; is between 1 and 250 amino acids; comprises one or more flexible amino acid residues, e.g., about 1 to about 250 flexible amino acid residues, further optionally the flexible amino acid residues comprise glycine, serine, or a combination thereof; and/or comprises 3 repeating amino acid subunits or more. In some embodiments, the dimerization fusion protein comprises a sequence selected from the sequences of SEQ ID NOs: 156, 158, 160-161, and 163.

In some embodiments, (i) the first polynucleotide encoding the dimerization fusion protein, and (ii) the second polynucleotide encoding the adapter fusion protein, are each present in a different nucleic acid molecule. In some embodiments, the amount of (i) the polynucleotide encoding the dimerization fusion protein; and (ii) the polynucleotide encoding the adapter fusion protein, are present in the composition at a molar ratio of about 9:1, 5:1, 1:1, 1:5, or 1:9. In some embodiments, (i) the first polynucleotide encoding the dimerization fusion protein, and (ii) the second polynucleotide encoding the adapter fusion protein, are present in the same nucleic acid molecule.

In some embodiments, the CSP is a targeting protein and comprises or is derived from one or more receptors and/or targeting moieties configured to bind a target molecule of a cell of a subject. In some embodiments, the one or more receptors and/or the one or more targeting moieties are selected from the group comprising mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic.

In some embodiments, the one or more receptors and/or targeting moieties comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, a receptor ligand, a chimeric antigen receptor (CAR), a T cell receptor (TCR), a targeted recognition of antigen-MHC complex reporter (TRACeR), and analogs and derivatives thereof.

In some embodiments, the antibody or antigen-binding fragment thereof comprises a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

In some embodiments, the one or more receptors and/or targeting moieties are configured to bind one or more of the following: CD3, CD4, CD5, CD6, CD7, CD8, CD9, CD10, CD11a, CD11b, CD11c, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin Al, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11 (C-C motif chemokine 11), CCR4 (C-C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSFIR (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA 16.88 tumor antigen, CXCR4 (CD 184), C—X—C chemokine receptor type 4, cyclic ADP ribose hydrolase, Cyclin B 1, CYPIB 1, Cytomegalovirus, Cytomegalovirus glycoprotein B, Dabigatran, DLL4 (delta-like-ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRvIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor α-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGFIR (IGF-1, insulin-like growth factor 1 receptor), IGHE, IFN-γ, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αιιβ3. ανβ3, αβ7, α5β1, αββ4, α7β7, α11β3, α5β5, ανβ5), Interferon gamma-induced protein, ITGA2, ITGB2, KIR2D, LCK, Le, Legumain, Lewis-Y antigen, LFA-1 (Lymphocyte function-associated antigen 1, CD11a), LHRH, LINGO-1, Lipoteichoic acid, LIVIA, LMP2, LTA, MAD-CT-1, MAD-CT-2, MAGE-1, MAGE-2, MAGE-3, MAGE AI, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUCI (Mucin 1, cell surface associated (MUC1) or polymorphic epithelial mucin (PEM)), MUCI-KLH, MUC16 (CA125), MCPI (monocyte chemotactic protein 1), MelanA/MARTI, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page4, PAP, Paratope of anti-(N-glycolylneuraminic acid), PAX3, PAX5, PCSK9, PDCDI (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PRI), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe (a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUCI, TWEAK receptor, TYRPI (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, T-cell receptors, viral surface proteins, peptide-MHC complexes, or cells expressing any insulin growth factor receptors, or any epidermal growth factor receptors. In some embodiments, the peptide of the peptide-MHC complex is associated with a disease or disorder. In some embodiments, the peptide of the peptide-MHC complex is an intracellular tumor antigen.

In some embodiments, the CSP is a targeting protein and comprises or is derived from an scFv. In some embodiments, the scFv comprises a transmembrane domain or is fused to a heterologous transmembrane domain.

In some embodiments, the scFv is capable of binding to: CD3, CD4, CD5, CD6, CD7, CD8, CD9, CD10, CD11a, CD11b, CD11c, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166,CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin Al, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11 (C-C motif chemokine 11), CCR4 (C-C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSFIR (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C—X—C chemokine receptor type 4, cyclic ADP ribose hydrolase, Cyclin B 1, CYPIB 1, Cytomegalovirus, Cytomegalovirus glycoprotein B, Dabigatran, DLL4 (delta-like-ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRVIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor α-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGFIR (IGF-1, insulin-like growth factor 1 receptor), IGHE, IFN-γ, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αμ. ανβ3, αβ7, α5β1, αββ4, α7β7, α11β3, α5β5, ανβ5), Interferon gamma-induced protein, ITGA2, ITGB2, KIR2D, LCK, Le, Legumain, Lewis-Y antigen, LFA-1 (Lymphocyte function-associated antigen 1, CD11a), LHRH, LINGO-1, Lipoteichoic acid, LIVIA, LMP2, LTA, MAD-CT-1, MAD-CT-2, MAGE-1, MAGE-2, MAGE-3, MAGE AI, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUCI (Mucin 1, cell surface associated (MUC1) or polymorphic epithelial mucin (PEM)), MUCI-KLH, MUC16 (CA125), MCPI (monocyte chemotactic protein 1), MelanA/MARTI, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page4, PAP, Paratope of anti-(N-glycolylneuraminic acid), PAX3, PAX5, PCSK9, PDCDI (PD-1, Programmed cell death protein 1, CD279), PDGF-Ra (Alpha-type platelet-derived growth factor receptor), PDGFR-B, PDL-1, PLAC1, PLAP-like testicular alkaline phosphatase, Platelet-derived growth factor receptor beta, Phosphate-sodium co-transporter, PMEL 17, Polysialic acid, Proteinase3 (PRI), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe (a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUCI, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, T-cell receptors, viral surface proteins, peptide-MHC complexes, or cells expressing any insulin growth factor receptors, or any epidermal growth factor receptors. In some embodiments, the peptide of the peptide-MHC complex is associated with a disease or disorder. In some embodiments, the peptide of the peptide-MHC complex is an intracellular tumor antigen.

In some embodiments, the scFv is capable of binding to CD19, CD4, CD3, or any combination thereof. In some embodiments, the heterologous transmembrane domain comprises CD8a chain transmembrane domain. In some embodiments, the CSP is a targeting protein comprising an scFv and comprises the sequence of any one of SEQ ID NOs: 178, 180, and 182. In some embodiments, the CSP is a targeting protein and comprises or is derived from SARS-CoV spike protein. In some embodiments, the CSP comprising or derived from SARS-COV spike protein is capable of targeting the ENP to a target cell expressing ACE2. In some embodiments, the CSP comprises SARS-COV-2 spike protein and comprises an amino acid sequence having at least 65%, 70%, 75%, 80%, 85%, 90%, 95%, 98%, or 99% sequence identity to the sequence of SEQ ID NO: 40. In some embodiments, the cell fusion protein comprises or is derived from VSV-G. In some embodiments, the VSV-G comprises one or more mutations thereby the VSV-G protein is not capable of binding to an LDL-receptor. In some embodiments, the one or more mutations comprise K47Q and/or R354A relative to wild type VSV-G. In some embodiments, the cell fusion protein comprises an amino acid sequence having at least 65%, 70%, 75%, 80%, 85%, 90%, 95%, 98%, or 99% sequence identity to the sequence of SEQ ID NO: 169.

In some embodiments, the cell fusion protein is, comprises, or is derived from a SNARE protein, a viral glycoprotein, an FF protein, dynamin, a FAST protein, synuclein, myomaker, myomerger, or any combination thereof.

In some embodiments, the viral glycoprotein is selected from the group comprising glycoprotein GP of Ebola or Marburg virus, glycoproteins HN and F of Newcastle virus, protein E and prM of Murray Valley encephalitis virus, El and/or E2 proteins of HCV, HA (hemaglutinin) and NA (neuraminidase) of Influenza, glycoprotein G of VSV, glycoproteins Gp120 (or a CD4-binding domain thereof) and Gp41 of lentiviruses, envelope protein (DENV E) and pre-membrane protein (prM DENV) of Dengue virus, the two envelope glycoproteins of Hantaan virus, glycoprotein E2 of Chikungunya virus, gp85 and gp37 of Rous sarcoma virus, HBsAg of HBV, or any combination thereof. In some embodiments, the viral glycoprotein is selected from the group comprising M-HBsAg, S-HBsAg or L-HBsAg. In some embodiments, the viral glycoprotein is a measles glycoprotein, a sindvis virus glycoprotein, baboon retroviral Env, or a Reovirus Fusion-Associated Small Transmembrane (FAST) protein. In some embodiments, the viral glycoprotein is a glycoprotein from hepatitis D virus, orthomyxoviridae, paramyxoviridae, filoviridae, retroviridae, herpesviridae, poxviridae, hepadnaviridae, flaviviridae, togavoridae, coronaviridae, rhabdoviridae, bunyaviridae, orthopoxivridae, measles virus, sindbis virus, baboon retroviral virus, or any combination thereof.

In some embodiments: (i) the dimerization fusion protein comprises the sequence of SEQ ID NO: 156, the adapter fusion protein comprises the sequence of SEQ ID NO: 157, the soluble RBP comprises the sequence of SEQ ID NO: 128, and the packing signal comprises the sequence of SEQ ID NO: 134; (ii) the dimerization fusion protein comprises the sequence of SEQ ID NO: 161, the adapter fusion protein comprises the sequence of SEQ ID NO: 162, the soluble RBP comprises the sequence of SEQ ID NO: 136, and the packing signal comprising the sequence of SEQ ID NO: 140; (iii) the dimerization fusion protein comprises the sequence of SEQ ID NO: 160, the adapter fusion protein comprises the sequence of SEQ ID NO: 162, the soluble RBP comprises the sequence of SEQ ID NO: 136, and the packing signal comprises the sequence of SEQ ID NO: 140; (iv) the dimerization fusion protein comprises the sequence of SEQ ID NO: 160, the adapter fusion protein comprises the sequence of SEQ ID NO: 162, the soluble RBP comprises the sequence of SEQ ID NO: 136, the packing signal comprises the sequence of SEQ ID NO: 140, and the cell fusion protein comprises the sequence of SEQ ID NO: 169, optionally the nucleic acid composition does not comprise the fourth polynucleotide encoding the soluble RBP; (v) the dimerization fusion protein comprises the sequence of SEQ ID NO: 160, the adapter fusion protein comprises the sequence of SEQ ID NO: 164, the soluble RBP comprises the sequence of SEQ ID NO: 142, the packing signal comprises the sequence of SEQ ID NO: 146, and the cell fusion protein comprises the sequence of SEQ ID NO: 169, optionally the nucleic acid composition does not comprise the fourth polynucleotide encoding the soluble RBP; (vi) the dimerization fusion protein comprises the sequence of SEQ ID NO: 179, the adapter fusion protein comprises the sequence of SEQ ID NO: 164, the packing signal comprises the sequence of SEQ ID NO: 146, and the cell fusion protein comprises the sequence of SEQ ID NO: 169; and/or (vii) the dimerization fusion protein comprises the sequence of SEQ ID NO: 181, the adapter fusion protein comprises the sequence of SEQ ID NO: 164, the packing signal comprises the sequence of SEQ ID NO: 146, and the cell fusion protein comprises the sequence of SEQ ID NO: 169.

In some embodiments, the one or more cargo RNA molecules each comprise a microRNA (miRNA), a messenger RNA (mRNA), a long non-coding RNA (lncRNA), a ribosomal RNA (rRNA), a transfer RNA (tRNA), a small nuclear RNA (snRNA), a small nucleolar RNA (snoRNA), a Piwi-interacting RNA (piRNA), a interfering RNA (siRNA), an antisense RNA (aRNA), a transfer messenger RNA (tmRNA), a tRNA-derived small RNA (tsRNA), a rDNA-derived small RNA (srRNA), a ribozyme, a viral RNA, a single-stranded RNA, a double-stranded RNA, self-amplifying RNA, circular RNA, an aptamer, or any combination thereof. In some embodiments, the miRNA or siRNA is capable of inhibiting the expression of a target mRNA in a cell. In some embodiments, the mRNA encodes a payload protein.

In some embodiments, the miRNA, the siRNA, and/or payload protein is a therapeutic miRNA, siRNA, and/or protein or a variant thereof, e.g., a therapeutic miRNA, siRNA, and/or protein configured to prevent or treat a disease or disorder of a subject. In some embodiments, the subject suffers from a deficiency of said therapeutic protein.

In some embodiments, the payload protein comprises fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. In some embodiments, the payload protein comprises nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. In some embodiments, the payload protein comprises a CRE recombinase, GCaMP, a cell therapy component, a knock-down gene therapy component, a cell-surface exposed epitope, or any combination thereof.

In some embodiments, the payload protein comprises a diagnostic agent. In some embodiments, the diagnostic agent comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCitrine, mCherry, mruby3, rsCherry, rsCherryRev, derivatives thereof, or any combination thereof.

In some embodiments, the payload protein comprises a tumor antigen. In some embodiments, the tumor antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECLI); CD33; epidermal growth factor receptor variant III (EGFRvIII); ganglioside G2 (GD2); ganglioside GD3 (aNeu5Ac (2-8) aNeu5Ac (2-3) bDGalp (1-4) bDGlcp (1-1) Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac (2-3) bDGalp (1-4) bDGlcp (1-1) Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYPIB1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the payload protein comprises a tumor antigen. In some embodiments, the tumor antigen is selected from the group comprising CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRVIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-IR, IL-11Ralpha, IL-13R-alpha2, IL-2, IL-22R-alpha, IL-6, IL-6R, Ia, Ii, LI-CAM, LI-cell adhesion molecule, Lewis Y, LI-CAM, MAGE A3, MAGE-A1, MART-1, MUCI, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-Microglobulin, Fc Receptor-like 5 (FcRL5), or molecules expressed by HIV, HCV, HBV, or other pathogens.

In some embodiments, the payload protein comprises a tumor antigen. In some embodiments, the tumor antigen comprises a peptide-MHC complex, the peptide complexed with a class I or class II MHC sequence. In some embodiments, the peptide of the peptide-MHC complex is associated with a disease or disorder. In some embodiments, the peptide of the peptide-MHC complex is an intracellular tumor antigen.

In some embodiments, the payload protein comprises a cytokine. In some embodiments, the cytokine is selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof. In some embodiments, the payload protein comprises a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5. In some embodiments, the payload protein comprises a member of the TNF family of cytokines selected from the group consisting of TNF-alpha, TNF-beta, LT-beta, CD40 ligand, Fas ligand, CD 27 ligand, CD 30 ligand, and 4-1 BBL. In some embodiments, the payload protein comprises a member of the immunoglobulin superfamily of cytokines selected from the group consisting of B7.1 (CD80) and B7.2 (B70). In some embodiments, the payload protein comprises an interferon. In some embodiments, the interferon is selected from interferon alpha, interferon beta, or interferon gamma. In some embodiments, the payload protein comprises a chemokine. In some embodiments, the chemokine is selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCLI), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15. In some embodiments, the payload protein comprises an interleukin. In some embodiments, the interleukin is selected from IL-10 IL-12, IL-1, IL-6, IL-7, IL-15, IL-2, IL-18 or IL-21. In some embodiments, the payload protein comprises a tumor necrosis factor (TNF). In some embodiments, the TNF is selected from TNF-alpha, TNF-beta, TNF-gamma, CD252, CD154, CD178, CD70, CD153, or 4-1BBL. In some embodiments, a payload protein comprises a factor locally down-regulating the activity of endogenous immune cells. In some embodiments, the payload protein is capable of remodeling a tumor microenvironment and/or reducing immunosuppression at a target site of a subject.

In some embodiments, the payload protein comprises a monoclonal antibody, a bispecific T-cell engager (BiTE), a chimeric antigen receptor (CAR), or T-cell receptor (TCR). In some embodiments, the CAR and/or TCR comprises one or more of an antigen binding domain, a transmembrane domain, and an intracellular signaling domain. In some embodiments, the monoclonal antibody and/or BiTE comprise an antigen binding domain. In some embodiments, the intracellular signaling domain comprises a primary signaling domain, a costimulatory domain, or both of a primary signaling domain and a costimulatory domain. In some embodiments, the primary signaling domain comprises a functional signaling domain of one or more proteins selected from the group consisting of CD3 zeta, CD3 gamma, CD3 delta, CD3 epsilon, common FcR gamma (FCER1G), FcR beta (Fc Epsilon R1b), CD79a, CD79b, Fcgamma RIIa, DAP10, and DAP12, or a functional variant thereof. In some embodiments, the costimulatory domain comprises a functional domain of one or more proteins selected from the group consisting of CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof.

In some embodiments, the antigen binding domain binds a tumor antigen. In some embodiments, the tumor antigen is a solid tumor antigen. In some embodiments, the tumor antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECLI); CD33; epidermal growth factor receptor variant III (EGFRvIII); ganglioside G2 (GD2); ganglioside GD3 (aNeu5Ac (2-8) aNeu5Ac (2-3) bDGalp (1-4) bDGlcp (1-1) Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin β2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac (2-3) bDGalp (1-4) bDGlcp (1-1) Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1). In some embodiments, the tumor antigen is selected from the group comprising CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRVIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-IR, IL-11Ralpha, IL-13R-alpha2, IL-2, IL-22R-alpha, IL-6, IL-6R, Ia, Ii, LI-CAM, LI-cell adhesion molecule, Lewis Y, LI-CAM, MAGE A3, MAGE-A1, MART-1, MUCI, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-Microglobulin, Fc Receptor-like 5 (FcRL5), or molecules expressed by HIV, HCV, HBV, or other pathogens. In some embodiments, the tumor antigen comprises a peptide-MHC complex, the peptide complexed with a class I or class II MHC sequence. In some embodiments, the peptide of the peptide-MHC complex is associated with a disease or disorder. In some embodiments, the peptide of the peptide-MHC complex is an intracellular tumor antigen.

In some embodiments, the antigen binding domain comprises an antibody, an antibody fragment, an scFv, a Fv, a Fab, a (Fab′)₂, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

In some embodiments, the antigen binding domain is connected to the transmembrane domain by a hinge region. In some embodiments, the transmembrane domain comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof.

In some embodiments, the CAR or TCR further comprises a leader peptide. In some embodiments, the TCR further comprises a constant region and/or CDR4.

In some embodiments, the payload protein comprises a programmable nuclease. In some embodiments, the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. In some embodiments, the programmable nuclease comprises a zinc finger nuclease (ZFN) and/or transcription activator-like effector nuclease (TALEN). In some embodiments, the programmable nuclease comprises Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof.

In some embodiments, the payload protein comprises an agonistic or antagonistic antibody or antigen-binding fragment thereof specific to: a checkpoint inhibitor or checkpoint stimulator molecule, e.g., PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, and/or TIM-3; a viral protein, e.g., Env or spike, e.g., HIV Env or SARS-COV-2 Spike; or an inflammatory cytokine. In some embodiments the inflammatory cytokine is selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF.

In some embodiments, the payload protein comprises a pro-death protein capable of halting cell growth and/or inducing cell death. In some embodiments, the pro-death protein comprises cytosine deaminase, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof. In some embodiments, the pro-death protein is capable of halting cell growth and/or inducing cell death in the presence of a pro-death agent. In some embodiments: the pro-death protein comprises Caspase-9 and the pro-death agent comprises AP1903; the pro-death protein comprises HSV thymidine kinase (TK) and the pro-death agent Ganciclovir (GCV), Ganciclovir elaidic acid ester, Penciclovir (PCV), Acyclovir (ACV), Valacyclovir (VCV), (E)-5-(2-bromovinyl)-2′-deoxyuridine (BVDU), Zidovuline (AZT), and/or 2′-exo-methanocarbathymidine (MCT); the pro-death protein comprises Cytosine Deaminase (CD) and the pro-death agent comprises 5-fluorocytosine (5-FC); the pro-death protein comprises Purine nucleoside phosphorylase (PNP) and the pro-death agent comprises 6-methylpurine deoxyriboside (MEP) and/or fludarabine (FAMP); the pro-death protein comprises a Cytochrome p450 enzyme (CYP) and the pro-death agent comprises Cyclophosphamide (CPA), Ifosfamide (IFO), and/or 4-ipomeanol (4-IM); the pro-death protein comprises a Carboxypeptidase (CP) and the pro-death agent comprises 4-[(2-chloroethyl) (2-mesyloxyethyl) amino]benzoyl-L-glutamic acid (CMDA), Hydroxy- and amino-aniline mustards, Anthracycline glutamates, and/or Methotrexate a-peptides (MTX-Phe); the pro-death protein comprises Carboxylesterase (CE) and the pro-death agent comprises Irinotecan (IRT), and/or Anthracycline acetals; the pro-death protein comprises Nitroreductase (NTR) and the pro-death agent comprises dinitroaziridinylbenzamide CB1954, dinitrobenzamide mustard SN23862, 4-Nitrobenzyl carbamates, and/or Quinones; the pro-death protein comprises Horse radish peroxidase (HRP) and the pro-death agent comprises Indole-3-acetic acid (IAA) and/or 5-Fluoroindole-3-acetic acid (FIAA); the pro-death protein comprises Guanine Ribosyltransferase (XGRTP) and the pro-death agent comprises 6-Thioxanthine (6-TX); the pro-death protein comprises a glycosidase enzyme and the pro-death agent comprises HM1826 and/or Anthracycline acetals; the pro-death protein comprises Methionine-a, y-lyase (MET) and the pro-death agent comprises Selenomethionine (SeMET); and/or the pro-death protein comprises thymidine phosphorylase (TP) and the pro-death agent comprises 5′-Deoxy-5-fluorouridine (5′-DFU).

In some embodiments, the payload protein is a cellular reprogramming factor capable of converting an at least partially differentiated cell to a less differentiated cell, e.g., Oct-3, Oct-4, Sox2, c-Myc, Klf4, Nanog, Lin28, ASCLI, MYTIL, TBX3b, SV40 large T, hTERT, miR-291, miR-294, miR-295, or any combinations thereof.

In some embodiments, the payload protein comprises a secretion tag. In some embodiments, the secretion tag is selected from the group comprising AbnA, AmyE, AprE, BgIC, BglS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC. In some embodiments, the payload protein comprises a constitutive signal peptide for protein degradation, e.g., PEST. In some embodiments, the payload protein comprises a nuclear localization signal (NLS) or a nuclear export signal (NES). In some embodiments, the payload protein comprises a degron.

In some embodiments, the one or more third polynucleotides comprise at least two third polynucleotides. In some embodiments, at least one of the one or more RNA cargo molecules of each of the at least two third polynucleotides are the same or different.

In some embodiments, at least one of the one or more third polynucleotides comprises a promoter operably linked to an RNA cargo molecule. In some embodiments, the promoter is capable of inducing the transcription of the RNA cargo molecule. In some embodiments, the at least one third polynucleotide comprises one or more of a 5′ UTR, 3′ UTR, a minipromoter, an enhancer, a splicing signal, a polyadenylation signal, a terminator, a protein degradation signal, and an internal ribosome-entry element (IRES) operably linked to the RNA cargo molecule. In some embodiments, the at least one of the one or more third polynucleotides further comprises a transcript stabilization element. In some embodiments, the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof.

In some embodiments, the promoter comprises a ubiquitous promoter. In some embodiments, the ubiquitous promoter is selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EFla) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof. In some embodiments, the promoter is an inducible promoter. In some embodiments, the inducible promoter is a tetracycline responsive promoter, a TRE promoter, a Tre3G promoter, an ecdysone responsive promoter, a cumate responsive promoter, a glucocorticoid responsive promoter, and estrogen responsive promoter, a PPAR-y promoter, or an RU-486 responsive promoter. In some embodiments, the promoter comprises a tissue-specific promoter and/or a lineage-specific promoter.

In some embodiments, the nucleic acid composition further comprises a polynucleotide comprising or encoding a tetherin inhibitor. In some embodiments, the tetherin inhibitor is capable of modulating expression, concentration, localization, stability, and/or activity of tetherin. In some embodiments, the tetherin inhibitor comprises a dsRNA, an siRNA, an shRNA, a pre-miRNA, a pri-miRNA, a miRNA, an stRNA, an lncRNA, a piRNA, a snoRNA, or a protein. In some embodiments, one or more of (i) the polynucleotide comprising or encoding the tetherin inhibitor, (ii) the first polynucleotide encoding the dimerization fusion protein, and (iii) the second polynucleotide encoding the adapter fusion protein, are present in a same or a different nucleic acid molecule. In some embodiments, the amount of (i) the polynucleotide comprising or encoding the tetherin inhibitor; and (ii) the first polynucleotide encoding the dimerization fusion protein, and/or the second polynucleotide encoding the adapter fusion protein, are present in the composition at a molar ratio of about 1:1, 1:5 or 1:25. In some embodiments, the polynucleotide comprising or encoding the tetherin inhibitor and the first polynucleotide encoding the dimerization fusion protein are present in the same nucleic acid. In some embodiments, the polynucleotide comprising or encoding the tetherin inhibitor and the second polynucleotide encoding the adapter fusion protein are present in the same nucleic acid.

In some embodiments, the tetherin inhibitor comprises or is derived from a viral protein. In some embodiments, the virus is HIV-1, HIV-2, SIV, Ebola virus, KSHV, SARS CoV, or SARS-COV-2. In some embodiments, the tetherin inhibitor comprises HIV-1 Vpu protein, KSHV K5 protein, SARS-COV-2 ORF7a, HIV-2 Env, Ebola GP, SIV Env, SIV Vpu, SIV Nef, or any portions, variants or derivatives thereof. In some embodiments, the tetherin inhibitor comprises an amino acid sequence having at least 65%, 70%, 75%, 80%, 85%, 90%, 95%, 98%, or 99% sequence identity to an amino acid sequence selected from the group consisting of SEQ ID NOs: 125-127. In some embodiments, the tetherin inhibitor comprises an amino acid sequence of any one of SEQ ID NOs: 125-127. In some embodiments, presence or expression of the tetherin inhibitor in the cell results in an increase in ENP production by the cell by at least 2-fold, relative to a cell that does not comprise or express the tetherin inhibitor.

In some embodiments: less than about 10% of the ENPs of the population of ENPs have a particle size smaller than about 10 nm; less than about 10% of the ENPs of the population of ENPs have a particle size exceeding about 80 nm; the average diameter of the ENPs of the population of ENPs range from about 5 nm to about 80 nm, from about 15 nm to about 50 nm, or from about 20 nm to about 40 nm; and/or the average diameter of the ENPs of the population of ENPs is about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm. In some embodiments, the average is the mean, median or mode. In some embodiments, the mean is the arithmetic mean, geometric mean, and/or harmonic mean. In some embodiments, the ENPs: have a minimum diameter of about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm; have a maximum diameter of about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, about 50 nm, about 52 nm, about 54 nm, about 56 nm, about 58 nm, about 60 nm, about 62 nm, about 64 nm, about 66 nm, about 68 nm, about 70 nm, about 72 nm, about 74 nm, about 76 nm, about 78 nm, or about 80 nm; and/or are derived from cell cultures transiently transfected with the nucleic acid composition, e.g., derived via ultracentrifugation and/or size exclusion chromatography, e.g., ultracentrifugation on a 20% sucrose cushion, e.g., transfected via calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof. In some embodiments, the composition is stable for at least about 2 weeks, about 1 month, about 2 months, about 3 months, about 4 months, about 5 months, about 6 months, or about 1 year, after storage as a liquid at a temperature of about 4° C.

In some embodiments, the nucleic acid composition is complexed or associated with one or more lipids or lipid-based carriers, thereby forming liposomes, lipid nanoparticles (LNPs), lipoplexes, and/or nanoliposomes, e.g., encapsulating the nucleic acid composition. In some embodiments, the nucleic acid composition is, comprises, or further comprises, one or more vectors. In some embodiments, at least one of the one or more vectors is a viral vector, a plasmid, a transposable element, a naked DNA vector, a lipid nanoparticle (LNP), or any combination thereof. In some embodiments, the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. In some embodiments, the transposable element is piggybac transposon or sleeping beauty transposon.

In some embodiments, the polynucleotide encoding the fusion protein, the first polynucleotide encoding the dimerization fusion protein, the second polynucleotide encoding the adapter fusion protein, the fourth polynucleotide encoding the soluble RBP, and/or the fifth polynucleotide encoding the cell fusion protein are comprised in the one or more vectors. In some embodiments, the polynucleotide encoding the fusion protein, the first polynucleotide encoding the dimerization fusion protein, the second polynucleotide encoding the adapter fusion protein, the fourth polynucleotide encoding the soluble RBP, and/or the fifth polynucleotide encoding the cell fusion protein are comprised in the same vector and/or different vectors, In some embodiments, the polynucleotide encoding the fusion protein, the first polynucleotide encoding the dimerization fusion protein, the second polynucleotide encoding the adapter fusion protein, the fourth polynucleotide encoding the soluble RBP, and/or the fifth polynucleotide encoding the cell fusion protein are situated on the same nucleic acid and/or different nucleic acids.

In some embodiments: the polynucleotide encoding the fusion protein, the first polynucleotide encoding the dimerization fusion protein, the second polynucleotide encoding the adapter fusion protein, the fourth polynucleotide encoding the soluble RBP, and/or the fifth polynucleotide encoding the cell fusion protein are operably linked to one or more promoters capable of inducing transcription of said polynucleotide(s). In some embodiments: the promoter comprises a ubiquitous promoter, an inducible promoter, a tissue-specific promoter and/or a lineage-specific promoter. In some embodiments, the ubiquitous promoter is selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EFla) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof. In some embodiments, the polynucleotide encoding the fusion protein, the first polynucleotide encoding the dimerization fusion protein, the second polynucleotide encoding the adapter fusion protein, the fourth polynucleotide encoding the soluble RBP, and/or the fifth polynucleotide encoding the cell fusion protein are operably linked to a tandem gene expression element. In some embodiments, the tandem gene expression element is an internal ribosomal entry site (IRES), foot- and-mouth disease virus 2A peptide (F2A), equine rhinitis A virus 2A peptide (E2A), porcine teschovirus 2A peptide (P2A) or Thosea asigna virus 2A peptide (T2A), or any combination thereof. In some embodiments, the polynucleotide encoding the fusion protein, the first polynucleotide encoding the dimerization fusion protein, the second polynucleotide encoding the adapter fusion protein, the fourth polynucleotide encoding the soluble RBP, and/or the fifth polynucleotide encoding the cell fusion protein comprises a transcript stabilization element. In some embodiments, the transcript stabilization element comprises woodchuck hepatitis post-translational regulatory element (WPRE), bovine growth hormone polyadenylation (bGH-polyA) signal sequence, human growth hormone polyadenylation (hGH-polyA) signal sequence, or any combination thereof.

In some embodiments, the nucleic acid composition is or comprises mRNA. In some embodiments, the mRNA is formulated in a lipid nanoparticle (LNP). In some embodiments, the mRNA comprises: a 5′ untranslated region (UTR), a 3′ UTR, and/or a cap; one or more modified nucleotides selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, and 2-thiocytidine; and/or a modified nucleotide in place of one or more uridines. In some embodiments, the modified nucleoside is selected from pseudouridine (ψ), N 1-methyl-pseudouridine (m 1ψ), and 5-methyl-uridine (m5U). In some embodiments, the LNP comprises: one or more of an ionizable cationic lipid, a non-cationic lipid, a sterol, and a PEG-modified lipid, optionally the non-cationic lipid is a neutral lipid; 0.5-15 mol % PEG-modified lipid, 5-25 mol % non-cationic lipid, 25-55 mol % sterol, and 20-60 mol % ionizable cationic lipid; and/or 40-55 mol % ionizable cationic lipid, 5-15 mol % neutral lipid, 35-45 mol % sterol, and 1-5 mol % PEG-modified lipid. In some embodiments, the LNP comprises: 47 mol % ionizable cationic lipid, 11.5 mol % neutral lipid, 38.5 mol % sterol, and 3.0 mol % PEG-modified lipid; 48 mol % ionizable cationic lipid, 11 mol % neutral lipid, 38.5 mol % sterol, and 2.5 mol % PEG-modified lipid; 49 mol % ionizable cationic lipid, 10.5 mol % neutral lipid, 38.5 mol % sterol, and 2.0 mol % PEG-modified lipid; 50 mol % ionizable cationic lipid, 10 mol % neutral lipid, 38.5 mol % sterol, and 1.5 mol % PEG-modified lipid; or 51 mol % ionizable cationic lipid, 9.5 mol % neutral lipid, 38.5 mol % sterol, and 1.0 mol % PEG-modified lipid. In some embodiments: the ionizable cationic lipid is heptadecan-9-yl 8 ((2 hydroxyethyl) (6 oxo 6-(undecyloxy) hexyl) amino) octanoate; the neutral lipid is 1,2 distearoyl-sn-glycero-3 phosphocholine (DSPC); the sterol is cholesterol; and/or the PEG-modified lipid is 1-monomethoxypolyethyleneglycol-2,3-dimyristylglycerol with polyethylene glycol of average molecular weight 2000 (PEG2000 DMG). In some embodiments, the wt/wt ratio of lipid to mRNA is from about 1:100 to about 100:1.

In some embodiments, the composition is a lyophilized composition. In some embodiments, the lyophilized composition has a water content of less than about 10%. In some embodiments, the composition is formulated or is to be formulated: as a liquid, a solid, or a combination thereof; for injection; for intramuscular administration, intranasal administration, transdermal administration, aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection; and/or as particles. In some embodiments, the particles are iron oxide particles, liposomes, micelles, polymer complexes, cationic peptide nanoemulsions, virus-like particles (VLPs), lipid nanoparticles (LNP) and/or lipoplex (LPX) particles. In some embodiments, the nucleic acid composition and the LNP-forming components are in separate vials. In some embodiments, the composition is a pharmaceutical composition. In some embodiments, the pharmaceutical composition further comprises one or more pharmaceutically acceptable carriers, diluents and/or excipients. The composition can comprise instructions for use of the composition for: treating or preventing a disease or disorder; and/or diagnosing a subject as a subject having a disease or disorder. Disclosed herein include kits. In some embodiments, the kit comprises any of the composition of the disclosure. Also disclosed herein include cells comprising any of the nucleic acid compositions described herein. In some embodiments, the cell comprises a stem cell, a fibroblast cell, a chondrocyte, a keratinocyte, a hepatocyte, a pancreatic islet cell, or an immune cell. In some embodiments, the immune cell comprises a T cell, a dendritic cell (DC), a natural killer (NK) cell, or a macrophage. In some embodiments, the T cell is a CAR-T cell.

Disclosed herein include methods of delivering one or more cargo RNA molecules to a cell or a population of cells. In some embodiments, the method comprises contacting a cell or a population of cells with a composition of the disclosure, thereby delivering the one or more cargo RNA molecules to the cell or the population of cells. In some embodiments, the contacting step is performed in vivo, in vitro, and/or ex vivo. In some embodiments, the cell or the population of cells comprise one or more cells of a subject or are comprised within a tissue of a subject. In some embodiments, the subject is suffering from a disease or disorder. In some embodiments, the cell or the population of cells comprise prokaryotic cells or eukaryotic cells. In some embodiments, the eukaryotic cells comprise plant or animal cells.

In some embodiments, the cell or population of cells comprise an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

Disclosed herein include methods of treating or preventing a disease or disorder in a subject in need thereof, comprising: administering to the subject a pharmaceutically effective amount of a composition of any the disclosure, thereby treating or preventing the disease or disorder in the subject.

In some embodiments, the subject is a mammalian subject, e.g., a human subject. In some embodiments, the disease or disorder is a blood disease, an immune disease, a neurological disease or disorder, a cardiovascular disease or disorder, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof. In some embodiments, the disease or disorder is a solid tumor.

In some embodiments, the disease or disorder is an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-COV-2), Creutzfeldt-Jacob Disease/transmissible spongiform encephalopathy, Cryptosporidiosis (Crypto), Cyclosporiasis, Dengue 1,2,3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-COV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

In some embodiments, the disease is associated with expression of a tumor-associated antigen. In some embodiments, the disease associated with expression of a tumor antigen-associated is selected from the group consisting of a proliferative disease, a precancerous condition, a cancer, and a non-cancer related indication associated with expression of the tumor antigen. In some embodiments, the tumor associated antigen comprises a peptide-MHC complex, the peptide complexed with a class I or class II MHC sequence. In some embodiments, the peptide of the peptide-MHC complex is associated with a disease or disorder. In some embodiments, the peptide of the peptide-MHC complex is an intracellular tumor antigen;

In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, the disease or disorder is a cardiovascular disease. In some embodiments, the cardiovascular disease comprises angina, arrhythmia, atherosclerosis, atrial fibrillation, cardiomyopathy, congenital heart disease, coronary artery disease, enlarged heart, heart failure, infective endocarditis, an inherited rhythm disorder, Kawasaki disease, long Q-T syndrome, Marfan syndrome, pericarditis, peripartum cardiomyopathy, rheumatic heart disease, valvular heart disease, vascular cognitive impairment, or any combination thereof.

In some embodiments, the administering comprises aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-FIG. 1C display schematic presentations of designed ENP delivery systems for RNA cargoes. Shown in FIG. 1A is initial self-assembling ENP delivery systems requiring co-expression of three components: i) a fusion construct containing a cell surface protein (CSP), the RNA-binding protein (RBP), and the ESCRT-recruiting domain (ERD); ii) the soluble RBP to allow RBP dimerization/oligomerization and promote RNA binding; iii) the RNA cargo that contains the packaging sequence (PS). The RBP-PS interaction results in incorporation of the RNA cargo into budding ENPs. RNA packaging efficiency was improved by using an adapter approach (FIG. 1B), where the adapter contains the RBP and ERD sequences and interacts with the cytoplasmic domain of the CSP displayed on the ENP surface. The adapter system takes advantage of the known interaction between the N-terminal unique domain of the human tyrosine kinase Lck and the cytoplasmic domain of the human CD4 protein (CD4 CT), which is used to replace the native cytoplasmic domain of the CSP. As shown in FIG. 1C, delivery of the RNA cargo to specific target cells is achieved through co-display of two proteins on the ENP surface: i) a targeting protein that binds to a cell surface receptor that is specifically expressed on target cells; ii) a fusion protein that promotes fusion between the ENP envelope and cellular membrane to facilitate entry of the RNA cargo into the target cell cytoplasm.

FIG. 2 displays exemplary data showing engineered ENPs efficiently package specific RNA cargo. Plasmids encoding the indicated constructs were co-transfected into Expi293 cells. After 72 hours, ENPs were purified from supernatants by sucrose ultracentrifugation. RNA was extracted from purified ENPs, and Luc mRNA was quantified by RT-qPCR. Luc mRNA levels are presented as fold increase compared to Luc-CsrB.

FIG. 3A-FIG. 3B display exemplary data showing various RBP-PS interactions promote efficient packaging of a specific RNA cargo into ENPs. Plasmids encoding the indicated constructs were co-transfected into Expi293 cells. After 72 hours, ENPs were purified from supernatants by sucrose ultracentrifugation. RNA was extracted from purified ENPs, and Luc mRNA was quantified by RT-qPCR. Luc mRNA levels are presented as fold increase compared to S-EPM-CsrA-EABR+sCsrA+Luc-CsrB ENPs (FIG. 3A) or S-EPM-N-EABR+Luc-PS9 ENPs (FIG. 3B).

FIG. 4A-FIG. 4B display exemplary data showing co-expression of S-EPM-CsrA and sCsrA-EABR promotes ENP budding and RNA packaging through CsrA dimerization. Plasmids encoding the indicated constructs were co-transfected into Expi293 cells. After 72 hours, ENPs were purified from supernatants by sucrose ultracentrifugation. In FIG. 4A, RNA was extracted from purified ENPs, and Luc mRNA was quantified by RT-qPCR. Luc mRNA levels are presented as fold change compared to S-EPM-CsrA-EABR+sCsrA+Luc-CsrB ENPs. To quantify ENP budding, purified ENP samples were serially diluted, and S protein levels were measured by ELISA (FIG. 4B).

FIG. 5A-FIG. 5C display exemplary data showing an ERD adapter system improves ENP packaging of a specific RNA cargo. Plasmids encoding the indicated constructs were co-transfected into Expi293 cells. After 72 hours, ENPs were purified from supernatants by sucrose ultracentrifugation. As shown in FIG. 5A, RNA was extracted from purified ENPs, and Luc mRNA was quantified by RT-qPCR. Luc mRNA levels are presented as fold change compared to S-EPM-CsrA-EABR+sCsrA+Luc-CsrB ENPs. To quantify ENP budding, purified ENP samples were serially diluted, and S protein levels were measured by ELISA (FIG. 5B). In FIG. 5C, RNA was extracted from purified ENPs, and Luc mRNA was quantified by RT-qPCR. Luc mRNA levels are presented as fold change compared to S-EPM-N-EABR+sN+Lucmin-PS9 ENPs.

FIG. 6 displays exemplary data showing co-display of targeting protein and fusion protein on ENPs promotes efficient delivery of RNA cargo to target cells. Plasmids encoding the indicated constructs were co-transfected into HEK293T cells. SARS-COV-2 S protein was used as targeting protein due to its high affinity for the human ACE2 receptor. Two previously described proline mutations were introduced to stabilize S protein in its pre-fusion conformation, which ensured that S would only mediate receptor attachment but not membrane fusion. VSV-Gₘᵤₜ was selected as fusion protein. After 72 hours, ENPs were harvested from supernatants, concentrated, and frozen at −80° C. The concentrated samples were diluted 6-fold in cell culture media and added to pre-seeded HEK293T cells or HEK293T cells expressing the human ACE2 receptor (HEK293T-ACE2 cells) that were seeded in 96-well plates. After 16 hours, bioluminescence was measured. All samples were analyzed in duplicates. Rectangles and vertical lines represent the mean and standard deviation for each sample, respectively.

FIG. 7 displays exemplary data showing the CsrA-CsrB interaction does not promote efficient delivery of a specific RNA cargo to target cells. Plasmids encoding the indicated constructs were co-transfected into HEK293T cells. SARS-COV-2 S protein was used as targeting protein due to its high affinity for the human ACE2 receptor. Two previously described proline mutations were introduced to stabilize S protein in its pre-fusion conformation, which ensured that S would only mediate receptor attachment but not membrane fusion. VSV-Gₘᵤₜ was selected as fusion protein. After 72 hours, ENPs were harvested from supernatants, concentrated, and frozen at −80° C. The concentrated samples were diluted 6-fold in cell culture media and added to pre-seeded HEK293T cells or HEK293T-ACE2 cells that were seeded in 96-well plates. After 16 hours, bioluminescence was measured. All samples were analyzed in duplicates. Rectangles and vertical lines represent the mean and standard deviation for each sample, respectively.

FIG. 8 displays exemplary data showing the L7Ae-Box C/D interaction promotes efficient delivery of a specific RNA cargo to target cells. Plasmids encoding the indicated constructs were co-transfected into HEK293T cells. SARS-COV-2 S protein was used as targeting molecule due to its high affinity for the human ACE2 receptor. Two previously described proline mutations were introduced to stabilize S protein in its pre-fusion conformation, which ensured that S would only mediate receptor attachment but not membrane fusion. VSV-Gₘᵤₜ was selected as fusion protein. After 72 hours, ENPs were harvested from supernatants, concentrated, and frozen at −80° C. The concentrated samples were diluted 6-fold in cell culture media and added to pre-seeded HEK293T cells or HEK293T-ACE2 cells that were seeded in 96-well plates. After 16 hours, bioluminescence was measured. All samples were analyzed in duplicates. Rectangles and vertical lines represent the mean and standard deviation for each sample, respectively.

FIG. 9A-FIG. 9D display non-limiting exemplary data showing tetherin expression blocks ENP budding for SARS-COV-2 spike-EPM-EABR and influenza HA-EPM-EABR fusion constructs. In FIG. 9A-FIG. 9B, mRNAs encoding SARS-COV-2 spike and spike-EPM-EABR were synthesized and transfected into HEK293T cells. Spike-EPM-EABR was also co-transfected with indicated amounts of mRNA-encoded tetherin. 48 hours post-transfection, cell surface expression of spike was analyzed by flow cytometry (FIG. 9A). To quantify ENP budding, transfected cell culture supernatants were harvested and ENPs were purified by ultracentrifugation on a 20% sucrose cushion. Purified ENP samples were serially diluted, and spike protein levels were measured by ELISA (FIG. 9B). In FIG. 9C-FIG. 9D, mRNAs encoding influenza HA and HA-EPM-EABR were synthesized and transfected into HEK293T cells. HA-EPM-EABR was also co-transfected with indicated amounts of mRNA-encoded tetherin. 48 hours post-transfection, cell surface expression of HA was analyzed by flow cytometry (FIG. 9C). To quantify ENP budding, transfected cell culture supernatants were harvested and ENPs were purified by ultracentrifugation on a 20% sucrose cushion. Purified ENP samples were serially diluted, and HA protein levels were measured by ELISA (FIG. 9D).

FIG. 10A-FIG. 10D display non-limiting exemplary data showing HIV-1 Vpu co-expression antagonizes tetherin-mediated blockage of ENP budding. In FIG. 10A-FIG. 10B, mRNAs encoding SARS-COV-2 spike and spike-EPM-EABR were synthesized and transfected into HEK293T cells. Spike-EPM-EABR was also co-transfected with 0.025 μg of mRNA-encoded tetherin, 0.05 μg of mRNA-encoded Vpu, 0.025 μg of mRNA-encoded tetherin and 0.05 ug of mRNA-encoded Vpu, 0.025 μg of mRNA-encoded Vpu, or 0.025 μg of mRNA-encoded tetherin and 0.025 μg of mRNA-encoded Vpu. 48 hours post-transfection, cell surface expression of spike was analyzed by flow cytometry (FIG. 10A). To quantify ENP budding, transfected cell culture supernatants were harvested and ENPs were purified by ultracentrifugation on a 20% sucrose cushion. Purified ENP samples were serially diluted, and spike protein levels were measured by ELISA (FIG. 10B). In FIG. 10C-FIG. 10D, mRNAs encoding influenza HA and HA-EPM-EABR were synthesized and transfected into HEK293T cells. HA-EPM-EABR was also co-transfected with 0.025 μg of mRNA-encoded tetherin, 0.05 μg of mRNA-encoded Vpu, 0.025 μg of mRNA-encoded tetherin and 0.05 μg of mRNA-encoded Vpu, 0.025 μg of mRNA-encoded Vpu, or 0.025 μg of mRNA-encoded tetherin and 0.025 μg of mRNA-encoded Vpu. 48 hours post-transfection, cell surface expression of HA was analyzed by flow cytometry (FIG. 10C). To quantify ENP budding, transfected cell culture supernatants were harvested and ENPs were purified by ultracentrifugation on a 20% sucrose cushion. Purified ENP samples were serially diluted, and HA protein levels were measured by ELISA (FIG. 10D).

FIG. 11A-FIG. 11B display non-limiting exemplary data showing KSHV K5 and SARS-COV-2 ORF7a do not antagonize tetherin-mediated blockage of ENP budding. mRNAs encoding influenza HA and HA-EPM-EABR were synthesized and transfected into HEK293T cells. HA-EPM-EABR was also co-transfected with 0.025 μg of mRNA-encoded tetherin, 0.025 ug of mRNA-encoded tetherin and 0.1 μg of mRNA-encoded Vpu, 0.025 μg of mRNA-encoded tetherin and 0.025 μg of mRNA-encoded Vpu, 0.025 μg of mRNA-encoded tetherin and 0.1 μg of mRNA-encoded K5, 0.025 μg of mRNA-encoded tetherin and 0.025 μg of mRNA-encoded K5, 0.025 μg of mRNA-encoded tetherin and 0.1 μg of mRNA-encoded ORF7a, 0.025 μg of mRNA-encoded tetherin and 0.025 μg of mRNA-encoded ORF7a. 48 hours post-transfection, cell surface expression of HA was analyzed by flow cytometry (FIG. 11A). To quantify ENP budding, transfected cell culture supernatants were harvested and ENPs were purified by ultracentrifugation on a 20% sucrose cushion. Purified ENP samples were serially diluted, and HA protein levels were measured by ELISA (FIG. 11B).

FIG. 12A-FIG. 12B display exemplary data showing the N-PS9 and L7Ae-Box C/D combinations don't require co-expression of sN and sL7Ae, respectively. Plasmids encoding the indicated constructs were co-transfected into HEK293T cells using the N-PS9 (FIG. 12A) and L7Ae-Box C/D RBP-PS (FIG. 12B) interactions. SARS-COV-2 S protein was used as targeting molecule due to its high affinity for the human ACE2 receptor. Two previously described proline mutations were introduced to stabilize S protein in its pre-fusion conformation, which ensured that S would only mediate receptor attachment but not membrane fusion. VSV-Gₘᵤₜ was selected as fusion protein. After 72 hours, ENPs were harvested from supernatants, concentrated, and frozen at −80° C. The concentrated samples were diluted 6-fold in cell culture media and added to pre-seeded HEK293T cells or HEK293T-ACE2 cells that were seeded in 96-well plates. After 16 hours, bioluminescence was measured. All samples were analyzed in duplicates. Rectangles and vertical lines represent the mean and standard deviation for each sample, respectively.

FIG. 13 shows a schematic presentation of ENP delivery systems using the L7Ae-Box C/D and N-PS9 RBP-PS combinations. Self-assembling ENP delivery systems using the L7Ae-Box C/D and N-PS9 RBP-PS combinations require co-expression of four components: i) a membrane-anchored targeting protein (e.g., SARS-COV-2 Spike) fused to the CD4 cytoplasmic domain (CD4 CT); ii) a Lck-RBP-ERD fusion protein that contains the N-terminal unique domain of Lck, the RBP (e.g., L7Ae or N), and the ERD. The Lck domain interacts with the CD4 CT to incorporate the targeting protein on the ENP surface, the RBP recruits the RNA cargo by interacting with the PS, and the ERD interacts with ESCRT proteins to induce ENP budding; iii) the RNA cargo that contains the PS (e.g., Box C/D or PS9); iv) a fusion protein that promotes fusion between the ENP envelope and cellular membrane to facilitate entry of the RNA cargo into the target cell cytoplasm (e.g., VSV-Gₘᵤₜ). Note that this schematic does not include the soluble RBP as shown in FIG. 1A-FIG. 1C since the L7Ae and N RBPs do not require co-expression of soluble L7Ae/N to promote efficient RNA packaging and delivery.

FIG. 14A-FIG. 14B display exemplary data showing ENPs efficiently deliver mRNA encoding the fluorescent protein tdTomato to target cells. Plasmids encoding the indicated constructs were co-transfected into HEK293T cells. SARS-COV-2 S protein was used as targeting molecule due to its high affinity for the human ACE2 receptor. Two previously described proline mutations were introduced to stabilize S protein in its pre-fusion conformation, which ensured that S would only mediate receptor attachment but not membrane fusion. VSV-Gₘᵤₜ was selected as fusion protein. After 72 hours, ENPs were harvested from supernatants, concentrated, and frozen at −80° C. The concentrated samples were diluted 6-fold in cell culture media and added to pre-seeded HEK293T cells or HEK293T-ACE2 cells that were seeded in 96-well plates. After 16 hours, tdTomato expression was analyzed by flow cytometry. Flow cytometry data are presented as histograms for HEK293T and HEK293T-ACE2 cells (FIG. 14A). Mean fluorescent intensities (arbitrary units) are shown for indicated conditions in HEK293T and HEK293T-ACE2 cells (FIG. 14B).

FIG. 15 displays exemplary data showing ENPs efficiently deliver mRNA encoding the fluorescent protein zsGreen to target cells. Plasmids encoding the indicated constructs were co-transfected into HEK293T cells. SARS-COV-2 S protein was used as targeting molecule due to its high affinity for the human ACE2 receptor. Two previously described proline mutations were introduced to stabilize S protein in its pre-fusion conformation, which ensured that S would only mediate receptor attachment but not membrane fusion. VSV-Gₘᵤₜ was selected as fusion protein. After 72 hours, ENPs were harvested from supernatants, concentrated, and frozen at −80° C. The concentrated samples were diluted 6-fold in cell culture media and added to pre-seeded HEK293T cells or HEK293T-ACE2 cells that were seeded in 96-well plates. After 16 hours, zsGreen expression was analyzed by fluorescence microscopy.

FIG. 16 shows a schematic presentation of ENPs displaying scFv targeting proteins to deliver RNA cargoes to a wide range of specific cell types. Self-assembling ENP delivery systems requires co-expression of four components: i) a membrane-anchored scFv-based targeting protein fused to the CD4 cytoplasmic domain (CD4 CT), the scFv targeting protein binds to a specific cell surface receptor on the target cell; ii) a Lck-RBP-ERD fusion protein that contains the N-terminal unique domain of Lck, the RBP, and the ERD, the Lck domain interacts with the CD4 CT to incorporate the scFv targeting protein on the ENP surface, the RBP recruits the RNA cargo by interacting with the PS, and the ERD interacts with ESCRT proteins to induce ENP budding; iii) the RNA cargo that contains the PS; iv) a fusion protein that promotes fusion between the ENP envelope and cellular membrane to facilitate entry of the RNA cargo into the target cell cytoplasm. Note that this schematic does not include the soluble RBP as shown in FIG. 1A-FIG. 1C since the L7Ae and N RBPs do not require co-expression of soluble L7Ae/N to promote efficient RNA packaging and delivery.

FIG. 17 displays exemplary data showing the aCD19 scFv-CD4 CT-EPM targeting protein promotes efficient mRNA cargo delivery to target cells that express CD19. Plasmids encoding the indicated constructs were co-transfected into HEK293T cells. The aCD19 scFv-CD4 CT-EPM targeting protein was designed to target ENPs to cells that express the human CD19 receptor. VSV-Gₘᵤₜ was selected as fusion protein. After 72 hours, ENPs were harvested from supernatants, concentrated, and frozen at −80° C. The concentrated samples were diluted 6-fold in cell culture media and added to pre-seeded HEK293T or HEK293T-CD19 cells that were seeded in 96-well plates. After 16 hours, bioluminescence was measured. All samples were analyzed in duplicates. Rectangles and vertical lines represent the mean and standard deviation for each sample, respectively.

FIG. 18 displays exemplary data showing ENPs co-displaying aCD3 and aCD4 scFv-CD4 CT-EPM targeting proteins efficiently deliver an mRNA cargo to CD4 T cells. Plasmids encoding the indicated constructs were co-transfected into HEK293T cells. The aCD3 and aCD4 scFv-CD4 CT-EPM targeting proteins were designed to target ENPs to CD4+ T cells that express the CD3 and CD4 receptors. VSV-Gₘᵤₜ was selected as fusion protein. After 72 hours, ENPs were harvested from supernatants, concentrated, and frozen at −80° C. The concentrated samples were diluted 6-fold in cell culture media and added to Jurkat CD4 T cells that were seeded in 96-well plates. After 16 hours, bioluminescence was measured. All samples were analyzed in duplicates. Rectangles and vertical lines represent the mean and standard deviation for each sample, respectively.

FIG. 19 displays exemplary data showing ENPs efficiently deliver mRNA-encoded IL 12 to target cells that express CD19. Plasmids encoding the indicated constructs were co-transfected into HEK293T cells. The aCD19 scFv-CD4 CT-EPM targeting protein was designed to target ENPs to cells that express the human CD19 receptor. VSV-Gₘᵤₜ was selected as fusion protein. After 72 hours, ENPs were harvested from supernatants, purified by ultracentrifugation on a 20% sucrose cushion, and frozen at −80° C. The concentrated samples were serially diluted in cell culture media and added to pre-seeded HEK293T or HEK293T-CD19 cells that were seeded in 24-well plates. After 21 hours, supernatants were collected and IL12 concentrations were measured by ELISA.

FIG. 20 displays exemplary data showing ENPs efficiently deliver mRNA-encoded single-chain IL 12 fusion protein to target cells that express CD19. Plasmids encoding the indicated constructs were co-transfected into HEK293T cells. The aCD19 scFv-CD4 CT-EPM targeting protein was designed to target ENPs to cells that express the human CD19 receptor. VSV-Gₘᵤₜ was selected as fusion protein. After 72 hours, ENPs were harvested from supernatants, purified by ultracentrifugation on a 20% sucrose cushion, and frozen at −80° C. The concentrated samples were serially diluted in cell culture media and added to pre-seeded HEK293T or HEK293T-CD19 cells that were seeded in 24-well plates. After 21 hours, supernatants were collected and IL 12 concentrations were measured by ELISA.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include compositions. In some embodiments, the composition comprises:

a nucleic acid composition comprising a polynucleotide encoding a fusion protein and one or more polynucleotides comprising one or more cargo RNA molecules each comprising a packing signal, wherein the fusion protein comprises a cell-surface protein (CSP), an RNA-binding protein (RBP), and an endosomal sorting complex required for transport (ESCRT)-recruiting domain (ERD), wherein the RBP is capable of binding the packing signal, and wherein a plurality of fusion proteins are capable of self-assembling into an enveloped nanoparticle (ENP) secreted from a cell in which the plurality of fusion proteins are expressed, thereby generating a population of ENPs comprising the fusion protein and the one or more cargo RNA molecules, optionally the nucleic acid composition further comprises a polynucleotide encoding a soluble RBP capable of binding the packing signal.

Disclosed herein include compositions. In some embodiments, the composition comprises: a population of enveloped nanoparticles (ENPs), wherein each of the ENPs comprises: (i) a plurality of fusion proteins each comprising a cell-surface protein (CSP), an RNA-binding protein (RBP), and an endosomal sorting complex required for transport (ESCRT)-recruiting domain (ERD); and (ii) one or more cargo RNA molecules each comprising a packing signal.

Disclosed herein include compositions. In some embodiments, the composition comprises: a nucleic acid composition comprising: (i) a first polynucleotide encoding a dimerization fusion protein, wherein the dimerization fusion protein comprises a cell surface protein (CSP) and a heterologous cytoplasmic tail, optionally the dimerization fusion protein further comprises an RNA-binding protein (RBP) and/or an endosomal sorting complex required for transport (ESCRT)-recruiting domain (ERD); (ii) a second polynucleotide encoding an adapter fusion protein comprising an adapter domain capable of binding the heterologous cytoplasmic tail to form a heterodimer, an optional RBP, and an optional endosomal sorting complex required for transport (ESCRT)-recruiting domain (ERD); and (iii) one or more third polynucleotides comprising one or more cargo RNA molecules each comprising a packing signal, wherein the RBP of (i) and (ii) are each capable of binding the packing signal, wherein binding of the adapter domain to the heterologous cytoplasmic tail is capable of recruiting one or more ESCRT proteins to the heterodimer, thereby inducing a plurality of dimerization fusion proteins to self-assemble into an enveloped nanoparticle (ENP) secreted from a cell in which the dimerization fusion protein and adapter fusion protein are expressed, thereby generating a population of ENPs comprising the dimerization fusion protein and the one or more cargo RNA molecules, optionally the nucleic acid composition further comprises a fourth polynucleotide encoding a soluble RBP capable of binding the packing signal.

Disclosed herein include compositions. In some embodiments, the composition comprises: a population of enveloped nanoparticles (ENPs), wherein each of the ENPs comprises: (i) a plurality of dimerization fusion proteins each comprising a heterologous cytoplasmic tail and a CSP, optionally the CSP is a targeting protein capable of targeting the ENPs to a target cell; (ii) one or more cargo RNA molecules each comprising a packing signal; and optionally (iii) a plurality of cell fusion proteins.

Disclosed herein include kits. In some embodiments, the kit comprises any of the composition of the disclosure. Also disclosed herein include cells comprising any of the nucleic acid compositions described herein.

Disclosed herein include methods of delivering one or more cargo RNA molecules to a cell or a population of cells. In some embodiments, the method comprises contacting a cell or a population of cells with a composition of the disclosure, thereby delivering the one or more cargo RNA molecules to the cell or the population of cells. Disclosed herein include methods of treating or preventing a disease or disorder in a subject in need thereof, comprising: administering to the subject a pharmaceutically effective amount of a composition of any the disclosure, thereby treating or preventing the disease or disorder in the subject.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “about” means plus or minus 5% of the provided value.

As used herein, “sequence identity” or “identity” in the context of two nucleic acid or polypeptide sequences makes reference to the nucleotide bases or residues in the two sequences that are the same when aligned for maximum correspondence over a specified comparison window. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; Pearson et al., Meth. Mol. Bio. 24:307-31, 1994; and Altschul et al., J. Mol. Biol. 215:403-10, 1990 (the content of each of these references is incorporated herein in its entirety).

When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule. A functionally equivalent residue of an amino acid used herein typically can refer to other amino acid residues having physiochemical and stereochemical characteristics substantially similar to the original amino acid. The physiochemical properties include water solubility (hydrophobicity or hydrophilicity), dielectric and electrochemical properties, physiological pH, partial charge of side chains (positive, negative or neutral) and other properties identifiable to one of skill in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

The term “substantially identical” as used herein in the context of two or more sequences refers to a specified percentage of amino acid residues or nucleotides that are identical or functionally equivalent, such as about, at least or at least about 65% identity, optionally, about, at least or at least about 70%, 75%, 80%, 85%, 90%, 95%, or 99% identity over a specified region or over the entire sequence.

As used herein, the term “variant” refers to a polynucleotide or polypeptide having a sequence substantially similar or identical to a reference (e.g., the parent) polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least, or at least about, 50%, 55%, 60%, 65%, 70%, 75%, 80%, 85%, 90%, 91%, 92%, 93%, 94%, 95%, 96%, 97%, 98%, 99%, or more sequence identity to the reference polynucleotide as determined by sequence alignment programs known in the art. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. A variant of a polypeptide can have, for example, at least, or at least about, 60%, 65%, 70%, 75%, 80%, 85%, 90%, 91%, 92%, 93%, 94%, 95%, 96%, 97%, 98%, 99%, or more sequence identity to the reference polypeptide as determined by sequence alignment programs known in the art.

As used herein in the term “derived from”, in the context of an amino acid sequence or polynucleotide sequence (e.g., an amino acid sequence “derived from” a mammalian protein), is meant to indicate that the polypeptide or nucleic acid has a sequence that is based on that of a reference polypeptide or nucleic acid (e.g., a naturally occurring mammalian protein), and is not meant to be limiting as to the source or method in which the protein or nucleic acid is made. By way of example, the term “derived from” includes homologs or variants of reference amino acid or DNA sequences.

As used herein, the terms “antigen” or “immunogen” are used interchangeably to refer to a substance, typically a protein, which is capable of inducing an immune response in a subject (e.g., a mammal, such as a human). The term also refers to proteins that are immunologically active in the sense that once administered to a subject, either directly or in the form of a nucleotide sequence or vector that encodes the protein, is able to evoke an immune response of the humoral and/or cellular type directed against that protein or a variant thereof.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those commonly known and used in the art. Standard techniques can be used for chemical syntheses, chemical analyses, pharmaceutical preparation, formulation, and delivery, and treatment of patients.

The term “construct,” as used herein, refers to a recombinant nucleic acid that has been generated for the purpose of the expression of a specific nucleotide sequence(s), or that is to be used in the construction of other recombinant nucleotide sequences.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

Disclosed herein are various enveloped nanoparticle (ENP) delivery systems that encapsulate specific RNA cargoes and efficiently deliver these cargoes to target cells. ENP encapsulation of specific RNA cargoes can be achieved by inserting an ESCRT-recruiting domain (ERD) and a sequence-specific RNA-binding protein (RBP) into the cytoplasmic domain of a cell surface protein (CSP). The ERD recruits proteins from the Endosomal Sorting Complex Required for Transport (ESCRT) pathway to induce ENP budding. The RBP recruits the RNA cargo that contains a packaging sequence (PS) that specifically interacts with the RBP to facilitate incorporation of the RNA cargo into budding ENPs. For RBPs that require dimerization or oligomerization for RNA-binding, co-expression of the soluble RBP facilitates dimerization/oligomerization and increases RNA packaging efficiency. The design of a multifunctional adapter construct further improves ENP encapsulation of specific RNA cargoes. This adapter construct is targeted to the cytoplasmic side of the plasma membrane and includes the RBP and ERD. The adapter also contains sequences that interact with the modified cytoplasmic domain of a CSP that is recruited to and displayed on the surface of the ENPs. Delivery of the RNA cargo to specific cell types requires display of CSPs that mediate ENP attachment to specific target cells and membrane fusion to facilitate entry of the RNA cargo into cells. For optimal programmability of ENP target cell tropism, ENP delivery systems were designed that co-display two CSPs: i) an ENP targeting protein that mediates attachment to cell surface receptors specifically presented on target cells; ii) a fusion protein that induces fusion between the ENP envelope and cellular membrane to facilitate entry of the RNA cargo into the target cell cytoplasm.

The designed modular ENP delivery systems can be tailored to package nucleic acid-based cargoes for a wide range of therapeutic, diagnostic, and biological applications. The RNA cargo can have therapeutic, diagnostic, or biological activity or encode a protein with therapeutic, diagnostic, or biological activity. Various targeting proteins can be displayed to target ENPs to specific cell types in vivo, thereby maximizing therapeutic concentrations of RNA cargoes in target tissues and minimizing adverse effects in other tissues. In comparison to EVs and other biological nanoparticle-based platforms, the described self-assembling ENP delivery system is more suitable for large-scale manufacturing due to the high production rate and lack of cellular toxicity.

Disclosed herein include compositions, methods, and kits for self-assembly of ENPs for delivery of nucleic acid cargoes. Disclosed herein include compositions. In some embodiments, the composition comprises: a nucleic acid composition comprising a polynucleotide encoding a fusion protein and one or more polynucleotides comprising one or more cargo RNA molecules each comprising a packing signal, wherein the fusion protein comprises a cell-surface protein (CSP), an RNA-binding protein (RBP), and an endosomal sorting complex required for transport (ESCRT)-recruiting domain (ERD), wherein the RBP is capable of binding the packing signal, and wherein a plurality of fusion proteins are capable of self-assembling into an enveloped nanoparticle (ENP) secreted from a cell in which the plurality of fusion proteins are expressed, thereby generating a population of ENPs comprising the fusion protein and the one or more cargo RNA molecules, optionally the nucleic acid composition further comprises a polynucleotide encoding a soluble RBP capable of binding the packing signal.

The fusion protein can be capable of being presented on the surface of the cell in which the fusion protein is expressed. In some embodiments, the self-assembly of an ENP does not require an exogenous nucleic acid other than the nucleic acid composition. In some embodiments, the cell is: a cell of a subject; an in vivo cell, an ex vivo cell, or an in situ cell; and/or an adherent cell or a suspension cell. Upon secretion from a cell of a subject, the ENPs can be capable of distributing within one or more tissues of the subject. In some embodiments, the one or more tissues comprise adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue, fat tissue, or any combination thereof. In some embodiments, the one or more tissues comprise diseased tissues, e.g., cancerous or infected tissues.

In some embodiments, the composition comprises: a population of enveloped nanoparticles (ENPs), wherein each of the ENPs comprises: (i) a plurality of fusion proteins each comprising a cell-surface protein (CSP), an RNA-binding protein (RBP), and an endosomal sorting complex required for transport (ESCRT)-recruiting domain (ERD); and (ii) one or more cargo RNA molecules each comprising a packing signal. In some embodiments, the population of ENPs is derived from expression of the nucleic acid composition of the disclosure. In some embodiments, the ENPs comprise a lipid bilayer, e.g., a lipid bilayer derived from the cell from which the ENP was secreted.

Provided herein are RNA packing signals and RNA binding proteins (RBPs). Any of the engineered proteins provided herein (e.g., fusion protein, dimerization fusion protein, and/or an adapter fusion protein) can comprise one or more RBPs. In some embodiments, a fusion protein comprises an RBP and the one or more cargo RNA molecules each comprise a packing signal.

The packing signal can be derived from any organism. The packing signal and the RBP can be derived from a viral, archaeal, bacterial, or mammalian packing signal and RBP, or variants thereof. The packing signal can comprise a Ku binding hairpin and the RBP and/or the soluble RBP can be Ku. The packing signal can comprise a telomerase Sm7 binding motif and the RBP and/or the soluble RBP can be Sm7. The packing signal can comprise an MS2 phage operator stem-loop and the RBP and/or the soluble RBP can be MS2 Coat Protein (MCP). The packing signal can comprise a PP7 phage operator stem-loop and the RBP and/or the soluble RBP can be PP7 Coat Protein (PCP). The packing signal can comprise an SfMu phage Com stem-loop and the RBP and/or the soluble RBP can be Com RNA binding protein. The packing signal can comprise a PUF binding site (PBS) and the RBP and/or the soluble RBP can be Pumilio/fem-3 mRNA binding factor (PUF). The packing signal can comprise Psi and the RBP and/or the soluble RBP can be gag, optionally derived from MMLV, HIV, SIV, FIV, HTLV, or Foamy viruses. The packing signal can comprise regulatory RNA CsrB and the RBP and/or the soluble RBP can be CsrA of E. coli. The packing signal can comprise PS9 and the RBP and/or the soluble RBP can be N protein of a coronavirus, optionally SARS-COV-2. The packing signal can comprise Box C/D binding motif and the RBP and/or the soluble RBP can be ribosomal protein L7Ae of archaea.

The packing signal can comprise regulatory RNA CsrB and the RBP and/or the soluble RBP can be CsrA of E. coli. The packing signal can comprise a nucleotide sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the nucleotide sequence of SEQ ID NO: 134 and the RBP and/or the soluble RBP can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the amino acid sequence of SEQ ID NO: 128. The packing signal can comprise a nucleotide sequence having one, two, or three mismatches relative to the nucleotide sequence of SEQ ID NO: 134 and the RBP and/or the soluble RBP can comprise an amino acid sequence having one, two, or three mismatches relative to the amino acid sequence of SEQ ID NO: 128.

The packing signal can comprise an MS2 phage operator stem-loop and the RNA binding protein can be MS2 Coat Protein (MCP). The packing signal can comprise a nucleotide sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the nucleotide sequence of SEQ ID NO: 152 and the RBP and/or the soluble RBP can comprise an amino acid sequence having at least 65%, 70%, 75%, 80%, 85%, 90%, 95%, 98%, or 99% sequence identity to the amino acid sequence of SEQ ID NO: 148. (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values). The packing signal can comprise a nucleotide sequence having one, two, or three mismatches relative to the nucleotide sequence of SEQ ID NO: 152 and the RBP and/or the soluble RBP can comprise an amino acid sequence having one, two, or three mismatches relative to the amino acid sequence of SEQ ID NO: 148.

The packing signal can comprise PS9 and the RBP and/or the soluble RBP can be N protein of SARS-COV-2. The packing signal can comprise a nucleotide sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the nucleotide sequence of SEQ ID NO: 140 and the RBP and/or the soluble RBP can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the amino acid sequence of SEQ ID NO: 136. The packing signal can comprise a nucleotide sequence having one, two, or three mismatches relative to the nucleotide sequence of SEQ ID NO: 140 and the RBP and/or the soluble RBP can comprise an amino acid sequence having one, two, or three mismatches relative to the amino acid sequence of SEQ ID NO: 136.

The packing signal can comprise Box C/D binding motif and the RBP and/or the soluble RBP can be ribosomal protein L7Ae of archaea. The packing signal can comprise a nucleotide sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the nucleotide sequence of SEQ ID NO: 146 and the RBP and/or the soluble RBP can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the amino acid sequence of SEQ ID NO: 142. The packing signal can comprise a nucleotide sequence having one, two, or three mismatches relative to the nucleotide sequence of SEQ ID NO: 146 and the RBP and/or the soluble RBP can comprise an amino acid sequence having one, two, or three mismatches relative to the amino acid sequence of SEQ ID NO: 142.

The packing signal can comprise regulatory RNA CsrB and the RBP and/or the soluble RBP can be CsrA of E. coli. The packing signal can comprise the nucleotide sequence of SEQ ID NO: 134 and the RBP and/or the soluble RBP can comprise the amino acid sequence of SEQ ID NO: 128. The packing signal can comprise an MS2 phage operator stem-loop and the RNA binding protein can be MS2 Coat Protein (MCP). The packing signal can comprise the nucleotide sequence of SEQ ID NO: 152 and the RBP and/or the soluble RBP can comprise the amino acid sequence of SEQ ID NO: 148. The packing signal can comprise PS9 and the RBP and/or the soluble RBP can be N protein of SARS-COV-2. The packing signal can comprise the nucleotide sequence of SEQ ID NO: 140 and the RBP and/or the soluble RBP can comprise the amino acid sequence of SEQ ID NO: 136. The packing signal can comprise Box C/D binding motif and the RBP and/or the soluble RBP can be ribosomal protein L7Ae of archaea. The packing signal can comprise the nucleotide sequence of SEQ ID NO: 146 and the RBP and/or the soluble RBP can comprise the amino acid sequence of SEQ ID NO: 142.

The packing signal can be placed at any position within a polynucleotide (e.g., the one or more polynucleotides comprising one or more cargo RNA molecules). The packing signal can be situated at the 5′ end or the 3′ end of at least one of the one or more RNA cargo molecules. At least one of the one or more RNA cargo molecules comprise an mRNA, and the packing signal is situated within the 5′ or 3′ UTR of the mRNA.

An engineered protein of the disclosure (e.g., a fusion protein), can comprise an endosomal sorting complex required for transport (ESCRT)-recruiting domain (ERD). The ERD can be capable of recruiting one or more ESCRT proteins to the cytoplasmic tail of the fusion protein. The recruitment of ESCRT proteins via the ERD can be capable of inducing the self-assembly and budding of ENPs. The ERD can be located at the C-terminus of the fusion protein, the N-terminus of the fusion protein, or between the N-terminus and the C-terminus of the fusion protein. The ERD can be capable of interacting with ESCRT proteins TSG101, NEDD4, and/or ALIX.

In some embodiments, the ERD comprises or is derived from a nonhuman protein. In some embodiments, the ERD comprises or is derived from a human protein. In some embodiments, the ERD comprises or is derived from a nonmammalian protein. In some embodiments, the ERD comprises or is derived from a chicken protein, a mouse protein, a lizard protein, a reptile protein, a hamster protein, or a goldfish protein. In some embodiments, the ERD comprises or is derived from the ESCRT and ALIX binding region (EABR) of the human CEP55 protein. In some embodiments, the ERD comprises or is derived from residues 170-213 of the human CEP55 protein. In some embodiments, the ERD comprises or is derived from Syntenin-1, rat Galectin-3 (rGalectin-3), Hrs, and/or CD2AP. In some embodiments, the ERD comprises or is derived from a viral protein. In some embodiments, the ERD comprises or is derived from a fragment of a viral protein. In some embodiments, the ERD comprises or is derived from a retroviral protein, herpes simplex viral protein, vaccinia viral protein, hepadnaviral protein, togaviral protein, flaviviral protein, arenaviral protein, coronaviral protein, orthomyxoviral protein, paramyxoviral protein, bunyaviral protein, bornaviral protein, rhabdoviral protein or filoviral protein. In some embodiments, the ERD comprises or is derived from a Gag protein. In some embodiments, the ERD comprises or is derived from EIAV, HTLV-1, MLV, or MPMV. In some embodiments, the ERD comprises or is derived from EIAV p9, SIV p6 and/or HIV-1 p6; and/or an Ebola protein. In some embodiments, the ERD comprises or is derived from EBOV VP40.

In some embodiments, the ERD comprises or is derived from at least a portion of CEP55 protein. The ERD can comprise an amino acid sequence having at least 65%, 70%, 75%, 80%, 85%, 90%, 95%, 98%, or 99% sequence identity (e.g., at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) to the amino acid sequence of SEQ ID NO: 2. In some embodiments, the ERD comprises a sequence having one, two, or three mismatches relative to the sequence of SEQ ID NO: 2.

The ERD can comprise one or more TSG101-binding motifs, one or more ALIX-binding motifs, one or more Nedd4-recruiting motifs, or any combination thereof. In some embodiments, any two of the one or more TSG101-binding motifs, the one or more ALIX-binding motifs, or the one or more Nedd4-recruiting motifs are the same or different. In some embodiments, the ERD comprises or is derived from HIV-1 p6 protein or SIV p6 protein. In some embodiments, the ERD comprises or is derived from the p6 protein of HIV-1 isolate ETH2220. In some embodiments, the ERD comprises or is derived from a non-human galectin protein. In some embodiments, the ERD comprises or is derived from a rat galectin protein.

The ERD can comprise one or more TSG101-binding motifs, one or more ALIX-binding motifs, one or more Nedd4-recruiting motifs, or any combination thereof. Any two of the one or more TSG101-binding motifs, the one or more ALIX-binding motifs, or the one or more Nedd4-recruiting motifs can be the same or different. The ERD can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the amino acid sequence of SEQ ID NO: 2. The ERD can comprise an amino acid sequence having one, two, or three mismatches relative to the amino acid sequence of SEQ ID NO: 2. The ERD can comprise the amino acid sequence of SEQ ID NO: 2. The ERD comprises or is derived from a non-human galectin protein, e.g., a rat galectin protein. The ERD can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to an amino acid sequence selected from the group consisting of SEQ ID NOs: 3-39. The ERD can comprise an amino acid sequence having one, two, or three mismatches relative to any one of SEQ ID NOs: 3-39. In some embodiments, the ERD comprises or consists of the sequence of any one of SEQ ID NOs: 3-39. Provided in Table 1 are exemplary ERDs that can be used, e.g., in any fusion protein of the disclosure (e.g., fusion proteins, dimerization fusion proteins, and/or adapter fusion proteins).

TABLE 1
Exemplary ERDs

| NAME | SEQ ID NO: | SEQUENCE |
|---|---|---|
| EPM | 1 | ALPGNPDHREMGETLPEEVGEYRQPSGGSVPVSPGPPSGLEPT |
| | | SSSPY |
| EABR | 2 | FNSSINNIHEMEIQLKDALEKNQQWLVYDQQREVYVKGLLAK |
| | | IFELEKKTETAAHSLP |
| EIAV p9 | 3 | PIQQKSQHNKSVVQETPQTQNLYPDLSEIKKEYNVKEKDQVE |
| | | DLNLDSLWE |
| HIV-1 p6 | 4 | LQSRPEPTAPPEESFRSGVETTTPPQKQEPIDKELYPLTSLRSLF |
| (HXB2) | | GNDPSSQ |
| p6v2.1 ERD | 5 | LQSRPEPTAPPESLRPEPTAPPPESFRFEEATPSPKQELKDREAL |
| | | TSLKSLFGNDHLLQ |
| p6v2.2 ERD | 6 | LQSRPEPTAPPESLRPEPTAPPPESFRFEEATPSPKQELKDREPY |
| | | KEALTSLKSLFGNDHLLQ |
| p6v2.3 ERD | 7 | LQSRPEPTAPPESLRPEPTAPPPESFRFEEATPSPKQELKDREAL |
| | | TSLKSLFGNDHLLQGGSQTQNLYPDLSEIKKE |
| p6v2.4 ERD | 8 | LQSRPEPTAPPESLRPEPTAPPPESFRFEEATPSPKQELKDREAL |
| | | TSLKSLFGNDHLLQGGSQTQNLYPDLSEIKKEQTQNLYPDLSEI |
| | | KKE |
| p6v2.5 ERD | 9 | LQSRPEPTAPPESLRPEPTAPPPESFRFEEATPSPKQELKDREPY |
| | | KEALTSLKSLFGNDHLLQGGSQTQNLYPDLSEIKKE |
| p6v2.6 ERD | 10 | LQSRPEPTAPPESLRPEPTAPPESLRPEPTAPPPESFRFEEATPSP |
| | | KQELKDREPYKEALTSLKSLFGNDHLLQ |
| p6v2.7 ERD | 11 | LQSRPEPTAPPESLRPEPTAPPESLRPEPTAPPPESFRFEEATPSP |
| | | KQELKDREPYKEALTSLKSLFGNDHLLQGGSQTQNLYPDLSEI |
| | | KKE |
| p6v3.1 ERD | 12 | LQSRPEPTAPPESLRPEPTAPPPESFRFEEATPSPKQELKDREPY |
| | | KEALTDREPYKEALTSLKSLFGNDHLLQ |
| p6v3.2 ERD | 13 | LQSRPEPTAPPESLRPEPTAPPPESFRFEEATPSPKQELKDREPY |
| | | KELYPLTSLKSLFGNDHLLQ |
| p6v3.3 ERD | 14 | LQSPPPYRPEPTAPPESLRPEPTAPPPESFRFEEATPSPKQELKDR |
| | | EPYKEALTSLKSLFGNDHLLQ |
| p6v3.4 ERD | 15 | LQSRPEPTAPPESLRPEPTAPPPYESFRFEEATPSPKQELKDREP |
| | | YKEALTSLKSLFGNDHLLQ |
| p6v3.5 ERD | 16 | LQSRPEPTAPPESLRPEPTAPPPESFRFEEATPSPKQELKDREPY |
| | | KELYPLTSLKSLYPLTSLKSLFGNDHLLQ |
| p6v4.1 ERD | 17 | LQSPPPYRPEPTAPPESLRPEPTAPPPESFRFEEATPSPKQELKDR |
| | | EPYKELYPLTSLKSLFGNDHLLQ |
| p6v4.2 ERD | 18 | LQSRPEPSAPPESLRPEPSAPPPESFRFEEATPSPKQELKDREPY |
| | | KELYPLTSLKSLFGNDHLLQ |
| p6v4.3 ERD | 19 | LQSPPPYRPEPSAPPESLRPEPSAPPPESFRFEEATPSPKQELKDR |
| | | EPYKELYPLTSLKSLFGNDHLLQ |
| p6v4.4 ERD | 20 | PMAQVHQGLTPTAPPEDGLTPTAPPEDPAVDLLKNYMQLGKQ |
| | | QRESREKPYKELYPLTSLVTEDLLHLNSLFGGDQ |
| p6v4.5 ERD | 21 | PMAQVPPPYHQGLTPTAPPEDGLTPTAPPEDPAVDLLKNYMQ |
| | | LGKQQRESREKPYKELYPLTSLVTEDLLHLNSLFGGDQ |
| p6v4.6 ERD | 22 | PMAQVHQGLTPSAPPEDGLTPSAPPEDPAVDLLKNYMQLGKQ |
| | | QRESREKPYKELYPLTSLVTEDLLHLNSLFGGDQ |
| p6v4.7 ERD | 23 | PMAQVPPPYHQGLTPSAPPEDGLTPSAPPEDPAVDLLKNYMQL |
| | | GKQQRESREKPYKELYPLTSLVTEDLLHLNSLFGGDQ |
| p6v5.1 ERD | 24 | LQSRPEPSAPPESLRPEPSAPPPESFRFEEATPSPKQELKDREPY |
| | | KELYPLTGLKSLFGNDHLLQ |
| p6v5.2 ERD | 25 | LQSRPEPSAPPESLRPEPSAPPPESFRFEEATPSPKQELKDREPY |
| | | KELYPLTSL |
| p6v5.3 ERD | 26 | LQSRPEPSAPPESLRPEPSAPPPESFRFEEATPSPKQELKDREPY |
| | | KELYPLTGL |
| p6v6.1 ERD | 27 | LQSRPEPTAPPESLRPEPTAPPPESFRFEEATPSPKQELKDREPY |
| | | KELYPLTGLKSLFGNDHLLQ |
| p6v6.2 ERD | 28 | LQSRPEPSAPPESLRPEPSAPPESLRPEPSAPPPESFRFEEATPSPK |
| | | QELKDREPYKELYPLTGLKSLFGNDHLLQ |
| p6v6.3 ERD | 29 | LQSRPEPSAPPESLRPEPSAPPPESFRFEEATPSPKQELKDREPY |
| | | KELYPLTGLKSLFGNDHLLQGGSQTQNLYPDLSEIKKE |
| p6v6.4 ERD | 30 | LQSRPEPSAPPESLRPEPSAPPPESFRFEEATPSPKQELKDREPY |
| | | KELYPLTGLKSLFGNDHL |
| p6v6.5 ERD | 31 | LQSRPEPSAPPESLRPEPSAPPPESFRFEEATPSPKQELKDREPY |
| | | KELYPLTGLKSLFGNDHLLQPPPY |
| p6v6.6 ERD | 32 | LQSRPEPSAPPESLRPEPSAPPPESFRFEEATPSPKQELKDREPY |
| | | KELYPLTGLKSLFGNDHLLQPPPYGGGSPPPY |
| p6v6.7 ERD | 33 | LQSRPEPSAPPESLRPEPSAPPPESFRFEEATPSPKQELKDREPY |
| | | KELYPLTGLKSLFGNDHLLQPPAY |
| p6v6.8 ERD | 34 | LQSRPEPSAPPESLRPEPSAPPESLRPEPSAPPPESFRFEEATPSPK |
| | | QELKDREPYKELYPLTGLKSLFGNDHLLQPPPY |
| p6v6.9 ERD | 35 | LQSRPEPSAPPESLRPEPSAPPPESFRFEEATPSPKQELKDREPY |
| | | KELYPLTGLKSLFGNDHLLQPPAYEPPAPAPLPPPSAP |
| p6v6.10 ERD | 36 | LQSRPEPSAPPESLRPEPSAPPPESFRFEEATPSPKQELKDREPY |
| | | KELYPLTGLKSLFGNDHLLQPSAAPTAPPTGAADSIRPPPYSP |
| p6v6.11 ERD | 37 | LQSRPEPSAPPESLRPEPSAPPPESFRFEEATPSPKQELKDREPY |
| | | KELYPLTGLKSLFGNDHLLQPSAPPPTAPPTGAADSIRPPPYSP |
| rGalectin-3min1 | 38 | MADGFSLNDALAGSGNPNPQGWPGAWGNQPGAGGYPGASY |
| | | PGAYPGQAPPGGYPGQAPPSAYPGPTGPSAYPGPTAPGAYPGP |
| | | TAPGAFPGQPGGPGAYPSAPGAYPSAPGAYPATGPFG |
| rGalectin-3min1- | 39 | MADGFSLNDALAGSGNPNPQGWPGAWGNQPGAGGYPGASY |
| ALIX2 | | PGAYPGQAPPGGYPGQAPPSAYPGPTGPSAYPGPTAPGAYPGP |
| | | TAPGAFPGQPGGPGAYPSAPGAYPSAPGAYPATGPFGPYKELY |
| | | PLTSL |

The fusion protein can comprise an endocytosis-preventing motif (EPM) capable of preventing endocytosis of the fusion protein. In some embodiments, the EPM: tethers the fusion protein to the cytoskeleton, thereby preventing localization to coated pits and endocytosis; enhances ENP assembly, ENP production, and/or ENP secretion; and/or prevents endocytosis of the fusion protein, thereby extending the time the fusion remains at the plasma membrane to interact with ESCRT proteins.

In some embodiments, the EPM: increases the abundance and/or density of fusion proteins on and/or in the ENP by at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) as compared to an ENP comprising fusion protein that does not comprise the EPM; and/or increases the number of ENPs secreted by a cell by at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) as compared to a cell expressing fusion protein that does not comprise the EPM.

In some embodiments, the EPM comprises or is derived from a portion of murine low-affinity gamma Fc region receptor II isoform FcRII-B1. The EPM can comprise all or a portion of the cytoplasmic tail of FcRII-B1. The EPM can comprise an amino acid sequence having at least 65%, 70%, 75%, 80%, 85%, 90%, 95%, 98%, or 99% sequence identity (e.g., at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) to the amino acid sequence of SEQ ID NO: 1. In some embodiments, the EPM comprises an amino acid sequence having one, two, or three mismatches relative to the sequence of SEQ ID NO: 1. In some embodiments, the EPM comprises the sequence of SEQ ID NO: 1.

In some embodiments, the fusion protein does not comprise an endocytosis-preventing motif (EPM).

The CSP, the RBP, and the ERD can be in any order (e.g., from N-terminus to C-terminus) within the fusion protein. The fusion protein can comprise, from N-terminus to C-terminus: the CSP, a first optional linker, the RBP, a second optional linker, and the ERD. The fusion protein can comprise, from N-terminus to C-terminus: the CSP, the EPM, the first optional linker, the RBP, the second optional linker, and the ERD.

In some embodiments, the first and/or second linker: is a flexible linker, a rigid linker, or a hybrid linker; is hydrophilic or hydrophobic; is between 1 and 250 amino acids; comprises one or more flexible amino acid residues, e.g., about 1 to about 250 flexible amino acid residues. In some embodiments, the flexible amino acid residues comprise glycine, serine, or a combination thereof; and/or comprises 3 repeating amino acid subunits or more.

The fusion protein can comprise: an amino acid sequence having at least 65%, 70%, 75%, 80%, 85%, 90%, 95%, 98%, or 99% sequence identity to any one of the amino acid sequences selected from the group consisting of SEQ ID NOs: 129, 137, 143, and 149; or an amino acid sequence selected from the group consisting of SEQ ID NOs: 129, 137, 143, and 149.

In some embodiments: (i) the fusion protein comprises the amino acid sequence of SEQ ID NO: 129, the packing signal comprises the sequence of SEQ ID NO: 134 and the soluble RBP comprises the sequence of SEQ ID NO: 128; (ii) the fusion protein comprises the amino acid sequence of SEQ ID NO: 149, the packing signal comprises the sequence of SEQ ID NO: 152, and the soluble RBP comprises the sequence of SEQ ID NO: 148; (iii) the fusion protein comprises the amino acid sequence of SEQ ID NO: 137, the packing signal comprises the sequence of SEQ ID NO: 140, and the soluble RBP comprises the sequence of SEQ ID NO: 136; and/or (iv) the fusion protein comprises the amino acid sequence of SEQ ID NO: 143, the packing signal comprises the sequence of SEQ ID NO: 146, and the soluble RBP comprises the sequence of SEQ ID NO: 142.

### Adapter-Based Systems

Disclosed herein include compositions. In some embodiments, the composition comprises: a nucleic acid composition comprising: (i) a first polynucleotide encoding a dimerization fusion protein, wherein the dimerization fusion protein comprises a cell surface protein (CSP) and a heterologous cytoplasmic tail, optionally the dimerization fusion protein further comprises an RNA-binding protein (RBP) and/or an endosomal sorting complex required for transport (ESCRT)-recruiting domain (ERD); (ii) a second polynucleotide encoding an adapter fusion protein comprising an adapter domain capable of binding the heterologous cytoplasmic tail to form a heterodimer, an optional RBP, and an optional endosomal sorting complex required for transport (ESCRT)-recruiting domain (ERD); and (iii) one or more third polynucleotides comprising one or more cargo RNA molecules each comprising a packing signal, wherein the RBP of (i) and (ii) are each capable of binding the packing signal, wherein binding of the adapter domain to the heterologous cytoplasmic tail is capable of recruiting one or more ESCRT proteins to the heterodimer, thereby inducing a plurality of dimerization fusion proteins to self-assemble into an enveloped nanoparticle (ENP) secreted from a cell in which the dimerization fusion protein and adapter fusion protein are expressed, thereby generating a population of ENPs comprising the dimerization fusion protein and the one or more cargo RNA molecules, optionally the nucleic acid composition further comprises a fourth polynucleotide encoding a soluble RBP capable of binding the packing signal.

The CSP can be a targeting protein capable of targeting the ENP to a target cell. In some embodiments, the nucleic acid composition further comprises a fifth polynucleotide encoding a cell fusion protein. The cell fusion protein can be capable of inducing the fusion of a lipid envelope of the ENP and a lipid bilayer of the target cell.

The dimerization fusion protein, the cell fusion protein, or both, can be capable of being presented on the surface of a cell in which the dimerization fusion protein and/or the cell fusion protein are expressed. In some embodiments, the self-assembly of an ENP does not require an exogenous nucleic acid other than the nucleic acid composition. In some embodiments, the cell is: a cell of a subject; an in vivo cell, an ex vivo cell, or an in situ cell; and/or an adherent cell or a suspension cell. Upon secretion from a cell of a subject, the ENPs can be capable of distributing within one or more tissues of the subject. The one or more tissues can comprise adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue, fat tissue, or any combination thereof. The one or more tissues can comprise diseased tissues, e.g., cancerous or infected tissues.

Disclosed herein include compositions. In some embodiments, the composition comprises: a population of enveloped nanoparticles (ENPs), wherein each of the ENPs comprises: (i) a plurality of dimerization fusion proteins each comprising a heterologous cytoplasmic tail and a CSP, optionally the CSP is a targeting protein capable of targeting the ENPs to a target cell; (ii) one or more cargo RNA molecules each comprising a packing signal; and optionally (iii) a plurality of cell fusion proteins. The ENPs can be derived from expression of the nucleic acid composition of the disclosure. The ENPs can comprise a lipid bilayer, e.g., a lipid bilayer derived from the cell from which the ENP was secreted.

Provided herein are RNA packing signals and RNA binding proteins (RBPs). Any of the engineered proteins provided herein (e.g., fusion protein, dimerization fusion protein, and/or an adapter fusion protein) can comprise one or more RBPs. In some embodiments, the dimerization fusion protein and/or the adapter fusion protein comprise an RBP, and the one or more cargo RNA molecules each comprise a packing signal.

The packing signal and the RBP can be derived from a viral, archaeal, bacterial, or mammalian packing signal and RBP, or variants thereof. The packing signal can comprise a Ku binding hairpin and the RBP and/or the soluble RBP can be Ku. The packing signal can comprise a telomerase Sm7 binding motif and the RBP and/or the soluble RBP can be Sm7. The packing signal can comprise an MS2 phage operator stem-loop and the RBP and/or the soluble RBP can be MS2 Coat Protein (MCP). The packing signal can comprise a PP7 phage operator stem-loop and the RBP and/or the soluble RBP can be PP7 Coat Protein (PCP). The packing signal can comprise an SfMu phage Com stem-loop and the RBP and/or the soluble RBP can be Com RNA binding protein. The packing signal can comprise a PUF binding site (PBS) and the RBP and/or the soluble RBP can be Pumilio/fem-3 mRNA binding factor (PUF). The packing signal can comprise Psi and the RBP and/or the soluble RBP can be gag, optionally derived from MMLV, HIV, SIV, FIV, HTLV, or Foamy viruses. The packing signal can comprise regulatory RNA CsrB and the RBP and/or the soluble RBP can be CsrA of E. coli. The packing signal can comprise PS9 and the RBP and/or the soluble RBP can be N protein of a coronavirus, optionally SARS-COV-2. The packing signal can comprise Box C/D binding motif and the RBP and/or the soluble RBP can be ribosomal protein L7Ae of archaea.

The packing signal can comprise regulatory RNA CsrB and the RBP and/or the soluble RBP can be CsrA of E. coli. The packing signal can comprise a nucleotide sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the nucleotide sequence of SEQ ID NO: 134 and the RBP and/or the soluble RBP can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the amino acid sequence of SEQ ID NO: 128. The packing signal can comprise a nucleotide sequence having one, two, or three mismatches relative to the nucleotide sequence of SEQ ID NO: 134 and the RBP and/or the soluble RBP can comprise an amino acid sequence having one, two, or three mismatches relative to the amino acid sequence of SEQ ID NO: 128.

The packing signal can comprise an MS2 phage operator stem-loop and the RBP can be MS2 Coat Protein (MCP). The packing signal can comprise a nucleotide sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the nucleotide sequence of SEQ ID NO: 152 and the RBP and/or the soluble RBP can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the amino acid sequence of SEQ ID NO: 148. The packing signal can comprise a nucleotide sequence having one, two, or three mismatches relative to the nucleotide sequence of SEQ ID NO: 152 and the RBP and/or the soluble RBP can comprise an amino acid sequence having one, two, or three mismatches relative to the amino acid sequence of SEQ ID NO: 148.

The packing signal can comprise PS9 and the RBP and/or the soluble RBP can be N protein of SARS-COV-2. The packing signal can comprise a nucleotide sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the nucleotide sequence of SEQ ID NO: 140 and the RBP and/or the soluble RBP can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the amino acid sequence of SEQ ID NO: 136. The packing signal can comprise a nucleotide sequence having one, two, or three mismatches relative to the nucleotide sequence of SEQ ID NO: 140 and the RBP and/or the soluble RBP can comprise an amino acid sequence having one, two, or three mismatches relative to the amino acid sequence of SEQ ID NO: 136.

The packing signal can comprise Box C/D binding motif and the RBP and/or the soluble RBP can be ribosomal protein L7Ae of archaea. The packing signal can comprise a nucleotide sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the nucleotide sequence of SEQ ID NO: 146 and the RBP and/or the soluble RBP can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the amino acid sequence of SEQ ID NO: 142. The packing signal can comprise a nucleotide sequence having one, two, or three mismatches relative to the nucleotide sequence of SEQ ID NO: 146 and the RBP and/or the soluble RBP can comprise an amino acid sequence having one, two, or three mismatches relative to the amino acid sequence of SEQ ID NO: 142.

The packing signal can comprise regulatory RNA CsrB and the RBP and/or the soluble RBP can be CsrA of E. coli. The packing signal can comprise the nucleotide sequence of SEQ ID NO: 134 and the RBP and/or the soluble RBP can comprise the amino acid sequence of SEQ ID NO: 128. The packing signal can comprise an MS2 phage operator stem-loop and the RNA binding protein can be MS2 Coat Protein (MCP). The packing signal can comprise the nucleotide sequence of SEQ ID NO: 152 and the RBP and/or the soluble RBP can comprise the amino acid sequence of SEQ ID NO: 148. The packing signal can comprise PS9 and the RBP and/or the soluble RBP can be N protein of SARS-COV-2. The packing signal can comprise the nucleotide sequence of SEQ ID NO: 140 and the RBP and/or the soluble RBP can comprise the amino acid sequence of SEQ ID NO: 136. The packing signal can comprise Box C/D binding motif and the RBP and/or the soluble RBP can be ribosomal protein L7Ae of archaea. The packing signal can comprise the nucleotide sequence of SEQ ID NO: 146 and the RBP and/or the soluble RBP can comprise the amino acid sequence of SEQ ID NO: 142.

The packing signal can be placed at any position within a polynucleotide (e.g., the one or more third polynucleotides comprising one or more cargo RNA molecules). The packing signal can be situated at the 5′ end or the 3′ end of at least one of the one or more RNA cargo molecules. In some embodiments, the at least one of the one or more RNA cargo molecules comprise an mRNA, and the packing signal can be situated within the 5′ or 3′ UTR of the mRNA.

An engineered protein of the disclosure (e.g., a dimerization fusion protein and/or adapter fusion protein), can comprise an endosomal sorting complex required for transport (ESCRT)-recruiting domain (ERD). The ERD can be capable of recruiting one or more ESCRT proteins to the cytoplasmic tail of the dimerization fusion protein. The recruitment of ESCRT proteins via the ERD can be capable of inducing the self-assembly and budding of ENPs.

There ERD can be situated within any region of a dimerization protein and/or adapter fusion protein. The ERD can be located at the C-terminus of the adapter fusion protein and/or the heterologous fusion protein, the N-terminus of the adapter fusion protein and/or the heterologous fusion protein, or between the C-terminus and the N-terminus of the adapter fusion protein and/or the heterologous fusion protein.

The ERD can be capable of interacting with the ESCRT proteins TSG101, NEDD4, and/or ALIX. In some embodiments, the ERD comprises or is derived from: a human protein; a nonhuman protein, optionally a nonmammalian protein, further optionally a chicken protein, a mouse protein, a lizard protein, a reptile protein, a hamster protein, or a goldfish protein; the ESCRT and ALIX binding region (EABR) of the human CEP55 protein, optionally residues 170-213; Syntenin-1, rat Galectin-3 (rGalectin-3), Hrs, and/or CD2AP; a viral protein, optionally a fragment of a viral protein, further optionally a retroviral protein, herpes simplex viral protein, vaccinia viral protein, hepadnaviral protein, togaviral protein, flaviviral protein, arenaviral protein, coronaviral protein, orthomyxoviral protein, paramyxoviral protein, bunyaviral protein, bornaviral protein, rhabdoviral protein or filoviral protein, optionally a Gag protein, further optionally derived from EIAV, HTLV-1, MLV, or MPMV, optionally EIAV p9 and/or HIV-1 p6; and/or an Ebola protein, optionally EBOV VP40.

The ERD can comprise one or more TSG101-binding motifs, one or more ALIX-binding motifs, one or more Nedd4-recruiting motifs, or any combination thereof. Any two of the one or more TSG101-binding motifs, the one or more ALIX-binding motifs, or the one or more Nedd4-recruiting motifs can be the same or different. The ERD can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the amino acid sequence of SEQ ID NO: 2. The ERD can comprise an amino acid sequence having one, two, or three mismatches relative to the amino acid sequence of SEQ ID NO: 2. The ERD can comprise the amino acid sequence of SEQ ID NO: 2. In some embodiments, the ERD comprises or is derived from a non-human galectin protein, e.g., a rat galectin protein. The ERD can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to an amino acid sequence selected from the group consisting of SEQ ID NOs: 3-39. The ERD can comprise an amino acid sequence having one, two, or three mismatches relative to any one of SEQ ID NOs: 3-39. In some embodiments, the ERD comprises or consists of the sequence of any one of SEQ ID NOs: 3-39. Also see Table 1 for exemplary ERD sequences.

In some embodiments, the dimerization fusion protein comprises a cell surface protein (CSP) and a heterologous cytoplasmic tail, optionally the dimerization fusion protein further comprises an RNA-binding protein (RBP) and/or an endosomal sorting complex required for transport (ESCRT)-recruiting domain (ERD). In some embodiments, the adapter fusion protein comprises an adapter domain capable of binding the heterologous cytoplasmic tail to form a heterodimer. The heterologous cytoplasmic tail and/or adapter domain can be derived from a mammalian, reptilian, avian, amphibian, or fish protein. In some embodiments, the heterologous cytoplasmic tail and/or adapter domain comprises or is derived from at least a portion of LAT, PAG, LCK, FYN, LAX, CD2, CD3, CD4, CD5, CD7, CD8a, PD1, SRC, or LYN. In some embodiments, the dimerization fusion protein and/or the adapter fusion protein further comprise an endogenous cytoplasmic tail, e.g., N-terminal to the heterologous cytoplasmic tail.

In some embodiments, the heterologous cytoplasmic tail and/or the adapter domain are each selected from the group comprising DHD9 heterodimer a, DHD13 XAAA heterodimer a, DHD13_XAXA heterodimer a, DHD13_XAAX heterodimer a, DHD13_2: 341 heterodimer a, DHD13_AAAA heterodimer a, DHD13_BAAA heterodimer a, DHD13 4:123 heterodimer a, DHD13_1: 234 heterodimer a, DHD15 heterodimer a, DHD20 heterodimer a, DHD21 heterodimer a, DHD25 heterodimer a, DHD27 heterodimer a, DHD30 heterodimer a, DHD33 heterodimer a, DHD34 XAAXA heterodimer a, DHD34 XAXXA heterodimer a, DHD34 XAAAA heterodimer a, DHD36 heterodimer a, DHD37_ABXB heterodimer a, DHD37_BBBB heterodimer a, DHD37_XBXB heterodimer a, DHD37_AXXB heterodimer a, DHD37_3: 124 heterodimer a, DHD37_1: 234 heterodimer a, DHD37_AXBB heterodimer a, DHD37 XBBA heterodimer a, DHD39 heterodimer a, DHD40 heterodimer a, DHD43 heterodimer a, DHD65 heterodimer a, DHD70 heterodimer a, DHD88 heterodimer a, DHD89 heterodimer a, DHD90 heterodimer a, DHD91 heterodimer a, DHD92 heterodimer a, DHD93 heterodimer a, DHD94 heterodimer a, DHD94_3: 214 heterodimer a, DHD94_2: 143 heterodimer a, DHD95 heterodimer a, DHD96 heterodimer a, DHD97 heterodimer a, DHD98 heterodimer a, DHD99 heterodimer a, DHD100 heterodimer a, DHD101 heterodimer a, DHD102 heterodimer a, DHD102 1:243 heterodimer a, DHD 103 heterodimer a, DHD103_1: 423 heterodimer a, DHD104 heterodimer a, DHD105 heterodimer a, DHD106 heterodimer a, DHD107 heterodimer a, DHD108 heterodimer a, DHD109 heterodimer a, DHD110 heterodimer a, DHD111 heterodimer a, DHD112 heterodimer a, DHD113 heterodimer a, DHD114 heterodimer a, DHD115 heterodimer a, DHD116 heterodimer a, DHD117 heterodimer a, DHD118 heterodimer a, DHD119 heterodimer a, DHD120 heterodimer a, DHD121 heterodimer a, DHD122 heterodimer a, DHD123 heterodimer a, DHD124 heterodimer a, DHD125 heterodimer a, DHD126 heterodimer a, DHD127 heterodimer a, DHD128 heterodimer a, DHD129 heterodimer a, DHD130 heterodimer a, DHD145 heterodimer a, DHD146 heterodimer a, DHD147 heterodimer a, DHD1 heterodimer a, DHD2 heterodimer a, DHD3 heterodimer a, DHD4 heterodimer a, DHD5 heterodimer a, DHD6 heterodimer a, DHD7 heterodimer a, DHD8 heterodimer a, DHD16 heterodimer a, DHD18 heterodimer a, DHD19 heterodimer a, DHD22 heterodimer a, DHD23 heterodimer a, DHD24 heterodimer a, DHD26 heterodimer a, DHD28 heterodimer a, DHD29 heterodimer a, DHD31 heterodimer a, DHD32 heterodimer a, DHD38 heterodimer a, DHD60 heterodimer a, DHD63 heterodimer a, DHD66 heterodimer a, DHD67 heterodimer a, DHD69 heterodimer a, DHD71 heterodimer a, DHD72 heterodimer a, DHD73 heterodimer a, DHD148 heterodimer a, DHD149 heterodimer a, DHD150 heterodimer a, DHD151 heterodimer a, DHD152 heterodimer a, DHD153 heterodimer a, DHD154 heterodimer a, DHD155 heterodimer a, DHD156 heterodimer a, DHD157 heterodimer a, DHD158 heterodimer a, DHD159 heterodimer a, DHD160 heterodimer a, DHD161 heterodimer a, DHD162 heterodimer a, DHD163 heterodimer a, DHD164 heterodimer a, DHD165 heterodimer a, DHD166 heterodimer a, DHS17 heterodimer a, DHD17 heterodimer a, DHD131 heterodimer a, DHD132 heterodimer a, DHD133 heterodimer a, DHD134 heterodimer a, DHD135 heterodimer a, DHD136 heterodimer a, DHD137 heterodimer a, DHD138 heterodimer a, DHD139 heterodimer a, DHD140 heterodimer a, DHD141 heterodimer a, DHD142 heterodimer a, DHD143 heterodimer a, DHD 144 heterodimer a, DHD9 heterodimer b, DHD13_XAAA heterodimer b, DHD13 XAXA heterodimer b, DHD13 XAAX heterodimer b, DHD13_2: 341 heterodimer b, DHD13 AAAA heterodimer b, DHD13 BAAA heterodimer b, DHD13_4: 123 heterodimer b, DHD13_1: 234 heterodimer b, DHD15 heterodimer b, DHD20 heterodimer b, DHD21 heterodimer b, DHD25 heterodimer b, DHD27 heterodimer b, DHD30 heterodimer b, DHD33 heterodimer b, DHD34 XAAXA heterodimer b, DHD34 XAXXA heterodimer b, DHD34 XAAAA heterodimer b, DHD36 heterodimer b, DHD37_ABXB heterodimer b, DHD37 BBBB heterodimer b, DHD37_XBXB heterodimer b, DHD37_AXXB heterodimer b, DHD37_3: 124 heterodimer b, DHD37_1: 234 heterodimer b, DHD37_AXBB heterodimer b, DHD37_XBBA heterodimer b, DHD39 heterodimer b, DHD40 heterodimer b, DHD43 heterodimer b, DHD65 heterodimer b, DHD70 heterodimer b, DHD88 heterodimer b, DHD89 heterodimer b, DHD90 heterodimer b, DHD91 heterodimer b, DHD92 heterodimer b, DHD93 heterodimer b, DHD94 heterodimer b, DHD94_3: 214 heterodimer b, DHD94_2: 143 heterodimer b, DHD95 heterodimer b, DHD96 heterodimer b, DHD97 heterodimer b, DHD98 heterodimer b, DHD99 heterodimer b, DHD100 heterodimer b, DHD101 heterodimer b, DHD102 heterodimer b, DHD102_1: 243 heterodimer b, DHD103 heterodimer b, DHD103_1: 423 heterodimer b, DHD104 heterodimer b, DHD105 heterodimer b, DHD 106 heterodimer b, DHD107 heterodimer b, DHD108 heterodimer b, DHD109 heterodimer b, DHD110 heterodimer b, DHD111 heterodimer b, DHD112 heterodimer b, DHD113 heterodimer b, DHD114 heterodimer b, DHD115 heterodimer b, DHD116 heterodimer b, DHD117 heterodimer b, DHD118 heterodimer b, DHD119 heterodimer b, DHD120 heterodimer b, DHD121 heterodimer b, DHD122 heterodimer b, DHD123 heterodimer b, DHD124 heterodimer b, DHD125 heterodimer b, DHD126 heterodimer b, DHD127 heterodimer b, DHD128 heterodimer b, DHD129 heterodimer b, DHD130 heterodimer b, DHD145 heterodimer b, DHD146 heterodimer b, DHD147 heterodimer b, DHD1 heterodimer b, DHD2 heterodimer b, DHD3 heterodimer b, DHD4 heterodimer b, DHD5 heterodimer b, DHD6 heterodimer b, DHD7 heterodimer b, DHD8 heterodimer b, DHD16 heterodimer b, DHD18 heterodimer b, DHD19 heterodimer b, DHD22 heterodimer b, DHD23 heterodimer b, DHD24 heterodimer b, DHD26 heterodimer b, DHD28 heterodimer b, DHD29 heterodimer b, DHD31 heterodimer b, DHD32 heterodimer b, DHD38 heterodimer b, DHD60 heterodimer b, DHD63 heterodimer b, DHD66 heterodimer b, DHD67 heterodimer b, DHD69 heterodimer b, DHD71 heterodimer b, DHD72 heterodimer b, DHD73 heterodimer b, DHD148 heterodimer b, DHD149 heterodimer b, DHD 150 heterodimer b, DHD151 heterodimer b, DHD152 heterodimer b, DHD153 heterodimer b, DHD154 heterodimer b, DHD155 heterodimer b, DHD156 heterodimer b, DHD157 heterodimer b, DHD158 heterodimer b, DHD159 heterodimer b, DHD160 heterodimer b, DHD161 heterodimer b, DHD162 heterodimer b, DHD163 heterodimer b, DHD164 heterodimer b, DHD165 heterodimer b, DHD166 heterodimer b, DHS17 heterodimer b, DHD 17 heterodimer b, DHD131 heterodimer b, DHD 132 heterodimer b, DHD133 heterodimer b, DHD134 heterodimer b, DHD135 heterodimer b, DHD136 heterodimer b, DHD137 heterodimer b, DHD138 heterodimer b, DHD139 heterodimer b, DHD140 heterodimer b, DHD141 heterodimer b, DHD142 heterodimer b, DHD143 heterodimer b, DHD144 heterodimer b, portions thereof, derivatives thereof, or any combination thereof.

In some embodiments, the heterologous cytoplasmic tail and/or the adapter domain comprises or is derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACHI, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PBI domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof.

In some embodiments, the heterologous cytoplasmic tail comprises or is derived from ACIDpl or BASEp1. In some embodiments, the heterologous cytoplasmic tail comprises or is derived from N5 or N6. The heterologous cytoplasmic tail can comprise the sequence of any one of SEQ ID NOs: 116-117 and 121-122. In some embodiments, the heterologous cytoplasmic tail comprises or is derived from ACIDpl or BASEp1. In some embodiments, the heterologous cytoplasmic tail comprises or is derived from N5 or N6. The heterologous cytoplasmic tail can comprise a sequence at least 65% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to any one of SEQ ID NOs: 116-117 and 121-122.

In some embodiments, the adapter domain comprises or is derived from ACIDpl or BASEp1. In some embodiments, the heterologous cytoplasmic tail comprises or is derived from N5 or N6. In some embodiments, the adapter domain comprises the sequence of any one of SEQ ID NOs: 116-117 and 121-122. In some embodiments, the adapter domain comprises a sequence at least 65% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to the sequence of any one of SEQ ID NOs: 116-117 and 121-122.

In some embodiments, the heterologous cytoplasmic tail comprises or is derived from a cytoplasmic tail (CT) of CD4. In some embodiments, the adapter domain comprises or is derived from Lck tyrosine kinase. In some embodiments, the CD4 CT comprises the sequence of SEQ ID NO: 95 or a sequence at least 65% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to the sequence of SEQ ID NO: 95. In some embodiments, the adapter domain comprises the sequence of SEQ ID NO: 43 or a sequence at least 65% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to the sequence of SEQ ID NO: 43. In some embodiments, the CD4 CT comprises a sequence having one, two, or three mismatches relative to the sequence of SEQ ID NO: 95. In some embodiments, the adapter domain comprises a sequence having one, two, or three mismatches relative to SEQ ID NO: 43.

The RBP and the ERD of the adapter fusion protein can be in any order (e.g. from N-terminus to C-terminus) of the adapter fusion protein. The adapter fusion protein can comprise, from N-terminus to C-terminus: the adapter domain, a first optional linker, the RBP, a second optional linker, and the ERD.

In some embodiments, the first and/or second linker: is a flexible linker, a rigid linker, or a hybrid linker; is hydrophilic or hydrophobic; is between 1 and 250 amino acids; comprises one or more flexible amino acid residues, e.g., about 1 to about 250 flexible amino acid residues, further optionally the flexible amino acid residues comprise glycine, serine, or a combination thereof; and/or comprises 3 repeating amino acid subunits or more.

The adapter fusion protein can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to any one of the amino acid sequences selected from the group consisting of SEQ ID NOs: 157, 159, 162, and 164. In some embodiments, the adapter fusion protein comprises the sequence of any one of SEQ ID NOs: 157, 159, 162, and 164.

In some embodiments, the heterologous cytoplasmic tail is derived from or comprises a cytoplasmic tail of a cell surface protein. In some embodiments, the adapter domain is (1) capable of binding the heterologous cytoplasmic tail derived from or comprising the cytoplasmic tail of said cell surface protein and (2) capable of targeting the adapter fusion protein to the plasma membrane.

In some embodiments, the adapter domain comprises or is derived from Lck tyrosine kinase. In some embodiments, the Lck tyrosine kinase comprises a myristolylation motif. In some embodiments, myristoylation drives membrane anchoring of the adapter fusion protein to the plasma membrane.

In some embodiments, the cell surface protein is or is derived from a human protein, a non-human mammalian protein, an avian protein, a reptile protein, a fish protein, an amphibian protein, a viral protein, or a bacterial protein and/or the adapter domain is or is derived from a human protein, a non-human mammalian protein, an avian protein, a reptile protein, a fish protein, an amphibian protein, a viral protein, or a bacterial protein.

Shown in Table 2-Table 3 below are exemplary adapter system sequences.

TABLE 2
Sequence Information for Components of Designed ERD Adapter Systems

| | SEQ | |
|---|---|---|
| Name | ID NO: | SEQUENCE* |
| SARS-COV-2 | 40 | MFVFLVLLPLVSSQCVNLTTRTQLPPAYTNSFTRGVYYPDKVFRSSVL |
| Spike | | HSTQDLFLPFFSNVTWFHAIHVSGTNGTKRFDNPVLPFNDGVYFASTE |
| | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLCCMTSCCSCLKGCCSCGS |
| SARS-COV-2 | 41 | MFVFLVLLPLVSSQCVNLTTRTQLPPAYTNSFTRGVYYPDKVFRSSVL |
| Spike-CD4 CT | | HSTQDLFLPFFSNVTWFHAIHVSGTNGTKRFDNPVLPFNDGVYFASTE |
| | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLCVRCRHRRRQAERMSQIKRLLSE |
| | | KKTCQCPHRFQKTCSPI |
| SARS-COV-2 | 42 | MFVFLVLLPLVSSQCVNLTTRTQLPPAYTNSFTRGVYYPDKVFRSSVL |
| Spike-CD4 CT-EPM | | HSTQDLFLPFFSNVTWFHAIHVSGTNGTKRFDNPVLPFNDGVYFASTE |
| | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | IDKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLCVRCRHRRRQAERMSQIKRLLSE |
| | | KKTCQCPHRFQKTCSPIALPGNPDHREMGETLPEEVGEYRQPSGGSVPVS |
| | | PGPPSGLEPTSSSPY |
| Lck | 43 | MGCGCSSHPEDDWMENIDVCENCHYPIVPLDGKGTLLIRNGSEVRDPL |
| | | VTYEGSNPPASPL |
| Lck-EABR | 44 | MGCGCSSHPEDDWMENIDVCENCHYPIVPLDGKGTLLIRNGSEVRDPL |
| | | VTYEGSNPPASPLGSKSGSGSDSGSFNSSINNIHEMEIQLKDALEKNQQW |
| | | LVYDQQREVYVKGLLAKIFELEKKTETAAHSLP |
| SARS-COV-2 | 45 | MFVFLVLLPLVSSQCVNLTTRTQLPPAYTNSFTRGVYYPDKVFRSSVL |
| Spike-muCD4 | | HSTQDLFLPFFSNVTWFHAIHVSGTNGTKRFDNPVLPFNDGVYFASTE |
| CT | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLCVRCRHQQRQAARMSQIKRLLSE |
| | | KKTCQCPHRMQKSHNLI |
| muLck-EABR | 46 | MGCVCSSNPEDDWMENIDVCENCHYPIVPLDSKISLPIRNGSEVRDPL |
| | | VTYEGSLPPASPLGSKSGSGSDSGSFNSSINNIHEMEIQLKDALEKNQQW |
| | | LVYDQQREVYVKGLLAKIFELEKKTETAAHSLP |
| Influenza HA | 47 | MKAILVVMLYTFTTANADTLCIGYHANNSTDTVDTVLEKNVTVTHSV |
| | | NLLEDKHNGKLCKLRGVAPLHLGKCNIAGWILGNPECESLSTARSWS |
| | | YIVETSNSDNGTCFPGDFINYEELREQLSSVSSFERFEIFPKTSSWPNHD |
| | | SDNGVTAACPHAGAKSFYKNLIWLVKKGKSYPKINQTYINDKGKEVL |
| | | VLWGIHHPPTIADQQSLYQNADAYVFVGTSRYSKKFKPEIATRPKVRD |
| | | QEGRMNYYWTLVEPGDKITFEATGNLVAPRYAFTMERDAGSGIIISDT |
| | | PVHDCNTTCQTPEGAINTSLPFQNVHPITIGKCPKYVKSTKLRLATGLR |
| | | NVPSIQSRGLFGAIAGFIEGGWTGMVDGWYGYHHQNEQGSGYAADL |
| | | KSTQNAIDKITNKVNSVIEKMNTQFTAVGKEFNHLEKRIENLNKKVDD |
| | | GFLDIWTYNAELLVLLENERTLDYHDSNVKNLYEKVRNQLKNNAKEI |
| | | GNGCFEFYHKCDNTCMESVKNGTYDYPKYSEEAKLNREKIDGVKLDS |
| | | TRIYQILAIYSTVASSLVLVVSLGAISFWMCSNGSLQCRICI |
| Influenza HA- | 48 | MKAILVVMLYTFTTANADTLCIGYHANNSTDTVDTVLEKNVTVTHSV |
| CD4 CT | | NLLEDKHNGKLCKLRGVAPLHLGKCNIAGWILGNPECESLSTARSWS |
| | | YIVETSNSDNGTCFPGDFINYEELREQLSSVSSFERFEIFPKTSSWPNHD |
| | | SDNGVTAACPHAGAKSFYKNLIWLVKKGKSYPKINQTYINDKGKEVL |
| | | VLWGIHHPPTIADQQSLYQNADAYVFVGTSRYSKKFKPEIATRPKVRD |
| | | QEGRMNYYWTLVEPGDKITFEATGNLVAPRYAFTMERDAGSGIIISDT |
| | | PVHDCNTTCQTPEGAINTSLPFQNVHPITIGKCPKYVKSTKLRLATGLR |
| | | NVPSIQSRGLFGAIAGFIEGGWTGMVDGWYGYHHQNEQGSGYAADL |
| | | KSTQNAIDKITNKVNSVIEKMNTQFTAVGKEFNHLEKRIENLNKKVDD |
| | | GFLDIWTYNAELLVLLENERTLDYHDSNVKNLYEKVRNQLKNNAKEI |
| | | GNGCFEFYHKCDNTCMESVKNGTYDYPKYSEEAKLNREKIDGVKLDS |
| | | TRIYQILAIYSTVASSLVLVVSLGAISFWMCVRCRHRRRQAERMSQIKR |
| | | LLSEKKTCQCPHRFQKTCSPI |
| Influenza HA- | 49 | MKAILVVMLYTFTTANADTLCIGYHANNSTDTVDTVLEKNVTVTHSV |
| muCD4 CT | | NLLEDKHNGKLCKLRGVAPLHLGKCNIAGWILGNPECESLSTARSWS |
| | | YIVETSNSDNGTCFPGDFINYEELREQLSSVSSFERFEIFPKTSSWPNHD |
| | | SDNGVTAACPHAGAKSFYKNLIWLVKKGKSYPKINQTYINDKGKEVL |
| | | VLWGIHHPPTIADQQSLYQNADAYVFVGTSRYSKKFKPEIATRPKVRD |
| | | QEGRMNYYWTLVEPGDKITFEATGNLVAPRYAFTMERDAGSGIIISDT |
| | | PVHDCNTTCQTPEGAINTSLPFQNVHPITIGKCPKYVKSTKLRLATGLR |
| | | NVPSIQSRGLFGAIAGFIEGGWTGMVDGWYGYHHQNEQGSGYAADL |
| | | KSTQNAIDKITNKVNSVIEKMNTQFTAVGKEFNHLEKRIENLNKKVDD |
| | | GFLDIWTYNAELLVLLENERTLDYHDSNVKNLYEKVRNQLKNNAKEI |
| | | GNGCFEFYHKCDNTCMESVKNGTYDYPKYSEEAKLNREKIDGVKLDS |
| | | TRIYQILAIYSTVASSLVLVVSLGAISFWMCVRCRHQQRQAARMSQIK |
| | | RLLSEKKTCQCPHRMQKSHNLI |
| SARS-COV-2 | 50 | MFVFLVLLPLVSSQCVNLTTRTQLPPAYTNSFTRGVYYPDKVFRSSVL |
| Spike-CD4 | | HSTQDLFLPFFSNVTWFHAIHVSGTNGTKRFDNPVLPFNDGVYFASTE |
| CT-delEM | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLCVRCRHRRRQAERMSQIKRAASE |
| | | KKTCQCPHRFQKTCSPI |
| SARS-COV-2 | 51 | MFVFLVLLPLVSSQCVNLTTRTQLPPAYTNSFTRGVYYPDKVFRSSVL |
| Spike-CD4 | | HSTQDLFLPFFSNVTWFHAIHVSGTNGTKRFDNPVLPFNDGVYFASTE |
| CT-delEM1 | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLCVRCRHRRRQAERMSQIKRALSE |
| | | KKTCQCPHRFQKTCSPI |
| SARS-COV-2 | 52 | MFVFLVLLPLVSSQCVNLTTRTQLPPAYTNSFTRGVYYPDKVFRSSVL |
| Spike-CD4 | | HSTQDLFLPFFSNVTWFHAIHVSGTNGTKRFDNPVLPFNDGVYFASTE |
| CT-delEM2 | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLCVRCRHRRRQAERMSQIKRLASE |
| | | KKTCQCPHRFQKTCSPI |
| SARS-COV-2 | 53 | MFVFLVLLPLVSSQCVNLTTRTQLPPAYTNSFTRGVYYPDKVFRSSVL |
| Spike-muCD4 | | HSTQDLFLPFFSNVTWFHAIHVSGTNGTKRFDNPVLPFNDGVYFASTE |
| CT-delEM | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLCVRCRHQQRQAARMSQIKRAASE |
| | | KKTCQCPHRMQKSHNLI |
| SARS-COV-2 | 54 | MFVFLVLLPLVSSQCVNLTTRTQLPPAYTNSFTRGVYYPDKVFRSSVL |
| Spike-muCD4 | | HSTQDLFLPFFSNVTWFHAIHVSGTNGTKRFDNPVLPFNDGVYFASTE |
| CT-delEM1 | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLCVRCRHQQRQAARMSQIKRALSE |
| | | KKTCQCPHRMQKSHNLI |
| SARS-COV-2 | 55 | MFVFLVLLPLVSSQCVNLTTRTQLPPAYTNSFTRGVYYPDKVFRSSVL |
| Spike-muCD4 | | HSTQDLFLPFFSNVTWFHAIHVSGTNGTKRFDNPVLPFNDGVYFASTE |
| CT-delEM2 | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLCVRCRHQQRQAARMSQIKRLASE |
| | | KKTCQCPHRMQKSHNLI |
| SARS-COV-2 | 56 | MFVFLVLLPLVSSQCVNLTTRTQLPPAYTNSFTRGVYYPDKVFRSSVL |
| Spike-CD8 CT | | HSTQDLFLPFFSNVTWFHAIHVSGTNGTKRFDNPVLPFNDGVYFASTE |
| | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLLYCNHRNRRRVCKCPRPVVKSGD |
| | | KPSLSARYV |
| SARS-COV-2 | 57 | MFVFLVLLPLVSSQCVNLTTRTQLPPAYTNSFTRGVYYPDKVFRSSVL |
| Spike-CD4 | | HSTQDLFLPFFSNVTWFHAIHVSGTNGTKRFDNPVLPFNDGVYFASTE |
| CT-A404C | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLCVRCRHRRRQCERMSQIKRLLSE |
| | | KKTCQCPHRFQKTCSPI |
| SARS-COV-2 | 58 | MFVFLVLLPLVSSQCVNLTTRTQLPPAYTNSFTRGVYYPDKVFRSSVL |
| Spike-CD4 | | HSTQDLFLPFFSNVTWFHAIHVSGTNGTKRFDNPVLPFNDGVYFASTE |
| CT-I410C | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLCVRCRHRRRQAERMSQCKRLLSE |
| | | KKTCQCPHRFQKTCSPI |
| SARS-COV-2 | 59 | MFVFLVLLPLVSSQCVNLTTRTQLPPAYTNSFTRGVYYPDKVFRSSVL |
| Spike-CD4 | | HSTQDLFLPFFSNVTWFHAIHVSGTNGTKRFDNPVLPFNDGVYFASTE |
| CT-T419C | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLCVRCRHRRRQAERMSQIKRLLSE |
| | | KKCCQCPHRFQKTCSPI |
| Lck-EABR- | 60 | MGCGCSSHPEDDWMCNIDVCENCHYPIVPLDGKGTLLIRNGSEVRDP |
| E15C | | LVTYEGSNPPASPLGSKSGSGSDSGSFNSSINNIHEMEIQLKDALEKNQQ |
| | | WLVYDQQREVYVKGLLAKIFELEKKTETAAHSLP |
| Lck-EABR- | 61 | MGCGCSSHPEDDWCENIDVCENCHYPIVPLDGKGTLLIRNGSEVRDPL |
| M14C | | VTYEGSNPPASPLGSKSGSGSDSGSFNSSINNIHEMEIQLKDALEKNQQW |
| | | LVYDQQREVYVKGLLAKIFELEKKTETAAHSLP |
| Lck-EABR- | 62 | MGCGCSSHPEDDWMENIDVCENCHYCIVPLDGKGTLLIRNGSEVRDP |
| P26C | | LVTYEGSNPPASPLGSKSGSGSDSGSFNSSINNIHEMEIQLKDALEKNQQ |
| | | WLVYDQQREVYVKGLLAKIFELEKKTETAAHSLP |
| SARS-COV-2 | 63 | MFVFLVLLPLVSSQCVNLTTRTQLPPAYTNSFTRGVYYPDKVFRSSVL |
| Spike-CD4 | | HSTQDLFLPFFSNVTWFHAIHVSGTNGTKRFDNPVLPFNDGVYFASTE |
| CT-delEM1- | | KSNIIRGWIFGTTLDSKTQSLLIVNNATNVVIKVCEFQFCNDPFLGVYY |
| A404C | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLCVRCRHRRRQCERMSQIKRALSE |
| | | KKTCQCPHRFQKTCSPI |
| Lck-p6v5.1 | 64 | MGCGCSSHPEDDWMENIDVCENCHYPIVPLDGKGTLLIRNGSEVRDPL |
| | | VTYEGSNPPASPLGSKSGSGSDSGSLQSRPEPSAPPESLRPEPSAPPPESF |
| | | RFEEATPSPKQELKDREPYKELYPLTGLKSLFGNDHLLQ |
| Lck-rGalectin- | 65 | MGCGCSSHPEDDWMENIDVCENCHYPIVPLDGKGTLLIRNGSEVRDPL |
| 3min1-ALIX2 | | VTYEGSNPPASPLGSKSGSGSDSGSMADGFSLNDALAGSGNPNPQGWP |
| | | GAWGNQPGAGGYPGASYPGAYPGQAPPGGYPGQAPPSAYPGPTGPSAYP |
| | | GPTAPGAYPGPTAPGAFPGQPGGPGAYPSAPGAYPSAPGAYPATGPFGPY |
| | | KELYPLTSL |
| ACP33-EABR | 66 | MGEIKVSPDYNWFRGTVPLKKIIVDDDDSKIWSLYDAGPRSIRCPLIFL |
| | | PPVSGTADVFFRQILALTGWGYRVIALQYPVYWDHLEFCDGFRKLLD |
| | | HLQLDKVHLFGASLGGFLAQKFAEYTHKSPRVHSLILCNSFSDTSIFNQ |
| | | TWTANSFWLMPAFMLKKIVLGNFSSGPVDPMMADAIDFMVDRLESL |
| | | GQSELASRLTLNCQNSYVEPHKIRDIPVTIMDVFDQSALSTEAKEEMY |
| | | KLYPNARRAHLKTGGNFPYLCRSAEVNLYVQIHLLQFHGTKYAAIDPS |
| | | MVSAEELEVQKGSLGISQEEQGSKSGSGSDSGSFNSSINNIHEMEIQLKD |
| | | ALEKNQQWLVYDQQREVYVKGLLAKIFELEKKTETAAHSLP |
| ACP33-p6v5.1 | 67 | MGEIKVSPDYNWFRGTVPLKKIIVDDDDSKIWSLYDAGPRSIRCPLIFL |
| | | PPVSGTADVFFRQILALTGWGYRVIALQYPVYWDHLEFCDGFRKLLD |
| | | HLQLDKVHLFGASLGGFLAQKFAEYTHKSPRVHSLILCNSFSDTSIFNQ |
| | | TWTANSFWLMPAFMLKKIVLGNFSSGPVDPMMADAIDFMVDRLESL |
| | | GQSELASRLTLNCQNSYVEPHKIRDIPVTIMDVFDQSALSTEAKEEMY |
| | | KLYPNARRAHLKTGGNFPYLCRSAEVNLYVQIHLLQFHGTKYAAIDPS |
| | | MVSAEELEVQKGSLGISQEEQGSKSGSGSDSGSLQSRPEPSAPPESLRPE |
| | | PSAPPPESFRFEEATPSPKQELKDREPYKELYPLTGLKSLFGNDHLLQ |
| Influenza HA- | 68 | MKAILVVMLYTFTTANADTLCIGYHANNSTDTVDTVLEKNVTVTHSV |
| CD4 CT- | | NLLEDKHNGKLCKLRGVAPLHLGKCNIAGWILGNPECESLSTARSWS |
| delEM1.1 | | YIVETSNSDNGTCFPGDFINYEELREQLSSVSSFERFEIFPKTSSWPNHD |
| | | SDNGVTAACPHAGAKSFYKNLIWLVKKGKSYPKINQTYINDKGKEVL |
| | | VLWGIHHPPTIADQQSLYQNADAYVFVGTSRYSKKFKPEIATRPKVRD |
| | | QEGRMNYYWTLVEPGDKITFEATGNLVAPRYAFTMERDAGSGIIISDT |
| | | PVHDCNTTCQTPEGAINTSLPFQNVHPITIGKCPKYVKSTKLRLATGLR |
| | | NVPSIQSRGLFGAIAGFIEGGWTGMVDGWYGYHHQNEQGSGYAADL |
| | | KSTQNAIDKITNKVNSVIEKMNTQFTAVGKEFNHLEKRIENLNKKVDD |
| | | GFLDIWTYNAELLVLLENERTLDYHDSNVKNLYEKVRNQLKNNAKEI |
| | | GNGCFEFYHKCDNTCMESVKNGTYDYPKYSEEAKLNREKIDGVKLDS |
| | | TRIYQILAIYSTVASSLVLVVSLGAISFWMCVRCRHRRRQAERMSQIKR |
| | | ALSEKKTCQCPHRFQKTCSPI |
| Influenza HA- | 69 | MKAILVVMLYTFTTANADTLCIGYHANNSTDTVDTVLEKNVTVTHSV |
| CD4 CT- | | NLLEDKHNGKLCKLRGVAPLHLGKCNIAGWILGNPECESLSTARSWS |
| delEM1.2 | | YIVETSNSDNGTCFPGDFINYEELREQLSSVSSFERFEIFPKTSSWPNHD |
| | | SDNGVTAACPHAGAKSFYKNLIWLVKKGKSYPKINQTYINDKGKEVL |
| | | VLWGIHHPPTIADQQSLYQNADAYVFVGTSRYSKKFKPEIATRPKVRD |
| | | QEGRMNYYWTLVEPGDKITFEATGNLVAPRYAFTMERDAGSGIIISDT |
| | | PVHDCNTTCQTPEGAINTSLPFQNVHPITIGKCPKYVKSTKLRLATGLR |
| | | NVPSIQSRGLFGAIAGFIEGGWTGMVDGWYGYHHQNEQGSGYAADL |
| | | KSTQNAIDKITNKVNSVIEKMNTQFTAVGKEFNHLEKRIENLNKKVDD |
| | | GFLDIWTYNAELLVLLENERTLDYHDSNVKNLYEKVRNQLKNNAKEI |
| | | GNGCFEFYHKCDNTCMESVKNGTYDYPKYSEEAKLNREKIDGVKLDS |
| | | TRIYQILAIYSTVASSLVLVVSLGAICVRCRHRRRQAERMSQIKRALSE |
| | | KKTCQCPHRFQKTCSPI |
| Lck-EPM- | 70 | MGCGCSSHPEDDWMENIDVCENCHYPIVPLDGKGTLLIRNGSEVRDPL |
| EABR | | VTYEGSNPPASPLGSKSGSGSDSGSALPGNPDHREMGETLPEEVGEYRQ |
| | | PSGGSVPVSPGPPSGLEPTSSSPYGGGSFNSSINNIHEMEIQLKDALEKNQQ |
| | | WLVYDQQREVYVKGLLAKIFELEKKTETAAHSLP |
| Lck-EPM- | 71 | MGCGCSSHPEDDWMENIDVCENCHYPIVPLDGKGTLLIRNGSEVRDPL |
| p6v5.1 | | VTYEGSNPPASPLGSKSGSGSDSGSALPGNPDHREMGETLPEEVGEYRQ |
| | | PSGGSVPVSPGPPSGLEPTSSSPYGGGSLQSRPEPSAPPESLRPEPSAPPPES |
| | | FRFEEATPSPKQELKDREPYKELYPLTGLKSLFGNDHLLQ |
| Lck-p6v6.1 | 72 | MGCGCSSHPEDDWMENIDVCENCHYPIVPLDGKGTLLIRNGSEVRDPL |
| | | VTYEGSNPPASPLGSKSGSGSDSGSLQSRPEPTAPPESLRPEPTAPPPESF |
| | | RFEEATPSPKQELKDREPYKELYPLTGLKSLFGNDHLLQ |
| Lck-p6v6.2 | 73 | MGCGCSSHPEDDWMENIDVCENCHYPIVPLDGKGTLLIRNGSEVRDPL |
| | | VTYEGSNPPASPLGSKSGSGSDSGSLQSRPEPSAPPESLRPEPSAPPESLR |
| | | PEPSAPPPESFRFEEATPSPKQELKDREPYKELYPLTGLKSLFGNDHLLQ |
| Lck-p6v6.3 | 74 | MGCGCSSHPEDDWMENIDVCENCHYPIVPLDGKGTLLIRNGSEVRDPL |
| | | VTYEGSNPPASPLGSKSGSGSDSGSLQSRPEPSAPPESLRPEPSAPPPESF |
| | | RFEEATPSPKQELKDREPYKELYPLTGLKSLFGNDHLLQGGSQTQNLYPD |
| | | LSEIKKE |
| Lck-p6v6.4 | 75 | MGCGCSSHPEDDWMENIDVCENCHYPIVPLDGKGTLLIRNGSEVRDPL |
| | | VTYEGSNPPASPLGSKSGSGSDSGSLQSRPEPSAPPESLRPEPSAPPPESF |
| | | RFEEATPSPKQELKDREPYKELYPLTGLKSLFGNDHL |
| Lck-p6v6.5 | 76 | MGCGCSSHPEDDWMENIDVCENCHYPIVPLDGKGTLLIRNGSEVRDPL |
| | | VTYEGSNPPASPLGSKSGSGSDSGSLQSRPEPSAPPESLRPEPSAPPPESF |
| | | RFEEATPSPKQELKDREPYKELYPLTGLKSLFGNDHLLQPPPY |
| Lck-p6v6.6 | 77 | MGCGCSSHPEDDWMENIDVCENCHYPIVPLDGKGTLLIRNGSEVRDPL |
| | | VTYEGSNPPASPLGSKSGSGSDSGSLQSRPEPSAPPESLRPEPSAPPPESF |
| | | RFEEATPSPKQELKDREPYKELYPLTGLKSLFGNDHLLQPPPYGGGSPPPY |
| Lck-p6v6.7 | 78 | MGCGCSSHPEDDWMENIDVCENCHYPIVPLDGKGTLLIRNGSEVRDPL |
| | | VTYEGSNPPASPLGSKSGSGSDSGSLQSRPEPSAPPESLRPEPSAPPPESF |
| | | RFEEATPSPKQELKDREPYKELYPLTGLKSLFGNDHLLQPPAY |
| Lck-p6v6.8 | 79 | MGCGCSSHPEDDWMENIDVCENCHYPIVPLDGKGTLLIRNGSEVRDPL |
| | | VTYEGSNPPASPLGSKSGSGSDSGSLQSRPEPSAPPESLRPEPSAPPESLR |
| | | PEPSAPPPESFRFEEATPSPKQELKDREPYKELYPLTGLKSLFGNDHLLQP |
| | | PPY |
| Lck-p6v6.9 | 80 | MGCGCSSHPEDDWMENIDVCENCHYPIVPLDGKGTLLIRNGSEVRDPL |
| | | VTYEGSNPPASPLGSKSGSGSDSGSLQSRPEPSAPPESLRPEPSAPPPESF |
| | | RFEEATPSPKQELKDREPYKELYPLTGLKSLFGNDHLLQPPAYEPPAPAPL |
| | | PPPSAP |
| Lck-p6v6.10 | 81 | MGCGCSSHPEDDWMENIDVCENCHYPIVPLDGKGTLLIRNGSEVRDPL |
| | | VTYEGSNPPASPLGSKSGSGSDSGSLQSRPEPSAPPESLRPEPSAPPPESF |
| | | RFEEATPSPKQELKDREPYKELYPLTGLKSLFGNDHLLQPSAAPTAPPTGA |
| | | ADSIRPPPYSP |
| Lck-p6v6.11 | 82 | MGCGCSSHPEDDWMENIDVCENCHYPIVPLDGKGTLLIRNGSEVRDPL |
| | | VTYEGSNPPASPLGSKSGSGSDSGSLQSRPEPSAPPESLRPEPSAPPPESF |
| | | RFEEATPSPKQELKDREPYKELYPLTGLKSLFGNDHLLQPSAPPPTAPPTG |
| | | AADSIRPPPYSP |
| SARS-COV-2 | 83 | MFVFLVLLPLVSSQCVNLTTRTQLPPAYTNSFTRGVYYPDKVFRSSVL |
| Spike- | | HSTQDLFLPFFSNVTWFHAIHVSGTNGTKRFDNPVLPFNDGVYFASTE |
| chickCD4 CT- | | KSNIIRGWIFGTTLDSKTQSLLIVNNATNVVIKVCEFQFCNDPFLGVYY |
| delEM1 | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLQRRRKRARRMAQAKQYALEKKT |
| | | CQCQRRMYK |
| SARS-COV-2 | 84 | MFVFLVLLPLVSSQCVNLTTRTQLPPAYTNSFTRGVYYPDKVFRSSVL |
| Spike-dragCD4 | | HSTQDLFLPFFSNVTWFHAIHVSGTNGTKRFDNPVLPFNDGVYFASTE |
| CT-delEM1 | | KSNIIRGWIFGTTLDSKTQSLLIVNNATNVVIKVCEFQFCNDPFLGVYY |
| | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLRRKRQQVKRMALAKQHALAKRT |
| | | CQCQRELTNDYYHT |
| SARS-COV-2 | 85 | MFVFLVLLPLVSSQCVNLTTRTQLPPAYTNSFTRGVYYPDKVFRSSVL |
| Spike- | | HSTQDLFLPFFSNVTWFHAIHVSGTNGTKRFDNPVLPFNDGVYFASTE |
| snakeCD4 CT- | | KSNIIRGWIFGTTLDSKTQSLLIVNNATNVVIKVCEFQFCNDPFLGVYY |
| delEM1 | | HKNNKSWMESEFRVYSSANNCTFEYVSQPFLMDLEGKQGNFKNLREF |
| | | VFKNIDGYFKIYSKHTPINLVRDLPQGFSALEPLVDLPIGINITRFQTLLA |
| | | LHRSYLTPGDSSSGWTAGAAAYYVGYLQPRTFLLKYNENGTITDAVD |
| | | CALDPLSETKCTLKSFTVEKGIYQTSNFRVQPTESIVRFPNITNLCPFGE |
| | | VFNATRFASVYAWNRKRISNCVADYSVLYNSASFSTFKCYGVSPTKL |
| | | NDLCFTNVYADSFVIRGDEVRQIAPGQTGKIADYNYKLPDDFTGCVIA |
| | | WNSNNLDSKVGGNYNYLYRLFRKSNLKPFERDISTEIYQAGSTPCNGV |
| | | EGFNCYFPLQSYGFQPTNGVGYQPYRVVVLSFELLHAPATVCGPKKST |
| | | NLVKNKCVNFNFNGLTGTGVLTESNKKFLPFQQFGRDIADTTDAVRD |
| | | PQTLEILDITPCSFGGVSVITPGTNTSNQVAVLYQGVNCTEVPVAIHAD |
| | | QLTPTWRVYSTGSNVFQTRAGCLIGAEHVNNSYECDIPIGAGICASYQ |
| | | TQTNSPRRARSVASQSIIAYTMSLGAENSVAYSNNSIAIPTNFTISVTTEI |
| | | LPVSMTKTSVDCTMYICGDSTECSNLLLQYGSFCTQLNRALTGIAVEQ |
| | | DKNTQEVFAQVKQIYKTPPIKDFGGFNFSQILPDPSKPSKRSFIEDLLFN |
| | | KVTLADAGFIKQYGDCLGDIAARDLICAQKFNGLTVLPPLLTDEMIAQ |
| | | YTSALLAGTITSGWTFGAGAALQIPFAMQMAYRFNGIGVTQNVLYEN |
| | | QKLIANQFNSAIGKIQDSLSSTASALGKLQDVVNQNAQALNTLVKQLS |
| | | SNFGAISSVLNDILSRLDPPEAEVQIDRLITGRLQSLQTYVTQQLIRAAEI |
| | | RASANLAATKMSECVLGQSKRVDFCGKGYHLMSFPQSAPHGVVFLH |
| | | VTYVPAQEKNFTTAPAICHDGKAHFPREGVFVSNGTHWFVTQRNFYE |
| | | PQIITTDNTFVSGNCDVVIGIVNNTVYDPLQPELDSFKEELDKYFKNHT |
| | | SPDVDLGDISGINASVVNIQKEIDRLNEVAKNLNESLIDLQELGKYEQY |
| | | IKWPWYIWLGFIAGLIAIVMVTIMLPTCKRIKQKQQQAKKMAQIKQHA |
| | | LAKRTCQCQRDLPIDYYHT |
| chickLck- | 86 | MGCCCSSDYDEDWIENIDICEHCNYPIDPDSKRQQLIRNVSEVRDPLVS |
| EABR | | YEAMSPPCSPLGSKSGSGSDSGSFNSSINNIHEMEIQLKDALEKNQQWLV |
| | | YDQQREVYVKGLLAKIFELEKKTETAAHSLP |
| dragLck-EABR | 87 | MGNCCSLDYDDDWLEDIDICEVCHYPIDPSTKPQRLALNGSEAYNPLL |
| | | SSEMDPPPSSPLGSKSGSGSDSGSFNSSINNIHEMEIQLKDALEKNQQWLV |
| | | YDQQREVYVKGLLAKIFELEKKTETAAHSLP |
| snakeLck- | 88 | MGCCYSLDYDDDWAIDAEICEVCHYPIDPATKPQRPTLNVSELHTPLL |
| EABR | | MTEGIPPPSSPLGSKSGSGSDSGSFNSSINNIHEMEIQLKDALEKNQQWLV |
| | | YDQQREVYVKGLLAKIFELEKKTETAAHSLP |
| HA-ACIDp1 | 89 | MKAILVVMLYTFTTANADTLCIGYHANNSTDTVDTVLEKNVTVTHSV |
| | | NLLEDKHNGKLCKLRGVAPLHLGKCNIAGWILGNPECESLSTARSWS |
| | | YIVETSNSDNGTCFPGDFINYEELREQLSSVSSFERFEIFPKTSSWPNHD |
| | | SDNGVTAACPHAGAKSFYKNLIWLVKKGKSYPKINQTYINDKGKEVL |
| | | VLWGIHHPPTIADQQSLYQNADAYVFVGTSRYSKKFKPEIATRPKVRD |
| | | QEGRMNYYWTLVEPGDKITFEATGNLVAPRYAFTMERDAGSGIIISDT |
| | | PVHDCNTTCQTPEGAINTSLPFQNVHPITIGKCPKYVKSTKLRLATGLR |
| | | NVPSIQSRGLFGAIAGFIEGGWTGMVDGWYGYHHQNEQGSGYAADL |
| | | KSTQNAIDKITNKVNSVIEKMNTQFTAVGKEFNHLEKRIENLNKKVDD |
| | | GFLDIWTYNAELLVLLENERTLDYHDSNVKNLYEKVRNQLKNNAKEI |
| | | GNGCFEFYHKCDNTCMESVKNGTYDYPKYSEEAKLNREKIDGVKLDS |
| | | TRIYQILAIYSTVASSLVLVVSLGAISFWMCSNGSLQCRICIGGGSAQLE |
| | | KELQALEKENAQLEWELQALEKELAQ |
| HA-BASEp1 | 90 | MKAILVVMLYTFTTANADTLCIGYHANNSTDTVDTVLEKNVTVTHSV |
| | | NLLEDKHNGKLCKLRGVAPLHLGKCNIAGWILGNPECESLSTARSWS |
| | | YIVETSNSDNGTCFPGDFINYEELREQLSSVSSFERFEIFPKTSSWPNHD |
| | | SDNGVTAACPHAGAKSFYKNLIWLVKKGKSYPKINQTYINDKGKEVL |
| | | VLWGIHHPPTIADQQSLYQNADAYVFVGTSRYSKKFKPEIATRPKVRD |
| | | QEGRMNYYWTLVEPGDKITFEATGNLVAPRYAFTMERDAGSGIIISDT |
| | | PVHDCNTTCQTPEGAINTSLPFQNVHPITIGKCPKYVKSTKLRLATGLR |
| | | NVPSIQSRGLFGAIAGFIEGGWTGMVDGWYGYHHQNEQGSGYAADL |
| | | KSTQNAIDKITNKVNSVIEKMNTQFTAVGKEFNHLEKRIENLNKKVDD |
| | | GFLDIWTYNAELLVLLENERTLDYHDSNVKNLYEKVRNQLKNNAKEI |
| | | GNGCFEFYHKCDNTCMESVKNGTYDYPKYSEEAKLNREKIDGVKLDS |
| | | TRIYQILAIYSTVASSLVLVVSLGAISFWMCSNGSLQCRICIGGGSAQLK |
| | | KKLQALKKKNAQLKWKLQALKKKLAQ |
| Fyn-ACIDp1- | 91 | MGCVQCKDKEGGGSAQLEKELQALEKENAQLEWELQALEKELAQGS |
| EABR | | KSGSGSDSGSFNSSINNIHEMEIQLKDALEKNQQWLVYDQQREVYVKGLL |
| | | AKIFELEKKTETAAHSLP |
| Fyn-BASEp1- | 92 | MGCVQCKDKEGGGSAQLKKKLQALKKKNAQLKWKLQALKKKLAQ |
| EABR | | GSKSGSGSDSGSFNSSINNIHEMEIQLKDALEKNQQWLVYDQQREVYVKG |
| | | LLAKIFELEKKTETAAHSLP |
| HA-N5 | 93 | MKAILVVMLYTFTTANADTLCIGYHANNSTDTVDTVLEKNVTVTHSV |
| | | NLLEDKHNGKLCKLRGVAPLHLGKCNIAGWILGNPECESLSTARSWS |
| | | YIVETSNSDNGTCFPGDFINYEELREQLSSVSSFERFEIFPKTSSWPNHD |
| | | SDNGVTAACPHAGAKSFYKNLIWLVKKGKSYPKINQTYINDKGKEVL |
| | | VLWGIHHPPTIADQQSLYQNADAYVFVGTSRYSKKFKPEIATRPKVRD |
| | | QEGRMNYYWTLVEPGDKITFEATGNLVAPRYAFTMERDAGSGIIISDT |
| | | PVHDCNTTCQTPEGAINTSLPFQNVHPITIGKCPKYVKSTKLRLATGLR |
| | | NVPSIQSRGLFGAIAGFIEGGWTGMVDGWYGYHHQNEQGSGYAADL |
| | | KSTQNAIDKITNKVNSVIEKMNTQFTAVGKEFNHLEKRIENLNKKVDD |
| | | GFLDIWTYNAELLVLLENERTLDYHDSNVKNLYEKVRNQLKNNAKEI |
| | | GNGCFEFYHKCDNTCMESVKNGTYDYPKYSEEAKLNREKIDGVKLDS |
| | | TRIYQILAIYSTVASSLVLVVSLGAISFWMCSNGSLQCRICIGGGSYEIA |
| | | ALEAKIAALKAKNAALKAEIAALEAGC |
| Fyn-N6-EABR | 94 | MGCVQCKDKEGGGSYKIAALKAEIAALEAENAALEAKIAALKAGCGS |
| | | KSGSGSDSGSFNSSINNIHEMEIQLKDALEKNQQWLVYDQQREVYVKGLL |
| | | AKIFELEKKTETAAHSLP |

*Amino acid sequences are shown for all designed constructs. EPM is indicated by underline italic, and ERD by italic sequences. Sequence elements of designed adapter systems that interact with a binding partner are underlined. delEM, delEM1, delEM2, A404C, I410C, T419C, E15C, M14C, and P26C mutations are shown in bold.

TABLE 3
Sequence Information for Dimerization and Other
Domains of ERD Adapter Systems

| | SEQ | |
|---|---|---|
| Name | ID NO: | SEQUENCE |
| CD4 CT | 95 | CVRCRHRRRQAERMSQIKRLLSEKKTCQCPHRFQKTCSPI |
| muCD4 CT | 96 | CVRCRHQQRQAARMSQIKRLLSEKKTCQCPHRMQKSHNLI |
| LcK | 43 | MGCGCSSHPEDDWMENIDVCENCHYPIVPLDGKGTLLIRNGSEVRDP |
| | | LVTYEGSNPPASPL |
| muLcK | 97 | MGCVCSSNPEDDWMENIDVCENCHYPIVPLDSKISLPIRNGSEVRDPL |
| | | VTYEGSLPPASPL |
| CD4 CT-delEM | 98 | CVRCRHRRRQAERMSQIKRAASEKKTCQCPHRFQKTCSPI |
| CD4 CT- | 99 | CVRCRHRRRQAERMSQIKRALSEKKTCQCPHRFQKTCSPI |
| delEM1 | | |
| CD4 CT- | 100 | CVRCRHRRRQAERMSQIKRLASEKKTCQCPHRFQKTCSPI |
| delEM2 | | |
| CD8 CT | 101 | LYCNHRNRRRVCKCPRPVVKSGDKPSLSARYV |
| CD4 CT-A404C102 | | CVRCRHRRRQCERMSQIKRLLSEKKTCQCPHRFQKTCSPI |
| CD4 CT-I410C | 103 | CVRCRHRRRQAERMSQCKRLLSEKKTCQCPHRFQKTCSPI |
| CD4 CT-T419C | 104 | CVRCRHRRRQAERMSQIKRLLSEKKCCQCPHRFQKTCSPI |
| Lck-E15C | 105 | MGCGCSSHPEDDWMCNIDVCENCHYPIVPLDGKGTLLIRNGSEVRDP |
| | | LVTYEGSNPPASPL |
| Lck-M14C | 106 | MGCGCSSHPEDDWCENIDVCENCHYPIVPLDGKGTLLIRNGSEVRDP |
| | | LVTYEGSNPPASPL |
| Lck-P26C | 107 | MGCGCSSHPEDDWMENIDVCENCHYCIVPLDGKGTLLIRNGSEVRDP |
| | | LVTYEGSNPPASPL |
| CD4 CT- | 108 | CVRCRHRRRQCERMSQIKRALSEKKTCQCPHRFQKTCSPI |
| delEM1-A404C | | |
| ACP33 | 109 | MGEIKVSPDYNWFRGTVPLKKIIVDDDDSKIWSLYDAGPRSIRCPLIFL |
| | | PPVSGTADVFFRQILALTGWGYRVIALQYPVYWDHLEFCDGFRKLLD |
| | | HLQLDKVHLFGASLGGFLAQKFAEYTHKSPRVHSLILCNSFSDTSIFN |
| | | QTWTANSFWLMPAFMLKKIVLGNFSSGPVDPMMADAIDFMVDRLES |
| | | LGQSELASRLTLNCQNSYVEPHKIRDIPVTIMDVFDQSALSTEAKEEM |
| | | YKLYPNARRAHLKTGGNFPYLCRSAEVNLYVQIHLLQFHGTKYAAID |
| | | PSMVSAEELEVQKGSLGISQEEQ |
| chickCD4 CT- | 110 | QRRRKRARRMAQAKQYALEKKTCQCQRRMYK |
| delEM1 | | |
| dragCD4 CT- | 111 | RRKRQQVKRMALAKQHALAKRTCQCQRELTNDYYHT |
| delEM1 | | |
| snakeCD4 CT- | 112 | PTCKRIKQKQQQAKKMAQIKQHALAKRTCQCQRDLPIDYYHT |
| delEM1 | | |
| chickLck | 113 | MGCCCSSDYDEDWIENIDICEHCNYPIDPDSKRQQLIRNVSEVRDPLV |
| | | SYEAMSPPCSPL |
| dragLck | 114 | MGNCCSLDYDDDWLEDIDICEVCHYPIDPSTKPQRLALNGSEAYNPLL |
| | | SSEMDPPPSSPL |
| snakeLck | 115 | MGCCYSLDYDDDWAIDAEICEVCHYPIDPATKPQRPTLNVSELHTPLL |
| | | MTEGIPPPSSPL |
| ACIDp1 | 116 | AQLEKELQALEKENAQLEWELQALEKELAQ |
| BASEp1 | 117 | AQLKKKLQALKKKNAQLKWKLQALKKKLAQ |
| Fyn | 118 | MGCVQCKDKE |
| Fyn-ACIDp1 | 119 | MGCVQCKDKEGGGSAQLEKELQALEKENAQLEWELQALEKELAQ |
| Fyn-BASEp1 | 120 | MGCVQCKDKEGGGSAQLKKKLQALKKKNAQLKWKLQALKKKLAQ |
| N5 | 121 | YEIAALEAKIAALKAKNAALKAEIAALEAGC |
| N6 | 122 | YKIAALKAEIAALEAENAALEAKIAALKAGC |
| Fyn-N6 | 123 | MGCVQCKDKEGGGSYKIAALKAEIAALEAENAALEAKIAALKAGC |

The dimerization fusion protein can comprise an endocytosis-preventing motif (EPM) capable of preventing endocytosis of the dimerization fusion protein. In some embodiments, the EPM: tethers the dimerization fusion protein to the cytoskeleton, thereby preventing localization to coated pits and endocytosis; enhances ENP assembly, ENP production, and/or ENP secretion; and/or prevents endocytosis of the dimerization fusion protein, and/or the adapter fusion protein, thereby extending the time the dimerization fusion protein, and/or the adapter fusion protein remains at the plasma membrane to interact with ESCRT proteins.

In some embodiments, the EPM: increases the abundance and/or density of dimerization fusion proteins, adapter fusion proteins, one or more RNA cargo molecules, and/or cell surface proteins on and/or in the ENP by at least about 2-fold as compared to an ENP comprising a dimerization fusion protein that does not comprise the EPM; and/or increases the number of ENPs secreted by a cell by at least about 2-fold as compared to a cell expressing a dimerization fusion protein that does not comprise the EPM. In some embodiments, the EPM comprises or is derived from a portion of murine low-affinity gamma Fc region receptor II isoform FcRII-B1. The EPM can comprise all or a portion of the cytoplasmic tail of FcRII-B1. The EPM can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the amino acid sequence of SEQ ID NO: 1. In some embodiments, the EPM comprises an amino acid sequence having one, two, or three mismatches relative to the sequence of SEQ ID NO: 1. The EPM can comprise the amino acid sequence of SEQ ID NO: 1.

In some embodiments, dimerization fusion protein does not comprise an endocytosis-preventing motif (EPM).

The CSP, the heterologous cytoplasmic tail, the RBP, and/or the EPM can be located within the dimerization fusion protein in any order (e.g., from N-terminus to C-terminus). The dimerization fusion protein can comprise, from N-terminus to C-terminus: the CSP, the heterologous cytoplasmic tail, a first flexible linker, and the EPM. The dimerization fusion protein can comprise, from N-terminus to C-terminus: the CSP, the heterologous cytoplasmic tail, a first flexible linker, the EPM, a second flexible linker, and the RBP.

In some embodiments, the first and/or second linker: is a flexible linker, a rigid linker, or a hybrid linker; is hydrophilic or hydrophobic; is between 1 and 250 amino acids; comprises one or more flexible amino acid residues, e.g., about 1 to about 250 flexible amino acid residues, further optionally the flexible amino acid residues comprise glycine, serine, or a combination thereof; and/or comprises 3 repeating amino acid subunits or more.

In some embodiments, the dimerization fusion protein comprises a sequence selected from the sequences of SEQ ID NOs: 156, 158, 160-161, and 163. In some embodiments, the dimerization fusion protein comprises a sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to any of SEQ ID NOs: 156, 158, 160-161, and 163.

The molar ratios of the first polynucleotide, the second polynucleotide, the one or more third polynucleotides, the fourth polynucleotide, and/or the fifth polynucleotide can vary. In some embodiments, the first polynucleotide, the second polynucleotide, the one or more third polynucleotides, the fourth polynucleotide, and/or the fifth polynucleotide are present in the nucleic acid composition at equimolar ratios. In some embodiments, the amount of the one or more third polynucleotides in the nucleic acid compositions is greater than each of the first, second, fourth, and/or fifth polynucleotides.

In some embodiments, (i) the first polynucleotide encoding the dimerization fusion protein, and (ii) the second polynucleotide encoding the adapter fusion protein, are each present in a different nucleic acid molecule. In some embodiments, the amount of (i) the polynucleotide encoding the dimerization fusion protein; and (ii) the polynucleotide encoding the adapter fusion protein, are present in the composition at a molar ratio of about 9:1, 5:1, 1:1, 1:5, or 1:9. In some embodiments, the amount of (i) the polynucleotide encoding the dimerization fusion protein; and (ii) the polynucleotide encoding the adapter fusion protein, are present in the composition at a molar ratio of or of about 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values. In some embodiments, the ratio can be at least, or be at most, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, or 10000:1. In some embodiments, (i) the first polynucleotide encoding the dimerization fusion protein, and (ii) the second polynucleotide encoding the adapter fusion protein, are present in the same nucleic acid molecule.

It is contemplated that any cell surface protein or cell surface domain of a protein can be used for the CSP. In some embodiments, the CSP is a targeting protein and comprises or is derived from one or more receptors and/or targeting moieties configured to bind a target molecule of a cell of a subject. In some embodiments, the one or more receptors and/or the one or more targeting moieties are selected from the group comprising mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic.

The one or more receptors and/or targeting moieties can comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, a receptor ligand, a chimeric antigen receptor (CAR), a T cell receptor (TCR), a targeted recognition of antigen-MHC complex reporter (TRACeR), and analogs and derivatives thereof. Exemplary methods and compositions related to TRACeRs are also described in “Du, H., Mallik, L., Hwang, D. et al. Targeting peptide antigens using a multiallelic MHC I-binding system. Nat Biotechnol (2024)”, which is hereby incorporated by reference in its entirety.

The antibody or antigen-binding fragment thereof can comprise a Fab, a Fab′, a F (ab′)₂, a Fv, a scFv, a dsFv, a diabody, a triabody, a tetrabody, a multispecific antibody formed from antibody fragments, a single-domain antibody (sdAb), a single chain comprising complementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

The one or more receptors and/or targeting moieties can be configured to bind one or more of the following: CD3, CD4, CD5, CD6, CD7, CD8, CD9, CD10, CD11a, CD11b, CD11c, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin Al, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL 11 (C-C motif chemokine 11), CCR4 (C-C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSFIR (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C—X—C chemokine receptor type 4, cyclic ADP ribose hydrolase, Cyclin B1, CYPIB 1, Cytomegalovirus, Cytomegalovirus glycoprotein B, Dabigatran, DLL4 (delta-like-ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRVIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor α-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGFIR (IGF-1, insulin-like growth factor 1 receptor), IGHE, IFN-γ, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αιιβ3. ανβ3, αβ7, α5β1, αββ4, a7B7, allß3, a5B5, avß5), Interferon gamma-induced protein, ITGA2, ITGB2, KIR2D, LCK, Le, Legumain, Lewis-Y antigen, LFA-1 (Lymphocyte function-associated antigen 1, CD11a), LHRH, LINGO-1, Lipoteichoic acid, LIVIA, LMP2, LTA, MAD-CT-1, MAD-CT-2, MAGE-1, MAGE-2, MAGE-3, MAGE AI, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUCI (Mucin 1, cell surface associated (MUC1) or polymorphic epithelial mucin (PEM)), MUCI-KLH, MUC16 (CA125), MCPI (monocyte chemotactic protein 1), MelanA/MARTI, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page4, PAP, Paratope of anti-(N-glycolylneuraminic acid), PAX3, PAX5, PCSK9, PDCDI (PD-1, Programmed cell death protein 1, CD279), PDGF-Ra (Alpha-type platelet-derived growth factor receptor), PDGFR-B, PDL-1, PLAC1, PLAP-like testicular alkaline phosphatase, Platelet-derived growth factor receptor beta, Phosphate-sodium co-transporter, PMEL 17, Polysialic acid, Proteinase3 (PRI), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe (a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-β1, TGF-β2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRPI (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, T-cell receptors, viral surface proteins, peptide-MHC complexes, or cells expressing any insulin growth factor receptors, or any epidermal growth factor receptors. The peptide of the peptide-MHC complex can be associated with a disease or disorder. The peptide of the peptide-MHC complex can be an intracellular tumor antigen.

In some embodiments, the CSP is a targeting protein and comprises or is derived from an scFv. In some embodiments, the scFv comprises a transmembrane domain or is fused to a heterologous transmembrane domain.

The scFv can be capable of binding to: CD3, CD4, CD5, CD6, CD7, CD8, CD9, CD10, CD11a, CD11b, CD11c, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin Al, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11 (C-C motif chemokine 11), CCR4 (C-C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSFIR (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C—X—C chemokine receptor type 4, cyclic ADP ribose hydrolase, Cyclin B 1, CYPIB 1, Cytomegalovirus, Cytomegalovirus glycoprotein B, Dabigatran, DLL4 (delta-like-ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRVIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor α-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGFIR (IGF-1, insulin-like growth factor 1 receptor), IGHE, IFN-γ, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αιιβ3. ανβ3, αιβι, α5β1, αββ4, α7β7, α11β3, α5β5, ανβ5), Interferon gamma-induced protein, ITGA2, ITGB2, KIR2D, LCK, Le, Legumain, Lewis-Y antigen, LFA-I (Lymphocyte function-associated antigen 1, CD11a), LHRH, LINGO-1, Lipoteichoic acid, LIVIA, LMP2, LTA, MAD-CT-1, MAD-CT-2, MAGE-1, MAGE-2, MAGE-3, MAGE Al, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUCI (Mucin 1, cell surface associated (MUC1) or polymorphic epithelial mucin (PEM)), MUCI-KLH, MUC16 (CA125), MCPI (monocyte chemotactic protein 1), MelanA/MARTI, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page4, PAP, Paratope of anti-(N-glycolylneuraminic acid), PAX3, PAX5, PCSK9, PDCDI (PD-1, Programmed cell death protein 1, CD279), PDGF-Ra (Alpha-type platelet-derived growth factor receptor), PDGFR-B, PDL-1, PLAC1, PLAP-like testicular alkaline phosphatase, Platelet-derived growth factor receptor beta, Phosphate-sodium co-transporter, PMEL 17, Polysialic acid, Proteinase3 (PRI), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe (a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-β1, TGF-2 (Transforming growth factor-beta 2), Tie (CD202b), Tie2, TIM-1 (CDX-014), Tn, TNF, TNF-α, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUCI, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, T-cell receptors, viral surface proteins, peptide-MHC complexes, or cells expressing any insulin growth factor receptors, or any epidermal growth factor receptors. The peptide of the peptide-MHC complex can be associated with a disease or disorder. The peptide of the peptide-MHC complex can be an intracellular tumor antigen.

The scFv can be capable of binding to CD19, CD4, CD3, or any combination thereof. The heterologous transmembrane domain can comprise CD8a chain transmembrane domain.

In some embodiments, the CSP is a targeting protein comprising an scFv and comprises the sequence of any one of SEQ ID NOs: 178, 180, and 182. In some embodiments, the CSP is a targeting protein comprising an scFv and comprises a sequence at least 65% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to the sequence of any one of SEQ ID NOs: 178, 180, and 182. In some embodiments, the CSP is a targeting protein and comprises or is derived from SARS-COV spike protein. The CSP comprising or derived from SARS-COV spike protein can be capable of targeting the ENP to a target cell expressing ACE2. The CSP comprises SARS-COV-2 spike protein and comprises an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the sequence of SEQ ID NO: 40.

Also provided are polynucleotides (e.g., a fifth polynucleotide) encoding a cell fusion protein. The cell fusion protein can be capable of inducing the fusion of a lipid envelope of the ENP and a lipid bilayer of the target cell. In some embodiments, the cell fusion protein comprises or is derived from VSV-G. The VSV-G can comprise one or more mutations thereby the VSV-G protein is not capable of binding to an LDL-receptor. The one or more mutations can comprise K47Q and/or R354A relative to wild type VSV-G. The cell fusion protein can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to the sequence of SEQ ID NO: 169.

In some embodiments, the cell fusion protein is, comprises, or is derived from a SNARE protein, a viral glycoprotein, an FF protein, dynamin, a FAST protein, synuclein, myomaker, myomerger, or any combination thereof. In some embodiments, the viral glycoprotein is selected from the group comprising glycoprotein GP of Ebola or Marburg virus, glycoproteins HN and F of Newcastle virus, protein E and prM of Murray Valley encephalitis virus, El and/or E2 proteins of HCV, HA (hemaglutinin) and NA (neuraminidase) of Influenza, glycoprotein G of VSV, glycoproteins Gp 120 (or a CD4-binding domain thereof) and Gp41 of lentiviruses, envelope protein (DENV E) and pre-membrane protein (prM DENV) of Dengue virus, the two envelope glycoproteins of Hantaan virus, glycoprotein E2 of Chikungunya virus, gp85 and gp37 of Rous sarcoma virus, HBsAg of HBV, or any combination thereof. In some embodiments, the viral glycoprotein is selected from the group comprising M-HBsAg, S-HBsAg or L-HBsAg. The viral glycoprotein can be a measles glycoprotein, a sindvis virus glycoprotein, baboon retroviral Env, or a Reovirus Fusion-Associated Small Transmembrane (FAST) protein. The viral glycoprotein can be a glycoprotein from hepatitis D virus, orthomyxoviridae, paramyxoviridae, filoviridae, retroviridae, herpesviridae, poxviridae, hepadnaviridae, flaviviridae, togavoridae, coronaviridae, rhabdoviridae, bunyaviridae, orthopoxivridae, measles virus, sindbis virus, baboon retroviral virus, or any combination thereof.

In some embodiments: (i) the dimerization fusion protein comprises the sequence of SEQ ID NO: 156, the adapter fusion protein comprises the sequence of SEQ ID NO: 157, the soluble RBP comprises the sequence of SEQ ID NO: 128, and the packing signal comprises the sequence of SEQ ID NO: 134; (ii) the dimerization fusion protein comprises the sequence of SEQ ID NO: 161, the adapter fusion protein comprises the sequence of SEQ ID NO: 162, the soluble RBP comprises the sequence of SEQ ID NO: 136, and the packing signal comprising the sequence of SEQ ID NO: 140; (iii) the dimerization fusion protein comprises the sequence of SEQ ID NO: 160, the adapter fusion protein comprises the sequence of SEQ ID NO: 162, the soluble RBP comprises the sequence of SEQ ID NO: 136, and the packing signal comprises the sequence of SEQ ID NO: 140; (iv) the dimerization fusion protein comprises the sequence of SEQ ID NO: 160, the adapter fusion protein comprises the sequence of SEQ ID NO: 162, the soluble RBP comprises the sequence of SEQ ID NO: 136, the packing signal comprises the sequence of SEQ ID NO: 140, and the cell fusion protein comprises the sequence of SEQ ID NO: 169, optionally the nucleic acid composition does not comprise the fourth polynucleotide encoding the soluble RBP; (v) the dimerization fusion protein comprises the sequence of SEQ ID NO: 160, the adapter fusion protein comprises the sequence of SEQ ID NO: 164, the soluble RBP comprises the sequence of SEQ ID NO: 142, the packing signal comprises the sequence of SEQ ID NO: 146, and the cell fusion protein comprises the sequence of SEQ ID NO: 169, optionally the nucleic acid composition does not comprise the fourth polynucleotide encoding the soluble RBP; (vi) the dimerization fusion protein comprises the sequence of SEQ ID NO: 179, the adapter fusion protein comprises the sequence of SEQ ID NO: 164, the packing signal comprises the sequence of SEQ ID NO: 146, and the cell fusion protein comprises the sequence of SEQ ID NO: 169; and/or (vii) the dimerization fusion protein comprises the sequence of SEQ ID NO: 181, the adapter fusion protein comprises the sequence of SEQ ID NO: 164, the packing signal comprises the sequence of SEQ ID NO: 146, and the cell fusion protein comprises the sequence of SEQ ID NO: 169.

### Cargoes

The compositions and methods of the disclosure can be used to deliver a wide variety of cargoes to a cell. Described below are non-limiting examples of cargoes that can be delivered (e.g., to cells) using the disclosed compositions and methods.

The one or more cargo RNA molecules each can comprise a microRNA (miRNA), a messenger RNA (mRNA), a long non-coding RNA (lncRNA), a ribosomal RNA (rRNA), a transfer RNA (tRNA), a small nuclear RNA (snRNA), a small nucleolar RNA (snoRNA), a Piwi-interacting RNA (piRNA), a interfering RNA (siRNA), an antisense RNA (aRNA), a transfer messenger RNA (tmRNA), a tRNA-derived small RNA (tsRNA), a rDNA-derived small RNA (srRNA), a ribozyme, a viral RNA, a single-stranded RNA, a double-stranded RNA, self-amplifying RNA, circular RNA, an aptamer, or any combination thereof. The miRNA or siRNA can be capable of inhibiting the expression of a target mRNA in a cell. In some embodiments, the mRNA encodes a payload protein.

The miRNA, the siRNA, and/or payload protein can be a therapeutic miRNA, siRNA, and/or protein or a variant thereof, e.g., a therapeutic miRNA, siRNA, and/or protein configured to prevent or treat a disease or disorder of a subject. In some embodiments, the subject suffers from a deficiency of said therapeutic protein.

The payload protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. The payload protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. The payload protein can comprise a CRE recombinase, GCaMP, a cell therapy component, a knock-down gene therapy component, a cell-surface exposed epitope, or any combination thereof.

The payload protein can comprise a diagnostic agent. The diagnostic agent can comprise green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCitrine, mCherry, mruby3, rsCherry, rsCherryRev, derivatives thereof, or any combination thereof.

The payload protein can comprise a tumor antigen. In some embodiments, the tumor antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac (2-8) aNeu5Ac (2-3) bDGalp (1-4) bDGlcp (1-1) Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin β2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac (2-3) bDGalp (1-4) bDGlcp (1-1) Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the tumor antigen is selected from the group comprising CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRVIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, LI-CAM, LI-cell adhesion molecule, Lewis Y, LI-CAM, MAGE A3, MAGE-A1, MART-1, MUCI, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp 100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-Microglobulin, Fc Receptor-like 5 (FcRL5), or molecules expressed by HIV, HCV, HBV, or other pathogens.

The tumor antigen can comprise a peptide-MHC complex, the peptide complexed with a class I or class II MHC sequence. The peptide of the peptide-MHC complex can be associated with a disease or disorder. The peptide of the peptide-MHC complex can be an intracellular tumor antigen.

The payload protein can comprise a cytokine. In some embodiments, the cytokine is selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof. The payload protein can comprise a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5. The payload protein can comprise a member of the TNF family of cytokines selected from the group consisting of TNF-alpha, TNF-beta, LT-beta, CD40 ligand, Fas ligand, CD 27 ligand, CD 30 ligand, and 4-1 BBL. The payload protein can comprise a member of the immunoglobulin superfamily of cytokines selected from the group consisting of B7.1 (CD80) and B7.2 (B70). The payload protein can comprise an interferon. In some embodiments, the interferon is selected from interferon alpha, interferon beta, or interferon gamma. The payload protein can comprise a chemokine. In some embodiments, the chemokine is selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15. The payload protein can comprise an interleukin. In some embodiments, the interleukin is selected from IL-10 IL-12, IL-1, IL-6, IL-7, IL-15, IL-2, IL-18 or IL-21. The payload protein can comprise a tumor necrosis factor (TNF). In some embodiments, the TNF is selected from TNF-alpha, TNF-beta, TNF-gamma, CD252, CD154, CD178, CD70, CD153, or 4-1BBL.

A payload protein can comprise a factor locally down-regulating the activity of endogenous immune cells. The payload protein can be capable of remodeling a tumor microenvironment and/or reducing immunosuppression at a target site of a subject.

The payload protein can comprise a monoclonal antibody, a bispecific T-cell engager (BiTE), a chimeric antigen receptor (CAR), or T-cell receptor (TCR). The CAR and/or TCR can comprise one or more of an antigen binding domain, a transmembrane domain, and an intracellular signaling domain. The monoclonal antibody and/or BiTE can comprise an antigen binding domain.

The intracellular signaling domain can comprise a primary signaling domain, a costimulatory domain, or both of a primary signaling domain and a costimulatory domain. The primary signaling domain can comprise a functional signaling domain of one or more proteins selected from the group consisting of CD3 zeta, CD3 gamma, CD3 delta, CD3 epsilon, common FcR gamma (FCER1G), FcR beta (Fc Epsilon R1b), CD79a, CD79b, Fcgamma RIIa, DAP10, and DAP12, or a functional variant thereof. The costimulatory domain can comprise a functional domain of one or more proteins selected from the group consisting of CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof.

In some embodiments, the antigen binding domain binds a tumor antigen. The tumor antigen can be a solid tumor antigen. In some embodiments, the tumor antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac (2-8) aNeu5Ac (2-3) bDGalp (1-4) bDGlcp (1-1) Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin β2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac (2-3) bDGalp (1-4) bDGlcp (1-1) Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RUI); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the tumor antigen is selected from the group comprising CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRVIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11Ralpha, IL-13R-alpha2, IL-2, IL-22R-alpha, IL-6, IL-6R, Ia, Ii, LI-CAM, L1-cell adhesion molecule, Lewis Y, LI-CAM, MAGE A3, MAGE-A1, MART-1, MUCI, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp 100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-Microglobulin, Fc Receptor-like 5 (FcRL5), or molecules expressed by HIV, HCV, HBV, or other pathogens.

The tumor antigen can comprise a peptide-MHC complex, the peptide complexed with a class I or class II MHC sequence. The peptide of the peptide-MHC complex can be associated with a disease or disorder. The peptide of the peptide-MHC complex can be an intracellular tumor antigen.

The antigen binding domain can comprise an antibody, an antibody fragment, an scFv, a Fv, a Fab, a (Fab′)₂, a single domain antibody (SDAB), a VH or VL domain, a camelid VHH domain, a Fab, a Fab′, a F(ab′)₂, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. The antigen binding domain can be connected to the transmembrane domain by a hinge region.

The transmembrane domain can comprise a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAMI (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof.

In some embodiments, the CAR or TCR further comprises a leader peptide. In some embodiments, the TCR further comprises a constant region and/or CDR4.

The payload protein can comprise a programmable nuclease. In some embodiments, the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof.

The payload protein can comprise an agonistic or antagonistic antibody or antigen-binding fragment thereof specific to: a checkpoint inhibitor or checkpoint stimulator molecule, e.g., PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, and/or TIM-3; a viral protein, e.g., Env or spike, e.g., HIV Env or SARS-COV-2 Spike; or an inflammatory cytokine. In some embodiments the inflammatory cytokine is selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF.

The payload protein can comprise a pro-death protein capable of halting cell growth and/or inducing cell death. The pro-death protein can comprise cytosine deaminase, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof. In some embodiments, the pro-death protein is capable of halting cell growth and/or inducing cell death in the presence of a pro-death agent. In some embodiments: the pro-death protein comprises Caspase-9 and the pro-death agent comprises AP1903; the pro-death protein comprises HSV thymidine kinase (TK) and the pro-death agent Ganciclovir (GCV), Ganciclovir elaidic acid ester, Penciclovir (PCV), Acyclovir (ACV), Valacyclovir (VCV), (E)-5-(2-bromovinyl)-2′-deoxyuridine (BVDU), Zidovuline (AZT), and/or 2′-exo-methanocarbathymidine (MCT); the pro-death protein comprises Cytosine Deaminase (CD) and the pro-death agent comprises 5-fluorocytosine (5-FC); the pro-death protein comprises Purine nucleoside phosphorylase (PNP) and the pro-death agent comprises 6-methylpurine deoxyriboside (MEP) and/or fludarabine (FAMP); the pro-death protein comprises a Cytochrome p450 enzyme (CYP) and the pro-death agent comprises Cyclophosphamide (CPA), Ifosfamide (IFO), and/or 4-ipomeanol (4-IM); the pro-death protein comprises a Carboxypeptidase (CP) and the pro-death agent comprises 4-[(2-chloroethyl) (2-mesyloxyethyl) amino]benzoyl-L-glutamic acid (CMDA), Hydroxy- and amino-aniline mustards, Anthracycline glutamates, and/or Methotrexate a-peptides (MTX-Phe); the pro-death protein comprises Carboxylesterase (CE) and the pro-death agent comprises Irinotecan (IRT), and/or Anthracycline acetals; the pro-death protein comprises Nitroreductase (NTR) and the pro-death agent comprises dinitroaziridinylbenzamide CB1954, dinitrobenzamide mustard SN23862, 4-Nitrobenzyl carbamates, and/or Quinones; the pro-death protein comprises Horse radish peroxidase (HRP) and the pro-death agent comprises Indole-3-acetic acid (IAA) and/or 5-Fluoroindole-3-acetic acid (FIAA); the pro-death protein comprises Guanine Ribosyltransferase (XGRTP) and the pro-death agent comprises 6-Thioxanthine (6-TX); the pro-death protein comprises a glycosidase enzyme and the pro-death agent comprises HM1826 and/or Anthracycline acetals; the pro-death protein comprises Methionine-a, y-lyase (MET) and the pro-death agent comprises Selenomethionine (SeMET); and/or the pro-death protein comprises thymidine phosphorylase (TP) and the pro-death agent comprises 5′-Deoxy-5-fluorouridine (5′-DFU).

In some embodiments, the payload protein is a cellular reprogramming factor capable of converting an at least partially differentiated cell to a less differentiated cell, e.g., Oct-3, Oct-4, Sox2, c-Myc, Klf4, Nanog, Lin28, ASCLI, MYTIL, TBX3b, SV40 large T, hTERT, miR-291, miR-294, miR-295, or any combinations thereof.

The payload protein can comprise a secretion tag. In some embodiments, the secretion tag is selected from the group comprising AbnA, AmyE, AprE, BgIC, Bg1S, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, Yqix, Yqxl, YrpD, YrpE, YuaB, Yurl, YvcE, YvgO, YvpA, YwaD, YweA, YwoF, YwtD, YwtF, YxaLk, YxiA, and YxkC. The payload protein can comprise a constitutive signal peptide for protein degradation, e.g., PEST. The payload protein can comprise a nuclear localization signal (NLS) or a nuclear export signal (NES). The payload protein can comprise a degron.

The one or more third polynucleotides can comprise at least two third polynucleotides. In some embodiments, at least one of the one or more RNA cargo molecules of each of the at least two third polynucleotides are the same or different. At least one of the one or more third polynucleotides can comprise a promoter operably linked to an RNA cargo molecule. The promoter can be capable of inducing the transcription of the RNA cargo molecule. The at least one third polynucleotide can comprise one or more of a 5′ UTR, 3′ UTR, a minipromoter, an enhancer, a splicing signal, a polyadenylation signal, a terminator, a protein degradation signal, and an internal ribosome-entry element (IRES) operably linked to the RNA cargo molecule.

In some embodiments, the at least one of the one or more third polynucleotides further comprises a transcript stabilization element. The transcript stabilization element can comprise woodchuck hepatitis post-translational regulatory element (WPRE), bovine growth hormone polyadenylation (bGH-polyA) signal sequence, human growth hormone polyadenylation (hGH-polyA) signal sequence, or any combination thereof.

The promoter can comprise a ubiquitous promoter. In some embodiments, the ubiquitous promoter is selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EFla) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

The promoter can be an inducible promoter. The inducible promoter can be a tetracycline responsive promoter, a TRE promoter, a Tre3G promoter, an ecdysone responsive promoter, a cumate responsive promoter, a glucocorticoid responsive promoter, and estrogen responsive promoter, a PPAR-y promoter, or an RU-486 responsive promoter. The promoter can comprise a tissue-specific promoter and/or a lineage-specific promoter.

### Tetherin Inhibitors

In some embodiments, the nucleic acid composition further comprises a polynucleotide comprising or encoding a tetherin inhibitor. The tetherin inhibitor can be capable of modulating expression, concentration, localization, stability, and/or activity of tetherin. In some embodiments, presence or expression of the tetherin inhibitor in the cell results in an increase in ENP production by the cell by at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values), relative to a cell that does not comprise or express the tetherin inhibitor

The tetherin inhibitor can comprise a dsRNA, an siRNA, an shRNA, a pre-miRNA, a pri-miRNA, a miRNA, an stRNA, an lncRNA, a piRNA, a snoRNA, or a protein. In some embodiments, one or more of (i) the polynucleotide comprising or encoding the tetherin inhibitor, (ii) the first polynucleotide encoding the dimerization fusion protein, and (iii) the second polynucleotide encoding the adapter fusion protein, are present in a same or a different nucleic acid molecule. In some embodiments, the amount of (i) the polynucleotide comprising or encoding the tetherin inhibitor; and (ii) the first polynucleotide encoding the dimerization fusion protein, and/or the second polynucleotide encoding the adapter fusion protein, are present in the composition at a molar ratio of about 1:1, 1:5 or 1:25. In some embodiments, the amount of (i) the polynucleotide comprising or encoding the tetherin inhibitor; and (ii) the first polynucleotide encoding the dimerization fusion protein, and/or the second polynucleotide encoding the adapter fusion protein, are present in the composition at a molar ratio of or of about 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values. In some embodiments, the ratio can be at least, or be at most, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, or 10000:1

In some embodiments, the polynucleotide comprising or encoding the tetherin inhibitor and the first polynucleotide encoding the dimerization fusion protein are present in the same nucleic acid. In some embodiments, the polynucleotide comprising or encoding the tetherin inhibitor and the second polynucleotide encoding the adapter fusion protein are present in the same nucleic acid.

In some embodiments, the tetherin inhibitor comprises or is derived from a viral protein. In some embodiments, the virus is HIV-1, HIV-2, SIV, Ebola virus, KSHV, SARS CoV, or SARS-COV-2. The tetherin inhibitor can comprise HIV-1 Vpu protein, KSHV K5 protein, SARS-COV-2 ORF7a, HIV-2 Env, Ebola GP, SIV Env, SIV Vpu, SIV Nef, or any portions, variants or derivatives thereof. The tetherin inhibitor can comprise an amino acid sequence having at least 65%, 70%, 75%, 80%, 85%, 90%, 95%, 98%, or 99% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) sequence identity to an amino acid sequence selected from the group consisting of SEQ ID NOs: 125-127. The tetherin inhibitor can comprise an amino acid sequence of any one of SEQ ID NOs: 125-127.

### Enveloped Nanoparticles

he ENPs can have one or more dimensions of a eukaryotic virus. In some embodiments, less than about 1%, 2%, 3%, 4%, 5%, 6%, 7%, 8%, 9%, or 10%, of the ENPs of the population of ENPs have a particle size smaller than about 10 nm. In some embodiments, less than about 1%, 2%, 3%, 4%, 5%, 6%, 7%, 8%, 9%, or 10%, of the ENPs of the population of ENPs have a particle size exceeding about 80 nm. In some embodiments, the average diameter of the ENPs of the population of ENPs range from about 5 nm to about 80 nm, from about 15 nm to about 50 nm, or from about 20 nm to about 40 nm. The average diameter of the ENPs of the population of ENPs can be about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm, optionally the average is the mean, median or mode, optionally the mean is the arithmetic mean, geometric mean, and/or harmonic mean.

In some embodiments, the ENPs have a minimum diameter of about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, or about 50 nm. In some embodiments, the ENPs have a maximum diameter of about 10 nm, about 12 nm, about 14 nm, about 16 nm, about 18 nm, about 20 nm, about 22 nm, about 24 nm, about 26 nm, about 28 nm, about 30 nm, about 32 nm, about 34 nm, about 36 nm, about 38 nm, about 40 nm, about 42 nm, about 44 nm, about 46 nm, about 48 nm, about 50 nm, about 52 nm, about 54 nm, about 56 nm, about 58 nm, about 60 nm, about 62 nm, about 64 nm, about 66 nm, about 68 nm, about 70 nm, about 72 nm, about 74 nm, about 76 nm, about 78 nm, or about 80 nm.

In some embodiments, storage of the ENPs at 4° C. for at least three months reduces immunogenicity less than about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%. The composition can be stable for at least about 2 weeks, about 1 month, about 2 months, about 3 months, about 4 months, about 5 months, about 6 months, or about 1 year, after storage as a liquid at a temperature of about 4° C. The ENPs can be derived from cell cultures transiently transfected with the nucleic acid composition, e.g., derived via ultracentrifugation and/or size exclusion chromatography, e.g., ultracentrifugation on a 20% sucrose cushion, e.g., transfected via calcium phosphate transfection, DEAE-dextran mediated transfection, cationic lipid-mediated transfection, electroporation, electrical nuclear transport, chemical transduction, electrotransduction, Lipofectamine-mediated transfection, Effectene-mediated transfection, lipid nanoparticle (LNP)-mediated transfection, or any combination thereof.

### Vectors and Carriers

The nucleic acid composition (e.g., comprising polynucleotides encoding any of the fusion protein, the dimerization fusion protein, the adapter fusion protein, the soluble RBP, and/or the cell fusion protein) can be complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition (e.g., comprising polynucleotides encoding any of the fusion protein, the dimerization fusion protein, the adapter fusion protein, the soluble RBP, and/or the cell fusion protein) is, comprises, or further comprises, one or more vectors. At least one of the one or more vectors can be a viral vector, a plasmid, a transposable element, a naked DNA vector, a lipid nanoparticle (LNP), or any combination thereof. The viral vector can be an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. The transposable element can be piggybac transposon or sleeping beauty transposon. The polynucleotide(s) encoding any of the fusion protein, the dimerization fusion protein, the adapter fusion protein, the soluble RBP, and/or the cell fusion protein can be comprised in the one or more vectors. The polynucleotide(s) encoding any of the fusion protein, the dimerization fusion protein, and/or the adapter fusion protein be comprised in the same vector and/or different vectors. The polynucleotide(s) encoding any of the fusion protein, the dimerization fusion protein, the adapter fusion protein, the soluble RBP, and/or the cell fusion protein can be situated on the same nucleic acid and/or different nucleic acids.

The polynucleotide(s) encoding any of the fusion protein, the dimerization fusion protein, the adapter fusion protein, the soluble RBP, and/or the cell fusion protein can be operably linked to one or more promoters capable of inducing transcription of said polynucleotide(s). The promoter can comprise a ubiquitous promoter, an inducible promoter, a tissue-specific promoter and/or a lineage-specific promoter. The ubiquitous promoter can be selected from the group comprising a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EFla) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The polynucleotide(s) encoding any of the fusion protein, the dimerization fusion protein, the adapter fusion protein, the soluble RBP, and/or the cell fusion protein can be present on the same or different nucleic acids. The polynucleotide(s) encoding any of the fusion protein, the dimerization fusion protein, the adapter fusion protein, the soluble RBP, and/or the cell fusion protein can be operably linked to a tandem gene expression element (e.g., an internal ribosomal entry site (IRES), foot- and-mouth disease virus 2A peptide (F2A), equine rhinitis A virus 2A peptide (E2A), porcine teschovirus 2A peptide (P2A) or Thosea asigna virus 2A peptide (T2A), or any combination thereof). The polynucleotide(s) encoding any of the fusion protein, the dimerization fusion protein, the adapter fusion protein, the soluble RBP, and/or the cell fusion protein can comprise a transcript stabilization element (e.g., woodchuck hepatitis post-translational regulatory element (WPRE), bovine growth hormone polyadenylation (bGH-polyA) signal sequence, human growth hormone polyadenylation (hGH-polyA) signal sequence, or any combination thereof).

The nucleic acid composition can be or can comprise mRNA. The mRNA can be formulated in a lipid nanoparticle (LNP). The term “lipid nanoparticle”, also referred to as LNP, refers to a particle having at least one dimension on the order of nanometers (e.g., 1-1,000 nm) which includes one or more lipids. In some embodiments, such lipid nanoparticles comprise a cationic lipid and one or more excipient selected from neutral lipids, charged lipids, steroids and polymer conjugated lipids (e.g., a pegylated lipid). In some embodiments, the mRNA, or a portion thereof, is encapsulated in the lipid portion of the lipid nanoparticle or an aqueous space enveloped by some or all of the lipid portion of the lipid nanoparticle, thereby protecting it from enzymatic degradation or other undesirable effects induced by the mechanisms of the host organism or cells e.g., an adverse immune response. In some embodiments, the mRNA or a portion thereof is associated with the lipid nanoparticles. An LNP may comprise any lipid capable of forming a particle to which the one or more nucleic acid molecules are attached, or in which the one or more nucleic acid molecules are encapsulated. The term “lipid” refers to a group of organic compounds that are derivatives of fatty acids (e.g., esters) and are generally characterized by being insoluble in water but soluble in many organic solvents. Lipids are usually divided in at least three classes: (1) “simple lipids” which include fats and oils as well as waxes; (2) “compound lipids” which include phospholipids and glycolipids; and (3) “derived lipids” such as steroids.

The LNP can comprise one or more of an ionizable cationic lipid, a non-cationic lipid (e.g., a neutral lipid), a sterol, and a PEG-modified lipid. The LNP can comprise 0.5-15 mol % PEG-modified lipid, 5-25 mol % non-cationic lipid, 25-55 mol % sterol, and 20-60 mol % ionizable cationic lipid. The LNP can comprise 40-55 mol % ionizable cationic lipid, 5-15 mol % neutral lipid, 35-45 mol % sterol, and 1-5 mol % PEG-modified lipid. In some embodiments, the RNA (e.g., mRNA) of the disclosure is formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016/000129; PCT/US2016/014280; PCT/US2016/014280; PCT/US2017/038426; PCT/US2014/027077; PCT/US2014/055394;
PCT/US2016/052117; PCT/US2012/069610; PCT/US2017/027492; PCT/US2016/059575 and PCT/US2016/069491 all of which are incorporated by reference herein in their entirety.

In some embodiments, the LNP comprises: 47 mol % ionizable cationic lipid, 11.5 mol % neutral lipid, 38.5 mol % sterol, and 3.0 mol % PEG-modified lipid; 48 mol % ionizable cationic lipid, 11 mol % neutral lipid, 38.5 mol % sterol, and 2.5 mol % PEG-modified lipid; 49 mol % ionizable cationic lipid, 10.5 mol % neutral lipid, 38.5 mol % sterol, and 2.0 mol % PEG-modified lipid; 50 mol % ionizable cationic lipid, 10 mol % neutral lipid, 38.5 mol % sterol, and 1.5 mol % PEG-modified lipid; or 51 mol % ionizable cationic lipid, 9.5 mol % neutral lipid, 38.5 mol % sterol, and 1.0 mol % PEG-modified lipid.

The ionizable cationic lipid can be heptadecan-9-yl 8 ((2 hydroxyethyl) (6 oxo 6-(undecyloxy) hexyl) amino) octanoate. The neutral lipid can be 1,2 distearoyl sn glycero-3 phosphocholine (DSPC). The sterol can be cholesterol. The PEG-modified lipid can be 1-monomethoxypolyethyleneglycol-2,3-dimyristylglycerol with polyethylene glycol of average molecular weight 2000 (PEG2000 DMG).

The wt/wt ratio of lipid to mRNA can be from about 1:100 to about 100:1 (e.g., 1:1, 1:1.1, 1:1.2, 1:1.3, 1:1.4, 1:1.5, 1:1.6, 1:1.7, 1:1.8, 1:1.9, 1:2, 1:2.5, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100 to 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, or a number or a range between any of these values).

The LNP can comprise a cationic lipid. The cationic lipid can be cationisable, i.e., it becomes protonated as the pH is lowered below the pKa of the ionizable group of the lipid, but is progressively more neutral at higher pH values. When positively charged, the lipid is then able to associate with negatively charged nucleic acids. In some embodiments, the cationic lipid comprises a zwitterionic lipid that assumes a positive charge on pH decrease. The LNP may comprise any lipid capable of forming a particle to which the one or more nucleic acid molecules are attached, or in which the one or more nucleic acid molecules are encapsulated. In some embodiments, the LNP may comprise any further cationic or cationisable lipid, i.e. any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy) propyl)-N,N,N-trimethylammonium chloride (DOTMA); N,N-distearyl-N,N-dimethylammonium bromide (DDAB); N-(2,3dioleoyloxy) propyl)-N,N,N-trimethylammonium chloride (DOTAP); 3-(N-(N′,N′dimethylaminoethane)-carbamoyl) cholesterol (DC-Chol), N-(1-(2,3-dioleoyloxy) propyl)N-2-(sperminecarboxamido) ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy) propylamine (DODMA), and N-(1,2dimyristyloxyprop-3-yl)-N,N-dimethyl-N-hydroxyethyl ammonium bromide (DMRIE).

Additionally, a number of commercial preparations of cationic lipids are available which can be used in embodiments provided herein. These include, for example, LIPOFECTIN® (commercially available cationic liposomes comprising DOTMA and 1,2-dioleoyl-sn-3phosphoethanolamine (DOPE), from GIBCO/BRL, Grand Island, N.Y.); LIPOFECTAMINE® (commercially available cationic liposomes comprising N-(1-(2,3dioleyloxy) propyl)-N-(2-(sperminecarboxamido) ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-lcarboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE). In some embodiments, the neutral lipid is 1,2-distearoyl-sn-glycero-3phosphocholine (DSPC).

In some embodiments, the cationic lipid is an amino lipid. Suitable amino lipids useful include those described in WO2012/016184, incorporated herein by reference in its entirety. Representative amino lipids include, but are not limited to, 1,2-dilinoleyoxy-3-(dimethylamino) acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.CI), 1,2-dilinoleyloxy-3-(N-methylpiperazino) propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino) ethoxypropane (DLin-EG-DMA), and 2,2-dilinoleyl-4-dimethylaminomethyl-[1,3]-dioxolane (DLin-K-DMA).

In some embodiments, a non-cationic lipid comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is DMG-PEG, PEG-c-DOMG (also referred to as PEG-DOMG), PEG-DSG and/or PEG-DPG. In some embodiments, a sterol comprises cholesterol, fecosterol, sitosterol, ergosterol, campesterol, stigmasterol, brassicasterol, tomatidine, ursolic acid, alpha-tocopherol, and mixtures thereof.

In some embodiments, the nucleic acid composition comprising an mRNA sequence is a modified mRNA sequence. In this context, a modification as defined herein can lead to a stabilization of the mRNA sequence provided herein. In some embodiments, there is thus provided a stabilized mRNA sequence comprising at least one coding region as defined herein (e.g., polynucleotide(s) encoding any of the fusion protein, the dimerization fusion protein, the adapter fusion protein, the soluble RBP, and/or the cell fusion protein). In some embodiments, the nucleic acid composition comprising an mRNA sequence may thus be provided as a “stabilized mRNA sequence”, that is to say as an mRNA that is essentially resistant to in vivo degradation (e.g., by an exo- or endo-nuclease). Such stabilization can be effected, for example, by a modified phosphate backbone of an mRNA provided herein. A backbone modification can be a modification in which phosphates of the backbone of the nucleotides contained in the mRNA are chemically modified. Nucleotides that can be used in this connection contain e.g., a phosphorothioate-modified phosphate backbone, such as at least one of the phosphate oxygens contained in the phosphate backbone being replaced by a sulfur atom. Stabilized mRNAs may further include, for example: non-ionic phosphate analogues, such as, for example, alkyl and aryl phosphonates, in which the charged phosphonate oxygen is replaced by an alkyl or aryl group, or phosphodiesters and alkylphosphotriesters, in which the charged oxygen residue is present in alkylated form. Such backbone modifications typically include, without implying any limitation, modifications from the group consisting of methylphosphonates, phosphoramidates and phosphorothioates (e.g., cytidine-5′-O-(1-thiophosphate)). The term “mRNA modification” as used herein may refer to chemical modifications comprising backbone modifications as well as sugar modifications or base modifications. In this context, a modified mRNA (sequence) as defined herein may contain nucleotide analogues/modifications, e.g., backbone modifications, sugar modifications or base modifications. A backbone modification can be a modification, in which phosphates of the backbone of the nucleotides contained in an mRNA compound comprising an mRNA sequence as defined herein are chemically modified. A sugar modification can be a chemical modification of the sugar of the nucleotides of the mRNA compound comprising an mRNA sequence as defined herein. Furthermore, a base modification can be a chemical modification of the base moiety of the nucleotides of the mRNA compound comprising an mRNA sequence. In this context, nucleotide analogues or modifications can be selected from nucleotide analogues, which are applicable for transcription and/or translation.

The mRNA provided herein can comprise a 5′ untranslated region (UTR), a 3′ UTR, and/or a cap (e.g., a CAP analogue). A modified mRNA sequence as defined herein, can be modified by the addition of a so-called “5′-CAP structure”, which can stabilize the mRNA as described herein. A 5′-CAP is an entity, typically a modified nucleotide entity, which generally “caps” the 5′-end of a mature mRNA. A 5′-CAP may typically be formed by a modified nucleotide, particularly by a derivative of a guanine nucleotide. In some embodiments, the 5′-CAP is linked to the 5′-terminus via a 5′-5′-triphosphate linkage. A 5′-CAP may be methylated, e.g., m7GpppN, wherein N is the terminal 5′ nucleotide of the nucleic acid carrying the 5′-CAP, typically the 5′-end of an mRNA. m7GpppN is the 5′-CAP structure, which naturally occurs in mRNA transcribed by polymerase II and is therefore in some embodiments is not considered as modification comprised in a modified mRNA in this context. Accordingly, a modified mRNA sequence may comprise a m7GpppN as 5′-cap, but additionally the modified mRNA sequence typically comprises at least one further modification as defined herein. A CAP analogue refers to a non-polymerizable di-nucleotide that has CAP functionality in that it facilitates translation or localization, and/or prevents degradation of the RNA molecule when incorporated at the 5′-end of the RNA molecule. Non-polymerizable means that the CAP analogue will be incorporated only at the 5′-terminus because it does not have a 5′ triphosphate and therefore cannot be extended in the 3′-direction by a template-dependent RNA polymerase. CAP analogues include, but are not limited to, a chemical structure selected from the group consisting of m7GpppG, m7GpppA, m7GpppC; unmethylated CAP analogues (e.g., GpppG); dimethylated CAP analogue (e.g., m2,7GpppG), trimethylated CAP analogue (e.g., m2,2,7GpppG), dimethylated symmetrical CAP analogues (e.g., m7Gpppm7G), or anti reverse CAP analogues (e.g., ARCA; m7,2′OmeGpppG, m7,2′dGpppG, m7,3′OmeGpppG, m7,3′dGpppG and their tetraphosphate derivatives) (Stepinski et al., 2001. RNA 7 (10): 1486-95). Further CAP analogues have been described previously (U.S. Pat. No. 7,074,596, WO2008/016473, WO2008/157688, WO2009/149253, WO2011/015347, and WO2013/059475).

The mRNA can comprise one or more modified nucleotides selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, and 2-thiocytidine. The mRNA can comprise a modified nucleotide in place of one or more uridines. The modified nucleoside can be selected from pseudouridine (ψ), N 1-methyl-pseudouridine (m 1Y), and 5-methyl-uridine (m5U). In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published PCT Patent Application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB 2017/051367 all of which are incorporated by reference herein.

### Pharmaceutical Compositions and Therapeutic Applications

### Pharmaceutical Compositions

Also provided herein include pharmaceutical compositions comprising compositions (e.g., a nucleic acid composition, a population of ENPs) as herein described. Pharmaceutical compositions can comprise the compositions provided herein (e.g., a nucleic acid composition, a population of ENPs) in combination with one or more compatible and pharmaceutically acceptable carriers.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In some embodiments, the pharmaceutically acceptable carrier comprises a pharmaceutical acceptable salt. As used herein, a “pharmaceutical acceptable salt” includes a salt of an acid form of one of the components of the compositions herein described. These include organic or inorganic acid salts of the amines. Preferred acid salts are the hydrochlorides, acetates, salicylates, nitrates and phosphates. Other suitable pharmaceutically acceptable salts are well known to those skilled in the art and include basic salts of a variety of inorganic and organic acids.

The pharmaceutical composition can be formulated for a variety of modes of administration. Techniques for formulation and administration can be found, for example, in “Remington's Pharmaceutical Sciences”, 18th ed., 1990, Mack Publishing Co., Easton, Pa. In some embodiments, the pharmaceutical compositions of the present disclosure may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension: (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the hydrogel composition. The pharmaceutical compositions can comprise one or more pharmaceutically-acceptable carriers.

Formulations useful in the methods of the present disclosure include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient which can be combined with a pharmaceutically acceptable carrier to produce a single dosage form will vary depending upon the host being treated, the particular mode of administration. The amount of active ingredient, which can be combined with a pharmaceutically acceptable carrier to produce a single dosage form will generally be that amount of the composition (e.g., a nucleic acid composition, a population of ENPs) which produces a therapeutic effect. Generally, out of one hundred percent, this amount will range from about 1% to about 99% of active ingredient, optionally from about 5% to about 70%, optionally from about 10% to about 30%.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like.

In solid dosage forms for oral administration (capsules, tablets, pills, dragees, powders, granules and the like), the composition (e.g., a nucleic acid composition, a population of ENPs) is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

The pharmaceutical composition can be formulated for parenteral administration by injection, e.g., by bolus injection or continuous infusion. Formulations for injection can be presented in a unit dosage form, e.g., in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

Also disclosed herein include cells comprising any of the nucleic acid compositions described herein. The cell can comprise a stem cell, a fibroblast cell, a chondrocyte, a keratinocyte, a hepatocyte, a pancreatic islet cell, or an immune cell. The immune cell can comprise a T cell, a dendritic cell (DC), a natural killer (NK) cell, or a macrophage. The T cell can be a CAR-T cell. In some embodiments, a composition, e.g., a pharmaceutical composition, comprises a population of cells (e.g., CAR T-cells) comprising a nucleic acid composition of the disclosure.

### Methods of Delivery and Methods of Treating

Disclosed herein include methods of delivering one or more cargo RNA molecules to a cell or a population of cells. In some embodiments, the method comprises contacting a cell or a population of cells with a composition of the disclosure, thereby delivering the one or more cargo RNA molecules to the cell or the population of cells.

In some embodiments, the contacting step can be performed in vivo, in vitro, and/or ex vivo. In some embodiments, the cell or the population of cells comprise one or more cells of a subject or are comprised within a tissue of a subject. In some embodiments, the subject is suffering from a disease or disorder. The cell or the population of cells can comprise prokaryotic cells or eukaryotic cells. The eukaryotic cells can comprise plant or animal cells. The cell or population of cells can comprise an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can can comprise an embryonic stem cell, an induced pluripotent stem cell (iPSC), a hematopoietic stem/progenitor cell (HSPC), or any combination thereof.

Disclosed herein include methods of treating or preventing a disease or disorder in a subject in need thereof, comprising: administering to the subject a pharmaceutically effective amount of a composition of any the disclosure, thereby treating or preventing the disease or disorder in the subject.

The subject can be a mammalian subject, e.g., a human subject. The disease or disorder can be a blood disease, an immune disease, a neurological disease or disorder, a cardiovascular disease or disorder, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof. The disease or disorder can be a solid tumor.

In some embodiments, the disease or disorder is an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-COV-2), Creutzfeldt-Jacob Disease/transmissible spongiform encephalopathy, Cryptosporidiosis (Crypto), Cyclosporiasis, Dengue 1,2,3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus Influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Melioidosis, Meningitis (Viral), Meningococcal Disease (Meningitis (Bacterial)), Middle East Respiratory Syndrome Coronavirus (MERS-COV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphyloccal Infection Methicillin-resistant (MRSA), Staphylococcal Food Poisoning Enterotoxin B Poisoning (Staph Food Poisoning), Saphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichonosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

The disease can be associated with expression of a tumor-associated antigen. The disease associated with expression of a tumor antigen-associated is selected from the group consisting of a proliferative disease, a precancerous condition, a cancer, and a non-cancer related indication associated with expression of the tumor antigen. The tumor associated antigen can comprise a peptide-MHC complex, the peptide complexed with a class I or class II MHC sequence. The peptide of the peptide-MHC complex can be associated with a disease or disorder. The peptide of the peptide-MHC complex can be an intracellular tumor antigen.

In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers.

In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

The disease or disorder can be a cardiovascular disease. The cardiovascular disease can comprise angina, arrhythmia, atherosclerosis, atrial fibrillation, cardiomyopathy, congenital heart disease, coronary artery disease, enlarged heart, heart failure, infective endocarditis, an inherited rhythm disorder, Kawasaki disease, long Q-T syndrome, Marfan syndrome, pericarditis, peripartum cardiomyopathy, rheumatic heart disease, valvular heart disease, vascular cognitive impairment, or any combination thereof.

The administering can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof.

### Kits

The compositions disclosed herein (e.g., a nucleic acid composition, a population of ENPs) can be provided as components of a kit.

Kits can include compositions disclosed herein (e.g., a nucleic acid composition, a population of ENPs, a pharmaceutical composition) as well components for making such compositions. As such, kits can include, for example, primers, nucleic acid molecules, expression vectors, nucleic acid constructs encoding protein antigens and/or particle-forming subunits described herein, cells, buffers, substrates, reagents, administration means (e.g., syringes), and instructions for using any of said components. It should be appreciated that a kit may comprise more than one container comprising any of the aforementioned, or related, components. For example, certain parts of the kit may require refrigeration, whereas other parts can be stored at room temperature. Thus, as used herein, a kit can comprise components sold in separate containers by one or more entity, with the intention that the components contained therein be used together.

The composition (e.g., a nucleic acid composition, a population of ENPs) can comprise Tris buffer, sucrose, and/or sodium acetate. The composition can comprise an adjuvant (e.g., aluminum hydroxide, alhydrogel, AddaVax, MF59, AS03, Freund's adjuvant, Montanide ISA51, CpG, Poly I: C, glucopyranosyl lipid A, flagellin, resiquimod, or any combination thereof). The composition can be a lyophilized composition. In some embodiments, the lyophilized composition has a water content of less than about 10%. The nucleic acid composition and the LNP-forming components can be in separate vials. The composition can be a pharmaceutical composition, and the pharmaceutical composition can comprise one or more pharmaceutically acceptable carriers, diluents and/or excipients. The composition can comprise instructions for use of the composition for: treating or preventing a disease or disorder; and/or diagnosing a subject as a subject having a disease or disorder.

In some embodiments, the composition is formulated or is to be formulated: as a liquid, a solid, or a combination thereof; for injection; for intramuscular administration, intranasal administration, transdermal administration, aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection; and/or as particles (e.g., iron oxide particles, liposomes, micelles, polymer complexes, cationic peptide nanoemulsions, virus-like particles (VLPs), lipid nanoparticles (LNP) and/or lipoplex (LPX) particles).

### EXAMPLES

Some aspects of the embodiments discussed above are disclosed in further detail in the following examples, which are not in any way intended to limit the scope of the present disclosure.

### Example 1

### Engineered Enveloped Nanoparticles (ENPs) as a Delivery System for Nucleic Acid-Based Cargoes

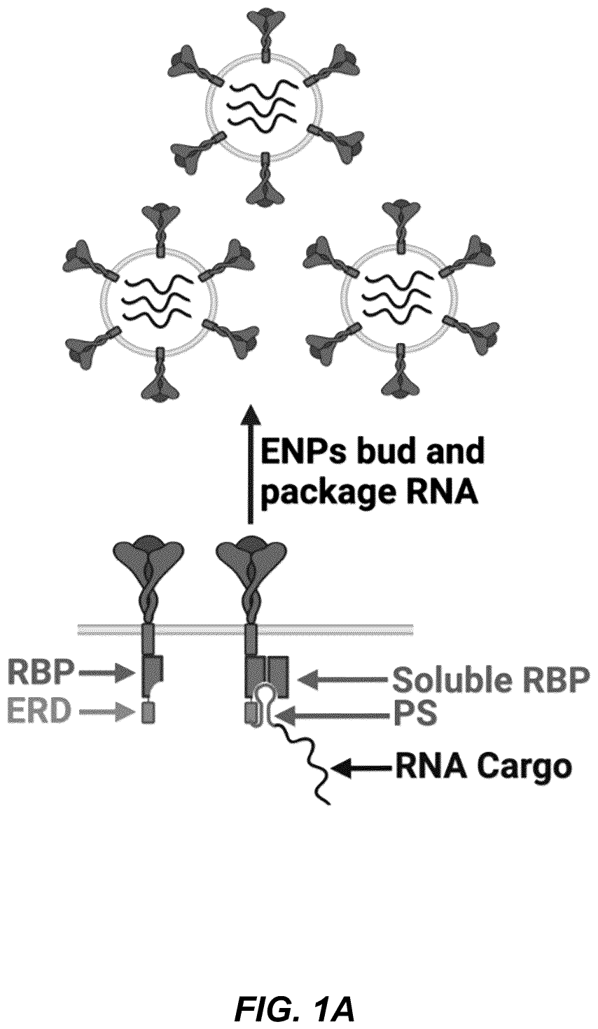

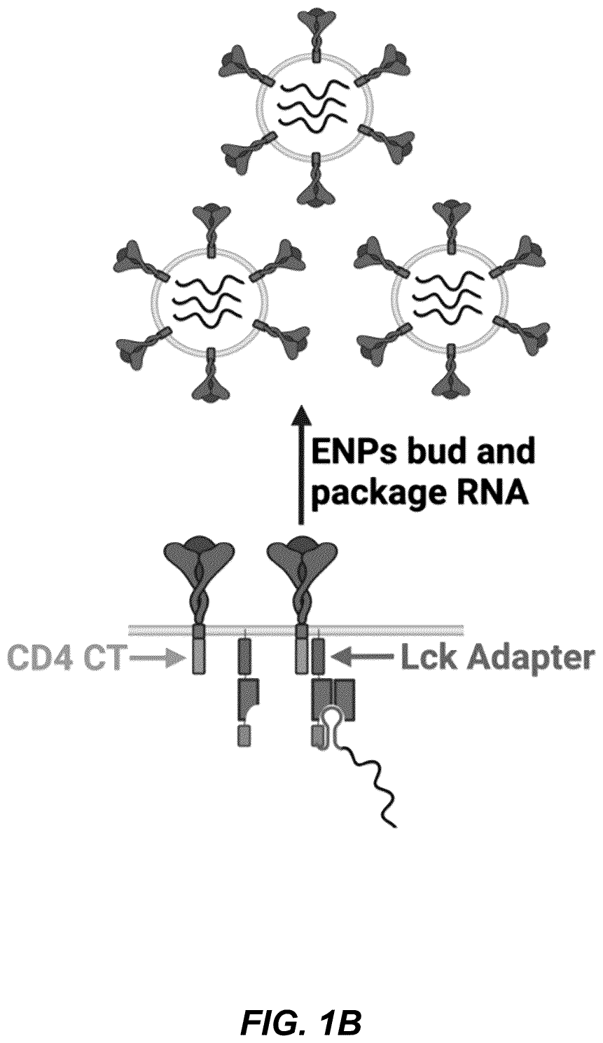

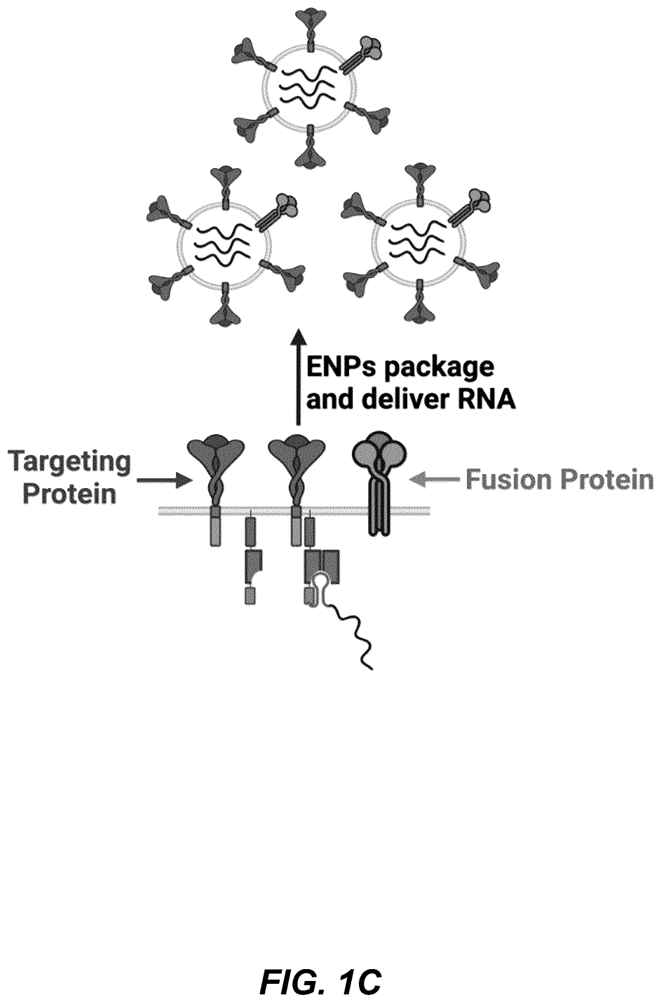

Described herein is a novel ENP delivery platform for nucleic acid-based cargoes based on the ERD technology. The ENP delivery system can be tailored to efficiently package and deliver nucleic acid-based therapeutic cargoes to specific target cells and is ideally suited for large-scale production. ENP packaging of RNA cargoes can be achieved by inserting an RNA-binding protein (RBP) into the CSP-ERD fusion construct that specifically interacts with a packaging sequence (PS) that was introduced into the RNA cargo (FIG. 1A). This interaction facilitates efficient incorporation of the RNA cargo into the budding ENP. For RBPs that require dimerization or oligomerization for RNA-binding, co-expression of the soluble RBP facilitates dimerization/oligomerization and increases RNA packaging efficiency. ENP encapsulation of specific RNA cargoes can be further improved by using an adapter approach, where the adapter contains the RBP and ERD sequences and interacts with a modified cytoplasmic domain of the CSP displayed on the ENP surface (FIG. 1B). Delivery of the RNA cargo to specific cell types can be achieved through co-display of two CSPs on the ENP surface: i) a targeting protein that binds to a cell surface receptor specifically expressed on target cells; ii) a fusion protein that promotes fusion between the ENP envelope and cellular membrane to facilitate entry of the RNA cargo into target cells (FIG. 1C). As demonstrated herein, the designed ENP delivery systems produce efficient and targeted delivery of a specific messenger RNA (mRNA) resulting in high cellular expression of the encoded protein in the target cells. The designed ENP delivery systems can be applied to deliver a wide range of nucleic acid-based cargoes for therapeutic, diagnostic, or biological applications.

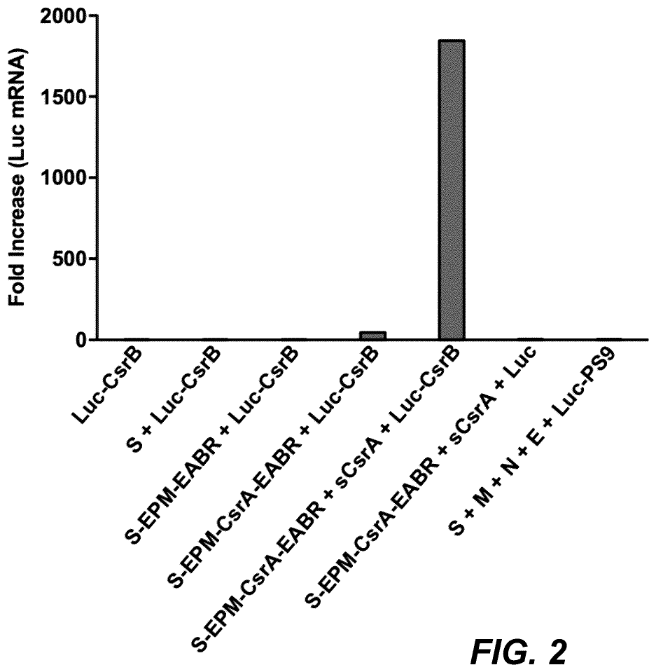

SARS-COV-2 spike(S)-based ERD ENPs were initially designed that package RNA by inserting the small RBP CsrA from E. coli into the cytoplasmic domain of S protein downstream of the previously described endocytosis preventing motif (EPM) and a 12-residue flexible linker, and upstream of a 4-residue flexible linker and the EABR sequence (Table 4). CsrA specifically interacts with the noncoding regulatory RNA CsrB, which was inserted into the 3′ untranslated region of a firefly luciferase (Luc)-encoding mRNA as PS (Table 4). Plasmids encoding S-EPM-CsrA-EABR and Luc-CsrB were co-transfected into Expi293 cells. After 72 hours, ENPs were purified from supernatants by sucrose ultracentrifugation. RNA was extracted from purified ENPs, and Luc mRNA was quantified by RT-qPCR. Purified S-EPM-CsrA-EABR+Luc-CsrB ENPs contained>40-fold more Luc-CsrB mRNA compared to various control conditions, including Luc-CsrB alone, S+Luc-CsrB, and S-EPM-EABR+Luc-CsrB, suggesting that the CsrA-CsrB interaction promotes packaging of a specific RNA molecule into ENPs (FIG. 2). It was hypothesized that co-expression of soluble CsrA (sCsrA) could enhance RNA packaging, because CsrA binds to CsrB as a homodimer5. Co-transfection of plasmids encoding S-EPM-CsrA-EABR+sCsrA+Luc-CsrB resulted in >40-fold higher Luc-CsrB mRNA levels compared to S-EPM-CsrA-EABR+Luc-CsrB and >1,800-fold higher mRNA levels compared to Luc-CsrB alone. Removal of the CsrB motif completely abolished Luc mRNA packaging, highlighting that the RBP and PS components are both required for RNA packaging, which ensures highly specific RNA encapsulation in self-assembling ENPs.

The engineered ERD ENPs were also compared to an alternative ENP platform based on the co-expression of the SARS-COV-2 structural proteins spike, nucleocapsid (N), membrane (M), and envelope (E), which has been shown to form ENPs and package RNAs that contain the SARS-COV-2 virus' minimal genomic packaging sequence PS97. Purified S-EPM-CsrA-EABR+sCsrA+Luc-CsrB ENPs contained 1,256-fold higher Luc mRNA levels compared to S+N+M+E+Luc-PS9 ENPs, suggesting that the engineered ERD ENPs package specific RNA cargo more efficiently. The ERD ENP production rate is ˜100-fold higher compared to S+N+M+E ENPs, which may have also contributed to the large observed difference in Luc mRNA levels in purified ENP samples.

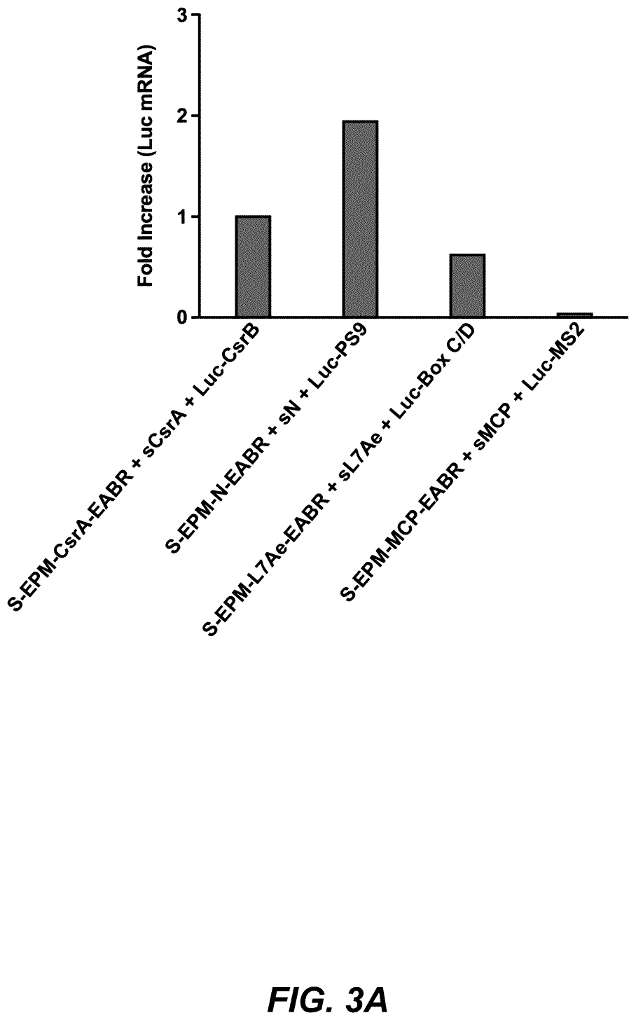

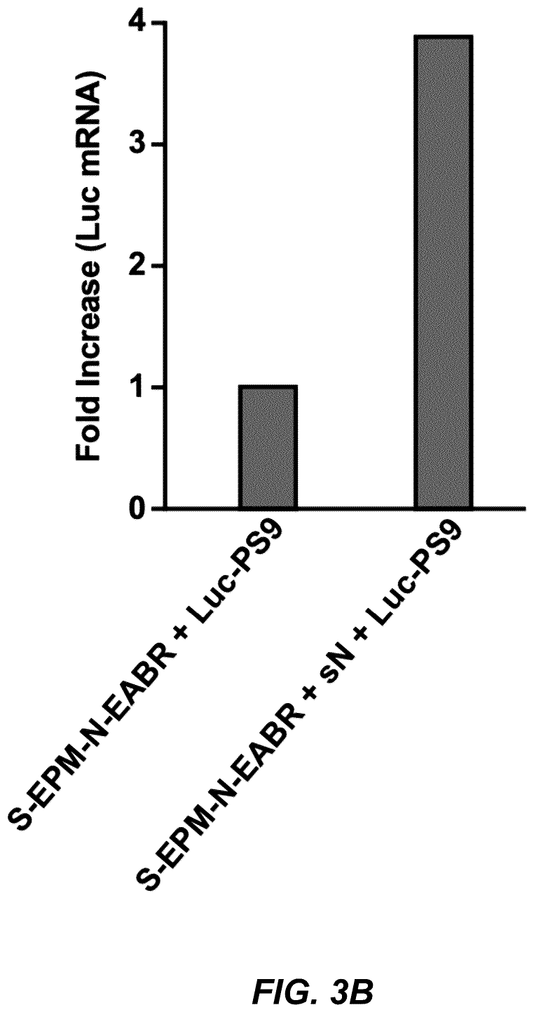

ENP constructs with various RBP and PS sequences were designed to evaluate RNA packaging. The CsrA-CsrB interaction was substituted with i) the SARS-COV-2 N protein and its genomic packaging sequence PS97; ii) the archaeal ribosomal protein L7Ae and its Box C/D binding motif8; or iii) the MS2 bacteriophage coat protein (MCP) and 12 repeats of its MS2 stem loop binding motif (Table 4). Purified S-EPM-N-EABR+sN+Luc-PS9 ENPs contained˜2-fold higher Luc mRNA levels compared to S-EPM-CsrA-EABR+sCsrA+Luc-CsrB ENPs (FIG. 3A). Similar to CsrA-based constructs, the addition of a plasmid encoding soluble N protein improved Luc mRNA packaging for S-EPM-N-EABR+sN+Luc-PS9 ENPs (FIG. 3B). S-EPM-L7Ae-EABR+sL7Ae+Luc-Box C/D ENPs achieved slightly lower Luc mRNA packaging than S-EPM-CsrA-EABR+sCsrA+Luc-CsrB ENPs, while 27-fold lower Luc mRNA levels were measured for S-EPM-MCP-EABR+sMCP+Luc-MS2. These results indicate that various RBP-PS interactions can promote efficient packaging of specific RNA cargoes into engineered ENPs.

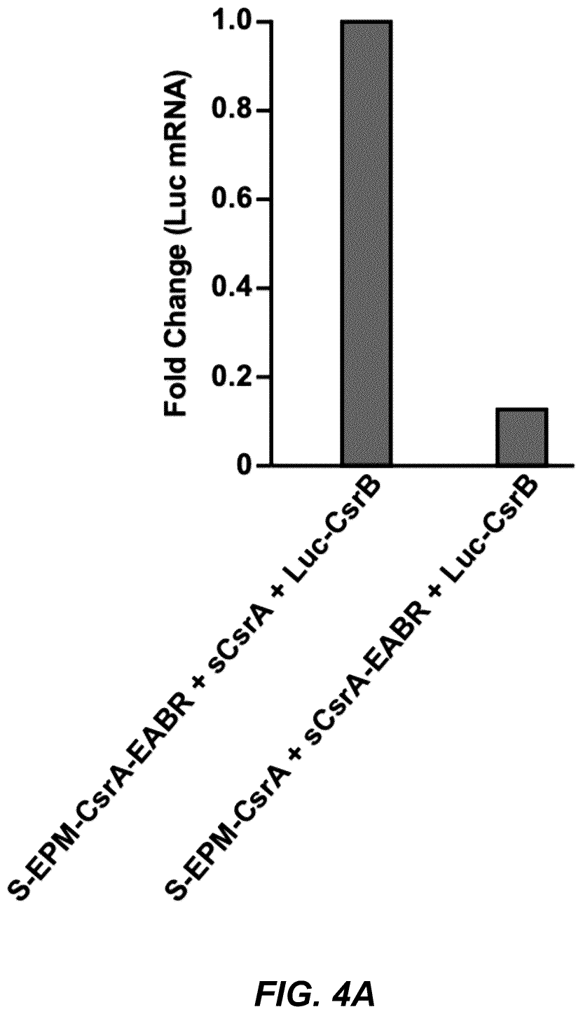

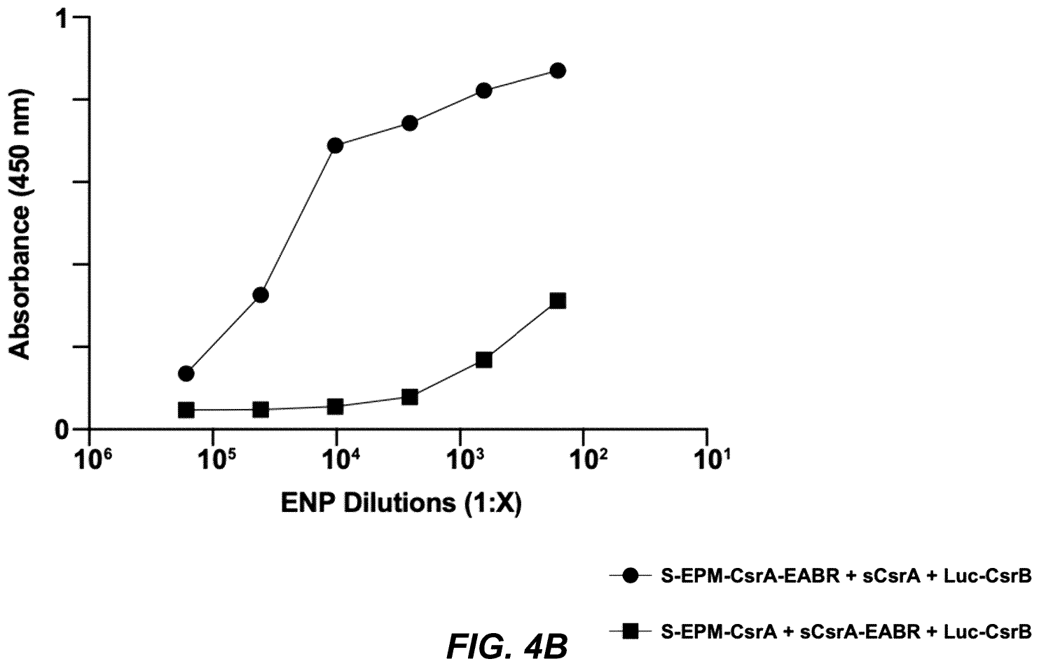

Direct fusion of the EABR sequence to the CSP could induce ENP budding in the absence of the RNA cargo, potentially resulting in the production of empty ENPs. To increase the likelihood that each ENP contains the RNA cargo, the EABR domain was removed from the S-EPM-CsrA-EABR construct and fused to the soluble CsrA protein to generate S-EPM-CsrA and sCsrA-EABR (Table 4). Co-expression of S-EPM-CsrA and sCsrA-EABR may result in ENP budding as the CsrA proteins could dimerize to connect the CSP and the EABR sequence. Although CsrA dimerization in the absence of the Luc-CsrB mRNA cargo could still result in ENP budding, this configuration might promote RNA packaging due to the strong interaction between the CsrA homodimer and CsrB PS. Co-transfection of plasmids encoding S-EPM-CsrA+sCsrA-EABR+Luc-CsrB resulted in 7.9-fold lower Luc mRNA levels in purified ENPs compared to S-EPM-CsrA-EABR+sCsrA+Luc-CsrB ENPs (FIG. 4A). ELISAs performed on purified ENP samples showed that purified S-EPM-CsrA+sCsrA-EABR+Luc-CsrB ENPs contained˜250-fold less S protein (FIG. 4B), suggesting that ENP production was markedly lower compared to S-EPM-CsrA-EABR+sCsrA+Luc-CsrB ENPs. These results suggest that a larger fraction of S-EPM-CsrA+sCsrA-EABR+Luc-CsrB ENPs contained the specific RNA cargo, but this configuration markedly decreased ENP production rate.

It was next investigated whether an ERD adapter system could further enhance RNA packaging into ENPs. For this adapter system, the ERD is directly fused to a small adapter protein that targets the ERD to the plasma membrane (FIG. 1B). Specific CSPs such as the ENP targeting protein can get incorporated and displayed on the ENP surface through a designed interaction between a modified cytoplasmic domain on the CSP and the adapter protein fused to the ERD, thereby connecting the CSP and the ERD (FIG. 1B). For this ERD adapter system, the known interaction between the cytoplasmic domain of the human CD4 protein and the N-terminal unique domain of the human tyrosine kinase Lck was used, which is involved in T cell activation and signaling. The cytoplasmic domain of the SARS-COV-2 S protein was replaced with the CD4 cytoplasmic tail to generate S-CD4 CT-EPM-CsrA (Table 4). The adapter was designed by fusing residues 1-61 of Lck, containing the SH4 and unique domains, to the CsrA protein and EABR sequence separated by a 12-residue flexible linker (Table 4). The S-CD4 CT-EPM-CsrA and Lck-CsrA-EABR constructs both contained CsrA proteins to maximize RNA encapsulation. The Lck-CsrA-EABR adapter can localize to the plasma membrane as the glycine residue at position 2 gets myristoylated and the cysteine residues at positions 3 and 5 get palmitoylated. S-CD4 CT-EPM-CsrA-EABR and Lck-CsrA constructs were also designed to determine whether it is more effective to have the EABR sequence fused to the CSP or the adapter (Table 4).

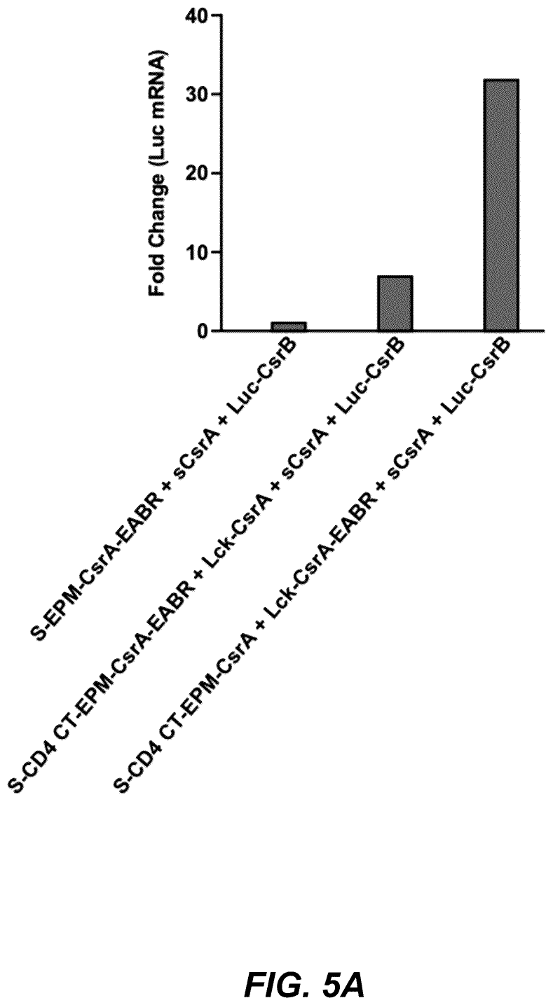

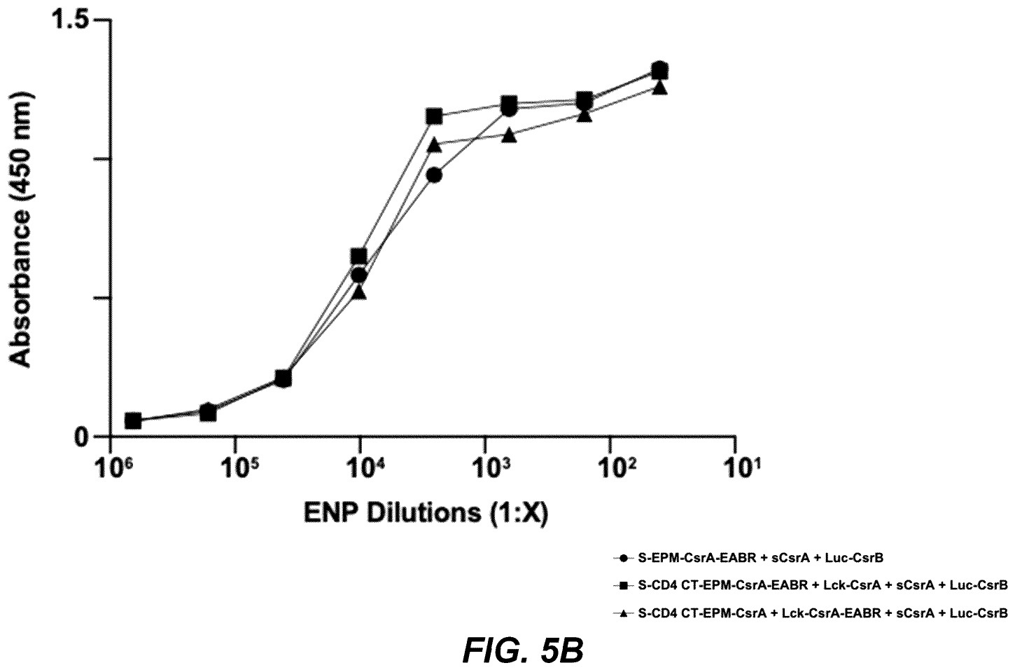

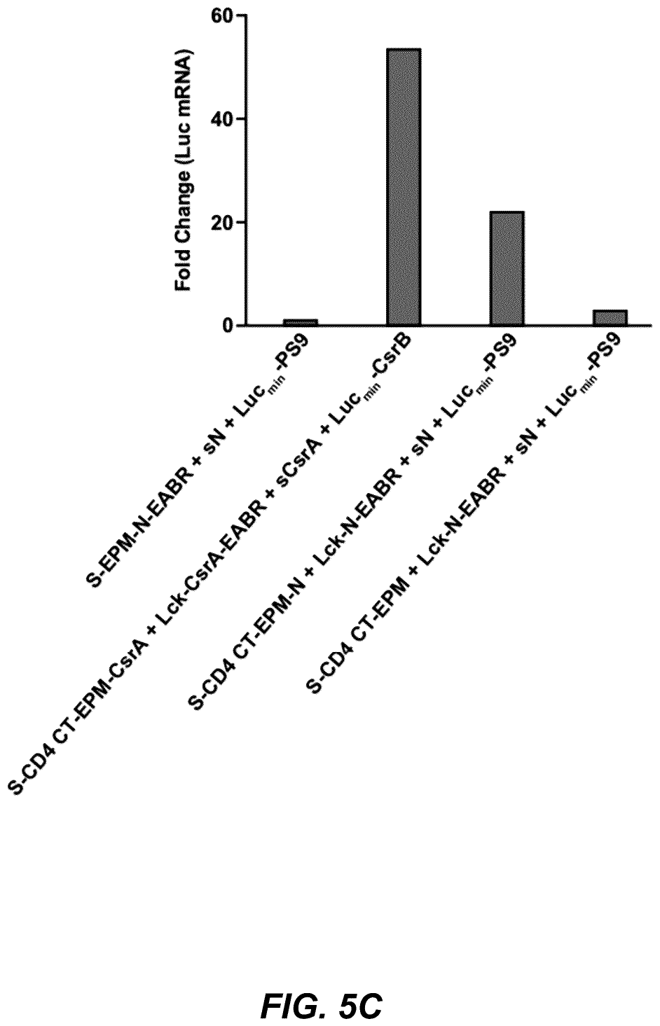

Co-transfection of plasmids encoding S-CD4 CT-EPM-CsrA+Lck-CsrA-EABR+sCsrA+Luc-CsrB resulted in 32-fold higher Luc mRNA levels in purified ENPs compared to S-EPM-CsrA-EABR+sCsrA+Luc-CsrB ENPs (FIG. 5A). Lower Luc mRNA levels were detected in purified S-CD4 CT-EPM-CsrA-EABR+Lck-CsrA+sCsrA+Luc-CsrB ENPs, suggesting that fusing the EABR sequence to the Lck-CsrA adapter was more effective for RNA packaging. ELISAs performed on purified ENP samples showed that ENP production was similar for all conditions (FIG. 5B), demonstrating that the Lck-CsrA-EABR adapter induces efficient ENP budding and recruitment of S-CD4 CT-EPM-CsrA to the ENP surface. Substituting the CsrA-CsrB interaction with the SARS-COV-2 N-PS9 interaction (Table 4) in the ERD adapter system was also investigated. Co-transfection of plasmids encoding S-CD4 CT-EPM-N+Lck-N-EABR+sN+Lucmin-PS9 resulted in efficient packaging of a minimal Luc mRNA sequence (Table 4), which was 22-fold higher compared to S-EPM-N-EABR+sN+Lucmin-PS9 ENPs (FIG. 5C). Interestingly, Lucmin MRNA levels dropped after removal of N from S-CD4 CT-EPM-N, indicating that RBP fusion to the CSP promotes incorporation of a specific RNA cargo for ERD adapter systems. Overall, these results demonstrate that the ERD adapter system promotes highly efficient ENP budding and RNA encapsulation.

Targeted delivery of the RNA cargo to specific cell types requires ENP surface display of proteins that mediate attachment to a specific cell surface receptor expressed by the target cell and subsequently induce fusion of the ENP envelope and cellular membrane to facilitate entry of the RNA cargo into the cytoplasm of the target cell. Viral glycoproteins presented on the surface of enveloped viruses normally mediate both functions, receptor attachment and membrane fusion. To better control the tropism of lentiviral vectors for gene delivery applications, modified viral fusion proteins have been designed that only mediate membrane fusion, but not receptor attachment, by introducing mutations into the receptor-binding sites of viral glycoproteins such as the vesicular stomatitis virus G protein (VSV-Gₘᵤₜ) or the Sindbis virus glycoprotein. Co-display of these modified fusion proteins together with a targeting protein on the surface of lentiviral vectors has been shown to target specific cell types for lentiviral transduction.

This modular approach was used to generate ERD adapter-based ENPs that co-displayed a targeting protein and a fusion protein (FIG. 1C). SARS-COV-2 S protein was used as targeting molecule due to its high affinity for the human ACE2 receptor. Two previously described proline mutations were introduced to stabilize S in its pre-fusion conformation, which ensured that S would only mediate receptor attachment but not membrane fusion (Table 4). The SARS-CoV-2 S cytoplasmic domain was again replaced with the CD4 CT sequence to facilitate incorporation of S into the budding ENP driven by the Lck-based ERD adapter. VSV-Gₘᵤₜ was selected as fusion protein resulting in a five-component ENP delivery system based on co-expression of VSV-Gₘᵤₜ+S-CD4 CT-EPM+Lck-N-EABR+sN+Luc-PS9 (FIG. 1C, Table 4). HEK293T cells seeded in 6-well plates were co-transfected with plasmids encoding these constructs at a ratio of 1:1:1:1:3. Media was replaced after 16 hours, and ENPs were harvested after 72 hours from transfected cell culture supernatants, concentrated in Amicon filters, and frozen at −80° C. Control conditions included no transfection (control), transfection of Luc-PS9 DNA alone, and co-transfection of S-CD4 CT-EPM+Lck-N-EABR+sN+Luc-PS9 DNA in the absence of VSV-Gₘᵤₜ. The concentrated samples were diluted 6-fold in cell culture media and added to pre-seeded HEK293T cells or HEK293T cells expressing the human ACE2 receptor (HEK293T-ACE2 cells) that were seeded in 96-well plates. 10 μg/mL of polybrene was added to promote cellular ENP uptake. After 16 hours, britelite plus was added to the cells and bioluminescence was measured.

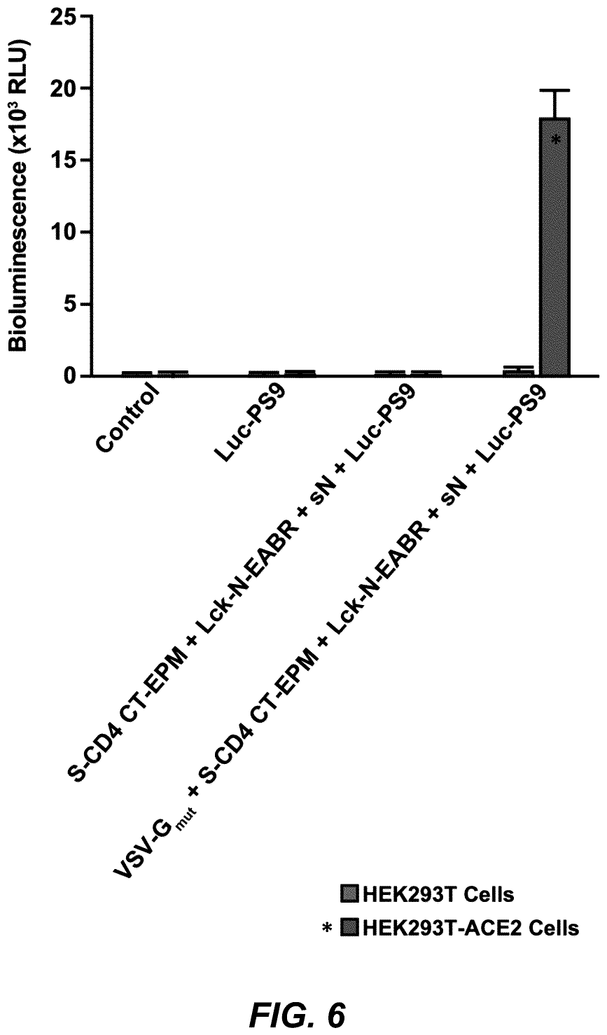

Only low levels of bioluminescence were detected for the control, Luc-PS9, and S-CD4 CT-EPM+Lck-N-EABR+sN+Luc-PS9 samples in HEK293T and HEK293T-ACE2 cells (FIG. 6). Bioluminescence induced by VSV-Gₘᵤₜ+S-CD4 CT-EPM+Lck-N-EABR+sN+Luc-PS9 ENPs in HEK293T cells was only slightly higher compared to S-CD4 CT-EPM+Lck-N-EABR+sN+Luc-PS9 ENPs, suggesting that the presence of the VSV-Gₘᵤₜ fusion protein produces minimal ENP uptake in cells that don't express the human ACE2 receptor. Importantly, VSV-Gₘᵤₜ+S-CD4 CT-EPM+Lck-N-EABR+sN+Luc-PS9 ENPs induced high bioluminescence in HEK293T-ACE2 cells, which was 319-fold higher compared to S-CD4 CT-EPM+Lck-N-EABR+sN+Luc-PS9 ENPs (FIG. 6). Bioluminescence detected for VSV-Gₘᵤₜ+S-CD4 CT-EPM+Lck-N-EABR+sN+Luc-PS9 ENPs was 58-fold higher in HEK293T-ACE2 cells compared to HEK293T cells. These results demonstrate that this five-component ENP delivery system can package a specific mRNA cargo and deliver this cargo to specific target cells resulting in high levels of protein expression.

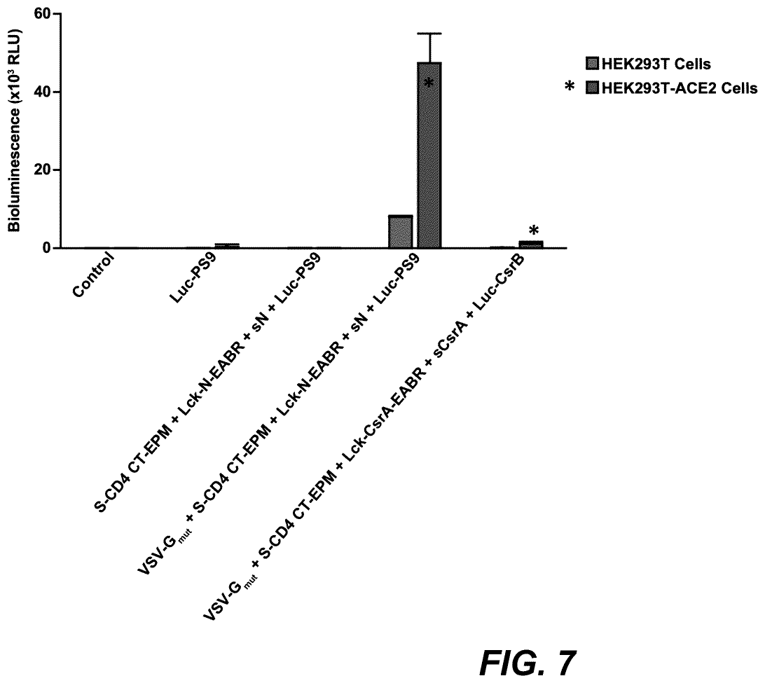

The effects of different RBP-PS combinations on ENP-mediated mRNA delivery to target cells was also studied. Interestingly, VSV-Gₘᵤₜ+S-CD4 CT-EPM+Lck-CsrA-EABR+sCsrA+Luc-CsrB ENPs induced 41-fold lower bioluminescence in HEK293T-ACE2 cells compared to VSV-Gₘᵤₜ+S-CD4 CT-EPM+Lck-N-EABR+sN+Luc-PS9 ENPs (FIG. 7). The CsrA-CsrB interaction has a high affinity and CsrB contains repeated stem loop motifs that can bind up to 18 CsrA protein copies, which, without being bound by any particular theory, could result in high-avidity interactions between Luc-CsrB and ENP-associated CsrA molecules. Without being bound by any particular theory, it is possible that the high affinity and/or avidity of the CsrA-CsrB interaction prevents effective diffusion of the Luc-CsrB mRNA cargo into the target cell cytoplasm following fusion of the ENP envelope and the endosomal membrane.

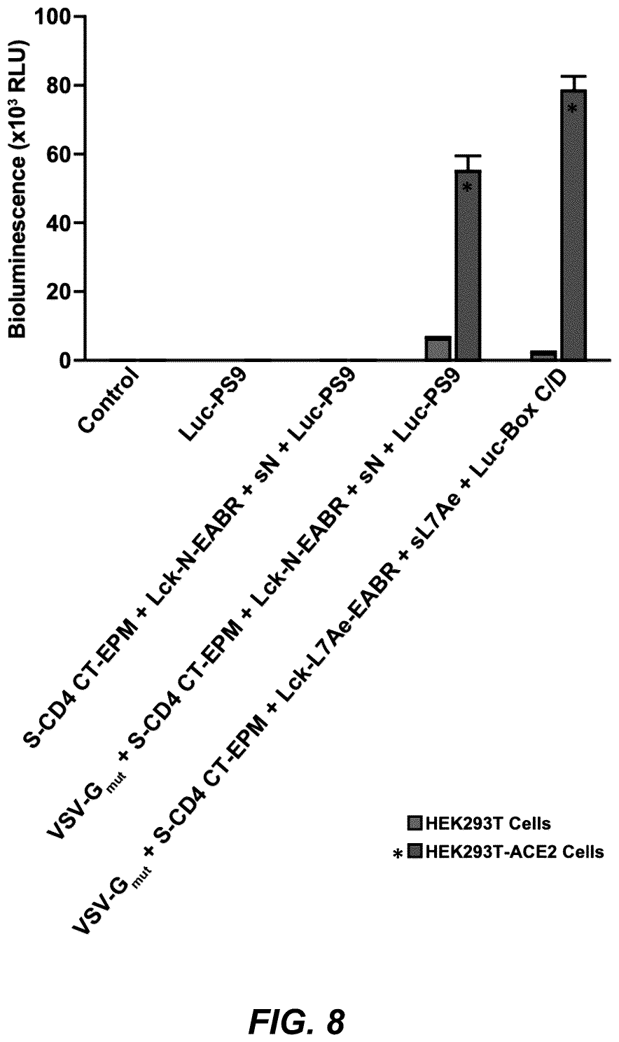

In contrast to the CsrA-CsrB interaction, the L7Ae-Box C/D interaction promoted efficient and targeted delivery of Luc-encoding mRNA. VSV-Gₘᵤₜ+S-CD4 CT-EPM+Lck-L7Ae-EABR+sL7Ae+Luc-Box C/D ENPs induced 1.4-fold higher bioluminescence in HEK293T-ACE2 cells compared to VSV-Gₘᵤₜ+S-CD4 CT-EPM+Lck-N-EABR+sN+Luc-PS9 ENPs (FIG. 8). Importantly, VSV-Gₘᵤₜ+S-CD4 CT-EPM+Lck-L7Ae-EABR+L7Ae+Luc-Box C/D ENPs also induced 3.4-fold lower bioluminescence in HEK293T cells, suggesting that L7Ae-Box C/D-based ENPs mediate highly efficient and targeted cellular delivery of a specific mRNA cargo resulting in high protein expression.

Described herein is data demonstrating that ERD ENPs can be engineered to encapsulate specific RNA cargoes and efficiently deliver these cargoes to specific target cells. ENP encapsulation of specific RNA cargoes can be achieved by inserting an ERD and a sequence-specific RBP into the cytoplasmic domain of a CSP. The ERD recruits proteins from the ESCRT pathway to induce ENP budding. The RBP recruits the RNA cargo that contains a PS that specifically interacts with the RBP to facilitate incorporation of the RNA cargo into budding ENPs. For RBPs that require dimerization or oligomerization for RNA-binding, co-expression of the soluble RBP facilitates dimerization/oligomerization and increases RNA packaging efficiency. The design of a multifunctional adapter construct further improves ENP encapsulation of specific RNA cargoes. This adapter construct is targeted to the cytoplasmic side of the plasma membrane and includes the RBP and ERD. The adapter also contains sequences that interact with the modified cytoplasmic domain of a CSP that is recruited to and displayed on the surface of the ENPs. Delivery of the RNA cargo to specific cell types requires display of CSPs that mediate ENP attachment to specific target cells and membrane fusion to facilitate entry of the RNA cargo into cells. For optimal programmability of ENP target cell tropism, ENP delivery systems were designed that co-display two CSPs: i) an ENP targeting protein that mediates attachment to cell surface receptors specifically presented on target cells; ii) a fusion protein that induces fusion between the ENP envelope and cellular membrane to facilitate entry of the RNA cargo into the target cell cytoplasm.

The designed modular ENP delivery systems can be tailored to package nucleic acid-based cargoes for a wide range of therapeutic, diagnostic, and biological applications. The RNA cargo can have therapeutic, diagnostic, or biological activity or encode a protein with therapeutic, diagnostic, or biological activity. Various targeting proteins could be displayed to target ENPs to specific cell types in vivo, thereby maximizing therapeutic concentrations of RNA cargoes in target tissues and minimizing adverse effects in other tissues. In comparison to EVs and other biological nanoparticle-based platforms, the described self-assembling ENP delivery system is more suitable for large-scale manufacturing due to the high production rate and lack of cellular toxicity. Table 4 below shows exemplary sequences related to the compositions and methods disclosed herein.

TABLE 4
Exemplary Sequences for Cargo Delivery

| | SEQ ID | |
|---|---|---|
| NAME | NO: | SEQUENCE* |
| SARS- | 40 | MFVFLVLLPLVSSQCVNLTTRTQLPPAYTNSFTRGVYYPDKVFRSSVLHSTQDLF |
| CoV-2 | | LPFFSNVTWFHAIHVSGTNGTKRFDNPVLPFNDGVYFASTEKSNIIRGWIFGTT |
| Spike | | LDSKTQSLLIVNNATNVVIKVCEFQFCNDPFLGVYYHKNNKSWMESEFRVYSS |
| | | ANNCTFEYVSQPFLMDLEGKQGNFKNLREFVFKNIDGYFKIYSKHTPINLVRDL |
| | | PQGFSALEPLVDLPIGINITRFQTLLALHRSYLTPGDSSSGWTAGAAAYYVGYL |
| | | QPRTFLLKYNENGTITDAVDCALDPLSETKCTLKSFTVEKGIYQTSNFRVQPTE |
| | | SIVRFPNITNLCPFGEVFNATRFASVYAWNRKRISNCVADYSVLYNSASFSTFK |
| | | CYGVSPTKLNDLCFTNVYADSFVIRGDEVRQIAPGQTGKIADYNYKLPDDFTG |
| | | CVIAWNSNNLDSKVGGNYNYLYRLFRKSNLKPFERDISTEIYQAGSTPCNGVE |
| | | GFNCYFPLQSYGFQPTNGVGYQPYRVVVLSFELLHAPATVCGPKKSTNLVKNK |
| | | CVNFNFNGLTGTGVLTESNKKFLPFQQFGRDIADTTDAVRDPQTLEILDITPCS |
| | | FGGVSVITPGTNTSNQVAVLYQGVNCTEVPVAIHADQLTPTWRVYSTGSNVFQT |
| | | RAGCLIGAEHVNNSYECDIPIGAGICASYQTQTNSPRRARSVASQSIIAYTMSLG |
| | | AENSVAYSNNSIAIPTNFTISVTTEILPVSMTKTSVDCTMYICGDSTECSNLLLQ |
| | | YGSFCTQLNRALTGIAVEQDKNTQEVFAQVKQIYKTPPIKDFGGFNFSQILPDPS |
| | | KPSKRSFIEDLLFNKVTLADAGFIKQYGDCLGDIAARDLICAQKFNGLTVLPPL |
| | | LTDEMIAQYTSALLAGTITSGWTFGAGAALQIPFAMQMAYRFNGIGVTQNVLYE |
| | | NQKLIANQFNSAIGKIQDSLSSTASALGKLQDVVNQNAQALNTLVKQLSSNFG |
| | | AISSVLNDILSRLDPPEAEVQIDRLITGRLQSLQTYVTQQLIRAAEIRASANLA |
| | | ATKMSECVLGQSKRVDFCGKGYHLMSFPQSAPHGVVFLHVTYVPAQEKNFTTAP |
| | | AICHDGKAHFPREGVFVSNGTHWFVTQRNFYEPQIITTDNTFVSGNCDVVIGIV |
| | | NNTVYDPLQPELDSFKEELDKYFKNHTSPDVDLGDISGINASVVNIQKEIDRLN |
| | | EVAKNLNESLIDLQELGKYEQYIKWPWYIWLGFIAGLIAIVMVTIMLCCMTSCC |
| | | SCLKGCCSCGS |
| EPM | 1 | ALPGNPDHREMGETLPEEVGEYRQPSGGSVPVSPGPPSGLEPTSSSPY |
| CsrA | 128 | MLILTRRVGETLMIGDEVTVTVLGVKGNQVRIGVNAPKEVSVHREEIYQRIQAEKSQ |
| | | QSSY |
| EABR | 2 | FNSSINNIHEMEIQLKDALEKNQQWLVYDQQREVYVKGLLAKIFELEKKTETAAHSL |
| | | P |
| EPM- | 129 | ALPGNPDHREMGETLPEEVGEYRQPSGGSVPVSPGPPSGLEPTSSSPYGSKSGSGS |
| CsrA- | | DSGSMLILTRRVGETLMIGDEVTVTVLGVKGNQVRIGVNAPKEVSVHREEIYQRIQA |
| EABR | | EKSQQSSYGGGSFNSSINNIHEMEIQLKDALEKNQQWLVYDQQREVYVKGLLAKIF |
| | | ELEKKTETAAHSLP |
| Luc | 130 | ATGGAAGATGCCAAGAACATCAAGAAGGGCCCTGCTCCATTCTACCCTCTG |
| | | GAAGATGGAACAGCCGGCGAGCAGCTGCACAAGGCCATGAAGAGATATGC |
| | | CCTGGTGCCTGGCACAATCGCCTTCACAGATGCCCACATCGAGGTGGACAT |
| | | CACCTACGCCGAGTACTTCGAGATGTCTGTGCGGCTGGCCGAAGCTATGAA |
| | | GCGCTACGGCCTGAACACCAACCACCGGATCGTCGTGTGCAGCGAGAACA |
| | | GCCTGCAGTTCTTCATGCCTGTGCTGGGCGCCCTGTTCATCGGAGTTGCTGT |
| | | GGCCCCTGCCAACGACATCTACAACGAGCGCGAGCTGCTGAACAGCATGG |
| | | GCATCTCTCAGCCCACCGTGGTGTTCGTGTCCAAGAAGGGACTGCAGAAAA |
| | | TCCTGAACGTGCAGAAGAAGCTGCCCATCATCCAGAAAATCATCATCATGG |
| | | ACAGCAAGACCGACTACCAGGGCTTCCAGAGCATGTACACCTTCGTGACCA |
| | | GCCATCTGCCACCTGGCTTCAACGAGTACGACTTCGTGCCCGAGAGCTTCG |
| | | ACCGGGACAAGACAATCGCCCTGATCATGAACAGCAGCGGCTCTACCGGA |
| | | CTGCCCAAAGGTGTTGCCCTGCCTCATAGAACTGCCTGCGTGCGGTTTAGC |
| | | CACGCCAGAGATCCCATCTTCGGCAACCAGATCATCCCCGACACAGCCATC |
| | | CTGAGCGTGGTGCCTTTTCACCACGGCTTCGGCATGTTCACCACACTGGGCT |
| | | ACCTGATCTGCGGCTTCAGAGTGGTGCTGATGTACCGCTTCGAGGAAGAAC |
| | | TGTTCCTGCGGAGCCTGCAGGACTACAAGATCCAGTCTGCTCTGCTGGTGC |
| | | CTACTCTGTTCAGCTTCTTTGCCAAGAGCACCCTGATCGATAAGTACGACCT |
| | | GAGCAACCTGCACGAGATCGCCTCTGGCGGAGCCCCTCTGTCTAAAGAAGT |
| | | GGGAGAAGCCGTCGCCAAGCGGTTCCATCTGCCTGGCATCAGACAAGGCTA |
| | | TGGCCTGACCGAGACAACCAGCGCCATTCTGATTACCCCTGAGGGCGACGA |
| | | TAAGCCTGGCGCTGTGGGAAAAGTGGTGCCATTCTTCGAGGCCAAGGTGGT |
| | | GGATCTGGACACCGGCAAAACACTGGGCGTTAACCAGAGGGGCGAGCTGT |
| | | GTGTTAGAGGCCCTATGATCATGAGCGGCTACGTGAACAACCCCGAGGCCA |
| | | CAAACGCTCTGATCGACAAGGATGGATGGCTGCACAGCGGCGACATTGCCT |
| | | ACTGGGACGAAGATGAGCACTTCTTCATCGTGGACCGGCTGAAGTCCCTGA |
| | | TCAAGTACAAGGGCTACCAGGTGGCCCCAGCCGAGCTGGAATCTATTCTGC |
| | | TGCAACACCCCAACATCTTCGATGCCGGCGTTGCAGGACTGCCCGATGATG |
| | | ATGCTGGCGAACTGCCAGCTGCTGTGGTGGTGCTGGAACACGGCAAGACCA |
| | | TGACCGAGAAAGAAATCGTGGACTACGTGGCCAGCCAAGTGACCACCGCC |
| | | AAGAAACTGAGAGGCGGCGTGGTGTTTGTGGACGAGGTGCCAAAAGGCCT |
| | | GACAGGCAAGCTGGACGCCCGGAAGATCAGAGAGATCCTGATTAAGGCCA |
| | | AGAAAGGCGGCAAGATCGCCGTG |
| CsrB | 131 | GTCGACAGGGAGTCAGACAACGAAGTGAACATCAGGATGATGACACTTCTGCAG |
| | (DNA), | GACACACCAGGATGGTGTTTCAGGGAAAGGCTTCTGGATGAAGCGAAGAGGATG |
| | 134 | ACGCAGGACGCGTTAAAGGACACCTCCAGGATGGAGAATGAGAACCGGTCAGG |
| | (RNA) | ATGATTCGGTGGGTCAGGAAGGCCAGGGACACTTCAGGATGAAGTATCACATCG |
| | | GGGTGGTGTGAGCAGGAAGCAATAGTTCAGGATGAACGATTGGCCGCAAGGCC |
| | | AGAGGAAAAGTTGTCAAGGATGAGCAGGGAGCAACAAAAGTAGCTGGAATGCTG |
| | | CGAAACGAACCGGGAGCGCTGTGAATACAGTGCTCCCTTTTTTTATT |
| Luc- | 132 | ATGGAAGATGCCAAGAACATCAAGAAGGGCCCTGCTCCATTCTACCCTCTGGA |
| CsrB | (DNA), | AGATGGAACAGCCGGCGAGCAGCTGCACAAGGCCATGAAGAGATATGCCCTG |
| | 135 | GTGCCTGGCACAATCGCCTTCACAGATGCCCACATCGAGGTGGACATCACCTA |
| | (RNA) | CGCCGAGTACTTCGAGATGTCTGTGCGGCTGGCCGAAGCTATGAAGCGCTACG |
| | | GCCTGAACACCAACCACCGGATCGTCGTGTGCAGCGAGAACAGCCTGCAGTTC |
| | | TTCATGCCTGTGCTGGGCGCCCTGTTCATCGGAGTTGCTGTGGCCCCTGCCAAC |
| | | GACATCTACAACGAGCGCGAGCTGCTGAACAGCATGGGCATCTCTCAGCCCAC |
| | | CGTGGTGTTCGTGTCCAAGAAGGGACTGCAGAAAATCCTGAACGTGCAGAAGA |
| | | AGCTGCCCATCATCCAGAAAATCATCATCATGGACAGCAAGACCGACTACCAG |
| | | GGCTTCCAGAGCATGTACACCTTCGTGACCAGCCATCTGCCACCTGGCTTCAAC |
| | | GAGTACGACTTCGTGCCCGAGAGCTTCGACCGGGACAAGACAATCGCCCTGAT |
| | | CATGAACAGCAGCGGCTCTACCGGACTGCCCAAAGGTGTTGCCCTGCCTCATA |
| | | GAACTGCCTGCGTGCGGTTTAGCCACGCCAGAGATCCCATCTTCGGCAACCAG |
| | | ATCATCCCCGACACAGCCATCCTGAGCGTGGTGCCTTTTCACCACGGCTTCGGC |
| | | ATGTTCACCACACTGGGCTACCTGATCTGCGGCTTCAGAGTGGTGCTGATGTAC |
| | | CGCTTCGAGGAAGAACTGTTCCTGCGGAGCCTGCAGGACTACAAGATCCAGTC |
| | | TGCTCTGCTGGTGCCTACTCTGTTCAGCTTCTTTGCCAAGAGCACCCTGATCGAT |
| | | AAGTACGACCTGAGCAACCTGCACGAGATCGCCTCTGGCGGAGCCCCTCTGTC |
| | | TAAAGAAGTGGGAGAAGCCGTCGCCAAGCGGTTCCATCTGCCTGGCATCAGAC |
| | | AAGGCTATGGCCTGACCGAGACAACCAGCGCCATTCTGATTACCCCTGAGGGC |
| | | GACGATAAGCCTGGCGCTGTGGGAAAAGTGGTGCCATTCTTCGAGGCCAAGGT |
| | | GGTGGATCTGGACACCGGCAAAACACTGGGCGTTAACCAGAGGGGCGAGCTGT |
| | | GTGTTAGAGGCCCTATGATCATGAGCGGCTACGTGAACAACCCCGAGGCCACA |
| | | AACGCTCTGATCGACAAGGATGGATGGCTGCACAGCGGCGACATTGCCTACTG |
| | | GGACGAAGATGAGCACTTCTTCATCGTGGACCGGCTGAAGTCCCTGATCAAGT |
| | | ACAAGGGCTACCAGGTGGCCCCAGCCGAGCTGGAATCTATTCTGCTGCAACAC |
| | | CCCAACATCTTCGATGCCGGCGTTGCAGGACTGCCCGATGATGATGCTGGCGA |
| | | ACTGCCAGCTGCTGTGGTGGTGCTGGAACACGGCAAGACCATGACCGAGAAAG |
| | | AAATCGTGGACTACGTGGCCAGCCAAGTGACCACCGCCAAGAAACTGAGAGGC |
| | | GGCGTGGTGTTTGTGGACGAGGTGCCAAAAGGCCTGACAGGCAAGCTGGACGC |
| | | CCGGAAGATCAGAGAGATCCTGATTAAGGCCAAGAAAGGCGGCAAGATCGCC |
| | | GTGTGAAAGCTTGGCGTCGACAGGGAGTCAGACAACGAAGTGAACATCAGGATGA |
| | | TGACACTTCTGCAGGACACACCAGGATGGTGTTTCAGGGAAAGGCTTCTGGATGAAG |
| | | CGAAGAGGATGACGCAGGACGCGTTAAAGGACACCTCCAGGATGGAGAATGAGAAC |
| | | CGGTCAGGATGATTCGGTGGGTCAGGAAGGCCAGGGACACTTCAGGATGAAGTATC |
| | | ACATCGGGGTGGTGTGAGCAGGAAGCAATAGTTCAGGATGAACGATTGGCCGCAAG |
| | | GCCAGAGGAAAAGTTGTCAAGGATGAGCAGGGAGCAACAAAAGTAGCTGGAATGCT |
| | | GCGAAACGAACCGGGAGCGCTGTGAATACAGTGCTCCCTTTTTTTATT |
| SN | 136 | MSDNGPQNQRNAPRITFGGPSDSTGSNQNGERSGARSKQRRPQGLPNNTASWFTA |
| | | LTQHGKEDLKFPRGQGVPINTNSSPDDQIGYYRRATRRIRGGDGKMKDLSPRWYFY |
| | | YLGTGPEAGLPYGANKDGIIWVATEGALNTPKDHIGTRNPANNAAIVLQLPQGTTLP |
| | | KGFYAEGSRGGSQASSRSSSRSRNSSRNSTPGSSRGTSPARMAGNGGDAALALLLLD |
| | | RLNQLESKMSGKGQQQQGQTVTKKSAAEASKKPRQKRTATKAYNVTQAFGRRGPE |
| | | QTQGNFGDQELIRQGTDYKHWPQIAQFAPSASAFFGMSRIGMEVTPSGTWLTYTG |
| | | AIKLDDKDPNFKDQVILLNKHIDAYKTFPPTEPKKDKKKKADETQALPQRQKKQQ |
| | | TVTLLPAADLDDFSKQLQQSMSSADSTQA |
| EPM-N- | 137 | ALPGNPDHREMGETLPEEVGEYRQPSGGSVPVSPGPPSGLEPTSSSPYGSKSGSGS |
| EABR | | DSGSMSDNGPQNQRNAPRITFGGPSDSTGSNQNGERSGARSKQRRPQGLPNNTAS |
| | | WFTALTQHGKEDLKFPRGQGVPINTNSSPDDQIGYYRRATRRIRGGDGKMKDLSPR |
| | | WYFYYLGTGPEAGLPYGANKDGIIWVATEGALNTPKDHIGTRNPANNAAIVLQLPQ |
| | | GTTLPKGFYAEGSRGGSQASSRSSSRSRNSSRNSTPGSSRGTSPARMAGNGGDAALA |
| | | LLLLDRLNQLESKMSGKGQQQQGQTVTKKSAAEASKKPRQKRTATKAYNVTQAFG |
| | | RRGPEQTQGNFGDQELIRQGTDYKHWPQIAQFAPSASAFFGMSRIGMEVTPSGTW |
| | | LTYTGAIKLDDKDPNFKDQVILLNKHIDAYKTFPPTEPKKDKKKKADETQALPQRQ |
| | | KKQQTVTLLPAADLDDFSKQLQQSMSSADSTQAGGGSFNSSINNIHEMEIQLKDAL |
| | | EKNQQWLVYDQQREVYVKGLLAKIFELEKKTETAAHSLP |
| PS9 | 138 | tctgtaggtcccaaacaagctagtcttaatggagtcacattaattggagaagccg |
| | (DNA); | taaaaacacagttcaattattataagaaagttgatggtgttgtccaacaattacc |
| | 140 | tgaaacttactttactcagagtagaaatttacaagaatttaaacccaggagtcaa |
| | (RNA) | atggaaattgatttcttagaattagctatggatgaattcattgaacggtataaat |
| | | tagaaggctatgccttcgaacatatcgtttatggagattttagtcatagtcagtt |
| | | aggtggtttacatctactgattggactagctaaacgttttaaggaatcacc |
| | | ttttgaattagaagattttattcctatggacagtacagttaaaaactattt |
| | | cataacagatgcgcaaacaggttcatctaagtgtgtgtgttctgttattga |
| | | tttattacttgatgattttgttgaaataataaaatcccaagatttatctgt |
| | | agtttctaaggttgtcaaagtgactattgactatacagaaatttcatttat |
| | | gctttggtgtaaagatggccatgtagaaacattttacccaaaattacaatc |
| | | tagtcaagcgtggcaaccgggtgttgctatgcctaatctttacaaaatgca |
| | | aagaatgctattagaaaagtgtgaccttcaaaattatggtgatagtgcaac |
| | | attacctaaaggcataatgatgaatgtcgcaaaatatactcaactgtgtca |
| | | atatttaaacacattaacattagctgtaccctataatatgagagttataca |
| | | ttttggtgctggttctgataaaggagttgcaccaggtacagctgttttaag |
| | | acagtggttgcctacgggtacgctgcttgtcgattcagatcttaatgactt |
| | | tgtctctgatgcagattcaactttgattggtgattgtgcaactgtacatac |
| | | agctaataaatgggatctcattattagtgatatgtacgaccctaagactaa |
| | | aaatgttacadaagaaaatgactctaaagagggttttttcacttacatttg |
| | | tgggtttatacaacaaaagctagctcttggaggttccgtggctataaagat |
| | | a |
| Luc-PS9 | 139 | ATGGAAGATGCCAAGAACATCAAGAAGGGCCCTGCTCCATTCTACCCTCTG |
| (DNA | (DNA), | GAAGATGGAACAGCCGGCGAGCAGCTGCACAAGGCCATGAAGAGATATGC |
| sequence) | 141 | CCTGGTGCCTGGCACAATCGCCTTCACAGATGCCCACATCGAGGTGGACAT |
| | (RNA) | CACCTACGCCGAGTACTTCGAGATGTCTGTGCGGCTGGCCGAAGCTATGAA |
| | | GCGCTACGGCCTGAACACCAACCACCGGATCGTCGTGTGCAGCGAGAACA |
| | | GCCTGCAGTTCTTCATGCCTGTGCTGGGCGCCCTGTTCATCGGAGTTGCTG |
| | | TGGCCCCTGCCAACGACATCTACAACGAGCGCGAGCTGCTGAACAGCATGG |
| | | GCATCTCTCAGCCCACCGTGGTGTTCGTGTCCAAGAAGGGACTGCAGAAAA |
| | | TCCTGAACGTGCAGAAGAAGCTGCCCATCATCCAGAAAATCATCATCATGG |
| | | ACAGCAAGACCGACTACCAGGGCTTCCAGAGCATGTACACCTTCGTGACCA |
| | | GCCATCTGCCACCTGGCTTCAACGAGTACGACTTCGTGCCCGAGAGCTTCG |
| | | ACCGGGACAAGACAATCGCCCTGATCATGAACAGCAGCGGCTCTACCGGA |
| | | CTGCCCAAAGGTGTTGCCCTGCCTCATAGAACTGCCTGCGTGCGGTTTAGC |
| | | CACGCCAGAGATCCCATCTTCGGCAACCAGATCATCCCCGACACAGCCATC |
| | | CTGAGCGTGGTGCCTTTTCACCACGGCTTCGGCATGTTCACCACACTGGGCT |
| | | ACCTGATCTGCGGCTTCAGAGTGGTGCTGATGTACCGCTTCGAGGAAGAAC |
| | | TGTTCCTGCGGAGCCTGCAGGACTACAAGATCCAGTCTGCTCTGCTGGTGC |
| | | CTACTCTGTTCAGCTTCTTTGCCAAGAGCACCCTGATCGATAAGTACGACCT |
| | | GAGCAACCTGCACGAGATCGCCTCTGGCGGAGCCCCTCTGTCTAAAGAAGT |
| | | GGGAGAAGCCGTCGCCAAGCGGTTCCATCTGCCTGGCATCAGACAAGGCTA |
| | | TGGCCTGACCGAGACAACCAGCGCCATTCTGATTACCCCTGAGGGCGACGA |
| | | TAAGCCTGGCGCTGTGGGAAAAGTGGTGCCATTCTTCGAGGCCAAGGTGGT |
| | | GGATCTGGACACCGGCAAAACACTGGGCGTTAACCAGAGGGGCGAGCTGT |
| | | GTGTTAGAGGCCCTATGATCATGAGCGGCTACGTGAACAACCCCGAGGCCA |
| | | CAAACGCTCTGATCGACAAGGATGGATGGCTGCACAGCGGCGACATTGCCT |
| | | ACTGGGACGAAGATGAGCACTTCTTCATCGTGGACCGGCTGAAGTCCCTGA |
| | | TCAAGTACAAGGGCTACCAGGTGGCCCCAGCCGAGCTGGAATCTATTCTGC |
| | | TGCAACACCCCAACATCTTCGATGCCGGCGTTGCAGGACTGCCCGATGATG |
| | | ATGCTGGCGAACTGCCAGCTGCTGTGGTGGTGCTGGAACACGGCAAGACCA |
| | | TGACCGAGAAAGAAATCGTGGACTACGTGGCCAGCCAAGTGACCACCGCC |
| | | AAGAAACTGAGAGGCGGCGTGGTGTTTGTGGACGAGGTGCCAAAAGGCCT |
| | | GACAGGCAAGCTGGACGCCCGGAAGATCAGAGAGATCCTGATTAAGGCCA |
| | | AGAAAGGCGGCAAGATCGCCGTG TGAAAGCTTGGCtctgtaggtcccaaa |
| | | caagctagtcttaatggagtcacattaattggagaagccgtaaaaacacag |
| | | ttcaattattataagaaagttgatggtgttgtccaacaattacctgaaact |
| | | tactttactcagagtagaaatttacaagaatttaaacccaggagtcaaatg |
| | | gaaattgatttcttagaattagctatggatgaattcattgaacggtataaa |
| | | ttagaaggctatgccttcgaacatatcgtttatggagattttagtcatagt |
| | | cagttaggtggtttacatctactgattggactagctaaacgttttaaggaa |
| | | tcaccttttgaattagaagattttattcctatggacagtacagttaaaaac |
| | | tatttcataacagatgcgcaaacaggttcatctaagtgtgtgtgttctgtt |
| | | attgatttattacttgatgattttgttgaaataataaaatcccaagattta |
| | | tctgtagtttctaaggttgtcaaagtgactattgactatacagaaatttca |
| | | tttatgctttggtgtaaagatggccatgtagaaacattttacccaaaatta |
| | | caatctagtcaagcgtggcaaccgggtgttgctatgcctaatctttacaaa |
| | | atgcaaagaatgctattagaaaagtgtgaccttcaaaattatggtgatagt |
| | | gcaacattacctaaaggcataatgatgaatgtcgcaaaatatactcaactg |
| | | tgtcaatatttaaacacattaacattagctgtaccctataatatgagagtt |
| | | atacattttggtgctggttctgataaaggagttgcaccaggtacagctgtt |
| | | ttaagacagtggttgcctacgggtacgctgcttgtcgattcagatcttaat |
| | | gactttgtctctgatgcagattcaactttgattggtgattgtgcaactgta |
| | | catacagctaataaatgggatctcattattagtgatatgtacgaccctaag |
| | | actaaaaatgttacaaaagaaaatgactctaaagagggttttttcacttac |
| | | atttgtgggtttatacaacaaaagctagctcttggaggttccgtggctata |
| | | aagata |
| SL7Ae | 142 | MYVRFEVPEDMQNEALSLLEKVRESGKVKKGTNETTKAVERGLAKLVYIAED |
| | | VDPPEIVAHLPLLCEEKNVPYIYVKSKNDLGRAVGIEVPCASAAIINEGELR |
| | | KELGSLVEKIKGLQK |
| EPM- | 143 | ALPGNPDHREMGETLPEEVGEYRQPSGGSVPVSPGPPSGLEPTSSSPYGSKS |
| L7Ae- | | GSGSDSGSMYVRFEVPEDMQNEALSLLEKVRESGKVKKGTNETTKAVERGLA |
| EABR | | KLVYIAEDVDPPEIVAHLPLLCEEKNVPYIYVKSKNDLGRAVGIEVPCASAA |
| | | IINEGELRKELGSLVEKIKGLQKGGGSFNSSINNIHEMEIQLKDALEKNQQW |
| | | LVYDQQREVYVKGLLAKIFELEKKTETAAHSLP |
| Box C/D | 144 | acagatctggatcgggcgtgatCcgaaagGtgacccctaggcttaagtata |
| | (DNA), | |
| | 146 | |
| | (RNA) | |
| Luc-Box | 145 | ATGGAAGATGCCAAGAACATCAAGAAGGGCCCTGCTCCATTCTACCCTCTG |
| C/D | (DNA), | GAAGATGGAACAGCCGGCGAGCAGCTGCACAAGGCCATGAAGAGATATGC |
| | 147 | CCTGGTGCCTGGCACAATCGCCTTCACAGATGCCCACATCGAGGTGGACAT |
| | (RNA) | CACCTACGCCGAGTACTTCGAGATGTCTGTGCGGCTGGCCGAAGCTATGAA |
| | | GCGCTACGGCCTGAACACCAACCACCGGATCGTCGTGTGCAGCGAGAACA |
| | | GCCTGCAGTTCTTCATGCCTGTGCTGGGCGCCCTGTTCATCGGAGTTGCTGT |
| | | GGCCCCTGCCAACGACATCTACAACGAGCGCGAGCTGCTGAACAGCATGG |
| | | GCATCTCTCAGCCCACCGTGGTGTTCGTGTCCAAGAAGGGACTGCAGAAAA |
| | | TCCTGAACGTGCAGAAGAAGCTGCCCATCATCCAGAAAATCATCATCATGG |
| | | ACAGCAAGACCGACTACCAGGGCTTCCAGAGCATGTACACCTTCGTGACCA |
| | | GCCATCTGCCACCTGGCTTCAACGAGTACGACTTCGTGCCCGAGAGCTTCG |
| | | ACCGGGACAAGACAATCGCCCTGATCATGAACAGCAGCGGCTCTACCGGA |
| | | CTGCCCAAAGGTGTTGCCCTGCCTCATAGAACTGCCTGCGTGCGGTTTAGC |
| | | CACGCCAGAGATCCCATCTTCGGCAACCAGATCATCCCCGACACAGCCATC |
| | | CTGAGCGTGGTGCCTTTTCACCACGGCTTCGGCATGTTCACCACACTGGGCT |
| | | ACCTGATCTGCGGCTTCAGAGTGGTGCTGATGTACCGCTTCGAGGAAGAAC |
| | | TGTTCCTGCGGAGCCTGCAGGACTACAAGATCCAGTCTGCTCTGCTGGTGC |
| | | CTACTCTGTTCAGCTTCTTTGCCAAGAGCACCCTGATCGATAAGTACGACCT |
| | | GAGCAACCTGCACGAGATCGCCTCTGGCGGAGCCCCTCTGTCTAAAGAAGT |
| | | GGGAGAAGCCGTCGCCAAGCGGTTCCATCTGCCTGGCATCAGACAAGGCTA |
| | | TGGCCTGACCGAGACAACCAGCGCCATTCTGATTACCCCTGAGGGCGACGA |
| | | TAAGCCTGGCGCTGTGGGAAAAGTGGTGCCATTCTTCGAGGCCAAGGTGGT |
| | | GGATCTGGACACCGGCAAAACACTGGGCGTTAACCAGAGGGGCGAGCTGT |
| | | GTGTTAGAGGCCCTATGATCATGAGCGGCTACGTGAACAACCCCGAGGCCA |
| | | CAAACGCTCTGATCGACAAGGATGGATGGCTGCACAGCGGCGACATTGCCT |
| | | ACTGGGACGAAGATGAGCACTTCTTCATCGTGGACCGGCTGAAGTCCCTGA |
| | | TCAAGTACAAGGGCTACCAGGTGGCCCCAGCCGAGCTGGAATCTATTCTGC |
| | | TGCAACACCCCAACATCTTCGATGCCGGCGTTGCAGGACTGCCCGATGATG |
| | | ATGCTGGCGAACTGCCAGCTGCTGTGGTGGTGCTGGAACACGGCAAGACCA |
| | | TGACCGAGAAAGAAATCGTGGACTACGTGGCCAGCCAAGTGACCACCGCC |
| | | AAGAAACTGAGAGGCGGCGTGGTGTTTGTGGACGAGGTGCCAAAAGGCCT |
| | | GACAGGCAAGCTGGACGCCCGGAAGATCAGAGAGATCCTGATTAAGGCCA |
| | | AGAAAGGCGGCAAGATCGCCGTGTGAAAGCTTGGCacagatctggatcgg |
| | | gcgtgatCcgaaagGtgacccctaggcttaagtata |
| SMCP | 148 | MASNFTQFVLVDNGGTGDVTVAPSNFANGVAEWISSNSRSQAYKVTCSVR |
| | | QSSAQNRKYTIKVEVPKVATQTVGGVELPVAAWRSYLNMELTIPIFATNS |
| | | DCELIVKAMQGLLKDGNPIPSAIAANSGIY |
| EPM- | 149 | ALPGNPDHREMGETLPEEVGEYRQPSGGSVPVSPGPPSGLEPTSSSPYGS |
| MCP- | | KSGSGSDSGSMASNFTQFVLVDNGGTGDVTVAPSNFANGVAEWISSNSRS |
| EABR | | QAYKVTCSVRQSSAQNRKYTIKVEVPKVATQTVGGVELPVAAWRSYLNME |
| | | LTIPIFATNSDCELIVKAMQGLLKDGNPIPSAIAANSGIYGGGSFNSSIN |
| | | NIHEMEIQLKDALEKNQQWLVYDQQREVYVKGLLAKIFELEKKTETAAHS |
| | | LP |
| MS2 | 150 | taaggtacctaattgcctagaaaacatgaggatcacccatgtctgcaggtcg |
| | | actctagaaaacatgaggatcacccatgtctgcagtattcccgggttcatta |
| | | gatcctaaggtacctaattgcctagaaaacatgaggatcacccatgtctgca |
| | | ggtcgactctagaaaacatgaggatcacccatgtctgcagtattcccgggtt |
| | | cattagatcctaaggtacctaattgcctagaaaacatgaggatcacccatgt |
| | | ctgcaggtcgactctagaaaacatgaggatcacccatgtctgcagtattccc |
| | | gggttcattagatcctaaggtacctaattgcctagaaaacatgaggatcacc |
| | | catgtctgcaggtcgactctagaaaacatgaggatcacccatgtctgcagta |
| | | ttcccgggttcattagatcctaaggtacctaattgcctagaaaacatgagga |
| | | tcacccatgtctgcaggtcgactctagaaaacatgaggatcacccatgtctg |
| | | cagtattcccgggttcattagatcctaaggtacctaattgcctagaaaacat |
| | | gaggatcacccatgtctgcaggtcgactctagaaaacatgaggatcacccat |
| | | gtctgcagtattcccgggttcattagatcctaa |
| Luc- | 151 | ATGGAAGATGCCAAGAACATCAAGAAGGGCCCTGCTCCATTCTACCCTCTGGA |
| MS2 | (DNA), | AGATGGAACAGCCGGCGAGCAGCTGCACAAGGCCATGAAGAGATATGCCCTG |
| | 153 | GTGCCTGGCACAATCGCCTTCACAGATGCCCACATCGAGGTGGACATCACCTA |
| | (RNA) | CGCCGAGTACTTCGAGATGTCTGTGCGGCTGGCCGAAGCTATGAAGCGCTACG |
| | | GCCTGAACACCAACCACCGGATCGTCGTGTGCAGCGAGAACAGCCTGCAGTTC |
| | | TTCATGCCTGTGCTGGGCGCCCTGTTCATCGGAGTTGCTGTGGCCCCTGCCAAC |
| | | GACATCTACAACGAGCGCGAGCTGCTGAACAGCATGGGCATCTCTCAGCCCAC |
| | | CGTGGTGTTCGTGTCCAAGAAGGGACTGCAGAAAATCCTGAACGTGCAGAAGA |
| | | AGCTGCCCATCATCCAGAAAATCATCATCATGGACAGCAAGACCGACTACCAG |
| | | GGCTTCCAGAGCATGTACACCTTCGTGACCAGCCATCTGCCACCTGGCTTCAAC |
| | | GAGTACGACTTCGTGCCCGAGAGCTTCGACCGGGACAAGACAATCGCCCTGAT |
| | | CATGAACAGCAGCGGCTCTACCGGACTGCCCAAAGGTGTTGCCCTGCCTCATA |
| | | GAACTGCCTGCGTGCGGTTTAGCCACGCCAGAGATCCCATCTTCGGCAACCAG |
| | | ATCATCCCCGACACAGCCATCCTGAGCGTGGTGCCTTTTCACCACGGCTTCGGC |
| | | ATGTTCACCACACTGGGCTACCTGATCTGCGGCTTCAGAGTGGTGCTGATGTAC |
| | | CGCTTCGAGGAAGAACTGTTCCTGCGGAGCCTGCAGGACTACAAGATCCAGTC |
| | | TGCTCTGCTGGTGCCTACTCTGTTCAGCTTCTTTGCCAAGAGCACCCTGATCGAT |
| | | AAGTACGACCTGAGCAACCTGCACGAGATCGCCTCTGGCGGAGCCCCTCTGTC |
| | | TAAAGAAGTGGGAGAAGCCGTCGCCAAGCGGTTCCATCTGCCTGGCATCAGAC |
| | | AAGGCTATGGCCTGACCGAGACAACCAGCGCCATTCTGATTACCCCTGAGGGC |
| | | GACGATAAGCCTGGCGCTGTGGGAAAAGTGGTGCCATTCTTCGAGGCCAAGGT |
| | | GGTGGATCTGGACACCGGCAAAACACTGGGCGTTAACCAGAGGGGCGAGCTGT |
| | | GTGTTAGAGGCCCTATGATCATGAGCGGCTACGTGAACAACCCCGAGGCCACA |
| | | AACGCTCTGATCGACAAGGATGGATGGCTGCACAGCGGCGACATTGCCTACTG |
| | | GGACGAAGATGAGCACTTCTTCATCGTGGACCGGCTGAAGTCCCTGATCAAGT |
| | | ACAAGGGCTACCAGGTGGCCCCAGCCGAGCTGGAATCTATTCTGCTGCAACAC |
| | | CCCAACATCTTCGATGCCGGCGTTGCAGGACTGCCCGATGATGATGCTGGCGA |
| | | ACTGCCAGCTGCTGTGGTGGTGCTGGAACACGGCAAGACCATGACCGAGAAAG |
| | | AAATCGTGGACTACGTGGCCAGCCAAGTGACCACCGCCAAGAAACTGAGAGGC |
| | | GGCGTGGTGTTTGTGGACGAGGTGCCAAAAGGCCTGACAGGCAAGCTGGACGC |
| | | CCGGAAGATCAGAGAGATCCTGATTAAGGCCAAGAAAGGCGGCAAGATCGCC |
| | | GTGTGAgatatcagctacggaactcttgtgcgtaaggatcctaaggtaccta |
| | | attgcctagaaaacatgaggatcacccatgtctgcaggtcgactctagaaaa |
| | | catgaggatcacccatgtctgcagtattcccgggttcattagatcctaaggt |
| | | acctaattgcctagaaaacatgaggatcacccatgtctgcaggtcgactcta |
| | | gaaaacatgaggatcacccatgtctgcagtattcccgggttcattagatcct |
| | | aaggtacctaattgcctagaaaacatgaggatcacccatgtctgcaggtcga |
| | | ctctagaaaacatgaggatcacccatgtctgcagtattcccgggttcattag |
| | | atcctaaggtacctaattgcctagaaaacatgaggatcacccatgtctgcag |
| | | gtcgactctagaaaacatgaggatcacccatgtctgcagtattcccgggttc |
| | | attagatcctaaggtacctaattgcctagaaaacatgaggatcacccatgtc |
| | | tgcaggtcgactctagaaaacatgaggatcacccatgtctgcagtattcccg |
| | | ggttcattagatcctaaggtacctaattgcctagaaaacatgaggatcaccc |
| | | atgtctgcaggtcgactctagaaaacatgaggatcacccatgtctgcagtat |
| | | tcccgggttcattagatcctaa |
| EPM- | 154 | ALPGNPDHREMGETLPEEVGEYRQPSGGSVPVSPGPPSGLEPTSSSPYGSKSGSGS |
| CsrA | | DSGSMLILTRRVGETLMIGDEVTVTVLGVKGNQVRIGVNAPKEVSVHREEIYQRIQA |
| | | EKSQQSSY |
| sCsrA- | 155 | MLILTRRVGETLMIGDEVTVTVLGVKGNQVRIGVNAPKEVSVHREEIYQRIQAEKSQ |
| EABR | | QSSYGGGSFNSSINNIHEMEIQLKDALEKNQQWLVYDQQREVYVKGLLAKIFELEK |
| | | KTETAAHSLP |
| CD4 CT-156 | | CVRCRHRRRQAERMSQIKRLLSEKKTCQCPHRFQKTCSPIALPGNPDHREMGET |
| EPM- | | LPEEVGEYRQPSGGSVPVSPGPPSGLEPTSSSPYGSKSGSGSDSGSMLILTRRV |
| CsrA | | GETLMIGDEVTVTVLGVKGNQVRIGVNAPKEVSVHREEIYQRIQAEKSQQSSY |
| Lck- | 157 | MGCGCSSHPEDDWMENIDVCENCHYPIVPLDGKGTLLIRNGSEVRDPLVTYEG |
| CsrA- | | SNPPASPLGSKSGSGSDSGSMLILTRRVGETLMIGDEVTVTVLGVKGNQVRIGVNA |
| EABR | | PKEVSVHREEIYQRIQAEKSQQSSYGGGSFNSSINNIHEMEIQLKDALEKNQQWLVY |
| V | | DQQREVYVKGLLAKIFELEKKTETAAHSLP |
| CD4 CT- | 158 | CVRCRHRRRQAERMSQIKRLLSEKKTCQCPHRFQKTCSPIALPGNPDHREMGET |
| EPM- | | LPEEVGEYRQPSGGSVPVSPGPPSGLEPTSSSPYGSKSGSGSDSGSMLILTRRV |
| CsrA | | GETLMIGDEVTVTVLGVKGNQVRIGVNAPKEVSVHREEIYQRIQAEKSQQSSYG |
| EABR | | GGSFNSSINNIHEMEIQLKDALEKNQWLVYDQQREVYVKGLLAKIFELEKKTET |
| | | AAHSLP |
| Lck- | 159 | MGCGCSSHPEDDWMENIDVCENCHYPIVPLDGKGTLLIRNGSEVRDPLVTYEG |
| CsrA | | SNPPASPLGSKSGSGSDSGSMLILTRRVGETLMIGDEVTVTVLGVKGNQVRIGVNA |
| | | PKEVSVHREEIYQRIQAEKSQQSSY |
| CD4 | 160 | CVRCRHRRRQAERMSQIKRLLSEKKTCQCPHRFQKTCSPIALPGNPDHREMGET |
| CT- | | LPEEVGEYRQPSGGSVPVSPGPPSGLEPTSSSPY |
| EPM | | |
| CD4 | 161 | CVRCRHRRRQAERMSQIKRLLSEKKTCQCPHRFQKTCSPIALPGNPDHREMGET |
| CT- | | LPEEVGEYRQPSGGSVPVSPGPPSGLEPTSSSPYGSKSGSGSDSGSMSDNGPQNQR |
| EPM-N | | NAPRITFGGPSDSTGSNQNGERSGARSKQRRPQGLPNNTASWFTALTQHGKEDLKF |
| | | PRGQGVPINTNSSPDDQIGYYRRATRRIRGGDGKMKDLSPRWYFYYLGTGPEAGLP |
| | | YGANKDGIIWVATEGALNTPKDHIGTRNPANNAAIVLQLPQGTTLPKGFYAEGSRG |
| | | GSQASSRSSSRSRNSSRNSTPGSSRGTSPARMAGNGGDAALALLLLDRLNQLESKMS |
| | | GKGQQQQGQTVTKKSAAEASKKPRQKRTATKAYNVTQAFGRRGPEQTQGNFGDQ |
| | | ELIRQGTDYKHWPQIAQFAPSASAFFGMSRIGMEVTPSGTWLTYTGAIKLDDKDPN |
| | | FKDQVILLNKHIDAYKTFPPTEPKKDKKKKADETQALPQRQKKQQTVTLLPAADLD |
| | | DESKQLQQSMSSADSTQA |
| Lck-N- | 162 | MGCGCSSHPEDDWMENIDVCENCHYPIVPLDGKGTLLIRNGSEVRDPLVTYEG |
| EABR | | SNPPASPLGSKSGSGSDSGSMSDNGPQNQRNAPRITFGGPSDSTGSNQNGERSGA |
| | | RSKQRRPQGLPNNTASWFTALTQHGKEDLKFPRGQGVPINTNSSPDDQIGYYRRAT |
| | | RRIRGGDGKMKDLSPRWYFYYLGTGPEAGLPYGANKDGIIWVATEGALNTPKDHIG |
| | | TRNPANNAAIVLQLPQGTTLPKGFYAEGSRGGSQASSRSSSRSRNSSRNSTPGSSRGT |
| | | SPARMAGNGGDAALALLLLDRLNQLESKMSGKGQQQQGQTVTKKSAAEASKKPRQ |
| | | KRTATKAYNVTQAFGRRGPEQTQGNFGDQELIRQGTDYKHWPQIAQFAPSASAFF |
| | | GMSRIGMEVTPSGTWLTYTGAIKLDDKDPNFKDQVILLNKHIDAYKTFPPTEPKKD |
| | | KKKKADETQALPQRQKKQQTVTLLPAADLDDESKQLQQSMSSADSTQAGGGSENS |
| | | SINNIHEMEIQLKDALEKNQQWLVYDQQREVYVKGLLAKIFELEKKTETAAHSLP |
| CD4 | 163 | CVRCRHRRRQAERMSQIKRLLSEKKTCQCPHRFQKTCSPIALPGNPDHREMGET |
| CT- | | LPEEVGEYRQPSGGSVPVSPGPPSGLEPTSSSPYGSKSGSGSDSGSMYVRFEVPED |
| EPM- | | MQNEALSLLEKVRESGKVKKGTNETTKAVERGLAKLVYIAEDVDPPEIVAHLPLL |
| L7Ae | | CEEKNVPYIYVKSKNDLGRAVGIEVPCASAAIINEGELRKELGSLVEKIKGLQK |
| Lck- | 164 | MGCGCSSHPEDDWMENIDVCENCHYPIVPLDGKGTLLIRNGSEVRDPLVTYEG |
| L7Ae- | | SNPPASPLGSKSGSGSDSGSMYVRFEVPEDMQNEALSLLEKVRESGKVKKGTNETT |
| EABR | | KAVERGLAKLVYIAEDVDPPEIVAHLPLLCEEKNVPYIYVKSKNDLGRAVGIEVPCAS |
| | | AAIINEGELRKELGSLVEKIKGLQKGGGSFNSSINNIHEMEIQLKDALEKNQQWLVY |
| | | DQQREVYVKGLLAKIFELEKKTETAAHSLP |
| Lucmin | 165 | TGCAACACCCCAACATCTTCGATGCCGGCGTTGCAGGACTGCCCGATGATG |
| | (DNA), | ATGCTGGCGAACTGCCAGCTGCTGTGGTGGTGCTGGAACACGGCAAGACCA |
| | 167 | TGACCGAGAAAGAAATCGTGGACTACGTGGCTGAAAGCTTGGC |
| | (RNA) | |
| Lucmin- | 166 | TGCAACACCCCAACATCTTCGATGCCGGCGTTGCAGGACTGCCCGATGATGAT |
| PS9 | (DNA), | GCTGGCGAACTGCCAGCTGCTGTGGTGGTGCTGGAACACGGCAAGACCATGAC |
| | 168 | CGAGAAAGAAATCGTGGACTACGTGGCTGAAAGCTTGGCtctgtaggtccca |
| | (RNA) | aacaagctagtcttaatggagtcacattaattggagaagccgtaaaaacaca |
| | | gttcaattattataagaaagttgatggtgttgtccaacaattacctgaaact |
| | | tactttactcagagtagaaatttacaagaatttaaacccaggagtcaaatgg |
| | | aaattgatttcttagaattagctatggatgaattcattgaacggtataaatt |
| | | agaaggctatgccttcgaacatatcgtttatggagattttagtcatagtcag |
| | | ttaggtggtttacatctactgattggactagctaaacgttttaaggaatcac |
| | | cttttgaattagaagattttattcctatggacagtacagttaaaaactattt |
| | | cataacagatgcgcaaacaggttcatctaagtgtgtgtgttctgttattgat |
| | | ttattacttgatgattttgttgaaataataaaatcccaagatttatctgtag |
| | | tttctaaggttgtcaaagtgactattgactatacagaaatttcatttatgct |
| | | ttggtgtaaagatggccatgtagaaacattttacccaaaattacaatctagt |
| | | caagcgtggcaaccgggtgttgctatgcctaatctttacaaaatgcaaagaa |
| | | tgctattagaaaagtgtgaccttcaaaattatggtgatagtgcaacattacc |
| | | taaaggcataatgatgaatgtcgcaaaatatactcaactgtgtcaatattta |
| | | aacacattaacattagctgtaccctataatatgagagttatacattttggtg |
| | | ctggttctgataaaggagttgcaccaggtacagctgttttaagacagtggtt |
| | | gcctacgggtacgctgcttgtcgattcagatcttaatgactttgtctctgat |
| | | gcagattcaactttgattggtgattgtgcaactgtacatacagctaataaat |
| | | gggatctcattattagtgatatgtacgaccctaagactaaaaatgttacaaa |
| | | agaaaatgactctaaagagggttttttcacttacatttgtgggtttatacaa |
| | | caaaagctagctcttggaggttccgtggctataaagata |
| VSV- | 169 | MKCLLYLAFLFIGVNCKFTIVFPHNQKGNWKNVPSNYHYCPSSSDLNWHNDLI |
| Gmut | | GTALQVKMPQSHKAIQADGWMCHASKWVTTCDFRWYGPKYITHSIRSFTPSV |
| | | EQCKESIEQTKQGTWLNPGFPPQSCGYATVTDAEAVIVQVTPHHVLVDEYTGE |
| | | WVDSQFINGKCSNYICPTVHNSTTWHSDYKVKGLCDSNLISMDITFFSEDGELS |
| | | SLGKEGTGFRSNYFAYETGGKACKMQYCKHWGVRLPSGVWFEMADKDLFA |
| | | AARFPECPEGSSISAPSQTSVDVSLIQDVERILDYSLCQETWSKIRAGLPISPVDL |
| | | SYLAPKNPGTGPAFTIINGTLKYFETRYIRVDIAAPILSRMVGMISGTTTEAELW |
| | | DDWAPYEDVEIGPNGVLRTSSGYKFPLYMIGHGMLDSDLHLSSKAQVFEHPHI |
| | | QDAASQLPDDESLFFGDTGLSKNPIELVEGWFSSWKSSIASFFFIIGLIIGLFLVLR |
| | | VGIHLCIKLKHTKKRQIYTDIEMNRLGK |

*EPM is indicated by underline italic, RBP by bold italic underline, ERD by italic, and PS by bold italic. For S-CD4 CT constructs, the underlined sequence in SARS-CoV-2 S was replaced with the underlined sequence in the CD4 CT sequences. For adapter fusion proteins, the adapter domain is underlined. For dimerization fusion proteins, the CSP heterologous cytoplasmic tail (e.g., CD4 CT) is underlined. Point mutations from wild type are shown in bold. The coding region of the Luc DNA sequence is shown in double underline. For cargo sequences, a DNA sequence is shown, and SEQ ID NOs for both RNA and DNA sequences are provided.

### Example 2

### Co-Delivery of a Tetherin Antagonist Rescues Budding of Enveloped Nanoparticles (ENPs) in Tetherin-Expressing Cells

Provided in this Example are data related to methods and compositions for improvement of ENP budding (e.g., production) from cells by inhibiting tetherin.

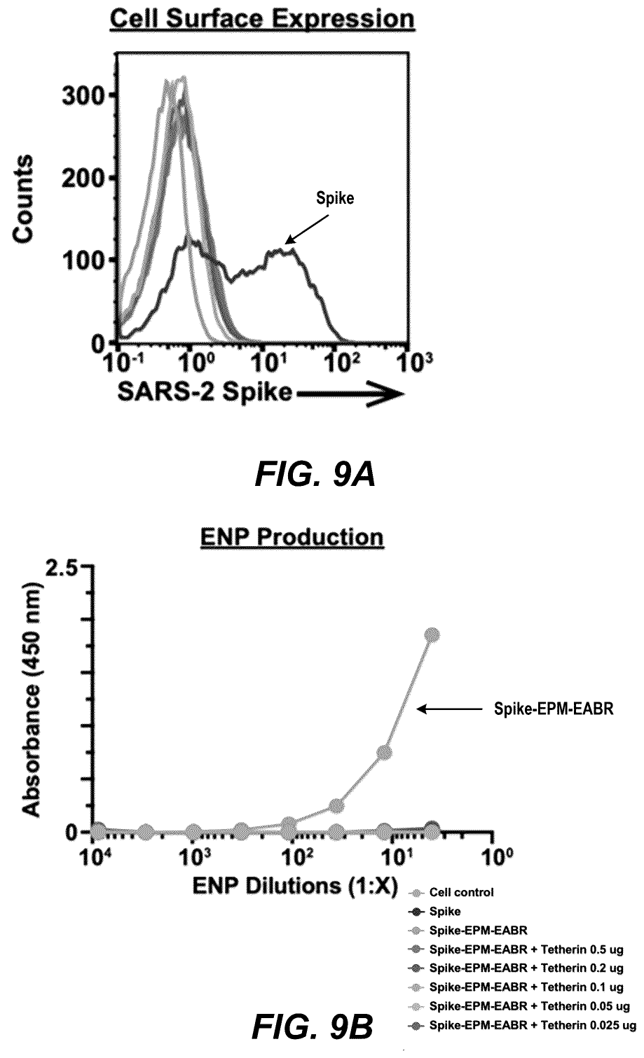

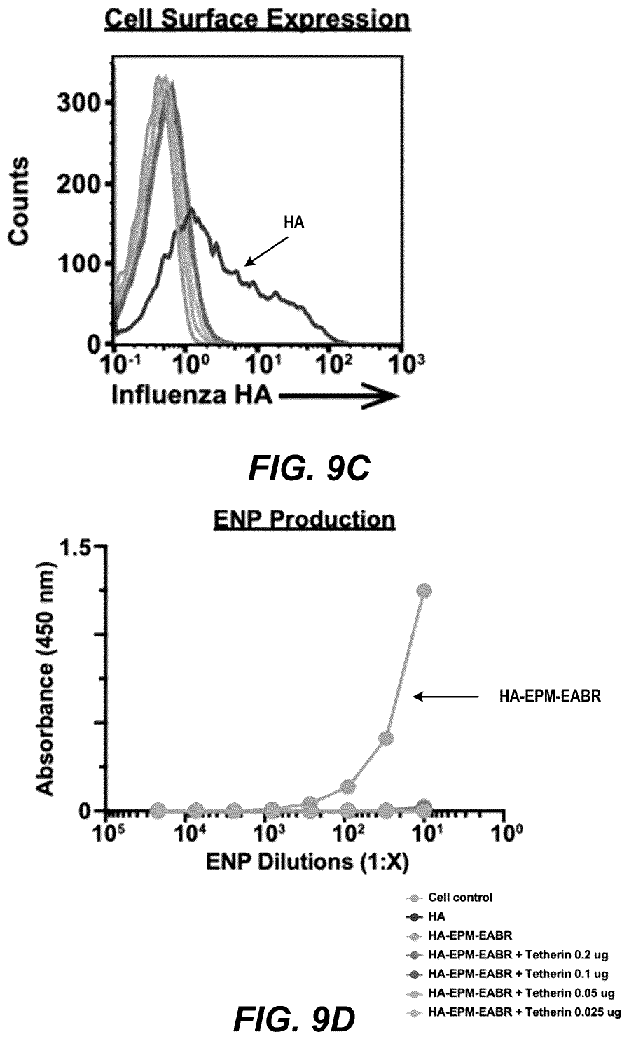

To investigate whether tetherin blocks ENP budding, mRNAs encoding SARS-CoV-2 spike, spike-EPM-EABR, influenza HA, HA-EPM-EABR, or tetherin (Table 5) were synthesized and transfection experiments were performed using HEK293T cells. Cells were transfected with 2 μg of Spike-EPM-EABR mRNA or 0.25 μg of HA-EPM-EABR mRNA in the absence or presence of tetherin. Various amounts of mRNA encoding tetherin (0.025-0.5 μg) were co-transfected to evaluate the effect of tetherin on ENP budding. 48 hours post-transfection, cell surface expression was analyzed by flow cytometry and ENPs were purified from transfected cell culture supernatants by ultracentrifugation on a 20% sucrose cushion. Cell surface expression of spike-EPM-EABR and HA-EPM-EABR was slightly increased in the presence of tetherin but remained much lower compared to spike and HA, respectively (FIG. 9A and FIG. 9C). Importantly, tetherin co-expression completely abolished ENP budding for spike-EPM-EABR and HA-EPM-EABR constructs at all tested tetherin mRNA transfection amounts (FIG. 9B and FIG. 9D). These results demonstrate that interferon-induced tetherin expression may, in some instances, inhibit ENP budding. Interestingly, SARS-COV-2 spike has been reported to antagonize tetherin, but this activity was insufficient to rescue ENP budding induced by the spike-EPM-EABR fusion construct.

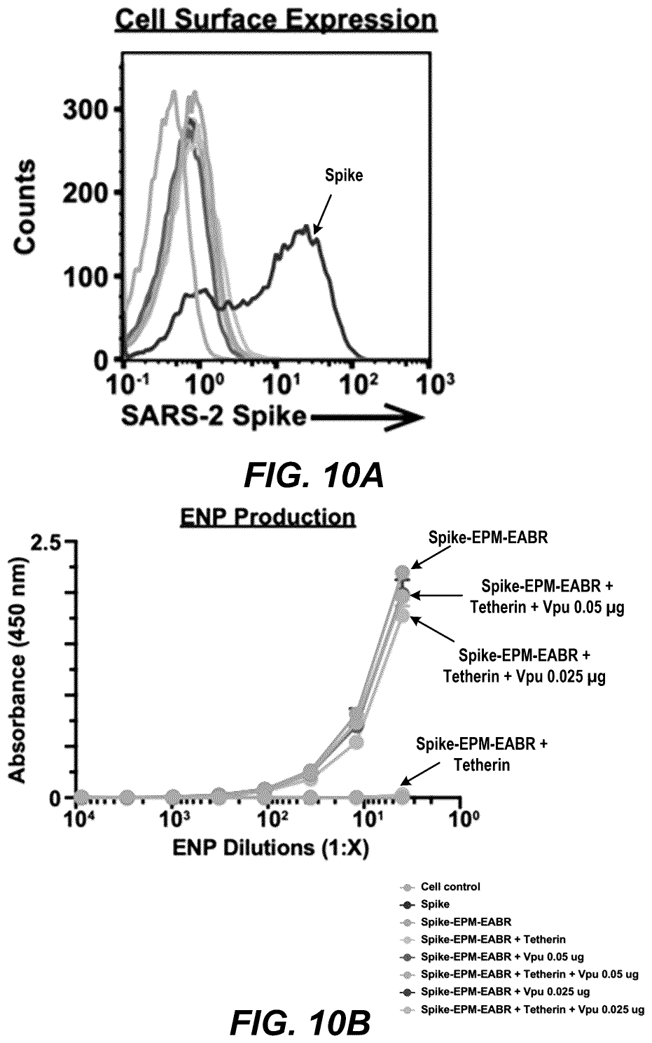

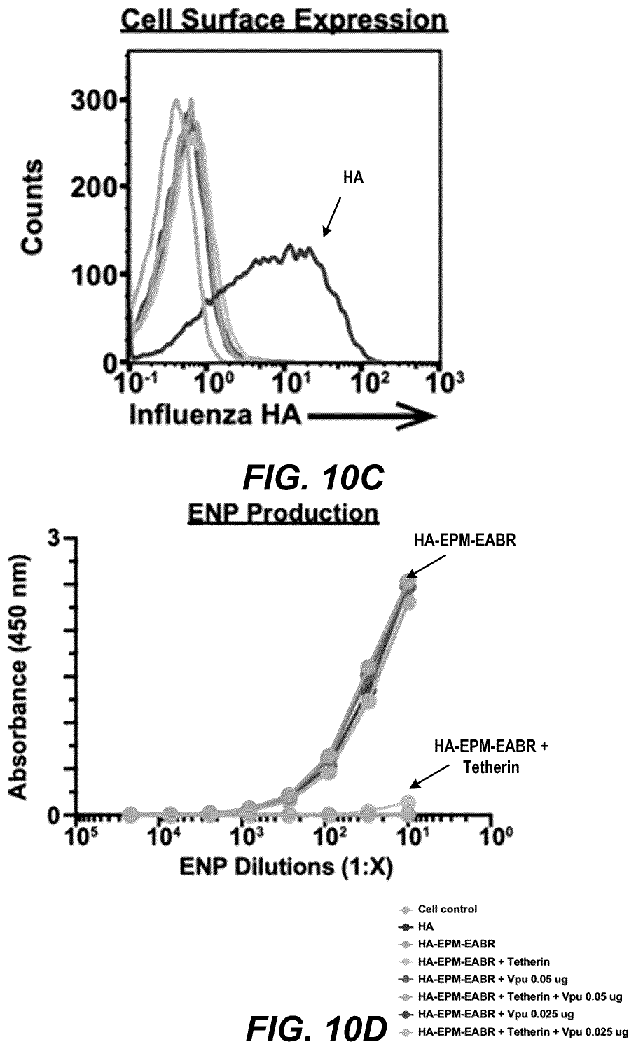

To counteract tetherin-mediated blockage of ENP budding, mRNA encoding the HIV-1 Vpu protein was synthesized (isolate BRU/LAI) (Table 5). Co-transfection of 0.05 or 0.025 μg Vpu mRNA completely restored ENP budding for SARS-COV-2 spike-EPM-EABR and influenza HA-EPM-EABR fusion constructs in the presence of 0.025 μg tetherin (FIG. 10A-FIG. 10D). These results demonstrate that co-delivery of a tetherin antagonist can prevent tetherin-mediated blockage of ENP budding.

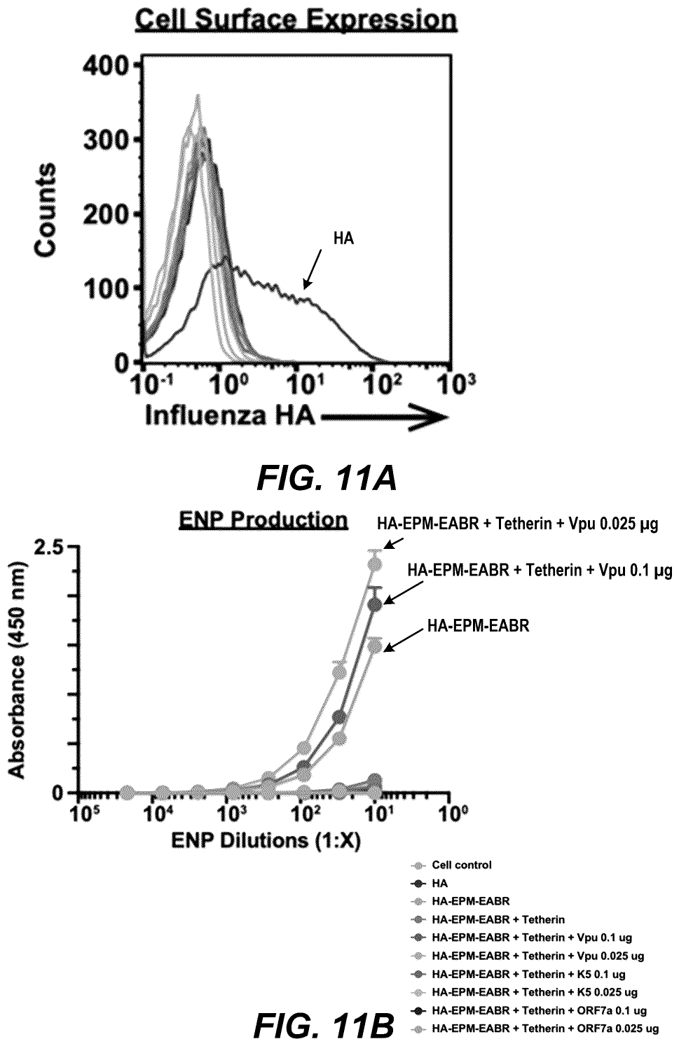

The effects of other viral tetherin antagonists on ENP budding were also evaluated. 0.1 or 0.025 μg of mRNAs encoding HIV-1 Vpu, KSHV K5 (GK18 strain), or SARS-CoV-2 ORF7a (Wuhan-Hu-1 isolate) (Table 5) were co-transfected with influenza HA-EPM-EABR in the absence or presence of 0.025 μg tetherin. While ENP budding was completely rescued by HIV-1 Vpu, tetherin-mediated blockage of ENP budding was not prevented by KSHV K5 and SARS-COV-2 ORF7a at both transfection amounts (FIG. 11A-FIG. 11B). It may be in some instances that higher doses of K5 and ORF7a were required to effectively counteract tetherin, suggesting that these tetherin antagonists are markedly less potent compared to HIV-1 Vpu. These results might also suggest that their modes of action are only effective in the context of a viral infection to facilitate viral egress but do not effectively inhibit tetherin-mediated blockage of ENP budding.

Demonstrated herein is that the antiviral restriction factor tetherin can block ENP budding. Tetherin expression increased the cell surface expression of immunogen-ERD fusion and ERD adapter constructs, likely because, without being bound by any particular theory, budding ENPs were tethered to the plasma membrane preventing their diffusion away from the cells. Tetherin-mediated linkage of viral particles to the cell has been shown to result in endocytosis of the virions, suggesting that the tethered ENPs might not be able to engage and activate immune cells.

Importantly, mRNA co-delivery of the viral tetherin antagonist HIV-1 Vpu completely rescued ENP budding in the presence of tetherin, suggesting that the addition of tetherin antagonists can improve ERD-based RNA cargo delivery via ENPs. Interestingly, the mRNA-encoded tetherin antagonists KSHV K5 and SARS-COV-2 ORF7a had no effect on rescuing ENP budding, suggesting that their modes of action do not effectively inhibit tetherin-mediated blockage of ENP budding.

Co-delivery of mRNA-encoded ERD immunogens and tetherin antagonists can be achieved in various ways. In some embodiments, the immunogen and the tetherin antagonist could be encoded by separate mRNAs. The mRNAs could then be mixed before lipid nanoparticle (LNP) encapsulation to ensure that both mRNAs get packaged into the same LNP resulting in cellular co-expression of the ERD immunogen and tetherin antagonist. In some embodiments, the ERD immunogen and tetherin antagonist could be delivered by bicistronic mRNAs using an internal ribosome entry site (IRES) or 2A peptide.

TABLE 5
Exemplary Tetherin and Tetherin Inhibitor Sequences

| NAME | SEQ ID NO: | SEQUENCE |
|---|---|---|
| Tetherin | 124 | MASTSYDYCRVPMEDGDKRCKLLLGIGILVLLIIVILGVPLII |
| | | FTIKANSEACRDGLRAVMECRNVTHLLQQELTEAQKGFQDVEA |
| | | QAATCNHTVMALMASLDAEKAQGQKKVEELEGEITTLNHKL |
| | | QDASAEVERLRRENQVLSVRIADKKYYPSSQDSSSAAAPQLLI |
| | | VLLGLSALLQ |
| HIV-1 Vpu | 125 | MQPIQIAIAALVVAIIIAIVVWSIVIIEYRKILRQRKIDRLI |
| | | DRLIERAEDSGNESEGEISALVEMGVEMGHHAPWDIDDL |
| KSHV K5 | 126 | MASKDVEEGVEGPICWICREEVGNEGIHPCACTGELDVVHPQ |
| | | CLSTWLTVSRNTACQMCRVIYRTRTQWRSRLNLWPEMERQE |
| | | IFELFLLMSVVVAGLVGVALCTWTLLVILTAPAGTFSPGAVL |
| | | GFLCFFGFYQIFIVFAFGGICRVSGTVRALYAANNTRVTVLPY |
| | | RRPRRPTANEDNIELTVLVGPAGGTDEEPTDESSEGDVASGD |
| | | KERDGSSGDEPDGGPNDRAGLRGTARTDLCAPTKKPVRKNH |
| | | PKNNG |
| SARS-CoV-2 | 127 | MKIILFLALITLATCELYHYQECVRGTTVLLKEPCSSGTYEGN |
| ORF7a | | SPFHPLADNKFALTCFSTQFAFACPDGVKHVYQLRARSVSPK |
| | | LFIRQEEVQELYSPIFLIVAAIVFITLCFTLKRKTE |

### Example 3

### Development of ENP Delivery System

Described in this Example is continued development of the ENP delivery systems by demonstrating that the L7Ae-Box C/D and N-PS9 RBP-PS combinations do not require co-expression of soluble RBP for efficient packaging and delivery of RNA cargoes. It is further described that ENP delivery systems can efficiently deliver mRNAs encoding fluorescent reporter proteins to specific target cells, which can be detected by flow cytometry in individual cells. Importantly, it is demonstrated herein that single-chain variable fragments (scFvs) can be displayed as targeting proteins on ENPs to program target cell tropism. scFvs that bind the CD19 (expressed on B cells) or the CD3/CD4 receptors (expressed on CD4⁺ T cells) promoted specific RNA cargo delivery to target cells expressing these receptors. ENPs were then demonstrated to efficiently deliver mRNAs encoding an cancer immunotherapeutic—the immunostimulatory cytokine IL12-to CD19+ cells as a potential therapy for B cell malignancies. This strategy can be particularly impactful for targeting solid tumors as the secretion of cytokines can modulate the immunosuppressive nature of the tumor microenvironment and enhance the anti-tumor activity of endogenous and/or genetically engineered immune cells.

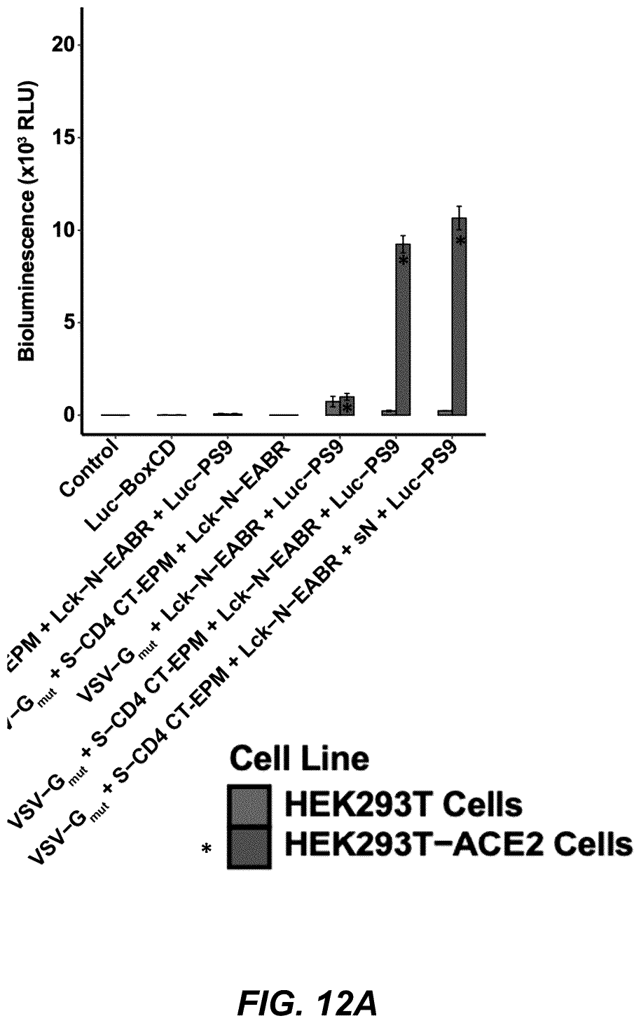

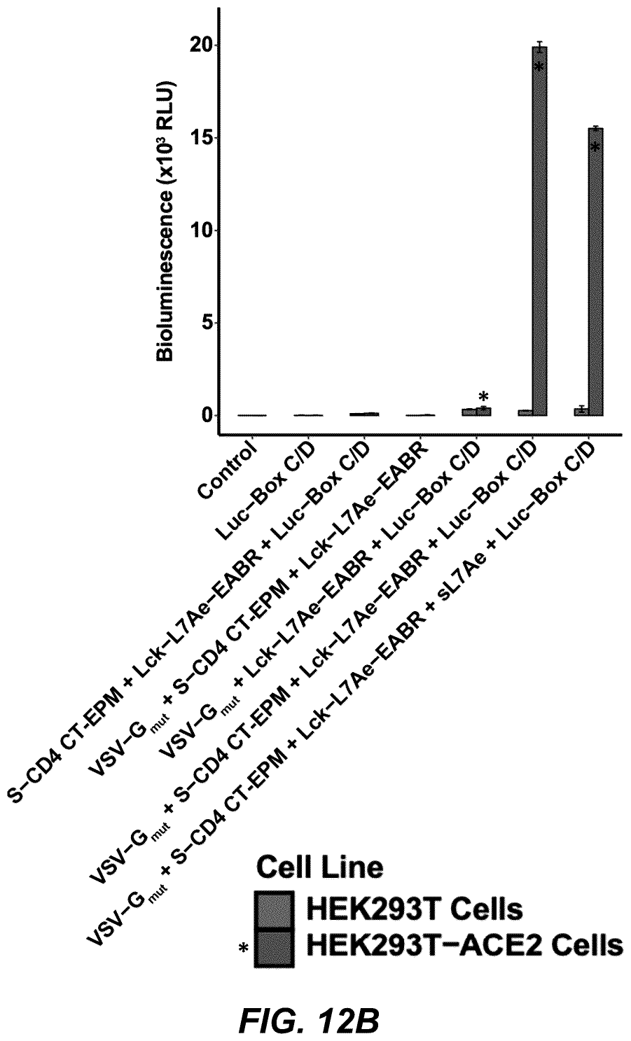

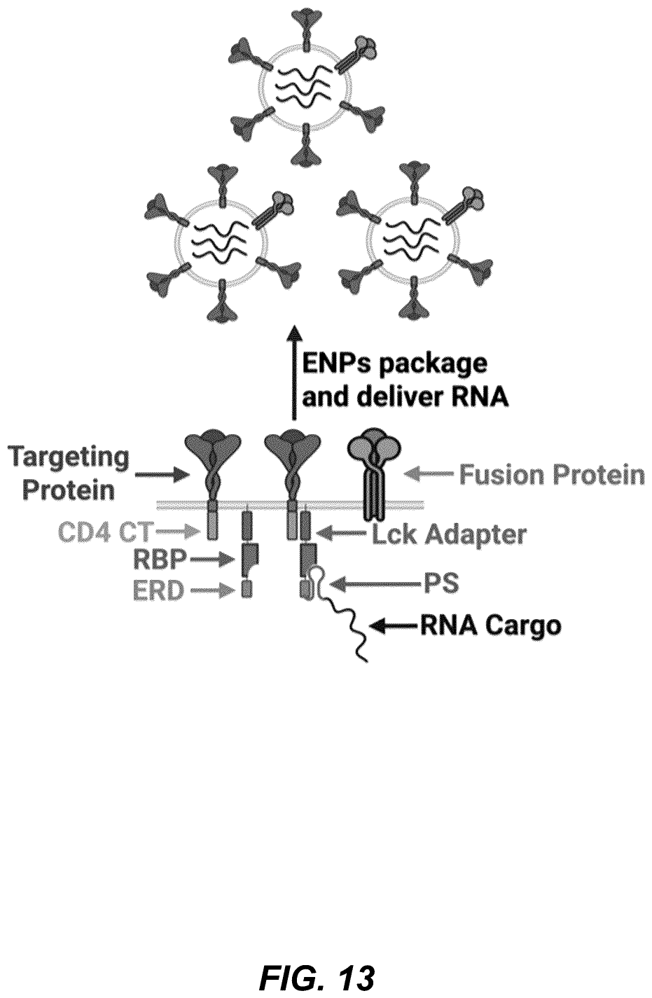

The CsrA-CsrB RBP-PS combination promoted more efficient ENP packaging of RNA cargoes when sCsrA was co-expressed to facilitate CsrA dimerization (FIG. 1A-FIG. 1C). It was next investigated whether the N-PS9 and L7Ae-Box C/D RBP-PS combinations also require co-expression of the soluble RBP (sN or sL7Ae). Interestingly, the VSV-Gₘᵤₜ+S-CD4 CT-EPM+Lck-N-EABR+Luc-PS9 ENPs and the VSV-Gₘᵤₜ+S-CD4 CT-EPM+Lck-L7Ae-EABR+Luc-Box C/D ENPs efficiently delivered the Luc-encoding mRNA cargo to HEK293T-ACE2 target cells in the presence or absence of sN or sL7Ae, respectively (FIG. 12A-FIG. 12B). The highest bioluminescence in HEK293T-ACE2 target cells was observed for VSV-Gₘᵤₜ+S-CD4 CT-EPM+Lck-L7Ae-EABR+Luc-Box C/D (no sL7Ae) ENPs. Bioluminescence in off-target HEK293T cells was low for all conditions demonstrating that mRNA delivery was highly specific. These results show that the L7Ae-Box C/D and N-PS9 RBP-PS combinations do not require co-expression of sL7Ae or sN for efficient ENP packaging and delivery (FIG. 13). Interestingly, these experiments were performed in the absence of polybrene, suggesting that polybrene is not needed to enhance cellular ENP uptake.

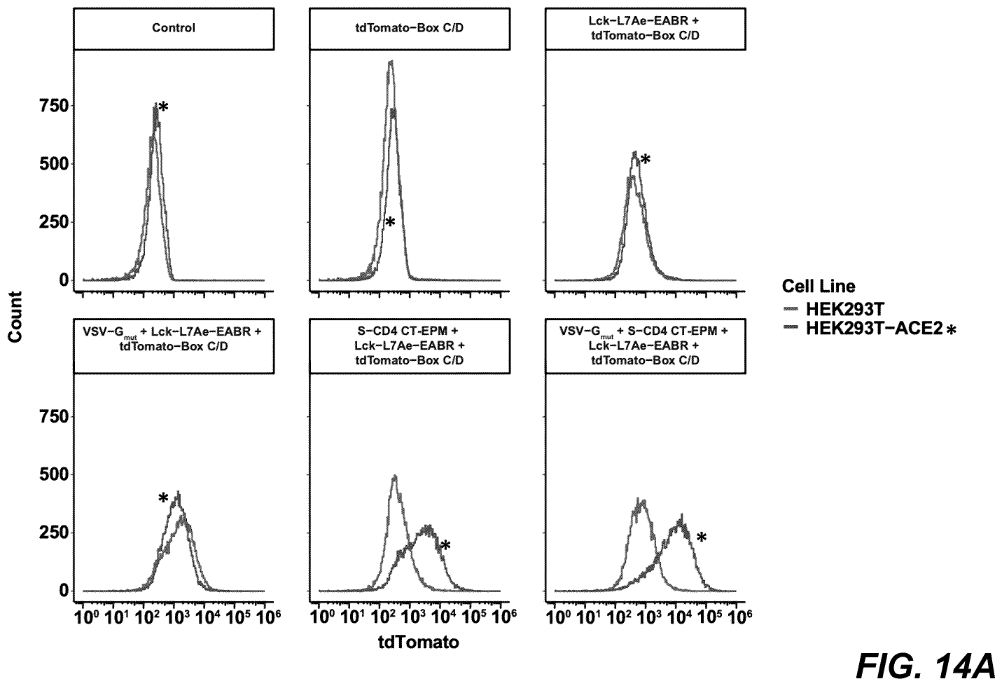

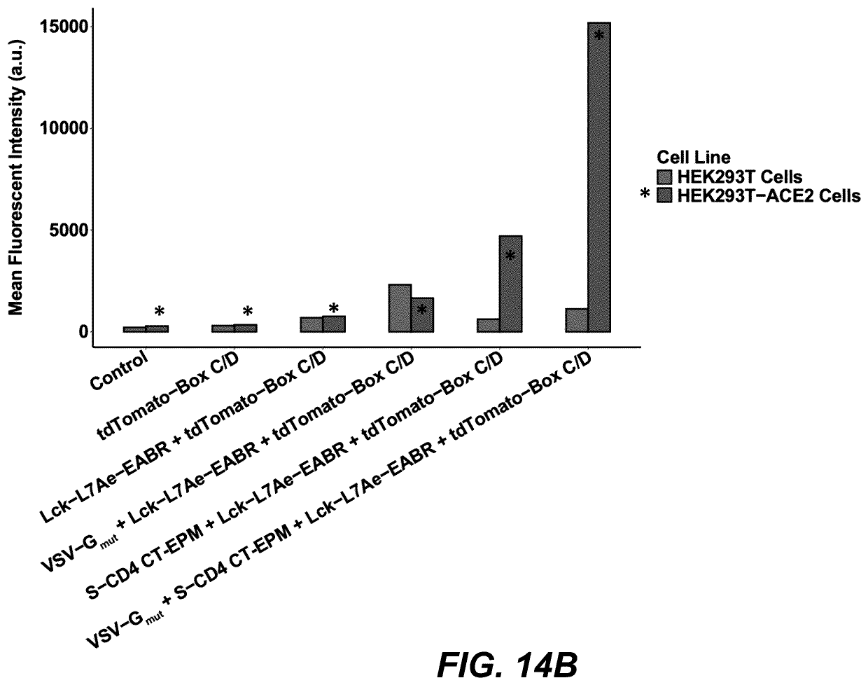

To demonstrate that ENPs can deliver a wide range of mRNA cargoes to specific target cells, ENPs that package mRNA encoding the fluorescent protein tdTomato were generated next. ENPs were produced in HEK293T cells as described above (See, e.g., Example 1) and added to pre-seeded HEK293T or HEK293T-ACE2 cells in 96-well plates. After 16 hours, tdTomato expression was analyzed by flow cytometry. Control conditions included no transfection (control), transfection of tdTomato-Box C/D DNA alone, co-transfection of Lck-L7Ae-EABR+tdTomato-Box C/D DNA (no surface proteins), co-transfection of VSV-Gₘᵤₜ+Lck-L7Ae-EABR+tdTomato-Box C/D DNA (no S-CD4 CT-EPM), and co-transfection of S-CD4 CT-EPM+Lck-L7Ae-EABR+tdTomato-Box C/D DNA (no VSV-Gₘᵤₜ). tdTomato expression in off-target HEK293T cells was low for all conditions (FIG. 14A-FIG. 14B). Similar to Luc delivery, the VSV-Gₘᵤₜ+S-CD4 CT-EPM+Lck-L7Ae-EABR+tdTomato-Box C/D ENPs induced the highest tdTomato expression in HEK293T-ACE2 target cells. Importantly, 89% of HEK293T-ACE2 target cells expressed tdTomato, demonstrating that ENPs can deliver mRNA cargoes efficiently to a large fraction of specific target cells.

Interestingly, tdTomato levels were also elevated for the S-CD4 CT-EPM+Lck-L7Ae-EABR+tdTomato-Box C/D ENPs in the absence of VSV-Gₘᵤₜ. Without being bound by any particular theory, this signal was likely the result of tdTomato protein getting encapsulated in ENPs during the ENP packaging process (tdTomato gets expressed in the packaging cells), which then bound to the cell surface of HEK293T-ACE2 cells due to the interaction between S-CD4 CT-EPM on the ENP surface and ACE2 on the cell surface.

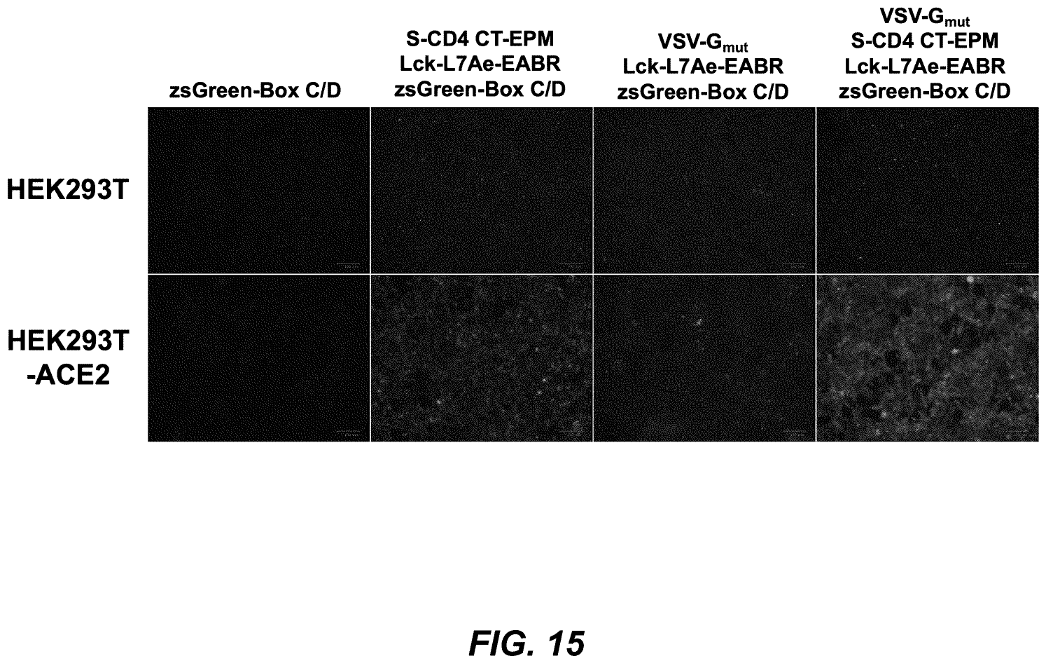

ENP-mediated delivery of mRNA encoding the fluorescent protein zsGreen was also evaluated by fluorescence microscopy imaging (FIG. 15). Low fluorescent signal was observed in off-target HEK293T cells for all ENPs. The presence of both surface proteins (S-CD4 CT-EPM+VSV-Gₘᵤₜ) on the ENP surface again induced the highest level of zsGreen expression in the HEK293T-ACE2 target cells. ENPs that only displayed S-CD4 CT-EPM also induced increased fluorescent signal, likely again due to zsGreen protein that was encapsulated in ENPs during the packaging process. Overall, these results show that ENPs efficiently deliver various mRNA cargoes to specific target cells.

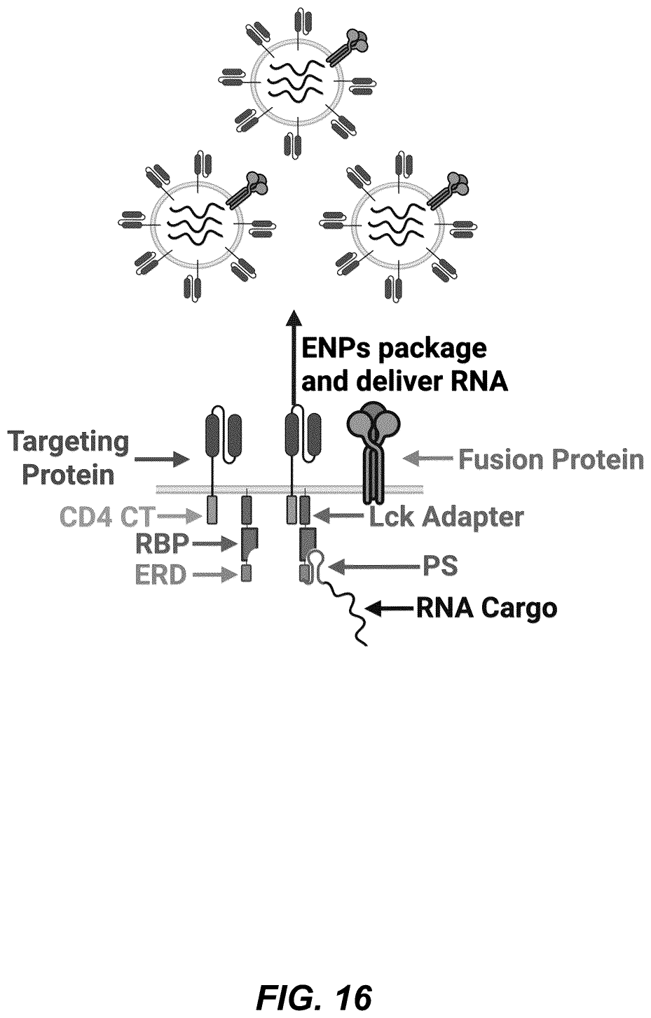

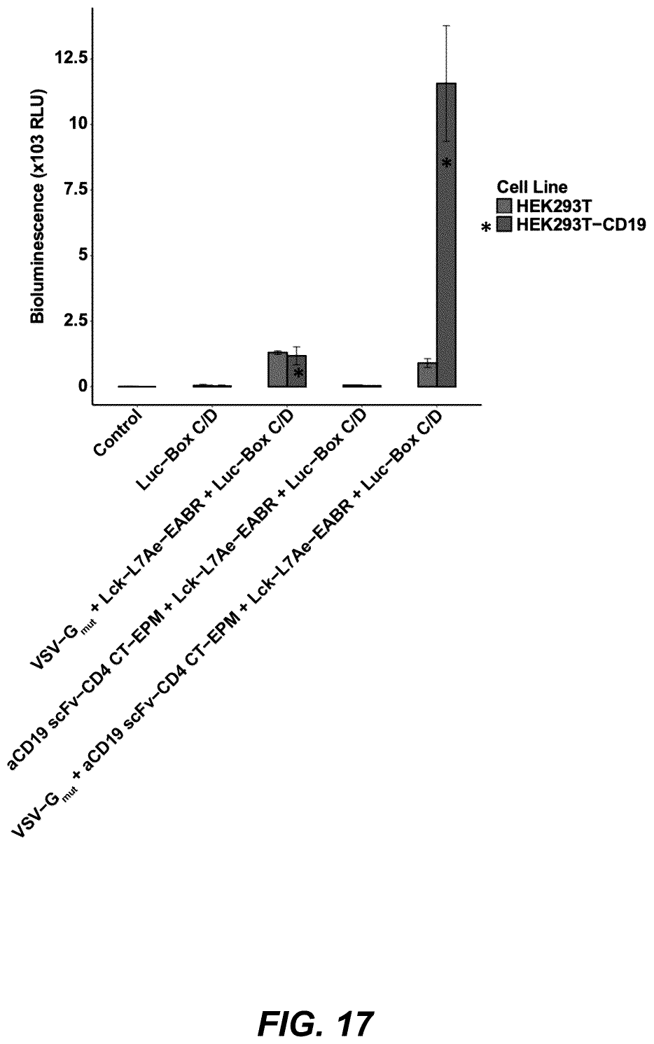

To program ENP targeting to a wide range of different cell types, it was investigated whether the S-CD4 CT-EPM targeting protein can be replaced with membrane-anchored single-chain variable fragments (scFv) that specifically bind cell surface receptors on target cells (FIG. 16). As a proof-of-concept, an scFv targeting protein that binds to the CD19 receptor specifically expressed on B cells was designed. The anti-CD19 scFv was fused to the hinge and transmembrane domains of the human CD8a chain as previously described. To promote interactions with the Lck domain of the Lck-L7Ae-EABR adapter and enhance cell surface expression, the CD4 CT and the EPM sequences were added to generate aCD19 scFv-CD4 CT-EPM (Table 6). ENPs were then generated by co-transfecting VSV-Gₘᵤₜ+aCD19 scFv-CD4 CT-EPM+Lck-L7Ae-EABR+Luc-Box C/D DNA in HEK293T cells. ENPs that co-displayed the VSV-Gₘᵤₜ and aCD19 scFv-CD4 CT-EPM surface proteins induced high bioluminescence in HEK293T target cells that expressed the CD19 receptor (HEK293T-CD19 cells), which was 13-fold higher than in off-target HEK293T control cells (FIG. 17). In contrast, ENPs that only displayed one of the surface proteins, VSV-Gₘᵤₜ or aCD19 scFv-CD4 CT-EPM, induced only low bioluminescence in both cell lines.

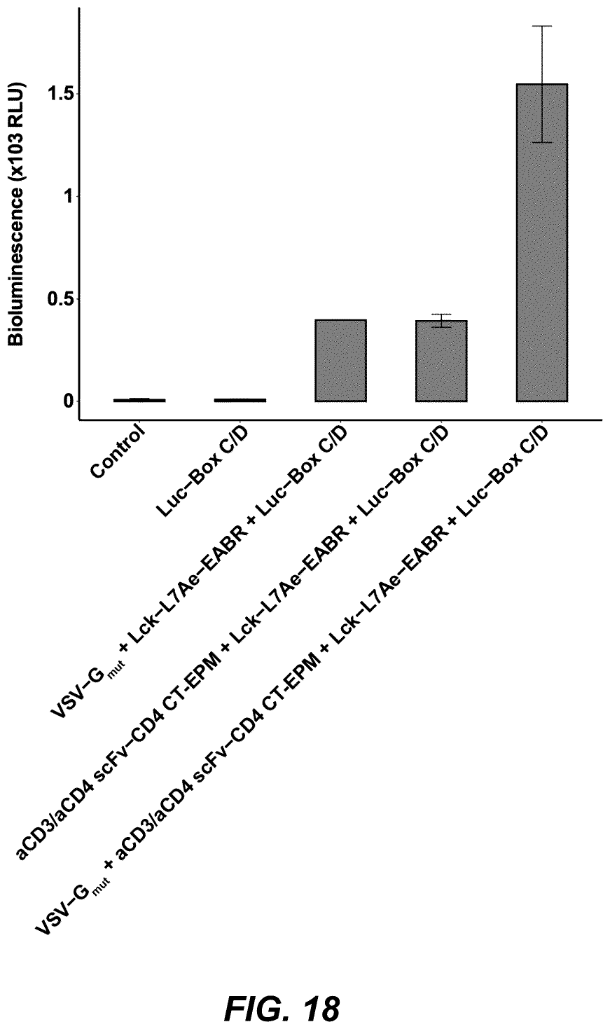

It was next evaluated whether ENPs can be programmed to deliver mRNA cargoes to CD4 T cells. These cells are an important target for drug and gene delivery approaches for the purposes of generating genetically engineered cancer-fighting T cells, as well as eradication efforts for HIV-1 that primarily infects these cells. For lentiviral vector-based delivery, co-display of scFv-based targeting proteins that target two cell surface receptors, CD3 and CD4, has been shown to facilitate efficient gene delivery to CD4 T cells. To evaluate if this strategy also promotes ENP-mediated mRNA delivery to CD4 T cells, we designed aCD3 scFv-CD4 CT-EPM and aCD4 scFv-CD4 CT-EPM targeting proteins by fusing the aCD3 and aCD4 scFv sequences to the CD8a hinge and transmembrane domains, the CD4 CT, and the EPM (Table 6). ENPs were generated by co-transfecting VSV-Gₘᵤₜ+aCD3 scFv-CD4 CT-EPM+aCD4 scFv-CD4 CT-EPM+Lck-L7Ae-EABR+Luc-Box C/D DNA in HEK293T cells. ENPs were then added to Jurkat CD4 T cells. ENPs that co-displayed the aCD3 and aCD4 scFv targeting proteins, as well as the VSV-Gₘᵤₜ fusion protein induced robust bioluminescence, demonstrating efficient mRNA cargo delivery to CD4 T cells (FIG. 18). Overall, these results show that ENPs can be programmed to target RNA cargo delivery to a wide range of cell types through co-display of a single or multiple scFv-based targeting proteins.

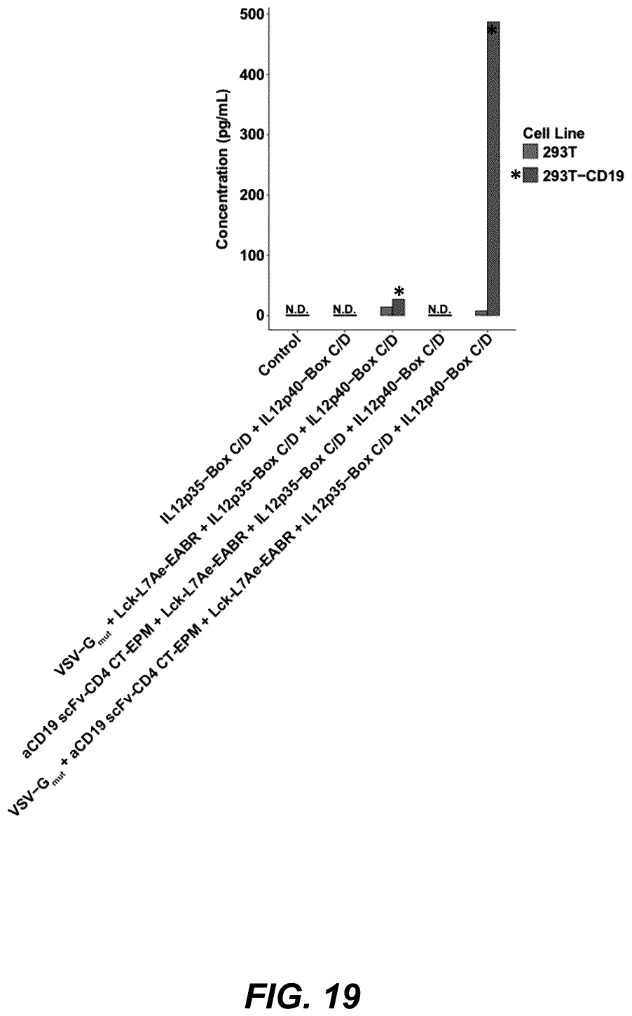

The described ENP delivery system can be programmed to deliver a wide range of RNA therapeutics to specific cell types. One important application of this system is the delivery of RNA therapeutics to cancer cells. As a proof-of-concept, we designed ENPs to deliver mRNAs encoding two cancer immunotherapeutics-the immunostimulatory cytokine IL 12 and an anti-PD-1 monoclonal antibody-based immune checkpoint inhibitor (ICI)-to CD19+ cells as a potential therapy for B cell malignancies. Intratumoral delivery of mRNAs encoding IL12 using lipid nanoparticles (LNPs) has been shown to suppress tumor growth and induce robust tumor infiltration of Natural Killer (NK) and CD8+ T cells. However, specific targeting of mRNA therapeutics to tumor cells presents a major roadblock to clinical development. IL12 is a heterodimer comprised of two subunits: p35 and p40. For initial experiments, the IL12p35 and IL12p40 subunits were delivered on separate mRNAs, which also assessed the platform's capability to deliver multiple mRNAs to target cells. ENPs were generated by co-transfecting VSV-Gₘᵤₜ+aCD19 scFv-CD4 CT-EPM+Lck-L7Ae-EABR+IL12p35-Box C/D+IL 12p40-Box C/D DNA in HEK293T cells. ENPs were purified by ultracentrifugation on a 20% sucrose cushion and added to HEK293T or HEK293T-CD19 cells. After 21 hours, supernatants were collected and IL 12 levels were quantified by ELISA. High levels of secreted IL 12 were detected in HEK293T-CD 19 target cell supernatants, which were 68-fold higher than IL 12 levels measured in HEK293T cell samples (FIG. 19). ENPs that only displayed one of the surface proteins, VSV-Gₘᵤₜ or aCD19 scFv-CD4 CT-EPM, induced only low IL 12 expression in both cell lines.

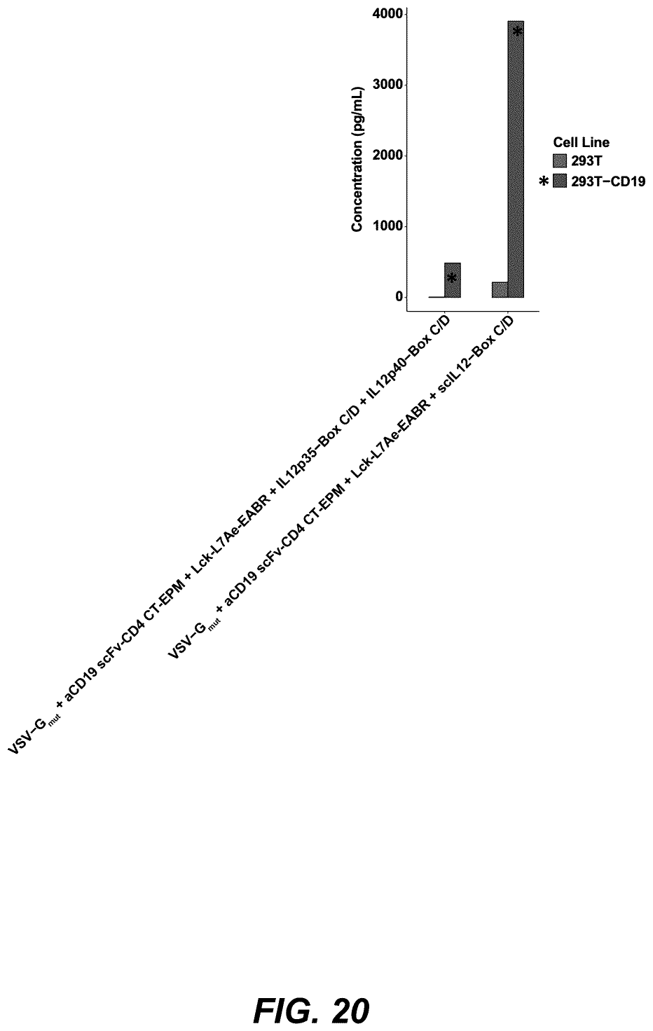

It was next evaluated whether mRNA-mediated IL 12 delivery can be enhanced by using a previously described single-chain IL 12 fusion protein (scIL12) in which the p35 and p40 subunits are linked by a short amino acid linker. Indeed, ENPs delivering a single mRNA encoding scIL 12-Box C/D induced ˜8-fold higher IL 12 levels than ENPs delivering IL12p35-Box C/D and IL 12p40-Box C/D encoded by separate mRNAs (FIG. 20).

Overall, this strategy could be particularly impactful for targeting solid tumors as the secretion of immunostimulatory cytokines can modulate the immunosuppressive nature of the tumor microenvironment and enhance the anti-tumor activity of endogenous and/or genetically engineered immune cells. Notably, genetically modified cells, such as CAR T-cells, can also be genetically engineered to generate the self-assembling ENPs to deliver mRNA-encoded immunotherapeutic agents to tumor cells, thereby potentially maximizing immunotherapeutic synergy and efficacy.

TABLE 6
Exemplary Sequences for Cargo Delivery

| NAME | SEQ ID NO: | SEQUENCE* |
|---|---|---|
| tdTomato | 170 (DNA), 172 | ATGGTGAGCAAGGGCGAGGAGGTCATCAAAGAGTTCATGC |
| | (RNA) | GCTTCAAGGTGCGCATGGAGGGCTCCATGAACGGCCACGA |
| | | GTTCGAGATCGAGGGCGAGGGCGAGGGCCGCCCCTACGAG |
| | | GGCACCCAGACCGCCAAGCTGAAGGTGACCAAGGGCGGCC |
| | | CCCTGCCCTTCGCCTGGGACATCCTGTCCCCCCAGTTCAT |
| | | GTACGGCTCCAAGGCGTACGTGAAGCACCCCGCCGACATC |
| | | CCCGATTACAAGAAGCTGTCCTTCCCCGAGGGCTTCAAGT |
| | | GGGAGCGCGTGATGAACTTCGAGGACGGCGGTCTGGTGAC |
| | | CGTGACCCAGGACTCCTCCCTGCAGGACGGCACGCTGATC |
| | | TACAAGGTGAAGATGCGCGGCACCAACTTCCCCCCCGACG |
| | | GCCCCGTAATGCAGAAGAAGACCATGGGCTGGGAGGCCTC |
| | | CACCGAGCGCCTGTACCCCCGCGACGGCGTGCTGAAGGGC |
| | | GAGATCCACCAGGCCCTGAAGCTGAAGGACGGCGGCCACT |
| | | ACCTGGTGGAGTTCAAGACCATCTACATGGCCAAGAAGCC |
| | | CGTGCAACTGCCCGGCTACTACTACGTGGACACCAAGCTG |
| | | GACATCACCTCCCACAACGAGGACTACACCATCGTGGAAC |
| | | AGTACGAGCGCTCCGAGGGCCGCCACCACCTGTTCCTGGG |
| | | GCATGGCACCGGCAGCACCGGCAGCGGCAGCTCCGGCACC |
| | | GCCTCCTCCGAGGACAACAACATGGCCGTCATCAAAGAGT |
| | | TCATGCGCTTCAAGGTGCGCATGGAGGGCTCCATGAACGG |
| | | CCACGAGTTCGAGATCGAGGGCGAGGGCGAGGGCCGCCCC |
| | | TACGAGGGCACCCAGACCGCCAAGCTGAAGGTGACCAAGG |
| | | GCGGCCCCCTGCCCTTCGCCTGGGACATCCTGTCCCCCCA |
| | | GTTCATGTACGGCTCCAAGGCGTACGTGAAGCACCCCGCC |
| | | GACATCCCCGATTACAAGAAGCTGTCCTTCCCCGAGGGCTT |
| | | CAAGTGGGAGCGCGTGATGAACTTCGAGGACGGCGGTCTG |
| | | GTGACCGTGACCCAGGACTCCTCCCTGCAGGACGGCACGC |
| | | TGATCTACAAGGTGAAGATGCGCGGCACCAACTTCCCCCC |
| | | CGACGGCCCCGTAATGCAGAAGAAGACCATGGGCTGGGA |
| | | GGCCTCCACCGAGCGCCTGTACCCCCGCGACGGCGTGCTG |
| | | AAGGGCGAGATCCACCAGGCCCTGAAGCTGAAGGACGGC |
| | | GGCCACTACCTGGTGGAGTTCAAGACCATCTACATGGCCA |
| | | AGAAGCCCGTGCAACTGCCCGGCTACTACTACGTGGACAC |
| | | CAAGCTGGACATCACCTCCCACAACGAGGACTACACCATC |
| | | GTGGAACAGTACGAGCGCTCCGAGGGCCGCCACCACCTGT |
| | | TCCTGTACGGCATGGACGAGCTGTACAAGTGAAAGCTTGG |
| | | C |
| tdTomato- | 171 (DNA), | ATGGTGAGCAAGGGCGAGGAGGTCATCAAAGAGTTCATGC |
| Box | 172 | GCTTCAAGGTGCGCATGGAGGGCTCCATGAACGGCCACGA |
| C/D (DNA | (RNA) | GTTCGAGATCGAGGGCGAGGGCGAGGGCCGCCCCTACGAG |
| sequence) | | GGCACCCAGACCGCCAAGCTGAAGGTGACCAAGGGCGGCC |
| | | CCCTGCCCTTCGCCTGGGACATCCTGTCCCCCCAGTTCAT |
| | | GTACGGCTCCAAGGCGTACGTGAAGCACCCCGCCGACATC |
| | | CCCGATTACAAGAAGCTGTCCTTCCCCGAGGGCTTCAAGT |
| | | GGGAGCGCGTGATGAACTTCGAGGACGGCGGTCTGGTGAC |
| | | CGTGACCCAGGACTCCTCCCTGCAGGACGGCACGCTGATC |
| | | TACAAGGTGAAGATGCGCGGCACCAACTTCCCCCCCGACG |
| | | GCCCCGTAATGCAGAAGAAGACCATGGGCTGGGAGGCCTC |
| | | CACCGAGCGCCTGTACCCCCGCGACGGCGTGCTGAAGGGC |
| | | GAGATCCACCAGGCCCTGAAGCTGAAGGACGGCGGCCACT |
| | | ACCTGGTGGAGTTCAAGACCATCTACATGGCCAAGAAGCC |
| | | CGTGCAACTGCCCGGCTACTACTACGTGGACACCAAGCTG |
| | | GACATCACCTCCCACAACGAGGACTACACCATCGTGGAAC |
| | | AGTACGAGCGCTCCGAGGGCCGCCACCACCTGTTCCTGGG |
| | | GCATGGCACCGGCAGCACCGGCAGCGGCAGCTCCGGCACC |
| | | GCCTCCTCCGAGGACAACAACATGGCCGTCATCAAAGAGT |
| | | TCATGCGCTTCAAGGTGCGCATGGAGGGCTCCATGAACGG |
| | | CCACGAGTTCGAGATCGAGGGCGAGGGCGAGGGCCGCCCC |
| | | TACGAGGGCACCCAGACCGCCAAGCTGAAGGTGACCAAG |
| | | GGCGGCCCCCTGCCCTTCGCCTGGGACATCCTGTCCCCCCA |
| | | GTTCATGTACGGCTCCAAGGCGTACGTGAAGCACCCCGCC |
| | | GACATCCCCGATTACAAGAAGCTGTCCTTCCCCGAGGGCTT |
| | | CAAGTGGGAGCGCGTGATGAACTTCGAGGACGGCGGTCTG |
| | | GTGACCGTGACCCAGGACTCCTCCCTGCAGGACGGCACGC |
| | | TGATCTACAAGGTGAAGATGCGCGGCACCAACTTCCCCCC |
| | | CGACGGCCCCGTAATGCAGAAGAAGACCATGGGCTGGGA |
| | | GGCCTCCACCGAGCGCCTGTACCCCCGCGACGGCGTGCTG |
| | | AAGGGCGAGATCCACCAGGCCCTGAAGCTGAAGGACGGC |
| | | GGCCACTACCTGGTGGAGTTCAAGACCATCTACATGGCCA |
| | | AGAAGCCCGTGCAACTGCCCGGCTACTACTACGTGGACAC |
| | | CAAGCTGGACATCACCTCCCACAACGAGGACTACACCATC |
| | | GTGGAACAGTACGAGCGCTCCGAGGGCCGCCACCACCTGT |
| | | TCCTGTACGGCATGGACGAGCTGTACAAGTGAAAGCTTGG |
| | | CacagatctggatcgggcgtgatCcgaaagGtgacccct |
| | | aggcttaagtata |
| zsGreen | 174 (DNA), 176 | ATGGCCACACATATGGCCCAGTCCAAGCACGGCCTGACCA |
| | (RNA) | AGGAGATGACCATGAAGTACCGCATGGAGGGCTGCGTGGA |
| | | CGGCCACAAGTTCGTGATCACCGGCGAGGGCATCGGCTAC |
| | | CCCTTCAAGGGCAAGCAGGCCATCAACCTGTGCGTGGTGG |
| | | AGGGCGGCCCCTTGCCCTTCGCCGAGGACATCTTGTCCGCC |
| | | GCCTTCATGTACGGCAACCGCGTGTTCACCGAGTACCCCCA |
| | | GGACATCGTCGACTACTTCAAGAACTCCTGCCCCGCCGGCT |
| | | ACACCTGGGACCGCTCCTTCCTGTTCGAGGACGGCGCCGT |
| | | GTGCATCTGCAACGCCGACATCACCGTGAGCGTGGAGGAG |
| | | AACTGCATGTACCACGAGTCCAAGTTCTACGGCGTGAACT |
| | | TCCCCGCCGACGGCCCCGTGATGAAGAAGATGACCGACAA |
| | | CTGGGAGCCCTCCTGCGAGAAGATCATCCCCGTGCCCAAG |
| | | CAGGGCATCTTGAAGGGCGACGTGAGCATGTACCTGCTGC |
| | | TGAAGGACGGTGGCCGCTTGCGCTGCCAGTTCGACACCGT |
| | | GTACAAGGCCAAGTCCGTGCCCCGCAAGATGCCCGACTGG |
| | | CACTTCATCCAGCACAAGCTGACCCGCGAGGACCGCAGCG |
| | | ACGCCAAGAACCAGAAGTGGCACCTGACCGAGCACGCCAT |
| | | CGCCTCCGGCTCCGCCTTGCCCTGAAAGCTTGGC |
| zsGreen-Box | 175 (DNA), 177 | ATGGCCACACATATGGCCCAGTCCAAGCACGGCCTGACCA |
| C/D | (RNA) | AGGAGATGACCATGAAGTACCGCATGGAGGGCTGCGTGGA |
| | | CGGCCACAAGTTCGTGATCACCGGCGAGGGCATCGGCTAC |
| | | CCCTTCAAGGGCAAGCAGGCCATCAACCTGTGCGTGGTGG |
| | | AGGGCGGCCCCTTGCCCTTCGCCGAGGACATCTTGTCCGCC |
| | | GCCTTCATGTACGGCAACCGCGTGTTCACCGAGTACCCCCA |
| | | GGACATCGTCGACTACTTCAAGAACTCCTGCCCCGCCGGCT |
| | | ACACCTGGGACCGCTCCTTCCTGTTCGAGGACGGCGCCGT |
| | | GTGCATCTGCAACGCCGACATCACCGTGAGCGTGGAGGAG |
| | | AACTGCATGTACCACGAGTCCAAGTTCTACGGCGTGAACT |
| | | TCCCCGCCGACGGCCCCGTGATGAAGAAGATGACCGACAA |
| | | CTGGGAGCCCTCCTGCGAGAAGATCATCCCCGTGCCCAAG |
| | | CAGGGCATCTTGAAGGGCGACGTGAGCATGTACCTGCTGC |
| | | TGAAGGACGGTGGCCGCTTGCGCTGCCAGTTCGACACCGT |
| | | GTACAAGGCCAAGTCCGTGCCCCGCAAGATGCCCGACTGG |
| | | CACTTCATCCAGCACAAGCTGACCCGCGAGGACCGCAGCG |
| | | ACGCCAAGAACCAGAAGTGGCACCTGACCGAGCACGCCAT |
| | | CGCCTCCGGCTCCGCCTTGCCCTGAAAGCTTGGCacagat |
| | | ctggatcgggcgtgatCcgaaagGtgacccctaggcttaa |
| | | gtata |
| aCD19 scFv | 178 | MALPVTALLLPLALLLHAARPEQKLISEEDLDIQMTQTTSSLS |
| | | ASLGDRVTISCRASQDISKYLNWYQQKPDGTVKLLIYHTSRL |
| | | HSGVPSRFSGSGSGTDYSLTISNLEQEDIATYFCQQGNTLPYTF |
| | | GGGTKLEITGGGGSGGGGSGGGGSEVKLQESGPGLVAPSQSL |
| | | SVTCTVSGVSLPDYGVSWIRQPPRKGLEWLGVIWGSETTYYN |
| | | SALKSRLTIIKDNSKSQVFLKMNSLQTDDTAIYYCAKHYYYG |
| | | GSYAMDYWGQGTSVTVSSTTTPAPRPPTPAPTIASQPLSLRPE |
| | | ACRPAAGGAVHTRGLDFACDIYIWAPLAGTCGVLLLSLVIT |
| aCD19 scFv- | 179 | MALPVTALLLPLALLLHAARPEQKLISEEDLDIQMTQTTSSLS |
| CD4 CT-EPM | | ASLGDRVTISCRASQDISKYLNWYQQKPDGTVKLLIYHTSRL |
| | | HSGVPSRFSGSGSGTDYSLTISNLEQEDIATYFCQQGNTLPYTF |
| | | GGGTKLEITGGGGSGGGGSGGGGSEVKLQESGPGLVAPSQSL |
| | | SVTCTVSGVSLPDYGVSWIRQPPRKGLEWLGVIWGSETTYYN |
| | | SALKSRLTIIKDNSKSQVFLKMNSLQTDDTAIYYCAKHYYYG |
| | | GSYAMDYWGQGTSVTVSSTTTPAPRPPTPAPTIASQPLSLRPE |
| | | ACRPAAGGAVHTRGLDFACDIYIWAPLAGTCGVLLLSLVITC |
| | | VRCRHRRRQAERMSQIKRLLSEKKTCQCPHRFQKTCSPIALPG |
| | | NPDHREMGETLPEEVGEYRQPSGGSVPVSPGPPSGLEPTSSSPY |
| aCD3 scFv | 180 | MALPVTALLLPLALLLHAARPEQKLISEEDLQVQLVESGGGL |
| | | VKPGESLRLSCAASGFTFSDYYMYWVRQAPGKCLEWVAIISD |
| | | GGYYTYYSDIIKGRFTISRDNAKNSLYLQMNSLKAEDTAVYY |
| | | CARGFPLLRHGAMDYWGQGTLVTVSSGGGGSGGGGSGGGG |
| | | SDIQMTQSPSSLSASVGDRVTITCKASQNVDTNVAWYQQKPG |
| | | QAPKSLIYSASYVYWDVPSRFSGSASGTDFTLTISSVQSEDFA |
| | | TYYCQQYDQQLITFGCGTKLEIKSGGGGSEVQLVESGGGLVQ |
| | | PGGSLKLSCAASGFTFNKYAMNWVRQAPGKGLEWVARIRSK |
| | | YNNYATYYADSVKDRFTISRDDSKNTAYLQMNNLKTEDTAV |
| | | YYCVRHGNFGNSYISYWAYWGQGTLVTVSSGGGGSGGGGS |
| | | GGGGSQTVVTQEPSLTVSPGGTVTLTCGSSTGAVTSGNYPNW |
| | | VQQKPGQAPRGLIGGTKFLAPGTPARFSGSLLGGKAALTLSG |
| | | VQPEDEAEYYCVLWYSNRWVFGGGTKLTVLTTTPAPRPPTP |
| | | APTIASQPLSLRPEACRPAAGGAVHTRGLDFACDIYIWAPLAG |
| | | TCGVLLLSLVIT |
| aCD3 scFv- | 181 | MALPVTALLLPLALLLHAARPEQKLISEEDLQVQLVESGGGL |
| CD4 CT-EPM | | VKPGESLRLSCAASGFTFSDYYMYWVRQAPGKCLEWVAIISD |
| | | GGYYTYYSDIIKGRFTISRDNAKNSLYLQMNSLKAEDTAVYY |
| | | CARGFPLLRHGAMDYWGQGTLVTVSSGGGGSGGGGSGGGG |
| | | SDIQMTQSPSSLSASVGDRVTITCKASQNVDTNVAWYQQKPG |
| | | QAPKSLIYSASYVYWDVPSRFSGSASGTDFTLTISSVQSEDFA |
| | | TYYCQQYDQQLITFGCGTKLEIKSGGGGSEVQLVESGGGLVQ |
| | | PGGSLKLSCAASGFTFNKYAMNWVRQAPGKGLEWVARIRSK |
| | | YNNYATYYADSVKDRFTISRDDSKNTAYLQMNNLKTEDTAV |
| | | YYCVRHGNFGNSYISYWAYWGQGTLVTVSSGGGGSGGGGS |
| | | GGGGSQTVVTQEPSLTVSPGGTVTLTCGSSTGAVTSGNYPNW |
| | | VQQKPGQAPRGLIGGTKFLAPGTPARFSGSLLGGKAALTLSG |
| | | VQPEDEAEYYCVLWYSNRWVFGGGTKLTVLTTTPAPRPPTP |
| | | APTIASQPLSLRPEACRPAAGGAVHTRGLDFACDIYIWAPLAG |
| | | TCGVLLLSLVITCVRCRHRRRQAERMSQIKRLLSEKKTCQCP |
| | | HRFQKTCSPIALPGNPDHREMGETLPEEVGEYRQPSGGSVPV |
| | | SPGPPSGLEPTSSSPY |
| aCD4 scFv | 182 | MALPVTALLLPLALLLHAARPEQKLISEEDLDIVMTQSPDSLA |
| | | VSLGERVTMNCKSSQSLLYSTNQKNYLAWYQQKPGQSPKLL |
| | | IYWASTRESGVPDRFSGSGSGTDFTLTISSVQAEDVAVYYCQ |
| | | QYYSYRTFGGGTKLEIKGGGGSGGGGSGGGGSQVQLQQSGP |
| | | EVVKPGASVKMSCKASGYTFTSYVIHWVRQKPGQGLDWIGY |
| | | INPYNDGTDYDEKFKGKATLTSDTSTSTAYMELSSLRSEDTA |
| | | VYYCAREKDNYATGAWFAYWGQGTLVTVSSTTTPAPRPPTP |
| | | APTIASQPLSLRPEACRPAAGGAVHTRGLDFACDIYIWAPLAG |
| | | TCGVLLLSLVIT |
| aCD4 scFv- | 183 | MALPVTALLLPLALLLHAARPEQKLISEEDLDIVMTQSPDSLA |
| CD4 CT-EPM | | VSLGERVTMNCKSSQSLLYSTNQKNYLAWYQQKPGQSPKLL |
| | | IYWASTRESGVPDRFSGSGSGTDFTLTISSVQAEDVAVYYCQ |
| | | QYYSYRTFGGGTKLEIKGGGGSGGGGSGGGGSQVQLQQSGP |
| | | EVVKPGASVKMSCKASGYTFTSYVIHWVRQKPGQGLDWIGY |
| | | INPYNDGTDYDEKFKGKATLTSDTSTSTAYMELSSLRSEDTA |
| | | VYYCAREKDNYATGAWFAYWGQGTLVTVSSTTTPAPRPPTP |
| | | APTIASQPLSLRPEACRPAAGGAVHTRGLDFACDIYIWAPLAG |
| | | TCGVLLLSLVITCVRCRHRRRQAERMSQIKRLLSEKKTCQCP |
| | | HRFQKTCSPIALPGNPDHREMGETLPEEVGEYRQPSGGSVPV |
| | | SPGPPSGLEPTSSSPY |
| IL12p35-Box | 184 (DNA), 187 | ATGTGCCCTGCCAGATCTCTGCTGCTGGTGGCTACACTGGT |
| C/D | (RNA) | GCTGCTGGATCATCTGAGCCTGGCCAGAAATCTGCCTGTG |
| | | GCCACTCCTGATCCTGGCATGTTCCCTTGTCTGCACCACAG |
| | | CCAGAACCTGCTGAGAGCCGTGTCCAACATGCTGCAGAAG |
| | | GCCAGACAGACCCTGGAATTCTACCCCTGCACCAGCGAGG |
| | | AAATCGACCACGAGGACATCACCAAGGATAAGACCAGCA |
| | | CCGTGGAAGCCTGCCTGCCTCTGGAACTGACCAAGAACGA |
| | | GAGCTGCCTGAACAGCCGGGAAACCAGCTTCATCACCAAC |
| | | GGCTCTTGCCTGGCCAGCAGAAAGACCTCCTTCATGATGG |
| | | CCCTGTGCCTGAGCAGCATCTACGAGGACCTGAAGATGTA |
| | | CCAGGTGGAATTCAAGACCATGAACGCCAAGCTGCTGATG |
| | | GACCCCAAGCGGCAGATCTTCCTGGACCAGAATATGCTGG |
| | | CCGTGATCGACGAGCTGATGCAGGCCCTGAACTTCAACAG |
| | | CGAGACAGTGCCCCAGAAGTCTAGCCTGGAAGAACCCGAC |
| | | TTCTACAAGACCAAGATCAAGCTGTGCATCCTGCTGCACG |
| | | CCTTCCGGATCAGAGCCGTGACCATCGACAGAGTGATGAG |
| | | CTACCTGAACGCCTCCTGAAAGCTTGGCacagatctggat |
| | | cgggcgtgatCcgaaagGtgacccctaggcttaagtata |
| IL12p40-Box | 185 (DNA), 188 | ATGTGTCACCAGCAGCTGGTCATCAGCTGGTTCAGCCTGGT |
| C/D | (RNA) | GTTCCTGGCCTCTCCTCTGGTGGCCATCTGGGAGCTGAAGA |
| | | AAGACGTGTACGTGGTGGAACTGGACTGGTATCCCGATGC |
| | | TCCTGGCGAGATGGTGGTGCTGACCTGCGATACCCCTGAA |
| | | GAGGACGGCATCACCTGGACACTGGATCAGTCTAGCGAGG |
| | | TGCTCGGCAGCGGCAAGACCCTGACCATCCAAGTGAAAGA |
| | | GTTTGGCGACGCCGGCCAGTACACCTGTCACAAAGGCGGA |
| | | GAAGTGCTGAGCCACAGCCTGCTGCTGCTCCACAAGAAAG |
| | | AGGATGGCATTTGGAGCACCGACATCCTGAAGGACCAGAA |
| | | AGAGCCCAAGAACAAGACCTTCCTGAGATGCGAGGCCAAG |
| | | AACTACAGCGGCCGGTTCACATGTTGGTGGCTGACCACCA |
| | | TCAGCACCGACCTGACCTTCAGCGTGAAGTCCAGCAGAGG |
| | | CAGCAGTGATCCTCAGGGCGTTACATGTGGCGCCGCTACA |
| | | CTGTCTGCCGAAAGAGTGCGGGGCGACAACAAAGAATACG |
| | | AGTACAGCGTGGAATGCCAAGAGGACAGCGCCTGTCCAGC |
| | | CGCCGAAGAGTCTCTGCCTATCGAAGTGATGGTGGACGCC |
| | | GTGCACAAGCTGAAGTACGAGAACTACACCTCCAGCTTTT |
| | | TCATCCGGGACATCATCAAGCCCGATCCTCCAAAGAACCT |
| | | GCAGCTGAAGCCTCTGAAGAACAGCAGACAGGTGGAAGT |
| | | GTCCTGGGAGTACCCCGACACCTGGTCTACACCCCACAGC |
| | | TACTTCAGCCTGACCTTTTGCGTGCAAGTGCAGGGCAAGTC |
| | | CAAGCGCGAGAAAAAGGACCGGGTGTTCACCGACAAGAC |
| | | CAGCGCCACCGTGATCTGCAGAAAGAACGCCAGCATCAGC |
| | | GTCAGAGCCCAGGACCGGTACTACAGCAGCTCTTGGAGCG |
| | | AATGGGCCAGCGTGCCATGTTCTTGAAAGCTTGGCacaga |
| | | tctggatcgggcgtgatCcgaaagGtgacccctaggctta |
| | | agtata |
| scIL12-Box | 186 (DNA), 189 | ATGTGTCACCAGCAGCTGGTCATCAGCTGGTTCAGCCTGGT |
| C/D | (RNA) | GTTCCTGGCCTCTCCTCTGGTGGCCATCTGGGAGCTGAAGA |
| | | AAGACGTGTACGTGGTGGAACTGGACTGGTATCCCGATGC |
| | | TCCTGGCGAGATGGTGGTGCTGACCTGCGATACCCCTGAA |
| | | GAGGACGGCATCACCTGGACACTGGATCAGTCTAGCGAGG |
| | | TGCTCGGCAGCGGCAAGACCCTGACCATCCAAGTGAAAGA |
| | | GTTTGGCGACGCCGGCCAGTACACCTGTCACAAAGGCGGA |
| | | GAAGTGCTGAGCCACAGCCTGCTGCTGCTCCACAAGAAAG |
| | | AGGATGGCATTTGGAGCACCGACATCCTGAAGGACCAGAA |
| | | AGAGCCCAAGAACAAGACCTTCCTGAGATGCGAGGCCAAG |
| | | AACTACAGCGGCCGGTTCACATGTTGGTGGCTGACCACCA |
| | | TCAGCACCGACCTGACCTTCAGCGTGAAGTCCAGCAGAGG |
| | | CAGCAGTGATCCTCAGGGCGTTACATGTGGCGCCGCTACA |
| | | CTGTCTGCCGAAAGAGTGCGGGGCGACAACAAAGAATACG |
| | | AGTACAGCGTGGAATGCCAAGAGGACAGCGCCTGTCCAGC |
| | | CGCCGAAGAGTCTCTGCCTATCGAAGTGATGGTGGACGCC |
| | | GTGCACAAGCTGAAGTACGAGAACTACACCTCCAGCTTTT |
| | | TCATCCGGGACATCATCAAGCCCGATCCTCCAAAGAACCT |
| | | GCAGCTGAAGCCTCTGAAGAACAGCAGACAGGTGGAAGT |
| | | GTCCTGGGAGTACCCCGACACCTGGTCTACACCCCACAGC |
| | | TACTTCAGCCTGACCTTTTGCGTGCAAGTGCAGGGCAAGTC |
| | | CAAGCGCGAGAAAAAGGACCGGGTGTTCACCGACAAGAC |
| | | CAGCGCCACCGTGATCTGCAGAAAGAACGCCAGCATCAGC |
| | | GTCAGAGCCCAGGACCGGTACTACAGCAGCTCTTGGAGCG |
| | | AATGGGCCAGCGTGCCATGTTCTGGTGGCGGAGGCGGAGG |
| | | CTCTAGAAATCTGCCTGTGGCCACTCCTGATCCTGGCATGT |
| | | TCCCTTGTCTGCACCACAGCCAGAACCTGCTGAGAGCCGT |
| | | GTCCAACATGCTGCAGAAGGCCAGACAGACCCTGGAATTC |
| | | TACCCCTGCACCAGCGAGGAAATCGACCACGAGGACATCA |
| | | CCAAGGATAAGACCAGCACCGTGGAAGCCTGCCTGCCTCT |
| | | GGAACTGACCAAGAACGAGAGCTGCCTGAACAGCCGGGA |
| | | AACCAGCTTCATCACCAACGGCTCTTGCCTGGCCAGCAGA |
| | | AAGACCTCCTTCATGATGGCCCTGTGCCTGAGCAGCATCTA |
| | | CGAGGACCTGAAGATGTACCAGGTGGAATTCAAGACCATG |
| | | AACGCCAAGCTGCTGATGGACCCCAAGCGGCAGATCTTCC |
| | | TGGACCAGAATATGCTGGCCGTGATCGACGAGCTGATGCA |
| | | GGCCCTGAACTTCAACAGCGAGACAGTGCCCCAGAAGTCT |
| | | AGCCTGGAAGAACCCGACTTCTACAAGACCAAGATCAAGC |
| | | TGTGCATCCTGCTGCACGCCTTCCGGATCAGAGCCGTGACC |
| | | ATCGACAGAGTGATGAGCTACCTGAACGCCTCCTGAAAGC |
| | | TTGGCacagatctggatcgggcgtgatCcgaaagGtgacc |
| | | cctaggcttaagtata |
| IL12p35 | 190 (DNA), 193 | ATGTGCCCTGCCAGATCTCTGCTGCTGGTGGCTACACTGGT |
| | (RNA) | GCTGCTGGATCATCTGAGCCTGGCCAGAAATCTGCCTGTG |
| | | GCCACTCCTGATCCTGGCATGTTCCCTTGTCTGCACCACAG |
| | | CCAGAACCTGCTGAGAGCCGTGTCCAACATGCTGCAGAAG |
| | | GCCAGACAGACCCTGGAATTCTACCCCTGCACCAGCGAGG |
| | | AAATCGACCACGAGGACATCACCAAGGATAAGACCAGCA |
| | | CCGTGGAAGCCTGCCTGCCTCTGGAACTGACCAAGAACGA |
| | | GAGCTGCCTGAACAGCCGGGAAACCAGCTTCATCACCAAC |
| | | GGCTCTTGCCTGGCCAGCAGAAAGACCTCCTTCATGATGG |
| | | CCCTGTGCCTGAGCAGCATCTACGAGGACCTGAAGATGTA |
| | | CCAGGTGGAATTCAAGACCATGAACGCCAAGCTGCTGATG |
| | | GACCCCAAGCGGCAGATCTTCCTGGACCAGAATATGCTGG |
| | | CCGTGATCGACGAGCTGATGCAGGCCCTGAACTTCAACAG |
| | | CGAGACAGTGCCCCAGAAGTCTAGCCTGGAAGAACCCGAC |
| | | TTCTACAAGACCAAGATCAAGCTGTGCATCCTGCTGCACG |
| | | CCTTCCGGATCAGAGCCGTGACCATCGACAGAGTGATGAG |
| | | CTACCTGAACGCCTCCTGAAAGCTTGGC |
| IL12p40 (DNA | 191 (DNA), 194 | ATGTGTCACCAGCAGCTGGTCATCAGCTGGTTCAGCCTGGT |
| sequence) | (RNA) | GTTCCTGGCCTCTCCTCTGGTGGCCATCTGGGAGCTGAAGA |
| | | AAGACGTGTACGTGGTGGAACTGGACTGGTATCCCGATGC |
| | | TCCTGGCGAGATGGTGGTGCTGACCTGCGATACCCCTGAA |
| | | GAGGACGGCATCACCTGGACACTGGATCAGTCTAGCGAGG |
| | | TGCTCGGCAGCGGCAAGACCCTGACCATCCAAGTGAAAGA |
| | | GTTTGGCGACGCCGGCCAGTACACCTGTCACAAAGGCGGA |
| | | GAAGTGCTGAGCCACAGCCTGCTGCTGCTCCACAAGAAAG |
| | | AGGATGGCATTTGGAGCACCGACATCCTGAAGGACCAGAA |
| | | AGAGCCCAAGAACAAGACCTTCCTGAGATGCGAGGCCAAG |
| | | AACTACAGCGGCCGGTTCACATGTTGGTGGCTGACCACCA |
| | | TCAGCACCGACCTGACCTTCAGCGTGAAGTCCAGCAGAGG |
| | | CAGCAGTGATCCTCAGGGCGTTACATGTGGCGCCGCTACA |
| | | CTGTCTGCCGAAAGAGTGCGGGGCGACAACAAAGAATACG |
| | | AGTACAGCGTGGAATGCCAAGAGGACAGCGCCTGTCCAGC |
| | | CGCCGAAGAGTCTCTGCCTATCGAAGTGATGGTGGACGCC |
| | | GTGCACAAGCTGAAGTACGAGAACTACACCTCCAGCTTTT |
| | | TCATCCGGGACATCATCAAGCCCGATCCTCCAAAGAACCT |
| | | GCAGCTGAAGCCTCTGAAGAACAGCAGACAGGTGGAAGT |
| | | GTCCTGGGAGTACCCCGACACCTGGTCTACACCCCACAGC |
| | | TACTTCAGCCTGACCTTTTGCGTGCAAGTGCAGGGCAAGTC |
| | | CAAGCGCGAGAAAAAGGACCGGGTGTTCACCGACAAGAC |
| | | CAGCGCCACCGTGATCTGCAGAAAGAACGCCAGCATCAGC |
| | | GTCAGAGCCCAGGACCGGTACTACAGCAGCTCTTGGAGCG |
| | | AATGGGCCAGCGTGCCATGTTCTTGAAAGCTTGGC |
| scIL12 (DNA | 192 (DNA), 195 | ATGTGTCACCAGCAGCTGGTCATCAGCTGGTTCAGCCTGGT |
| sequence) | (RNA) | GTTCCTGGCCTCTCCTCTGGTGGCCATCTGGGAGCTGAAGA |
| | | AAGACGTGTACGTGGTGGAACTGGACTGGTATCCCGATGC |
| | | TCCTGGCGAGATGGTGGTGCTGACCTGCGATACCCCTGAA |
| | | GAGGACGGCATCACCTGGACACTGGATCAGTCTAGCGAGG |
| | | TGCTCGGCAGCGGCAAGACCCTGACCATCCAAGTGAAAGA |
| | | GTTTGGCGACGCCGGCCAGTACACCTGTCACAAAGGCGGA |
| | | GAAGTGCTGAGCCACAGCCTGCTGCTGCTCCACAAGAAAG |
| | | AGGATGGCATTTGGAGCACCGACATCCTGAAGGACCAGAA |
| | | AGAGCCCAAGAACAAGACCTTCCTGAGATGCGAGGCCAAG |
| | | AACTACAGCGGCCGGTTCACATGTTGGTGGCTGACCACCA |
| | | TCAGCACCGACCTGACCTTCAGCGTGAAGTCCAGCAGAGG |
| | | CAGCAGTGATCCTCAGGGCGTTACATGTGGCGCCGCTACA |
| | | CTGTCTGCCGAAAGAGTGCGGGGCGACAACAAAGAATACG |
| | | AGTACAGCGTGGAATGCCAAGAGGACAGCGCCTGTCCAGC |
| | | CGCCGAAGAGTCTCTGCCTATCGAAGTGATGGTGGACGCC |
| | | GTGCACAAGCTGAAGTACGAGAACTACACCTCCAGCTTTT |
| | | TCATCCGGGACATCATCAAGCCCGATCCTCCAAAGAACCT |
| | | GCAGCTGAAGCCTCTGAAGAACAGCAGACAGGTGGAAGT |
| | | GTCCTGGGAGTACCCCGACACCTGGTCTACACCCCACAGC |
| | | TACTTCAGCCTGACCTTTTGCGTGCAAGTGCAGGGCAAGTC |
| | | CAAGCGCGAGAAAAAGGACCGGGTGTTCACCGACAAGAC |
| | | CAGCGCCACCGTGATCTGCAGAAAGAACGCCAGCATCAGC |
| | | GTCAGAGCCCAGGACCGGTACTACAGCAGCTCTTGGAGCG |
| | | AATGGGCCAGCGTGCCATGTTCTGGTGGCGGAGGCGGAGG |
| | | CTCTAGAAATCTGCCTGTGGCCACTCCTGATCCTGGCATGT |
| | | TCCCTTGTCTGCACCACAGCCAGAACCTGCTGAGAGCCGT |
| | | GTCCAACATGCTGCAGAAGGCCAGACAGACCCTGGAATTC |
| | | TACCCCTGCACCAGCGAGGAAATCGACCACGAGGACATCA |
| | | CCAAGGATAAGACCAGCACCGTGGAAGCCTGCCTGCCTCT |
| | | GGAACTGACCAAGAACGAGAGCTGCCTGAACAGCCGGGA |
| | | AACCAGCTTCATCACCAACGGCTCTTGCCTGGCCAGCAGA |
| | | AAGACCTCCTTCATGATGGCCCTGTGCCTGAGCAGCATCTA |
| | | CGAGGACCTGAAGATGTACCAGGTGGAATTCAAGACCATG |
| | | AACGCCAAGCTGCTGATGGACCCCAAGCGGCAGATCTTCC |
| | | TGGACCAGAATATGCTGGCCGTGATCGACGAGCTGATGCA |
| | | GGCCCTGAACTTCAACAGCGAGACAGTGCCCCAGAAGTCT |
| | | AGCCTGGAAGAACCCGACTTCTACAAGACCAAGATCAAGC |
| | | TGTGCATCCTGCTGCACGCCTTCCGGATCAGAGCCGTGACC |
| | | ATCGACAGAGTGATGAGCTACCTGAACGCCTCCTGAAAGC |
| | | TTGGC |

*EPM is indicated by underline italic, RBP by bold italic underline, ERD by italic, and PS by bold italic. For S-CD4 CT constructs, the underlined sequence in SARS-CoV-2 S was replaced with the underlined sequence in the CD4 CT sequences. For adapter fusion proteins, the adapter domain is underlined. For dimerization fusion proteins, the CSP heterologous cytoplasmic tail (e.g., CD4 CT) is underlined. Point mutations from wild type are shown in bold. The coding region of the Luc DNA sequence is shown in double underline. For cargo sequences, a DNA sequence is shown, and SEQ ID NOs for both RNA and DNA sequences are provided.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting. with the true scope and spirit being indicated by the following claims.

1.-17. (canceled)

18. A composition, comprising:
a nucleic acid composition comprising:
(i) a first polynucleotide encoding a dimerization fusion protein,
wherein the dimerization fusion protein comprises two or more of: a cell surface protein (CSP), a heterologous cytoplasmic tail, an RNA-binding protein (RBP), and an endosomal sorting complex required for transport (ESCRT)-recruiting domain (ERD);
(ii) a second polynucleotide encoding an adapter fusion protein comprising an adapter domain capable of binding the heterologous cytoplasmic tail to form a heterodimer, or encoding an adapter fusion protein comprising an adapter domain capable of binding the heterologous cytoplasmic tail to form a heterodimer and one or both of an RBP and an endosomal sorting complex required for transport (ESCRT)-recruiting domain (ERD); and
(iii) one or more third polynucleotides comprising one or more cargo RNA molecules each comprising a packing signal, wherein the RBP of (i) and (ii) are each capable of binding the packing signal,
wherein binding of the adapter domain to the heterologous cytoplasmic tail is capable of recruiting one or more ESCRT proteins to the heterodimer, thereby inducing a plurality of dimerization fusion proteins to self-assemble into an enveloped nanoparticle (ENP) secreted from a cell in which the dimerization fusion protein and adapter fusion protein are expressed, thereby generating a population of ENPs comprising the dimerization fusion protein and the one or more cargo RNA molecules.

19. The composition of claim 18, wherein the CSP is a targeting protein capable of targeting the ENP to a target cell, and wherein;
the nucleic acid composition further comprises a fourth polynucleotide encoding a soluble RBP capable of binding the packing signal;
the nucleic acid composition further comprises a fifth polynucleotide encoding a cell fusion protein, wherein the cell fusion protein is capable of inducing the fusion of a lipid envelope of the ENP and a lipid bilayer of the target cell; or
wherein the nucleic acid composition comprises the fourth polynucleotide and the fifth polynucleotide.

20-21. (canceled)

22. A composition, comprising:
a population of enveloped nanoparticles (ENPs), wherein each of the ENPs comprises:
(i) a plurality of dimerization fusion proteins each comprising a heterologous cytoplasmic tail and a CSP;
(ii) one or more cargo RNA molecules each comprising a packing signal; and
(iii) a plurality of cell fusion proteins.

23. (canceled)

24. The composition of claim 18,
wherein:
the packing signal comprises a Ku binding hairpin and the RBP is Ku;
the packing signal comprises a telomerase Sm7 binding motif and the RBP is Sm7;
the packing signal comprises an MS2 phage operator stem-loop and the RBP is MS2 Coat Protein (MCP);
the packing signal comprises a PP7 phage operator stem-loop and the RBP is PP7 Coat Protein (PCP);
the packing signal comprises an SfMu phage Com stem-loop and the RBP is Com RNA binding protein;
the packing signal comprises a PUF binding site (PBS) and the RBP is Pumilio/fem-3 mRNA binding factor (PUF);
the packing signal comprises Psi and the RBP is gag;
the packing signal comprises regulatory RNA CsrB and the RBP is CsrA of E. coli;
the packing signal comprises PS9 and the RBP is N protein of a coronavirus, optionally SARS-COV 2; and/or
the packing signal comprises Box C/D binding motif and the RBP is ribosomal protein L7Ae of archaea.

25. The composition of claim 18, wherein:
the packing signal comprises a nucleotide sequence having at least 65%, 70%, 75%, 80%, 85%, 90%, 95%, 98%, or 99% sequence identity to the nucleotide sequence of SEQ ID NO: 134 and the RBP comprises an amino acid sequence having at least 65%, 70%, 75%, 80%, 85%, 90%, 95%, 98%, or 99% sequence identity to the amino acid sequence of SEQ ID NO: 128;
the packing signal comprises a nucleotide sequence having at least 65%, 70%, 75%, 80%, 85%, 90%, 95%, 98%, or 99% sequence identity to the nucleotide sequence of SEQ ID NO: 152 and the RBP comprises an amino acid sequence having at least 65%, 70%, 75%, 80%, 85%, 90%, 95%, 98%, or 99% sequence identity to the amino acid sequence of SEQ ID NO: 148;
the packing signal comprises a nucleotide sequence having at least 65%, 70%, 75%, 80%, 85%, 90%, 95%, 98%, or 99% sequence identity to the nucleotide sequence of SEQ ID NO: 140 and the RBP comprises an amino acid sequence having at least 65%, 70%, 75%, 80%, 85%, 90%, 95%, 98%, or 99% sequence identity to the amino acid sequence of SEQ ID NO: 136; and/or
the packing signal comprises a nucleotide sequence having at least 65%, 70%, 75%, 80%, 85%, 90%, 95%, 98%, or 99% sequence identity to the nucleotide sequence of SEQ ID NO: 146 and the RBP comprises an amino acid sequence having at least 65%, 70%, 75%, 80%, 85%, 90%, 95%, 98%, or 99% sequence identity to the amino acid sequence of SEQ ID NO: 142.

26-27. (canceled)

28. The composition of claim 18, wherein the ERD comprises or is derived from:
a human protein, a nonhuman protein, or a nonmammalian protein;
a chicken protein, a mouse protein, a lizard protein, a reptile protein, a hamster protein, or a goldfish protein;
the ESCRT and ALIX binding region (EABR) of the human CEP55 protein;
Syntenin-1, rat Galectin-3 (rGalectin-3), Hrs, and/or CD2AP;
a viral protein or a fragment of a viral protein,
an Ebola protein.

29. The composition of claim 18, wherein the ERD:
comprises an amino acid sequence having at least 65%, 70%, 75%, 80%, 85%, 90%, 95%, 98%, or 99% sequence identity to the amino acid sequence of SEQ ID NO: 2 or wherein the ERD comprises the amino acid sequence of SEQ ID NO: 2; or
comprises an amino acid sequence having at least 65%, 70%, 75%, 80%, 85%, 90%, 95%, 98%, or 99% sequence identity to an amino acid sequence selected from the group consisting of SEQ ID NOs: 3-39.

30-33. (canceled)

34. The composition of claim 18, wherein the heterologous cytoplasmic tail comprises or is derived from a cytoplasmic tail (CT) of CD4 and/or wherein the adapter domain comprises or is derived from Lck tyrosine kinase.

35. (canceled)

36. The composition of claim 18, wherein the adapter fusion protein comprises an amino acid sequence having at least 65%, 70%, 75%, 80%, 85%, 90%, 95%, 98%, or 99% sequence identity to any one of the amino acid sequences selected from the group consisting of SEQ ID NOs: 157, 159, 162, and 164.

37. The composition of claim 18, wherein the heterologous cytoplasmic tail is derived from or comprises a cytoplasmic tail of a cell surface protein, and
wherein,
the adapter domain comprises or is derived from Lck tyrosine kinase, wherein the Lck tyrosine kinase comprises a myristolylation motif, and wherein myristoylation drives membrane anchoring of the adapter fusion protein to the plasma membrane.

38. (canceled)

39. The composition of claim 18, wherein the dimerization fusion protein comprises an endocytosis-preventing motif (EPM) capable of preventing endocytosis of the dimerization fusion protein and wherein the EPM comprises an amino acid sequence having at least 65%, 70%, 75%, 80%, 85%, 90%, 95%, 98%, or 99% sequence identity to the amino acid sequence of SEQ ID NO: 1 or the EPM comprises the amino acid sequence of SEQ ID NO: 1.

40-42. (canceled)

43. The composition of claim 18, wherein the dimerization fusion protein comprises a sequence selected from the sequences of SEQ ID NOs: 156, 158, 160-161, and 163.

44-48. (canceled)

49. The composition of claim 18, wherein the CSP is a targeting protein and comprises or is derived from an scFv and the scFv comprises a transmembrane domain or is fused to a heterologous transmembrane domain.

50. (canceled)

51. The composition of claim 18, wherein
the CSP is a targeting protein comprising an scFv and comprises the sequence of any one of SEQ ID NOs: 178, 180, and 182; or
wherein the CSP is a targeting protein and comprises or is derived from SARS-COV spike protein.

52. (canceled)

53. The composition of claim 19, wherein the cell fusion protein comprises or is derived from VSV-G.

54. (canceled)

55. The composition of claim 19, wherein:
(i) the dimerization fusion protein comprises the sequence of SEQ ID NO: 156, the adapter fusion protein comprises the sequence of SEQ ID NO: 157, the soluble RBP comprises the sequence of SEQ ID NO: 128, and the packing signal comprises the sequence of SEQ ID NO: 134;
(ii) the dimerization fusion protein comprises the sequence of SEQ ID NO: 161, the adapter fusion protein comprises the sequence of SEQ ID NO: 162, the soluble RBP comprises the sequence of SEQ ID NO: 136, and the packing signal comprising the sequence of SEQ ID NO: 140;
(iii) the dimerization fusion protein comprises the sequence of SEQ ID NO: 160, the adapter fusion protein comprises the sequence of SEQ ID NO: 162, the soluble RBP comprises the sequence of SEQ ID NO: 136, and the packing signal comprises the sequence of SEQ ID NO: 140;
(iv) the dimerization fusion protein comprises the sequence of SEQ ID NO: 160, the adapter fusion protein comprises the sequence of SEQ ID NO: 162, the soluble RBP comprises the sequence of SEQ ID NO: 136, the packing signal comprises the sequence of SEQ ID NO: 140, and the cell fusion protein comprises the sequence of SEQ ID NO: 169, or the dimerization fusion protein comprises the sequence of SEQ ID NO: 160, the adapter fusion protein comprises the sequence of SEQ ID NO: 162, the packing signal comprises the sequence of SEQ ID NO: 140, and the cell fusion protein comprises the sequence of SEQ ID NO: 169;
(v) the dimerization fusion protein comprises the sequence of SEQ ID NO: 160, the adapter fusion protein comprises the sequence of SEQ ID NO: 164, the soluble RBP comprises the sequence of SEQ ID NO: 142, the packing signal comprises the sequence of SEQ ID NO: 146, and the cell fusion protein comprises the sequence of SEQ ID NO: 169, or the dimerization fusion protein comprises the sequence of SEQ ID NO: 160, the adapter fusion protein comprises the sequence of SEQ ID NO: 164, the packing signal comprises the sequence of SEQ ID NO: 146, and the cell fusion protein comprises the sequence of SEQ ID NO: 169;
(vi) the dimerization fusion protein comprises the sequence of SEQ ID NO: 179, the adapter fusion protein comprises the sequence of SEQ ID NO: 164, the packing signal comprises the sequence of SEQ ID NO: 146, and the cell fusion protein comprises the sequence of SEQ ID NO: 169; and/or
(vii) the dimerization fusion protein comprises the sequence of SEQ ID NO: 181, the adapter fusion protein comprises the sequence of SEQ ID NO: 164, the packing signal comprises the sequence of SEQ ID NO: 146, and the cell fusion protein comprises the sequence of SEQ ID NO: 169.

56-57. (canceled)

58. The composition of claim 18, wherein the one or more cargo RNA molecules each comprise a microRNA (miRNA), a messenger RNA (mRNA), a long non-coding RNA (lncRNA), a ribosomal RNA (rRNA), a transfer RNA (tRNA), a small nuclear RNA (snRNA), a small nucleolar RNA (snoRNA), a Piwi-interacting RNA (piRNA), a interfering RNA (siRNA), an antisense RNA (aRNA), a transfer messenger RNA (tmRNA), a tRNA-derived small RNA (tsRNA), a rDNA-derived small RNA (srRNA), a ribozyme, a viral RNA, a single-stranded RNA, a double-stranded RNA, self-amplifying RNA, circular RNA, an aptamer, or any combination thereof,
wherein the mRNA encodes a payload protein and wherein the payload protein:
comprises fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof;
comprises nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof;
comprises a CRE recombinase, GCaMP, a cell therapy component, a knock-down gene therapy component, a cell-surface exposed epitope, or any combination thereof; and/or
comprises a diagnostic agent.

59-69. (canceled)

70. The composition of claim 18, wherein the one or more cargo RNA molecules each comprise an mRNA, wherein the mRNA encodes a payload protein and wherein the payload protein comprises an agonistic or antagonistic antibody or antigen-binding fragment thereof specific to:
a checkpoint inhibitor or checkpoint stimulator molecule;
a viral protein; or
an inflammatory cytokine.

71-93. (canceled)

94. A method of delivering one or more cargo RNA molecules to a cell or a population of cells, comprising contacting a cell or a population of cells with the composition of claim 18, thereby delivering the one or more cargo RNA molecules to the cell or the population of cells.

95-96. (canceled)

97. A method of treating or preventing a disease or disorder in a subject in need thereof, comprising:
administering to the subject a pharmaceutically effective amount of the composition of claim 18, thereby treating or preventing the disease or disorder in the subject.

98-99. (canceled)


SEQUENCE LISTING (195 sequences)

SEQ ID NO: 1 - protein, 48 aa, synthetic construct
ALPGNPDHREMGETLPEEVGEYRQPSGGSVPVSPGPPSGLEPTSSSPY

SEQ ID NO: 2 - protein, 58 aa, synthetic construct
FNSSINNIHEMEIQLKDALEKNQQWLVYDQQREVYVKGLLAKIFELEKKTETAAHSLP

SEQ ID NO: 3 - protein, 51 aa, synthetic construct
PIQQKSQHNKSVVQETPQTQNLYPDLSEIKKEYNVKEKDQVEDLNLDSLWE

SEQ ID NO: 4 - protein, 52 aa, synthetic construct
LQSRPEPTAPPEESFRSGVETTTPPQKQEPIDKELYPLTSLRSLFGNDPSSQ

SEQ ID NO: 5 - protein, 59 aa, synthetic construct
LQSRPEPTAPPESLRPEPTAPPPESFRFEEATPSPKQELKDREALTSLKSLFGNDHLLQ

SEQ ID NO: 6 - protein, 63 aa, synthetic construct
LQSRPEPTAPPESLRPEPTAPPPESFRFEEATPSPKQELKDREPYKEALTSLKSLFGNDHLLQ

SEQ ID NO: 7 - protein, 77 aa, synthetic construct
LQSRPEPTAPPESLRPEPTAPPPESFRFEEATPSPKQELKDREALTSLKSLFGNDHLLQGGSQTQNLYPDLSEIKKE

SEQ ID NO: 8 - protein, 92 aa, synthetic construct
LQSRPEPTAPPESLRPEPTAPPPESFRFEEATPSPKQELKDREALTSLKSLFGNDHLLQGGSQTQNLYPDLSEIKKEQTQNLYPDLSEIKKE

SEQ ID NO: 9 - protein, 81 aa, synthetic construct
LQSRPEPTAPPESLRPEPTAPPPESFRFEEATPSPKQELKDREPYKEALTSLKSLFGNDHLLQGGSQTQNLYPDLSEIKKE

SEQ ID NO: 10 - protein, 74 aa, synthetic construct
LQSRPEPTAPPESLRPEPTAPPESLRPEPTAPPPESFRFEEATPSPKQELKDREPYKEALTSLKSLFGNDHLLQ

SEQ ID NO: 11 - protein, 92 aa, synthetic construct
LQSRPEPTAPPESLRPEPTAPPESLRPEPTAPPPESFRFEEATPSPKQELKDREPYKEALTSLKSLFGNDHLLQGGSQTQNLYPDLSEIKKE

SEQ ID NO: 12 - protein, 73 aa, synthetic construct
LQSRPEPTAPPESLRPEPTAPPPESFRFEEATPSPKQELKDREPYKEALTDREPYKEALTSLKSLFGNDHLLQ

SEQ ID NO: 13 - protein, 65 aa, synthetic construct
LQSRPEPTAPPESLRPEPTAPPPESFRFEEATPSPKQELKDREPYKELYPLTSLKSLFGNDHLLQ

SEQ ID NO: 14 - protein, 67 aa, synthetic construct
LQSPPPYRPEPTAPPESLRPEPTAPPPESFRFEEATPSPKQELKDREPYKEALTSLKSLFGNDHLLQ

SEQ ID NO: 15 - protein, 64 aa, synthetic construct
LQSRPEPTAPPESLRPEPTAPPPYESFRFEEATPSPKQELKDREPYKEALTSLKSLFGNDHLLQ

SEQ ID NO: 16 - protein, 74 aa, synthetic construct
LQSRPEPTAPPESLRPEPTAPPPESFRFEEATPSPKQELKDREPYKELYPLTSLKSLYPLTSLKSLFGNDHLLQ

SEQ ID NO: 17 - protein, 69 aa, synthetic construct
LQSPPPYRPEPTAPPESLRPEPTAPPPESFRFEEATPSPKQELKDREPYKELYPLTSLKSLFGNDHLLQ

SEQ ID NO: 18 - protein, 65 aa, synthetic construct
LQSRPEPSAPPESLRPEPSAPPPESFRFEEATPSPKQELKDREPYKELYPLTSLKSLFGNDHLLQ

SEQ ID NO: 19 - protein, 69 aa, synthetic construct
LQSPPPYRPEPSAPPESLRPEPSAPPPESFRFEEATPSPKQELKDREPYKELYPLTSLKSLFGNDHLLQ

SEQ ID NO: 20 - protein, 76 aa, synthetic construct
PMAQVHQGLTPTAPPEDGLTPTAPPEDPAVDLLKNYMQLGKQQRESREKPYKELYPLTSLVTEDLLHLNSLFGGDQ

SEQ ID NO: 21 - protein, 80 aa, synthetic construct
PMAQVPPPYHQGLTPTAPPEDGLTPTAPPEDPAVDLLKNYMQLGKQQRESREKPYKELYPLTSLVTEDLLHLNSLFGGDQ

SEQ ID NO: 22 - protein, 76 aa, synthetic construct
PMAQVHQGLTPSAPPEDGLTPSAPPEDPAVDLLKNYMQLGKQQRESREKPYKELYPLTSLVTEDLLHLNSLFGGDQ

SEQ ID NO: 23 - protein, 80 aa, synthetic construct
PMAQVPPPYHQGLTPSAPPEDGLTPSAPPEDPAVDLLKNYMQLGKQQRESREKPYKELYPLTSLVTEDLLHLNSLFGGDQ

SEQ ID NO: 24 - protein, 65 aa, synthetic construct
LQSRPEPSAPPESLRPEPSAPPPESFRFEEATPSPKQELKDREPYKELYPLTGLKSLFGNDHLLQ

SEQ ID NO: 25 - protein, 54 aa, synthetic construct
LQSRPEPSAPPESLRPEPSAPPPESFRFEEATPSPKQELKDREPYKELYPLTSL

SEQ ID NO: 26 - protein, 54 aa, synthetic construct
LQSRPEPSAPPESLRPEPSAPPPESFRFEEATPSPKQELKDREPYKELYPLTGL

SEQ ID NO: 27 - protein, 65 aa, synthetic construct
LQSRPEPTAPPESLRPEPTAPPPESFRFEEATPSPKQELKDREPYKELYPLTGLKSLFGNDHLLQ

SEQ ID NO: 28 - protein, 76 aa, synthetic construct
LQSRPEPSAPPESLRPEPSAPPESLRPEPSAPPPESFRFEEATPSPKQELKDREPYKELYPLTGLKSLFGNDHLLQ

SEQ ID NO: 29 - protein, 83 aa, synthetic construct
LQSRPEPSAPPESLRPEPSAPPPESFRFEEATPSPKQELKDREPYKELYPLTGLKSLFGNDHLLQGGSQTQNLYPDLSEIKKE

SEQ ID NO: 30 - protein, 63 aa, synthetic construct
LQSRPEPSAPPESLRPEPSAPPPESFRFEEATPSPKQELKDREPYKELYPLTGLKSLFGNDHL

SEQ ID NO: 31 - protein, 69 aa, synthetic construct
LQSRPEPSAPPESLRPEPSAPPPESFRFEEATPSPKQELKDREPYKELYPLTGLKSLFGNDHLLQPPPY

SEQ ID NO: 32 - protein, 77 aa, synthetic construct
LQSRPEPSAPPESLRPEPSAPPPESFRFEEATPSPKQELKDREPYKELYPLTGLKSLFGNDHLLQPPPYGGGSPPPY

SEQ ID NO: 33 - protein, 69 aa, synthetic construct
LQSRPEPSAPPESLRPEPSAPPPESFRFEEATPSPKQELKDREPYKELYPLTGLKSLFGNDHLLQPPAY

SEQ ID NO: 34 - protein, 80 aa, synthetic construct
LQSRPEPSAPPESLRPEPSAPPESLRPEPSAPPPESFRFEEATPSPKQELKDREPYKELYPLTGLKSLFGNDHLLQPPPY

SEQ ID NO: 35 - protein, 83 aa, synthetic construct
LQSRPEPSAPPESLRPEPSAPPPESFRFEEATPSPKQELKDREPYKELYPLTGLKSLFGNDHLLQPPAYEPPAPAPLPPPSAP

SEQ ID NO: 36 - protein, 88 aa, synthetic construct
LQSRPEPSAPPESLRPEPSAPPPESFRFEEATPSPKQELKDREPYKELYPLTGLKSLFGNDHLLQPSAAPTAPPTGAADSIRPPPYSP

SEQ ID NO: 37 - protein, 89 aa, synthetic construct
LQSRPEPSAPPESLRPEPSAPPPESFRFEEATPSPKQELKDREPYKELYPLTGLKSLFGNDHLLQPSAPPPTAPPTGAADSIRPPPYSP

SEQ ID NO: 38 - protein, 120 aa, synthetic construct
MADGFSLNDALAGSGNPNPQGWPGAWGNQPGAGGYPGASYPGAYPGQAPPGGYPGQAPPSAYPGPTGPSAYPGPTAPGAYPGPTAPGAFPGQPGGPGAYPSAPGAYPSAPGAYPATGPFG

SEQ ID NO: 39 - protein, 131 aa, synthetic construct
MADGFSLNDALAGSGNPNPQGWPGAWGNQPGAGGYPGASYPGAYPGQAPPGGYPGQAPPSAYPGPTGPSAYPGPTAPGAYPGPTAPGAFPGQPGGPGAYPSAPGAYPSAPGAYPATGPFGPYKELYPLTSL

SEQ ID NO: 40 - protein, 1252 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGS

SEQ ID NO: 41 - protein, 1274 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCVRCRHRRRQAERMSQIKRLLSEKKTCQCPHRFQKTCSPI

SEQ ID NO: 42 - protein, 1322 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCVRCRHRRRQAERMSQIKRLLSEKKTCQCPHRFQKTCSPIALPGNPDHREMGETLPEEVGEYRQPSGGSVPVSPGPPSGLEPTSSSPY

SEQ ID NO: 43 - protein, 61 aa, synthetic construct
MGCGCSSHPEDDWMENIDVCENCHYPIVPLDGKGTLLIRNGSEVRDPLVTYEGSNPPASPL

SEQ ID NO: 44 - protein, 131 aa, synthetic construct
MGCGCSSHPEDDWMENIDVCENCHYPIVPLDGKGTLLIRNGSEVRDPLVTYEGSNPPASPLGSKSGSGSDSGSFNSSINNIHEMEIQLKDALEKNQQWLVYDQQREVYVKGLLAKIFELEKKTETAAHSLP

SEQ ID NO: 45 - protein, 1274 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCVRCRHQQRQAARMSQIKRLLSEKKTCQCPHRMQKSHNLI

SEQ ID NO: 46 - protein, 131 aa, synthetic construct
MGCVCSSNPEDDWMENIDVCENCHYPIVPLDSKISLPIRNGSEVRDPLVTYEGSLPPASPLGSKSGSGSDSGSFNSSINNIHEMEIQLKDALEKNQQWLVYDQQREVYVKGLLAKIFELEKKTETAAHSLP

SEQ ID NO: 47 - protein, 566 aa, synthetic construct
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCFPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 48 - protein, 594 aa, synthetic construct
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCFPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCVRCRHRRRQAERMSQIKRLLSEKKTCQCPHRFQKTCSPI

SEQ ID NO: 49 - protein, 594 aa, synthetic construct
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCFPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCVRCRHQQRQAARMSQIKRLLSEKKTCQCPHRMQKSHNLI

SEQ ID NO: 50 - protein, 1274 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCVRCRHRRRQAERMSQIKRAASEKKTCQCPHRFQKTCSPI

SEQ ID NO: 51 - protein, 1274 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCVRCRHRRRQAERMSQIKRALSEKKTCQCPHRFQKTCSPI

SEQ ID NO: 52 - protein, 1274 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCVRCRHRRRQAERMSQIKRLASEKKTCQCPHRFQKTCSPI

SEQ ID NO: 53 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCVRCRHQQRQAARMSQIKRAASEKKTCQCPHRMQKSHNL

SEQ ID NO: 54 - protein, 1274 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCVRCRHQQRQAARMSQIKRALSEKKTCQCPHRMQKSHNLI

SEQ ID NO: 55 - protein, 1274 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCVRCRHQQRQAARMSQIKRLASEKKTCQCPHRMQKSHNLI

SEQ ID NO: 56 - protein, 1266 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLLYCNHRNRRRVCKCPRPVVKSGDKPSLSARYV

SEQ ID NO: 57 - protein, 1274 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCVRCRHRRRQCERMSQIKRLLSEKKTCQCPHRFQKTCSPI

SEQ ID NO: 58 - protein, 1274 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCVRCRHRRRQAERMSQCKRLLSEKKTCQCPHRFQKTCSPI

SEQ ID NO: 59 - protein, 1274 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCVRCRHRRRQAERMSQIKRLLSEKKCCQCPHRFQKTCSPI

SEQ ID NO: 60 - protein, 131 aa, synthetic construct
MGCGCSSHPEDDWMCNIDVCENCHYPIVPLDGKGTLLIRNGSEVRDPLVTYEGSNPPASPLGSKSGSGSDSGSFNSSINNIHEMEIQLKDALEKNQQWLVYDQQREVYVKGLLAKIFELEKKTETAAHSLP

SEQ ID NO: 61 - protein, 131 aa, synthetic construct
MGCGCSSHPEDDWCENIDVCENCHYPIVPLDGKGTLLIRNGSEVRDPLVTYEGSNPPASPLGSKSGSGSDSGSFNSSINNIHEMEIQLKDALEKNQQWLVYDQQREVYVKGLLAKIFELEKKTETAAHSLP

SEQ ID NO: 62 - protein, 131 aa, synthetic construct
MGCGCSSHPEDDWMENIDVCENCHYCIVPLDGKGTLLIRNGSEVRDPLVTYEGSNPPASPLGSKSGSGSDSGSFNSSINNIHEMEIQLKDALEKNQQWLVYDQQREVYVKGLLAKIFELEKKTETAAHSLP

SEQ ID NO: 63 - protein, 1274 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCVRCRHRRRQCERMSQIKRALSEKKTCQCPHRFQKTCSPI

SEQ ID NO: 64 - protein, 138 aa, synthetic construct
MGCGCSSHPEDDWMENIDVCENCHYPIVPLDGKGTLLIRNGSEVRDPLVTYEGSNPPASPLGSKSGSGSDSGSLQSRPEPSAPPESLRPEPSAPPPESFRFEEATPSPKQELKDREPYKELYPLTGLKSLFGNDHLLQ

SEQ ID NO: 65 - protein, 204 aa, synthetic construct
MGCGCSSHPEDDWMENIDVCENCHYPIVPLDGKGTLLIRNGSEVRDPLVTYEGSNPPASPLGSKSGSGSDSGSMADGFSLNDALAGSGNPNPQGWPGAWGNQPGAGGYPGASYPGAYPGQAPPGGYPGQAPPSAYPGPTGPSAYPGPTAPGAYPGPTAPGAFPGQPGGPGAYPSAPGAYPSAPGAYPATGPFGPYKELYPLTSL

SEQ ID NO: 66 - protein, 378 aa, synthetic construct
MGEIKVSPDYNWFRGTVPLKKIIVDDDDSKIWSLYDAGPRSIRCPLIFLPPVSGTADVFFRQILALTGWGYRVIALQYPVYWDHLEFCDGFRKLLDHLQLDKVHLFGASLGGFLAQKFAEYTHKSPRVHSLILCNSFSDTSIFNQTWTANSFWLMPAFMLKKIVLGNFSSGPVDPMMADAIDFMVDRLESLGQSELASRLTLNCQNSYVEPHKIRDIPVTIMDVFDQSALSTEAKEEMYKLYPNARRAHLKTGGNFPYLCRSAEVNLYVQIHLLQFHGTKYAAIDPSMVSAEELEVQKGSLGISQEEQGSKSGSGSDSGSFNSSINNIHEMEIQLKDALEKNQQWLVYDQQREVYVKGLLAKIFELEKKTETAAHSLP

SEQ ID NO: 67 - protein, 385 aa, synthetic construct
MGEIKVSPDYNWFRGTVPLKKIIVDDDDSKIWSLYDAGPRSIRCPLIFLPPVSGTADVFFRQILALTGWGYRVIALQYPVYWDHLEFCDGFRKLLDHLQLDKVHLFGASLGGFLAQKFAEYTHKSPRVHSLILCNSFSDTSIFNQTWTANSFWLMPAFMLKKIVLGNFSSGPVDPMMADAIDFMVDRLESLGQSELASRLTLNCQNSYVEPHKIRDIPVTIMDVFDQSALSTEAKEEMYKLYPNARRAHLKTGGNFPYLCRSAEVNLYVQIHLLQFHGTKYAAIDPSMVSAEELEVQKGSLGISQEEQGSKSGSGSDSGSLQSRPEPSAPPESLRPEPSAPPPESFRFEEATPSPKQELKDREPYKELYPLTGLKSLFGNDHLLQ

SEQ ID NO: 68 - protein, 594 aa, synthetic construct
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCFPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCVRCRHRRRQAERMSQIKRALSEKKTCQCPHRFQKTCSPI

SEQ ID NO: 69 - protein, 590 aa, synthetic construct
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCFPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAICVRCRHRRRQAERMSQIKRALSEKKTCQCPHRFQKTCSPI

SEQ ID NO: 70 - protein, 183 aa, synthetic construct
MGCGCSSHPEDDWMENIDVCENCHYPIVPLDGKGTLLIRNGSEVRDPLVTYEGSNPPASPLGSKSGSGSDSGSALPGNPDHREMGETLPEEVGEYRQPSGGSVPVSPGPPSGLEPTSSSPYGGGSFNSSINNIHEMEIQLKDALEKNQQWLVYDQQREVYVKGLLAKIFELEKKTETAAHSLP

SEQ ID NO: 71 - protein, 190 aa, synthetic construct
MGCGCSSHPEDDWMENIDVCENCHYPIVPLDGKGTLLIRNGSEVRDPLVTYEGSNPPASPLGSKSGSGSDSGSALPGNPDHREMGETLPEEVGEYRQPSGGSVPVSPGPPSGLEPTSSSPYGGGSLQSRPEPSAPPESLRPEPSAPPPESFRFEEATPSPKQELKDREPYKELYPLTGLKSLFGNDHLLQ

SEQ ID NO: 72 - protein, 138 aa, synthetic construct
MGCGCSSHPEDDWMENIDVCENCHYPIVPLDGKGTLLIRNGSEVRDPLVTYEGSNPPASPLGSKSGSGSDSGSLQSRPEPTAPPESLRPEPTAPPPESFRFEEATPSPKQELKDREPYKELYPLTGLKSLFGNDHLLQ

SEQ ID NO: 73 - protein, 149 aa, synthetic construct
MGCGCSSHPEDDWMENIDVCENCHYPIVPLDGKGTLLIRNGSEVRDPLVTYEGSNPPASPLGSKSGSGSDSGSLQSRPEPSAPPESLRPEPSAPPESLRPEPSAPPPESFRFEEATPSPKQELKDREPYKELYPLTGLKSLFGNDHLLQ

SEQ ID NO: 74 - protein, 156 aa, synthetic construct
MGCGCSSHPEDDWMENIDVCENCHYPIVPLDGKGTLLIRNGSEVRDPLVTYEGSNPPASPLGSKSGSGSDSGSLQSRPEPSAPPESLRPEPSAPPPESFRFEEATPSPKQELKDREPYKELYPLTGLKSLFGNDHLLQGGSQTQNLYPDLSEIKKE

SEQ ID NO: 75 - protein, 136 aa, synthetic construct
MGCGCSSHPEDDWMENIDVCENCHYPIVPLDGKGTLLIRNGSEVRDPLVTYEGSNPPASPLGSKSGSGSDSGSLQSRPEPSAPPESLRPEPSAPPPESFRFEEATPSPKQELKDREPYKELYPLTGLKSLFGNDHL

SEQ ID NO: 76 - protein, 142 aa, synthetic construct
MGCGCSSHPEDDWMENIDVCENCHYPIVPLDGKGTLLIRNGSEVRDPLVTYEGSNPPASPLGSKSGSGSDSGSLQSRPEPSAPPESLRPEPSAPPPESFRFEEATPSPKQELKDREPYKELYPLTGLKSLFGNDHLLQPPPY

SEQ ID NO: 77 - protein, 150 aa, synthetic construct
MGCGCSSHPEDDWMENIDVCENCHYPIVPLDGKGTLLIRNGSEVRDPLVTYEGSNPPASPLGSKSGSGSDSGSLQSRPEPSAPPESLRPEPSAPPPESFRFEEATPSPKQELKDREPYKELYPLTGLKSLFGNDHLLQPPPYGGGSPPPY

SEQ ID NO: 78 - protein, 142 aa, synthetic construct
MGCGCSSHPEDDWMENIDVCENCHYPIVPLDGKGTLLIRNGSEVRDPLVTYEGSNPPASPLGSKSGSGSDSGSLQSRPEPSAPPESLRPEPSAPPPESFRFEEATPSPKQELKDREPYKELYPLTGLKSLFGNDHLLQPPAY

SEQ ID NO: 79 - protein, 153 aa, synthetic construct
MGCGCSSHPEDDWMENIDVCENCHYPIVPLDGKGTLLIRNGSEVRDPLVTYEGSNPPASPLGSKSGSGSDSGSLQSRPEPSAPPESLRPEPSAPPESLRPEPSAPPPESFRFEEATPSPKQELKDREPYKELYPLTGLKSLFGNDHLLQPPPY

SEQ ID NO: 80 - protein, 156 aa, synthetic construct
MGCGCSSHPEDDWMENIDVCENCHYPIVPLDGKGTLLIRNGSEVRDPLVTYEGSNPPASPLGSKSGSGSDSGSLQSRPEPSAPPESLRPEPSAPPPESFRFEEATPSPKQELKDREPYKELYPLTGLKSLFGNDHLLQPPAYEPPAPAPLPPPSAP

SEQ ID NO: 81 - protein, 161 aa, synthetic construct
MGCGCSSHPEDDWMENIDVCENCHYPIVPLDGKGTLLIRNGSEVRDPLVTYEGSNPPASPLGSKSGSGSDSGSLQSRPEPSAPPESLRPEPSAPPPESFRFEEATPSPKQELKDREPYKELYPLTGLKSLFGNDHLLQPSAAPTAPPTGAADSIRPPPYSP

SEQ ID NO: 82 - protein, 162 aa, synthetic construct
MGCGCSSHPEDDWMENIDVCENCHYPIVPLDGKGTLLIRNGSEVRDPLVTYEGSNPPASPLGSKSGSGSDSGSLQSRPEPSAPPESLRPEPSAPPPESFRFEEATPSPKQELKDREPYKELYPLTGLKSLFGNDHLLQPSAPPPTAPPTGAADSIRPPPYSP

SEQ ID NO: 83 - protein, 1265 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLQRRRKRARRMAQAKQYALEKKTCQCQRRMYK

SEQ ID NO: 84 - protein, 1270 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLRRKRQQVKRMALAKQHALAKRTCQCQRELTNDYYHT

SEQ ID NO: 85 - protein, 1276 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLPTCKRIKQKQQQAKKMAQIKQHALAKRTCQCQRDLPIDYYHT

SEQ ID NO: 86 - protein, 130 aa, synthetic construct
MGCCCSSDYDEDWIENIDICEHCNYPIDPDSKRQQLIRNVSEVRDPLVSYEAMSPPCSPLGSKSGSGSDSGSFNSSINNIHEMEIQLKDALEKNQQWLVYDQQREVYVKGLLAKIFELEKKTETAAHSLP

SEQ ID NO: 87 - protein, 130 aa, synthetic construct
MGNCCSLDYDDDWLEDIDICEVCHYPIDPSTKPQRLALNGSEAYNPLLSSEMDPPPSSPLGSKSGSGSDSGSFNSSINNIHEMEIQLKDALEKNQQWLVYDQQREVYVKGLLAKIFELEKKTETAAHSLP

SEQ ID NO: 88 - protein, 130 aa, synthetic construct
MGCCYSLDYDDDWAIDAEICEVCHYPIDPATKPQRPTLNVSELHTPLLMTEGIPPPSSPLGSKSGSGSDSGSFNSSINNIHEMEIQLKDALEKNQQWLVYDQQREVYVKGLLAKIFELEKKTETAAHSLP

SEQ ID NO: 89 - protein, 600 aa, synthetic construct
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCFPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICIGGGSAQLEKELQALEKENAQLEWELQALEKELAQ

SEQ ID NO: 90 - protein, 600 aa, synthetic construct
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCFPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICIGGGSAQLKKKLQALKKKNAQLKWKLQALKKKLAQ

SEQ ID NO: 91 - protein, 114 aa, synthetic construct
MGCVQCKDKEGGGSAQLEKELQALEKENAQLEWELQALEKELAQGSKSGSGSDSGSFNSSINNIHEMEIQLKDALEKNQQWLVYDQQREVYVKGLLAKIFELEKKTETAAHSLP

SEQ ID NO: 92 - protein, 114 aa, synthetic construct
MGCVQCKDKEGGGSAQLKKKLQALKKKNAQLKWKLQALKKKLAQGSKSGSGSDSGSFNSSINNIHEMEIQLKDALEKNQQWLVYDQQREVYVKGLLAKIFELEKKTETAAHSLP

SEQ ID NO: 93 - protein, 601 aa, synthetic construct
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCFPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICIGGGSYEIAALEAKIAALKAKNAALKAEIAALEAGC

SEQ ID NO: 94 - protein, 115 aa, synthetic construct
MGCVQCKDKEGGGSYKIAALKAEIAALEAENAALEAKIAALKAGCGSKSGSGSDSGSFNSSINNIHEMEIQLKDALEKNQQWLVYDQQREVYVKGLLAKIFELEKKTETAAHSLP

SEQ ID NO: 95 - protein, 40 aa, synthetic construct
CVRCRHRRRQAERMSQIKRLLSEKKTCQCPHRFQKTCSPI

SEQ ID NO: 96 - protein, 40 aa, synthetic construct
CVRCRHQQRQAARMSQIKRLLSEKKTCQCPHRMQKSHNLI

SEQ ID NO: 97 - protein, 61 aa, synthetic construct
MGCVCSSNPEDDWMENIDVCENCHYPIVPLDSKISLPIRNGSEVRDPLVTYEGSLPPASPL

SEQ ID NO: 98 - protein, 40 aa, synthetic construct
CVRCRHRRRQAERMSQIKRAASEKKTCQCPHRFQKTCSPI

SEQ ID NO: 99 - protein, 40 aa, synthetic construct
CVRCRHRRRQAERMSQIKRALSEKKTCQCPHRFQKTCSPI

SEQ ID NO: 100 - protein, 40 aa, synthetic construct
CVRCRHRRRQAERMSQIKRLASEKKTCQCPHRFQKTCSPI

SEQ ID NO: 101 - protein, 32 aa, synthetic construct
LYCNHRNRRRVCKCPRPVVKSGDKPSLSARYV

SEQ ID NO: 102 - protein, 40 aa, synthetic construct
CVRCRHRRRQCERMSQIKRLLSEKKTCQCPHRFQKTCSPI

SEQ ID NO: 103 - protein, 40 aa, synthetic construct
CVRCRHRRRQAERMSQCKRLLSEKKTCQCPHRFQKTCSPI

SEQ ID NO: 104 - protein, 40 aa, synthetic construct
CVRCRHRRRQAERMSQIKRLLSEKKCCQCPHRFQKTCSPI

SEQ ID NO: 105 - protein, 61 aa, synthetic construct
MGCGCSSHPEDDWMCNIDVCENCHYPIVPLDGKGTLLIRNGSEVRDPLVTYEGSNPPASPL

SEQ ID NO: 106 - protein, 61 aa, synthetic construct
MGCGCSSHPEDDWCENIDVCENCHYPIVPLDGKGTLLIRNGSEVRDPLVTYEGSNPPASPL

SEQ ID NO: 107 - protein, 61 aa, synthetic construct
MGCGCSSHPEDDWMENIDVCENCHYCIVPLDGKGTLLIRNGSEVRDPLVTYEGSNPPASPL

SEQ ID NO: 108 - protein, 40 aa, synthetic construct
CVRCRHRRRQCERMSQIKRALSEKKTCQCPHRFQKTCSPI

SEQ ID NO: 109 - protein, 308 aa, synthetic construct
MGEIKVSPDYNWFRGTVPLKKIIVDDDDSKIWSLYDAGPRSIRCPLIFLPPVSGTADVFFRQILALTGWGYRVIALQYPVYWDHLEFCDGFRKLLDHLQLDKVHLFGASLGGFLAQKFAEYTHKSPRVHSLILCNSFSDTSIFNQTWTANSFWLMPAFMLKKIVLGNFSSGPVDPMMADAIDFMVDRLESLGQSELASRLTLNCQNSYVEPHKIRDIPVTIMDVFDQSALSTEAKEEMYKLYPNARRAHLKTGGNFPYLCRSAEVNLYVQIHLLQFHGTKYAAIDPSMVSAEELEVQKGSLGISQEEQ

SEQ ID NO: 110 - protein, 31 aa, synthetic construct
QRRRKRARRMAQAKQYALEKKTCQCQRRMYK

SEQ ID NO: 111 - protein, 36 aa, synthetic construct
RRKRQQVKRMALAKQHALAKRTCQCQRELTNDYYHT

SEQ ID NO: 112 - protein, 42 aa, synthetic construct
PTCKRIKQKQQQAKKMAQIKQHALAKRTCQCQRDLPIDYYHT

SEQ ID NO: 113 - protein, 60 aa, synthetic construct
MGCCCSSDYDEDWIENIDICEHCNYPIDPDSKRQQLIRNVSEVRDPLVSYEAMSPPCSPL

SEQ ID NO: 114 - protein, 60 aa, synthetic construct
MGNCCSLDYDDDWLEDIDICEVCHYPIDPSTKPQRLALNGSEAYNPLLSSEMDPPPSSPL

SEQ ID NO: 115 - protein, 60 aa, synthetic construct
MGCCYSLDYDDDWAIDAEICEVCHYPIDPATKPQRPTLNVSELHTPLLMTEGIPPPSSPL

SEQ ID NO: 116 - protein, 30 aa, synthetic construct
AQLEKELQALEKENAQLEWELQALEKELAQ

SEQ ID NO: 117 - protein, 30 aa, synthetic construct
AQLKKKLQALKKKNAQLKWKLQALKKKLAQ

SEQ ID NO: 118 - protein, 10 aa, synthetic construct
MGCVQCKDKE

SEQ ID NO: 119 - protein, 44 aa, synthetic construct
MGCVQCKDKEGGGSAQLEKELQALEKENAQLEWELQALEKELAQ

SEQ ID NO: 120 - protein, 44 aa, synthetic construct
MGCVQCKDKEGGGSAQLKKKLQALKKKNAQLKWKLQALKKKLAQ

SEQ ID NO: 121 - protein, 31 aa, synthetic construct
YEIAALEAKIAALKAKNAALKAEIAALEAGC

SEQ ID NO: 122 - protein, 31 aa, synthetic construct
YKIAALKAEIAALEAENAALEAKIAALKAGC

SEQ ID NO: 123 - protein, 45 aa, synthetic construct
MGCVQCKDKEGGGSYKIAALKAEIAALEAENAALEAKIAALKAGC

SEQ ID NO: 124 - protein, 180 aa, synthetic construct
MASTSYDYCRVPMEDGDKRCKLLLGIGILVLLIIVILGVPLIIFTIKANSEACRDGLRAVMECRNVTHLLQQELTEAQKGFQDVEAQAATCNHTVMALMASLDAEKAQGQKKVEELEGEITTLNHKLQDASAEVERLRRENQVLSVRIADKKYYPSSQDSSSAAAPQLLIVLLGLSALLQ

SEQ ID NO: 125 - protein, 81 aa, synthetic construct
MQPIQIAIAALVVAIIIAIVVWSIVIIEYRKILRQRKIDRLIDRLIERAEDSGNESEGEISALVEMGVEMGHHAPWDIDDL

SEQ ID NO: 126 - protein, 256 aa, synthetic construct
MASKDVEEGVEGPICWICREEVGNEGIHPCACTGELDVVHPQCLSTWLTVSRNTACQMCRVIYRTRTQWRSRLNLWPEMERQEIFELFLLMSVVVAGLVGVALCTWTLLVILTAPAGTFSPGAVLGFLCFFGFYQIFIVFAFGGICRVSGTVRALYAANNTRVTVLPYRRPRRPTANEDNIELTVLVGPAGGTDEEPTDESSEGDVASGDKERDGSSGDEPDGGPNDRAGLRGTARTDLCAPTKKPVRKNHPKNNG

SEQ ID NO: 127 - protein, 121 aa, synthetic construct
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 128 - protein, 61 aa, synthetic construct
MLILTRRVGETLMIGDEVTVTVLGVKGNQVRIGVNAPKEVSVHREEIYQRIQAEKSQQSSY

SEQ ID NO: 129 - protein, 183 aa, synthetic construct
ALPGNPDHREMGETLPEEVGEYRQPSGGSVPVSPGPPSGLEPTSSSPYGSKSGSGSDSGSMLILTRRVGETLMIGDEVTVTVLGVKGNQVRIGVNAPKEVSVHREEIYQRIQAEKSQQSSYGGGSFNSSINNIHEMEIQLKDALEKNQQWLVYDQQREVYVKGLLAKIFELEKKTETAAHSLP

SEQ ID NO: 130 - DNA, 1650 bp, synthetic construct
  source 1..1650: mol_type = other DNA
atggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtg

SEQ ID NO: 131 - DNA, 369 bp, synthetic construct
  source 1..369: mol_type = other DNA
gtcgacagggagtcagacaacgaagtgaacatcaggatgatgacacttctgcaggacacaccaggatggtgtttcagggaaaggcttctggatgaagcgaagaggatgacgcaggacgcgttaaaggacacctccaggatggagaatgagaaccggtcaggatgattcggtgggtcaggaaggccagggacacttcaggatgaagtatcacatcggggtggtgtgagcaggaagcaatagttcaggatgaacgattggccgcaaggccagaggaaaagttgtcaaggatgagcagggagcaacaaaagtagctggaatgctgcgaaacgaaccgggagcgctgtgaatacagtgctccctttttttatt

SEQ ID NO: 132 - DNA, 2031 bp, synthetic construct
  source 1..2031: mol_type = other DNA
atggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgaaagcttggcgtcgacagggagtcagacaacgaagtgaacatcaggatgatgacacttctgcaggacacaccaggatggtgtttcagggaaaggcttctggatgaagcgaagaggatgacgcaggacgcgttaaaggacacctccaggatggagaatgagaaccggtcaggatgattcggtgggtcaggaaggccagggacacttcaggatgaagtatcacatcggggtggtgtgagcaggaagcaatagttcaggatgaacgattggccgcaaggccagaggaaaagttgtcaaggatgagcagggagcaacaaaagtagctggaatgctgcgaaacgaaccgggagcgctgtgaatacagtgctccctttttttatt

SEQ ID NO: 133 - RNA, 1650 nt, synthetic construct
  source 1..1650: mol_type = other RNA
atggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtg

SEQ ID NO: 134 - RNA, 369 nt, synthetic construct
  source 1..369: mol_type = other RNA
gtcgacagggagtcagacaacgaagtgaacatcaggatgatgacacttctgcaggacacaccaggatggtgtttcagggaaaggcttctggatgaagcgaagaggatgacgcaggacgcgttaaaggacacctccaggatggagaatgagaaccggtcaggatgattcggtgggtcaggaaggccagggacacttcaggatgaagtatcacatcggggtggtgtgagcaggaagcaatagttcaggatgaacgattggccgcaaggccagaggaaaagttgtcaaggatgagcagggagcaacaaaagtagctggaatgctgcgaaacgaaccgggagcgctgtgaatacagtgctccctttttttatt

SEQ ID NO: 135 - RNA, 2031 nt, synthetic construct
  source 1..2031: mol_type = other RNA
atggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgaaagcttggcgtcgacagggagtcagacaacgaagtgaacatcaggatgatgacacttctgcaggacacaccaggatggtgtttcagggaaaggcttctggatgaagcgaagaggatgacgcaggacgcgttaaaggacacctccaggatggagaatgagaaccggtcaggatgattcggtgggtcaggaaggccagggacacttcaggatgaagtatcacatcggggtggtgtgagcaggaagcaatagttcaggatgaacgattggccgcaaggccagaggaaaagttgtcaaggatgagcagggagcaacaaaagtagctggaatgctgcgaaacgaaccgggagcgctgtgaatacagtgctccctttttttatt

SEQ ID NO: 136 - protein, 419 aa, synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 137 - protein, 541 aa, synthetic construct
ALPGNPDHREMGETLPEEVGEYRQPSGGSVPVSPGPPSGLEPTSSSPYGSKSGSGSDSGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGGGSFNSSINNIHEMEIQLKDALEKNQQWLVYDQQREVYVKGLLAKIFELEKKTETAAHSLP

SEQ ID NO: 138 - DNA, 1092 bp, synthetic construct
  source 1..1092: mol_type = other DNA
tctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagata

SEQ ID NO: 139 - DNA, 2754 bp, synthetic construct
  source 1..2754: mol_type = other DNA
atggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgaaagcttggctctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagata

SEQ ID NO: 140 - RNA, 1092 nt, synthetic construct
  source 1..1092: mol_type = other RNA
tctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagata

SEQ ID NO: 141 - RNA, 2754 nt, synthetic construct
  source 1..2754: mol_type = other RNA
atggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgaaagcttggctctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagata

SEQ ID NO: 142 - protein, 119 aa, synthetic construct
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 143 - protein, 241 aa, synthetic construct
ALPGNPDHREMGETLPEEVGEYRQPSGGSVPVSPGPPSGLEPTSSSPYGSKSGSGSDSGSMYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKGGGSFNSSINNIHEMEIQLKDALEKNQQWLVYDQQREVYVKGLLAKIFELEKKTETAAHSLP

SEQ ID NO: 144 - DNA, 51 bp, synthetic construct
  source 1..51: mol_type = other DNA
acagatctggatcgggcgtgatccgaaaggtgacccctaggcttaagtata

SEQ ID NO: 145 - DNA, 1713 bp, synthetic construct
  source 1..1713: mol_type = other DNA
atggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgaaagcttggcacagatctggatcgggcgtgatccgaaaggtgacccctaggcttaagtata

SEQ ID NO: 146 - RNA, 51 nt, synthetic construct
  source 1..51: mol_type = other RNA
acagatctggatcgggcgtgatccgaaaggtgacccctaggcttaagtata

SEQ ID NO: 147 - RNA, 1713 nt, synthetic construct
  source 1..1713: mol_type = other RNA
atggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgaaagcttggcacagatctggatcgggcgtgatccgaaaggtgacccctaggcttaagtata

SEQ ID NO: 148 - protein, 130 aa, synthetic construct
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 149 - protein, 252 aa, synthetic construct
ALPGNPDHREMGETLPEEVGEYRQPSGGSVPVSPGPPSGLEPTSSSPYGSKSGSGSDSGSMASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIYGGGSFNSSINNIHEMEIQLKDALEKNQQWLVYDQQREVYVKGLLAKIFELEKKTETAAHSLP

SEQ ID NO: 150 - DNA, 657 bp, synthetic construct
  source 1..657: mol_type = other DNA
taaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaa

SEQ ID NO: 151 - DNA, 2345 bp, synthetic construct
  source 1..2345: mol_type = other DNA
atggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgagatatcagctacggaactcttgtgcgtaaggatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaa

SEQ ID NO: 152 - RNA, 657 nt, synthetic construct
  source 1..657: mol_type = other RNA
taaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaa

SEQ ID NO: 153 - RNA, 2345 nt, synthetic construct
  source 1..2345: mol_type = other RNA
atggaagatgccaagaacatcaagaagggccctgctccattctaccctctggaagatggaacagccggcgagcagctgcacaaggccatgaagagatatgccctggtgcctggcacaatcgccttcacagatgcccacatcgaggtggacatcacctacgccgagtacttcgagatgtctgtgcggctggccgaagctatgaagcgctacggcctgaacaccaaccaccggatcgtcgtgtgcagcgagaacagcctgcagttcttcatgcctgtgctgggcgccctgttcatcggagttgctgtggcccctgccaacgacatctacaacgagcgcgagctgctgaacagcatgggcatctctcagcccaccgtggtgttcgtgtccaagaagggactgcagaaaatcctgaacgtgcagaagaagctgcccatcatccagaaaatcatcatcatggacagcaagaccgactaccagggcttccagagcatgtacaccttcgtgaccagccatctgccacctggcttcaacgagtacgacttcgtgcccgagagcttcgaccgggacaagacaatcgccctgatcatgaacagcagcggctctaccggactgcccaaaggtgttgccctgcctcatagaactgcctgcgtgcggtttagccacgccagagatcccatcttcggcaaccagatcatccccgacacagccatcctgagcgtggtgccttttcaccacggcttcggcatgttcaccacactgggctacctgatctgcggcttcagagtggtgctgatgtaccgcttcgaggaagaactgttcctgcggagcctgcaggactacaagatccagtctgctctgctggtgcctactctgttcagcttctttgccaagagcaccctgatcgataagtacgacctgagcaacctgcacgagatcgcctctggcggagcccctctgtctaaagaagtgggagaagccgtcgccaagcggttccatctgcctggcatcagacaaggctatggcctgaccgagacaaccagcgccattctgattacccctgagggcgacgataagcctggcgctgtgggaaaagtggtgccattcttcgaggccaaggtggtggatctggacaccggcaaaacactgggcgttaaccagaggggcgagctgtgtgttagaggccctatgatcatgagcggctacgtgaacaaccccgaggccacaaacgctctgatcgacaaggatggatggctgcacagcggcgacattgcctactgggacgaagatgagcacttcttcatcgtggaccggctgaagtccctgatcaagtacaagggctaccaggtggccccagccgagctggaatctattctgctgcaacaccccaacatcttcgatgccggcgttgcaggactgcccgatgatgatgctggcgaactgccagctgctgtggtggtgctggaacacggcaagaccatgaccgagaaagaaatcgtggactacgtggccagccaagtgaccaccgccaagaaactgagaggcggcgtggtgtttgtggacgaggtgccaaaaggcctgacaggcaagctggacgcccggaagatcagagagatcctgattaaggccaagaaaggcggcaagatcgccgtgtgagatatcagctacggaactcttgtgcgtaaggatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaaggtacctaattgcctagaaaacatgaggatcacccatgtctgcaggtcgactctagaaaacatgaggatcacccatgtctgcagtattcccgggttcattagatcctaa

SEQ ID NO: 154 - protein, 121 aa, synthetic construct
ALPGNPDHREMGETLPEEVGEYRQPSGGSVPVSPGPPSGLEPTSSSPYGSKSGSGSDSGSMLILTRRVGETLMIGDEVTVTVLGVKGNQVRIGVNAPKEVSVHREEIYQRIQAEKSQQSSY

SEQ ID NO: 155 - protein, 123 aa, synthetic construct
MLILTRRVGETLMIGDEVTVTVLGVKGNQVRIGVNAPKEVSVHREEIYQRIQAEKSQQSSYGGGSFNSSINNIHEMEIQLKDALEKNQQWLVYDQQREVYVKGLLAKIFELEKKTETAAHSLP

SEQ ID NO: 156 - protein, 161 aa, synthetic construct
CVRCRHRRRQAERMSQIKRLLSEKKTCQCPHRFQKTCSPIALPGNPDHREMGETLPEEVGEYRQPSGGSVPVSPGPPSGLEPTSSSPYGSKSGSGSDSGSMLILTRRVGETLMIGDEVTVTVLGVKGNQVRIGVNAPKEVSVHREEIYQRIQAEKSQQSSY

SEQ ID NO: 157 - protein, 196 aa, synthetic construct
MGCGCSSHPEDDWMENIDVCENCHYPIVPLDGKGTLLIRNGSEVRDPLVTYEGSNPPASPLGSKSGSGSDSGSMLILTRRVGETLMIGDEVTVTVLGVKGNQVRIGVNAPKEVSVHREEIYQRIQAEKSQQSSYGGGSFNSSINNIHEMEIQLKDALEKNQQWLVYDQQREVYVKGLLAKIFELEKKTETAAHSLP

SEQ ID NO: 158 - protein, 223 aa, synthetic construct
CVRCRHRRRQAERMSQIKRLLSEKKTCQCPHRFQKTCSPIALPGNPDHREMGETLPEEVGEYRQPSGGSVPVSPGPPSGLEPTSSSPYGSKSGSGSDSGSMLILTRRVGETLMIGDEVTVTVLGVKGNQVRIGVNAPKEVSVHREEIYQRIQAEKSQQSSYGGGSFNSSINNIHEMEIQLKDALEKNQQWLVYDQQREVYVKGLLAKIFELEKKTETAAHSLP

SEQ ID NO: 159 - protein, 134 aa, synthetic construct
MGCGCSSHPEDDWMENIDVCENCHYPIVPLDGKGTLLIRNGSEVRDPLVTYEGSNPPASPLGSKSGSGSDSGSMLILTRRVGETLMIGDEVTVTVLGVKGNQVRIGVNAPKEVSVHREEIYQRIQAEKSQQSSY

SEQ ID NO: 160 - protein, 88 aa, synthetic construct
CVRCRHRRRQAERMSQIKRLLSEKKTCQCPHRFQKTCSPIALPGNPDHREMGETLPEEVGEYRQPSGGSVPVSPGPPSGLEPTSSSPY

SEQ ID NO: 161 - protein, 519 aa, synthetic construct
CVRCRHRRRQAERMSQIKRLLSEKKTCQCPHRFQKTCSPIALPGNPDHREMGETLPEEVGEYRQPSGGSVPVSPGPPSGLEPTSSSPYGSKSGSGSDSGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 162 - protein, 554 aa, synthetic construct
MGCGCSSHPEDDWMENIDVCENCHYPIVPLDGKGTLLIRNGSEVRDPLVTYEGSNPPASPLGSKSGSGSDSGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGGGSFNSSINNIHEMEIQLKDALEKNQQWLVYDQQREVYVKGLLAKIFELEKKTETAAHSLP

SEQ ID NO: 163 - protein, 219 aa, synthetic construct
CVRCRHRRRQAERMSQIKRLLSEKKTCQCPHRFQKTCSPIALPGNPDHREMGETLPEEVGEYRQPSGGSVPVSPGPPSGLEPTSSSPYGSKSGSGSDSGSMYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 164 - protein, 254 aa, synthetic construct
MGCGCSSHPEDDWMENIDVCENCHYPIVPLDGKGTLLIRNGSEVRDPLVTYEGSNPPASPLGSKSGSGSDSGSMYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQKGGGSFNSSINNIHEMEIQLKDALEKNQQWLVYDQQREVYVKGLLAKIFELEKKTETAAHSLP

SEQ ID NO: 165 - DNA, 145 bp, synthetic construct
  source 1..145: mol_type = other DNA
tgcaacaccccaacatcttcgatgccggcgttgcaggactgcccgatgatgatgctggcgaactgccagctgctgtggtggtgctggaacacggcaagaccatgaccgagaaagaaatcgtggactacgtggctgaaagcttggc

SEQ ID NO: 166 - DNA, 1237 bp, synthetic construct
  source 1..1237: mol_type = other DNA
tgcaacaccccaacatcttcgatgccggcgttgcaggactgcccgatgatgatgctggcgaactgccagctgctgtggtggtgctggaacacggcaagaccatgaccgagaaagaaatcgtggactacgtggctgaaagcttggctctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagata

SEQ ID NO: 167 - RNA, 145 nt, synthetic construct
  source 1..145: mol_type = other RNA
tgcaacaccccaacatcttcgatgccggcgttgcaggactgcccgatgatgatgctggcgaactgccagctgctgtggtggtgctggaacacggcaagaccatgaccgagaaagaaatcgtggactacgtggctgaaagcttggc

SEQ ID NO: 168 - RNA, 1237 nt, synthetic construct
  source 1..1237: mol_type = other RNA
tgcaacaccccaacatcttcgatgccggcgttgcaggactgcccgatgatgatgctggcgaactgccagctgctgtggtggtgctggaacacggcaagaccatgaccgagaaagaaatcgtggactacgtggctgaaagcttggctctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagata

SEQ ID NO: 169 - protein, 511 aa, synthetic construct
MKCLLYLAFLFIGVNCKFTIVFPHNQKGNWKNVPSNYHYCPSSSDLNWHNDLIGTALQVKMPQSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTEAELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 170 - DNA, 1440 bp, synthetic construct
  source 1..1440: mol_type = other DNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtgaaagcttggc

SEQ ID NO: 171 - DNA, 1491 bp, synthetic construct
  source 1..1491: mol_type = other DNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtgaaagcttggcacagatctggatcgggcgtgatccgaaaggtgacccctaggcttaagtata

SEQ ID NO: 172 - RNA, 1440 nt, synthetic construct
  source 1..1440: mol_type = other RNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtgaaagcttggc

SEQ ID NO: 173 - RNA, 1491 nt, synthetic construct
  source 1..1491: mol_type = other RNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctggggcatggcaccggcagcaccggcagcggcagctccggcaccgcctcctccgaggacaacaacatggccgtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaagtgaaagcttggcacagatctggatcgggcgtgatccgaaaggtgacccctaggcttaagtata

SEQ ID NO: 174 - DNA, 717 bp, synthetic construct
  source 1..717: mol_type = other DNA
atggccacacatatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgaaagcttggc

SEQ ID NO: 175 - DNA, 768 bp, synthetic construct
  source 1..768: mol_type = other DNA
atggccacacatatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgaaagcttggcacagatctggatcgggcgtgatccgaaaggtgacccctaggcttaagtata

SEQ ID NO: 176 - RNA, 717 nt, synthetic construct
  source 1..717: mol_type = other RNA
atggccacacatatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgaaagcttggc

SEQ ID NO: 177 - RNA, 768 nt, synthetic construct
  source 1..768: mol_type = other RNA
atggccacacatatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctgaaagcttggcacagatctggatcgggcgtgatccgaaaggtgacccctaggcttaagtata

SEQ ID NO: 178 - protein, 339 aa, synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVIT

SEQ ID NO: 179 - protein, 427 aa, synthetic construct
MALPVTALLLPLALLLHAARPEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITCVRCRHRRRQAERMSQIKRLLSEKKTCQCPHRFQKTCSPIALPGNPDHREMGETLPEEVGEYRQPSGGSVPVSPGPPSGLEPTSSSPY

SEQ ID NO: 180 - protein, 595 aa, synthetic construct
MALPVTALLLPLALLLHAARPEQKLISEEDLQVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLTTTPAPRPPTPAPTIASQPLSLRPEACRPAAGGAVHTRGLDFACDIYIWAPLAGTCGVLLLSLVIT

SEQ ID NO: 181 - protein, 683 aa, synthetic construct
MALPVTALLLPLALLLHAARPEQKLISEEDLQVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLTTTPAPRPPTPAPTIASQPLSLRPEACRPAAGGAVHTRGLDFACDIYIWAPLAGTCGVLLLSLVITCVRCRHRRRQAERMSQIKRLLSEKKTCQCPHRFQKTCSPIALPGNPDHREMGETLPEEVGEYRQPSGGSVPVSPGPPSGLEPTSSSPY

SEQ ID NO: 182 - protein, 346 aa, synthetic construct
MALPVTALLLPLALLLHAARPEQKLISEEDLDIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKGGGGSGGGGSGGGGSQVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSTTTPAPRPPTPAPTIASQPLSLRPEACRPAAGGAVHTRGLDFACDIYIWAPLAGTCGVLLLSLVIT

SEQ ID NO: 183 - protein, 434 aa, synthetic construct
MALPVTALLLPLALLLHAARPEQKLISEEDLDIVMTQSPDSLAVSLGERVTMNCKSSQSLLYSTNQKNYLAWYQQKPGQSPKLLIYWASTRESGVPDRFSGSGSGTDFTLTISSVQAEDVAVYYCQQYYSYRTFGGGTKLEIKGGGGSGGGGSGGGGSQVQLQQSGPEVVKPGASVKMSCKASGYTFTSYVIHWVRQKPGQGLDWIGYINPYNDGTDYDEKFKGKATLTSDTSTSTAYMELSSLRSEDTAVYYCAREKDNYATGAWFAYWGQGTLVTVSSTTTPAPRPPTPAPTIASQPLSLRPEACRPAAGGAVHTRGLDFACDIYIWAPLAGTCGVLLLSLVITCVRCRHRRRQAERMSQIKRLLSEKKTCQCPHRFQKTCSPIALPGNPDHREMGETLPEEVGEYRQPSGGSVPVSPGPPSGLEPTSSSPY

SEQ ID NO: 184 - DNA, 720 bp, synthetic construct
  source 1..720: mol_type = other DNA
atgtgccctgccagatctctgctgctggtggctacactggtgctgctggatcatctgagcctggccagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaaagcttggcacagatctggatcgggcgtgatccgaaaggtgacccctaggcttaagtata

SEQ ID NO: 185 - DNA, 1047 bp, synthetic construct
  source 1..1047: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttcttgaaagcttggcacagatctggatcgggcgtgatccgaaaggtgacccctaggcttaagtata

SEQ ID NO: 186 - DNA, 1659 bp, synthetic construct
  source 1..1659: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggcggaggctctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaaagcttggcacagatctggatcgggcgtgatccgaaaggtgacccctaggcttaagtata

SEQ ID NO: 187 - RNA, 720 nt, synthetic construct
  source 1..720: mol_type = other RNA
atgtgccctgccagatctctgctgctggtggctacactggtgctgctggatcatctgagcctggccagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaaagcttggcacagatctggatcgggcgtgatccgaaaggtgacccctaggcttaagtata

SEQ ID NO: 188 - RNA, 1047 nt, synthetic construct
  source 1..1047: mol_type = other RNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttcttgaaagcttggcacagatctggatcgggcgtgatccgaaaggtgacccctaggcttaagtata

SEQ ID NO: 189 - RNA, 1659 nt, synthetic construct
  source 1..1659: mol_type = other RNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggcggaggctctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaaagcttggcacagatctggatcgggcgtgatccgaaaggtgacccctaggcttaagtata

SEQ ID NO: 190 - DNA, 669 bp, synthetic construct
  source 1..669: mol_type = other DNA
atgtgccctgccagatctctgctgctggtggctacactggtgctgctggatcatctgagcctggccagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaaagcttggc

SEQ ID NO: 191 - DNA, 996 bp, synthetic construct
  source 1..996: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttcttgaaagcttggc

SEQ ID NO: 192 - DNA, 1608 bp, synthetic construct
  source 1..1608: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggcggaggctctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaaagcttggc

SEQ ID NO: 193 - RNA, 669 nt, synthetic construct
  source 1..669: mol_type = other RNA
atgtgccctgccagatctctgctgctggtggctacactggtgctgctggatcatctgagcctggccagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaaagcttggc

SEQ ID NO: 194 - RNA, 996 nt, synthetic construct
  source 1..996: mol_type = other RNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttcttgaaagcttggc

SEQ ID NO: 195 - RNA, 1608 nt, synthetic construct
  source 1..1608: mol_type = other RNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggtggcggaggcggaggctctagaaatctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctcctgaaagcttggc